(19) United States
(12) Patent Application Publication
(10) Pub. No.: US 2024/0158404 A1
(43) Pub. Date: May 16, 2024

(54) MODULATORS OF ALPHA-1 ANTITRYPSIN

(71) Applicant: Vertex Pharmaceuticals Incorporated, Boston, MA (US)

(72) Inventors: Ronald Lee Grey, JR., Mansfield, MA (US); Emily Elizabeth Allen, Boston, MA (US); Michael J. Boyd, Sharon, MA (US); Robert F. Fimognari, JR., Brookline, MA (US); Simon Giroux, Cambridge, MA (US); Michelle Lai-Chen, Waltham, MA (US); Ales Medek, Winchester, MA (US); Mengqi Li, Chelsea, MA (US); Christopher David Poff, North Andover, MA (US); Daniel Tyler Richter, Boston, MA (US); Tony Z. Scott, Cambridge, MA (US); Kathleen Paige Sokolowsky, Boston, MA (US); Jeffrey Braden Sperry, Hopkinton, MA (US); Charlene Tsay, Boston, MA (US); Xiaoxu Wang, Allston, MA (US); Mariam Zaky, Boston, MA (US); Chenlong Zhang, Waltham, MA (US)

(21) Appl. No.: 18/463,796

(22) Filed: Sep. 8, 2023

Related U.S. Application Data
(60) Provisional application No. 63/489,543, filed on Mar. 10, 2023.
(60) Provisional application No. 63/405,080, filed on Sep. 9, 2022.

(51) Int. Cl. C07D 487/04

(52) U.S. Cl. CPC C07D 487/04; C07B 2200/05; C07B 2200/13

(57) ABSTRACT

Novel compounds, compositions, and methods of using and preparing the same, which may be useful for treating alpha-1 antitrypsin deficiency (AATD).

This application claims the benefit of priority of U.S. Provisional Application No. 63/405,080, filed Sep. 9, 2022, and U.S. Provisional Application No. 63/489,543, filed Mar. 10, 2023, the contents of which are incorporated by reference herein in their entirety.

The disclosure provides compounds that are capable of modulating alpha-1 antitrypsin (AAT) activity and methods of treating alpha-1 antitrypsin deficiency (AATD) by administering one or more such compounds.

AATD is a genetic disorder characterized by low circulating levels of AAT. While treatments for AATD exist, there is currently no cure. AAT is produced primarily in liver cells and secreted into the blood, but it is also made by other cell types including lung epithelial cells and certain white blood cells. AAT inhibits several serine proteases secreted by inflammatory cells (most notably neutrophil elastase (NE) proteinase 3, and cathepsin G) and thus protects organs such as the lung from protease-induced damage, especially during periods of inflammation.

The mutation most commonly associated with AATD involves a substitution of lysine for glutamic acid (E342K) in the SERPINA1 gene that encodes the AAT protein. This mutation, known as the Z mutation or the Z allele, leads to misfolding of the translated protein, which is therefore not secreted into the bloodstream and can polymerize within the producing cell. Consequently, circulating AAT levels in individuals homozygous for the Z allele (PiZZ) are markedly reduced; only approximately 15% of mutant Z-AAT protein folds correctly and is secreted by the cell. An additional consequence of the Z mutation is that the secreted Z-AAT has reduced activity compared to wild-type protein, with 40% to 80% of normal antiprotease activity (American Thoracic Society/European Respiratory Society, Am J Respir Crit Care Med. 2003; 168(7):818-900; and Ogushi et al. J Clin Invest. 1987; 80(5):1366-74).

The accumulation of polymerized Z-AAT protein within hepatocytes results in a gain-of-function cytotoxicity that can result in cirrhosis or liver cancer later in life and neonatal liver disease in 12% of patients. This accumulation may spontaneously remit but can be fatal in a small number of children. The deficiency of circulating AAT results in unregulated protease activity that degrades lung tissue over time, resulting in emphysema, a form of chronic obstructive pulmonary disease (COPD). This effect is severe in PiZZ individuals and typically manifests in middle age, resulting in a decline in quality of life and shortened lifespan (mean 68 years of age) (Tanash et al. Int J Chron Obstruct Pulm Dis. 2016; 11:1663-9). The effect is more pronounced in PiZZ individuals who smoke, resulting in an even further shortened lifespan (58 years). (Piitulainen and Tanash, COPD 2015; 12(1):36-41). PiZZ individuals account for the majority of those with clinically relevant AATD lung disease. Accordingly, there is a need for additional and effective treatments for AATD.

A milder form of AATD is associated with the SZ genotype in which the Z-allele is combined with an S-allele. The S allele is associated with somewhat reduced levels of circulating AAT but causes no cytotoxicity in liver cells. The result is clinically significant lung disease but not liver disease. (Fregonese and Stolk, Orphanet J Rare Dis. 2008; 33:16). As with the ZZ genotype, the deficiency of circulating AAT in subjects with the SZ genotype results in unregulated protease activity that degrades lung tissue over time and can result in emphysema, particularly in smokers.

The current standard of care for AAT-deficient individuals who have or show signs of developing significant lung or liver disease is augmentation therapy or protein replacement therapy. Augmentation therapy involves administration of a human AAT protein concentrate purified from pooled donor plasma to augment the missing AAT. Although infusions of the plasma protein have been shown to improve survival or slow the rate of emphysema progression, augmentation therapy is often not sufficient under challenging conditions such as during an active lung infection. Similarly, although protein replacement therapy shows promise in delaying progression of disease, augmentation does not restore the normal physiological regulation of AAT in patients and efficacy has been difficult to demonstrate. In addition, augmentation therapy requires weekly visits for treatment and augmentation therapy cannot address liver disease, which is driven by the toxic gain-of-function of the Z allele. Thus, there is a continuing need for new and more effective treatments for AATD.

A key consideration for the selection of compounds suitable for clinical development is the projection of human dose. Two key parameters that factor into the projection of human dose are the unbound clearance of a compound and the plasma efficacious exposure (also referred to herein as potency). Together, these parameters are known to one skilled in the art as measurements of Compound Quality (as defined herein) and suitability for advancement into clinical development. Thus, in evaluating AAT modulator compounds, it is necessary to consider both potency and unbound clearance parameters. The relationship between these two parameters can be very difficult to predict.

The compounds of the invention exhibit an unanticipated improvement in the compound potency and unbound clearance relative to the closest prior art, WO 2020/247160. The compounds of the invention are characterized by both high potency and low unbound clearance. Thus, the compounds of the invention exhibit improved Compound Quality scores relative to the prior art. Compounds with high projected human doses (e.g., a compound with a high unbound clearance) may limit the ability to reach efficacious exposures in clinical trials. In contrast, a higher-quality compound may result in a greater potential to test the full range of the predicted efficacious exposure of a compound. Exploration of higher efficacious exposures may lead to greater clinical benefit for a compound.

A separate but related consideration for the selection of AAT-modulator compounds involves exposure multiples, which are an assessment of relative compound exposure in toxicology studies relative to the predicted plasma efficacious exposure. Higher exposure multiples in preclinical toxicology studies can provide the opportunity to explore higher exposures in clinical development. The larger the exposure multiple, the more suitable the compound may be for clinical development. However, exposures that result in an adverse toxicological outcome is unpredictable.

Prior art publication, WO 2020/247160, leaves the relationship between compound potency and unbound clearance, as well as exposure multiples, for compounds of the disclosed scaffold challenging to predict. Experimentation on compounds of WO 2020/247160 shows no identifiable structure activity relationship (SAR) that can be used to predict which compounds might possess the appropriate efficacious exposure/unbound clearance relationship to make them better candidates for clinical development. Nor does WO 2020/247160 suggest any means for improving exposure multiples in disclosed compounds. In fact, SAR from WO 2020/247160 did not support exploration of 8F analogs of the WO 2020/247160 compounds for any reason.

One aspect of the invention provides compounds that unexpectedly exhibit a significantly superior Compound Quality compared to compounds disclosed in WO 2020/247160. The compounds of the invention also exhibit unexpectedly enhanced exposure multiples due to the substitution of fluorine for hydrogen at C8 of the core ring structure. Thus, one aspect of the disclosure provides compounds of Formula I:

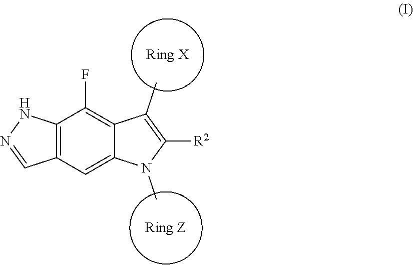

as well as tautomers of those compounds, deuterated derivatives of those compounds and tautomers, and pharmaceutically acceptable salts of those compounds, tautomers, and deuterated derivatives that can be employed in the treatment of AATD, wherein:
Ring X is selected from:

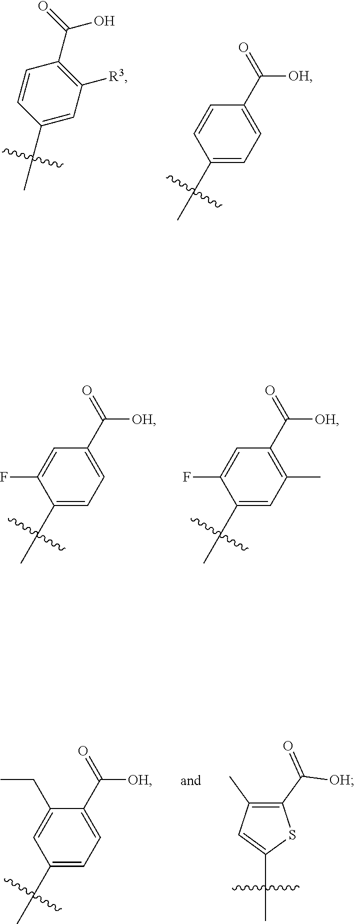

Ring Z is selected from:

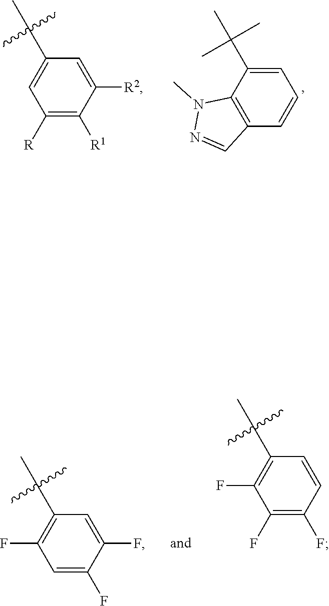

Each R is independently selected from: F, H, Cl, —CH₃, —OCH₃, and —OCD₃;
R¹ is H or F;
R² is selected from:

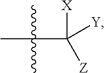

wherein
   one of X, Y, and Z is —OH;
   one of X, Y, and Z is chosen from —CH₂CH₃ and cyclopropyl;
   and one of X, Y, and Z is chosen from —CH₂OCH₃, —CH₂CH₂OCH₃, —CH₂OCH₂CH₃, —CH₂O-cyclopropyl, and —CH₂O-isopropyl;

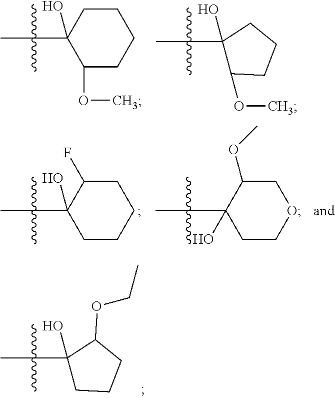

and
R³ is chosen from H and —CH₃.

Another aspect of the disclosure provides compounds of Formula Ia:

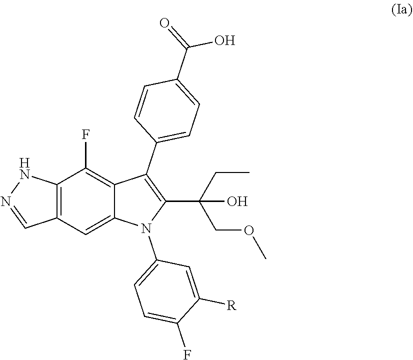

as well as tautomers of those compounds, deuterated derivatives of those compounds and tautomers, and pharmaceutically acceptable salts of those compounds, tautomers, and deuterated derivatives that can be employed in the treatment of AATD, wherein:
R is selected from F, H, Cl, CH₃, and OCH₃.

The compounds of Formula Ia have unexpectedly and significantly lower anticipated human dose projection (as measured by the Compound Quality score) compared to prior art compounds sharing this same scaffold. In one embodiment, the compounds of Formula Ia are selected from:

| | |
|---|---|
| 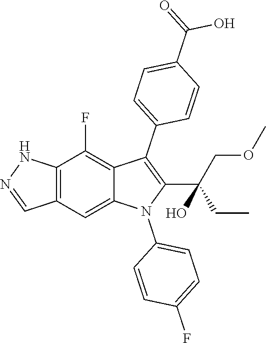 [SMILES: CC[C@@](O)(COC)c1c(-c2ccc(C(=O)O)cc2)c2c(F)c3[nH]ncc3cc2n1-c1ccc(F)cc1] | Compound 1 |
| 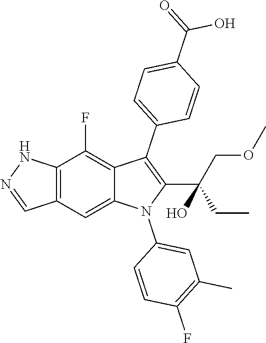 [SMILES: CC[C@@](O)(COC)c1c(-c2ccc(C(=O)O)cc2)c2c(F)c3[nH]ncc3cc2n1-c1ccc(F)c(C)c1] | Compound 2 |
| 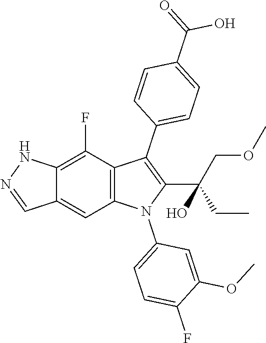 [SMILES: CC[C@@](O)(COC)c1c(-c2ccc(C(=O)O)cc2)c2c(F)c3[nH]ncc3cc2n1-c1ccc(F)c(OC)c1] | Compound 3 |
| 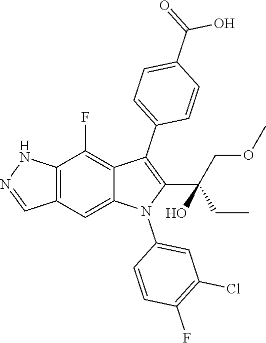 [SMILES: CC[C@@](O)(COC)c1c(-c2ccc(C(=O)O)cc2)c2c(F)c3[nH]ncc3cc2n1-c1ccc(F)c(Cl)c1] | Compound 4 |
| 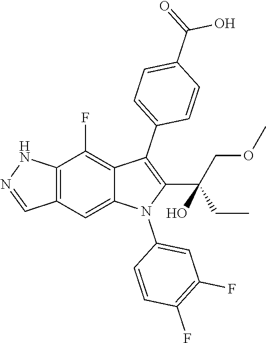 [SMILES: CC[C@@](O)(COC)c1c(-c2ccc(C(=O)O)cc2)c2c(F)c3[nH]ncc3cc2n1-c1ccc(F)c(F)c1] | Compound 5 |

and tautomers of those compounds, deuterated derivatives of those compounds and tautomers, and pharmaceutically acceptable salts of those compounds, tautomers, and deuterated derivatives that can be employed in the treatment of AATD.

Another aspect of the disclosure provides compounds of Formula Ib:

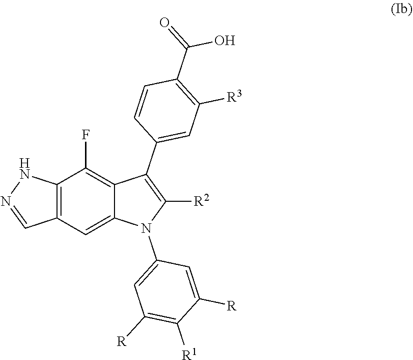

as well as tautomers of those compounds, deuterated derivatives of those compounds and tautomers, and pharmaceutically acceptable salts of those compounds, tautomers, and deuterated derivatives that can be employed in the treatment of AATD, wherein:
each R is independently selected from F, H, Cl, —CH₃, —OCH₃, and —OCD₃;
R¹ is H or F;
R² is selected from

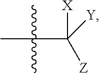

wherein
   one of X, Y, and Z is —OH;
   one of X, Y, and Z is chosen from —CH₂CH₃ and cyclopropyl;
   and one of X, Y, and Z is chosen from —CH₂OCH₃, —CH₂CH₂OCH₃, —CH₂OCH₂CH₃, —CH₂O-cyclopropyl, and —CH₂O-isopropyl;

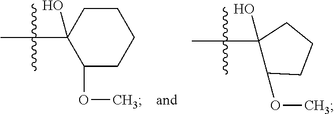 [SMILES: *C1(O)CCCCC1OC ; "and" ; *C1(O)CCCC1OC]

and
R³ is chosen from H and —CH₃.

Formula Ib encompasses compounds of Formula Ib-i:

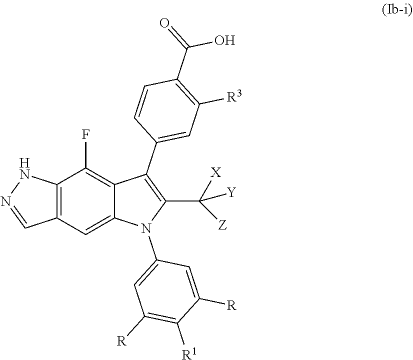

as well as tautomers of those compounds, deuterated derivatives of those compounds and tautomers, and pharmaceutically acceptable salts of those compounds, tautomers, and deuterated derivatives that can be employed in the treatment of AATD, wherein:
each R is independently selected from F, H, Cl, CH₃, —OCH₃, and —OCD₃;
R¹ is H or F;
X, Y, and Z are defined as follows:
   one of X, Y, and Z is —OH;
   one of X, Y, and Z is chosen from —CH₂CH₃ and cyclopropyl;
   and one of X, Y, and Z is chosen from —CH₂OCH₃, —CH₂CH₂OCH₃, —CH₂OCH₂CH₃, —CH₂O-cyclopropyl, and —CH₂O-isopropyl; and
R³ is chosen from H and CH₃.

Formula Ib also encompasses compounds of Formula Ib-ii:

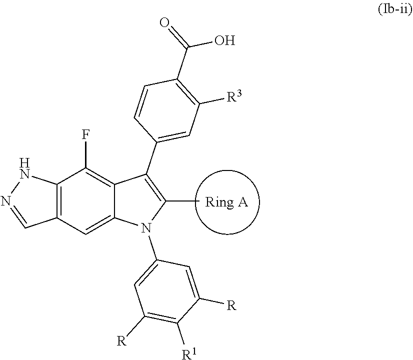

as well as tautomers of those compounds, deuterated derivatives of those compounds and tautomers, and pharmaceutically acceptable salts of those compounds, tautomers, and deuterated derivatives that can be employed in the treatment of AATD, wherein:
each R is independently selected from F, H, Cl, CH₃, —OCH₃, and —OCD₃;
R¹ is H or F; and
R³ is chosen from H and —CH₃; and
Ring A is selected from

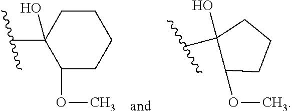

The compounds of Formula Ib, including compounds of Formulae Ib-i and Ib-ii, have unexpectedly and significantly lower anticipated efficacious dose in humans (Compound Quality) than prior art compounds sharing this same scaffold. In one embodiment, the compounds of Formula Ib are selected from Compounds 6 to 57:

| | |
|---|---|
| 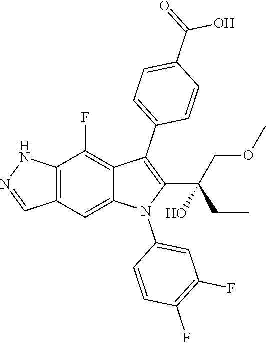 [SMILES: CC[C@](O)(COC)c1c(-c2ccc(C(=O)O)cc2)c2c(F)c3[nH]ncc3cc2n1-c1ccc(F)c(F)c1] | Compound 6 |
| 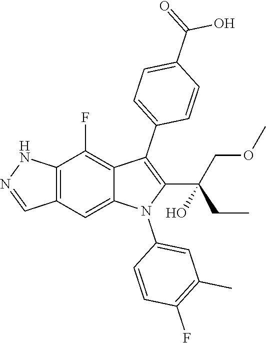 [SMILES: CC[C@](O)(COC)c1c(-c2ccc(C(=O)O)cc2)c2c(F)c3[nH]ncc3cc2n1-c1ccc(F)c(C)c1] | Compound 7 |
| 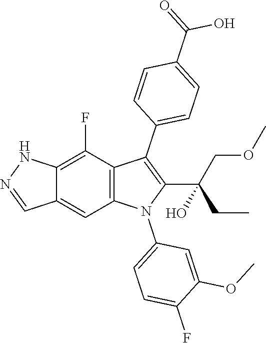 [SMILES: CC[C@](O)(COC)c1c(-c2ccc(C(=O)O)cc2)c2c(F)c3[nH]ncc3cc2n1-c1ccc(F)c(OC)c1] | Compound 8 |
| 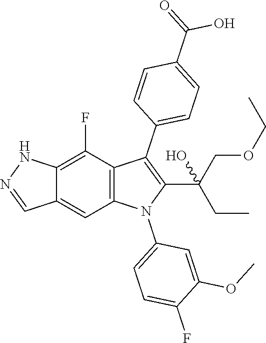 [SMILES: CCOCC(O)(CC)c1c(-c2ccc(C(=O)O)cc2)c2c(F)c3[nH]ncc3cc2n1-c1ccc(F)c(OC)c1] | Compound 9 |
| 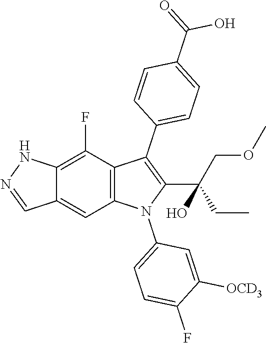 | Compound 10 |
| 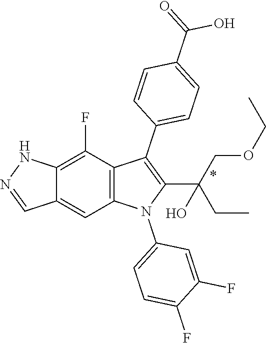 [SMILES: CCOCC(O)(CC)c1c(-c2ccc(C(=O)O)cc2)c2c(F)c3[nH]ncc3cc2n1-c1ccc(F)c(F)c1] | Compound 11 |
| 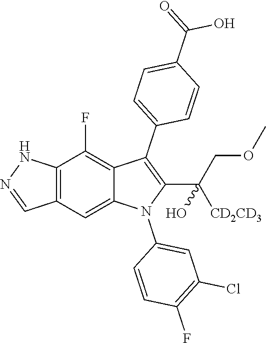 | Compound 12 |
| 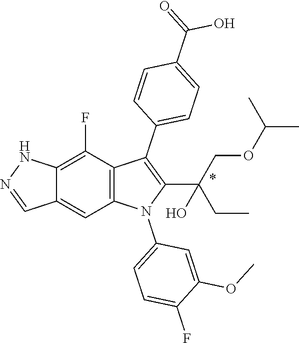 [SMILES: CCC(O)(COC(C)C)c1c(-c2ccc(C(=O)O)cc2)c2c(F)c3[nH]ncc3cc2n1-c1ccc(F)c(OC)c1] | Compound 13 |
| 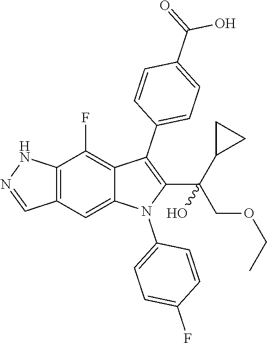 [SMILES: CCOCC(O)(c1c(-c2ccc(C(=O)O)cc2)c2c(F)c3[nH]ncc3cc2n1-c1ccc(F)cc1)C1CC1] | Compound 14 |
| 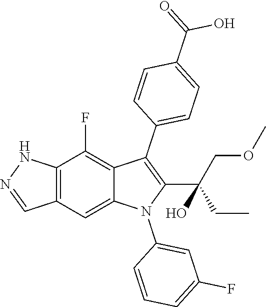 [SMILES: CC[C@@](O)(COC)c1c(-c2ccc(C(=O)O)cc2)c2c(F)c3[nH]ncc3cc2n1-c1cccc(F)c1] | Compound 15 |
| 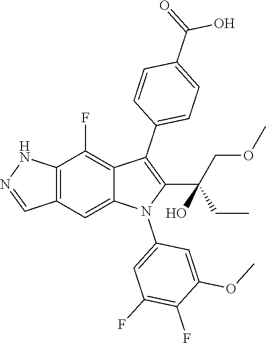 [SMILES: CC[C@@](O)(COC)c1c(-c2ccc(C(=O)O)cc2)c2c(F)c3[nH]ncc3cc2n1-c1cc(F)c(F)c(OC)c1] | Compound 16 |
| 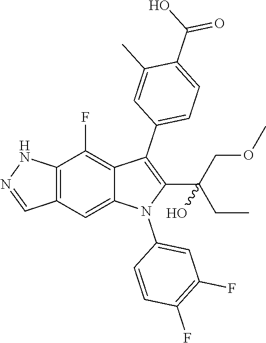 [SMILES: CCC(O)(COC)c1c(-c2ccc(C(=O)O)c(C)c2)c2c(F)c3[nH]ncc3cc2n1-c1ccc(F)c(F)c1] | Compound 17 |
| 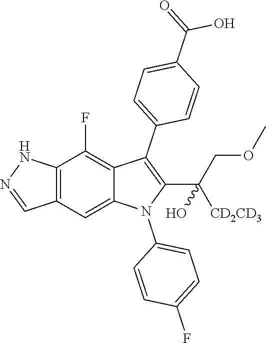 | Compound 18 |
| 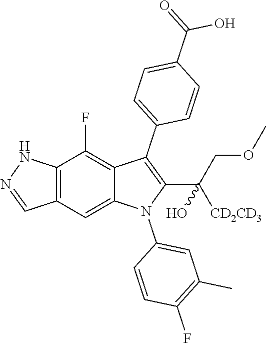 | Compound 19 |
| 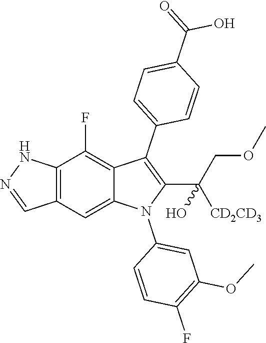 | Compound 20 |
| 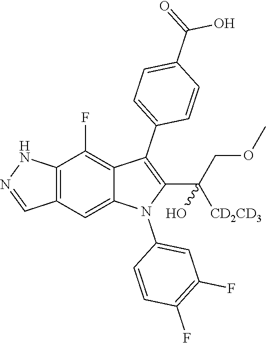 | Compound 21 |
| 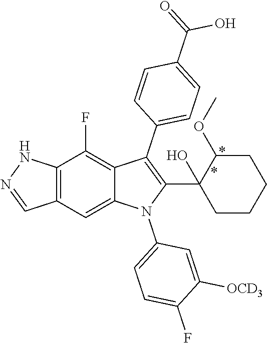 | Compound 22 |
| 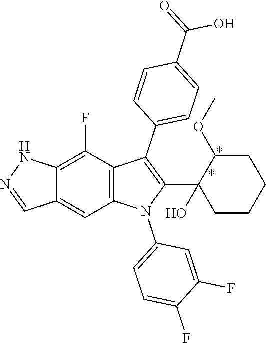 [SMILES: COC1CCCCC1(O)c1c(-c2ccc(C(=O)O)cc2)c2c(F)c3[nH]ncc3cc2n1-c1ccc(F)c(F)c1] | Compound 23 |
| 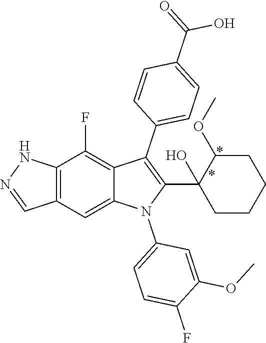 [SMILES: COc1cc(-n2c(C3(O)CCCCC3OC)c(-c3ccc(C(=O)O)cc3)c3c(F)c4[nH]ncc4cc32)ccc1F] | Compound 24 |
| 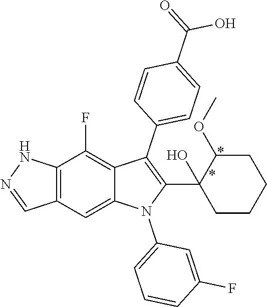 [SMILES: COC1CCCCC1(O)c1c(-c2ccc(C(=O)O)cc2)c2c(F)c3[nH]ncc3cc2n1-c1cccc(F)c1] | Compound 25 |
| 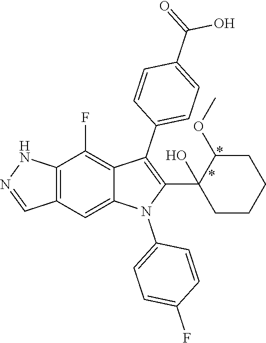 [SMILES: COC1CCCCC1(O)c1c(-c2ccc(C(=O)O)cc2)c2c(F)c3[nH]ncc3cc2n1-c1ccc(F)cc1] | Compound 26 |
| 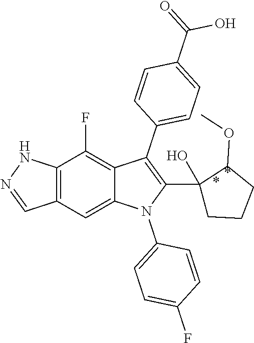 [SMILES: COC1CCCC1(O)c1c(-c2ccc(C(=O)O)cc2)c2c(F)c3[nH]ncc3cc2n1-c1ccc(F)cc1] | Compound 27 |
| 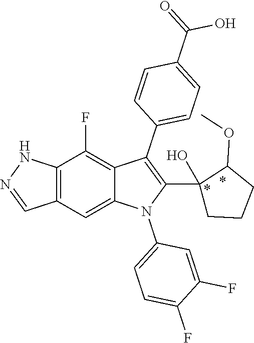 [SMILES: COC1CCCC1(O)c1c(-c2ccc(C(=O)O)cc2)c2c(F)c3[nH]ncc3cc2n1-c1ccc(F)c(F)c1] | Compound 28 |
| 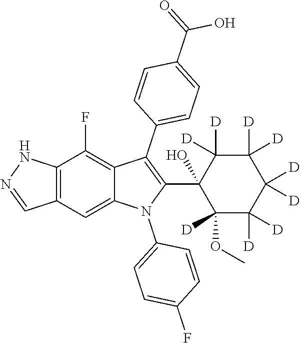 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)cc2)[C@@]([2H])(OC)C1([2H])[2H]] | Compound 29 |
| 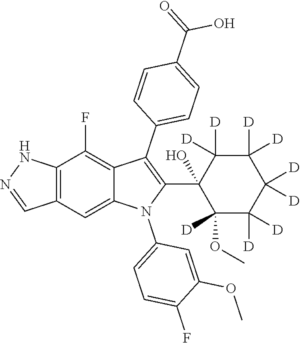 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)c(OC)c2)[C@@]([2H])(OC)C1([2H])[2H]] | Compound 30 |
| 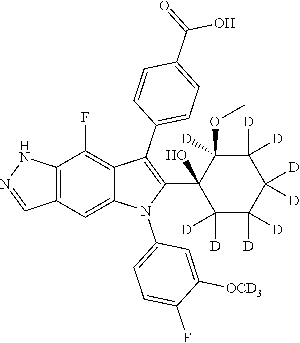 | Compound 31 |
| 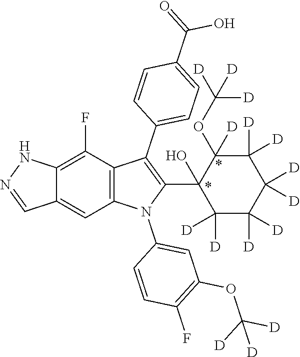 [SMILES: [2H]C([2H])([2H])Oc1cc(-n2c(C3(O)C([2H])([2H])C([2H])([2H])C([2H])([2H])C([2H])([2H])C3([2H])OC([2H])([2H])[2H])c(-c3ccc(C(=O)O)cc3)c3c(F)c4[nH]ncc4cc32)ccc1F] | Compound 32 |
| 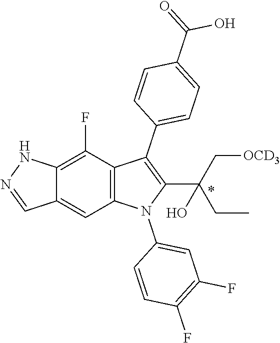 | Compound 33 |
| 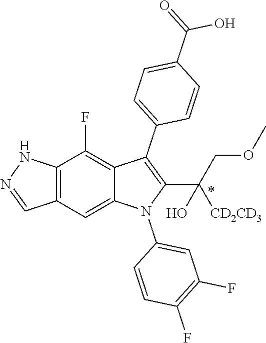 | Compound 34 |
| 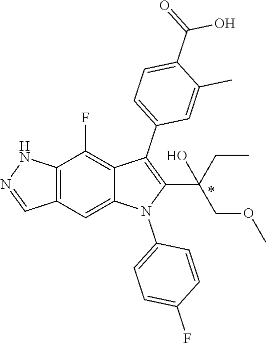 [SMILES: CCC(O)(COC)c1c(-c2ccc(C(=O)O)c(C)c2)c2c(F)c3[nH]ncc3cc2n1-c1ccc(F)cc1] | Compound 35 |
| 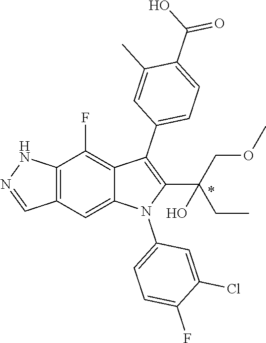 [SMILES: CCC(O)(COC)c1c(-c2ccc(C(=O)O)c(C)c2)c2c(F)c3[nH]ncc3cc2n1-c1ccc(F)c(Cl)c1] | Compound 36 |
| 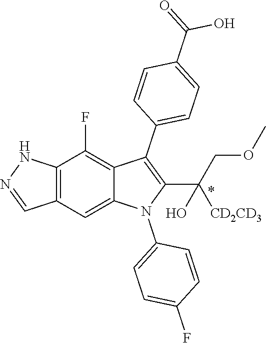 | Compound 37 |
| 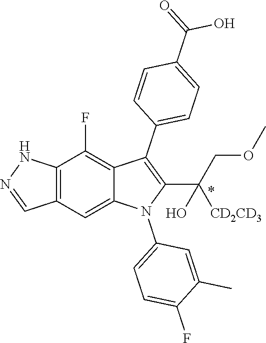 | Compound 38 |
| 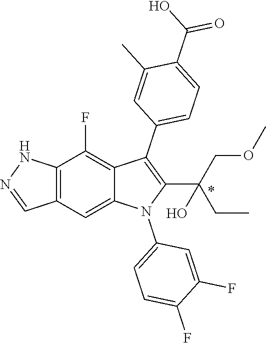 [SMILES: CCC(O)(COC)c1c(-c2ccc(C(=O)O)c(C)c2)c2c(F)c3[nH]ncc3cc2n1-c1ccc(F)c(F)c1] | Compound 39 |
| 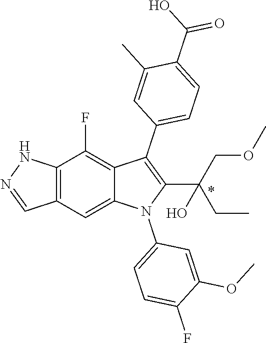 [SMILES: CCC(O)(COC)c1c(-c2ccc(C(=O)O)c(C)c2)c2c(F)c3[nH]ncc3cc2n1-c1ccc(F)c(OC)c1] | Compound 40 |
| 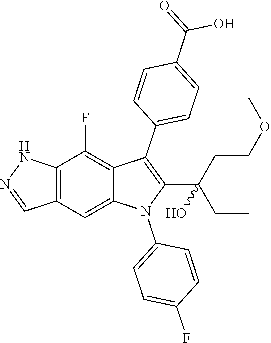 [SMILES: CCC(O)(CCOC)c1c(-c2ccc(C(=O)O)cc2)c2c(F)c3[nH]ncc3cc2n1-c1ccc(F)cc1] | Compound 41 |
| 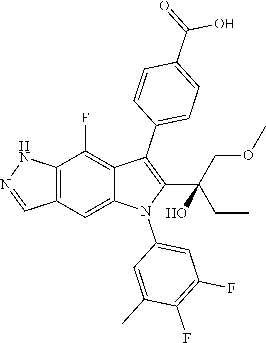 [SMILES: CC[C@@](O)(COC)c1c(-c2ccc(C(=O)O)cc2)c2c(F)c3[nH]ncc3cc2n1-c1cc(C)c(F)c(F)c1] | Compound 42 |
| 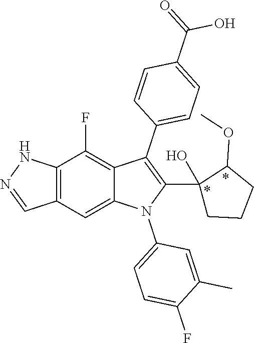 [SMILES: COC1CCCC1(O)c1c(-c2ccc(C(=O)O)cc2)c2c(F)c3[nH]ncc3cc2n1-c1ccc(F)c(C)c1] | Compound 43 |
| 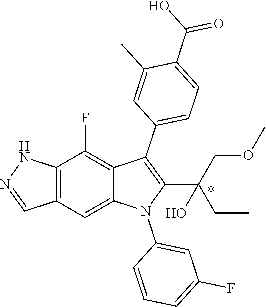 [SMILES: CCC(O)(COC)c1c(-c2ccc(C(=O)O)c(C)c2)c2c(F)c3[nH]ncc3cc2n1-c1cccc(F)c1] | Compound 44 |
| 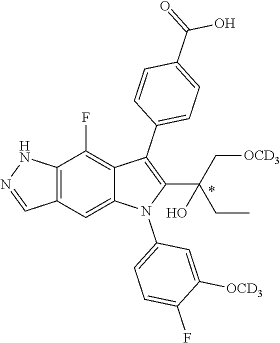 | Compound 45 |
| 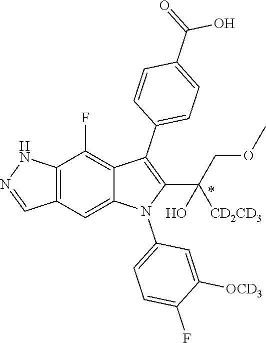 | Compound 46 |
| 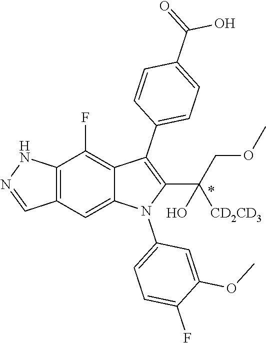 | Compound 47 |
| 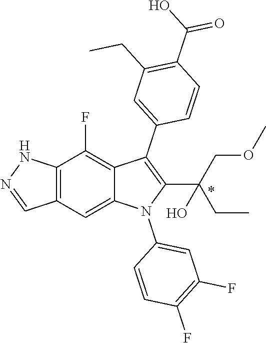 [SMILES: CCc1cc(-c2c(C(O)(CC)COC)n(-c3ccc(F)c(F)c3)c3cc4cn[nH]c4c(F)c23)ccc1C(=O)O] | Compound 48 |
| 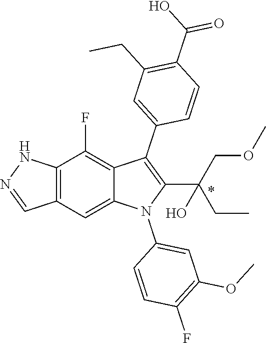 [SMILES: CCc1cc(-c2c(C(O)(CC)COC)n(-c3ccc(F)c(OC)c3)c3cc4cn[nH]c4c(F)c23)ccc1C(=O)O] | Compound 49 |
| 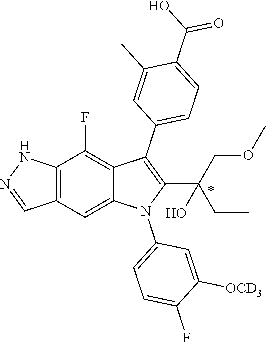 | Compound 50 |
| 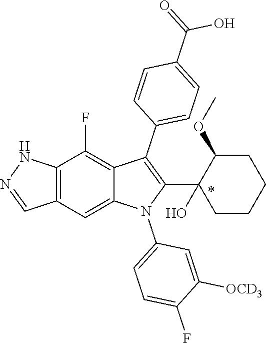 | Compound 51 |
| 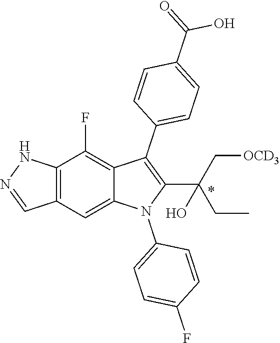 | Compound 52 |
| 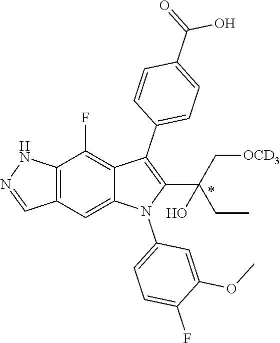 | Compound 53 |
| 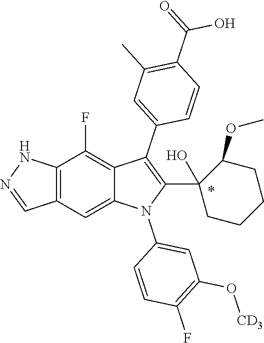 [SMILES: [2H]C([2H])([2H])Oc1cc(-n2c(C3(O)CCCC[C@@H]3OC)c(-c3ccc(C(=O)O)c(C)c3)c3c(F)c4[nH]ncc4cc32)ccc1F] | Compound 54 |
| 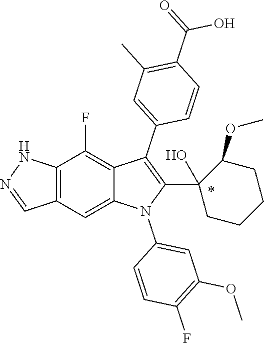 [SMILES: COc1cc(-n2c(C3(O)CCCC[C@@H]3OC)c(-c3ccc(C(=O)O)c(C)c3)c3c(F)c4[nH]ncc4cc32)ccc1F] | Compound 55 |
| 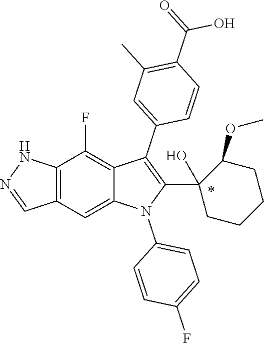 [SMILES: CO[C@H]1CCCCC1(O)c1c(-c2ccc(C(=O)O)c(C)c2)c2c(F)c3[nH]ncc3cc2n1-c1ccc(F)cc1] | Compound 56 |
| 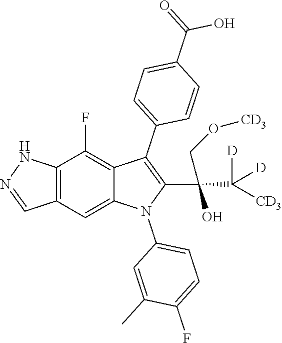 [SMILES: [2H]C([2H])([2H])OC[C@@](O)(c1c(-c2ccc(C(=O)O)cc2)c2c(F)c3[nH]ncc3cc2n1-c1ccc(F)c(C)c1)C([2H])([2H])C([2H])([2H])[2H]] | Compound 57 |

and tautomers of those compounds, deuterated derivatives of those compounds and tautomers, and pharmaceutically acceptable salts of those compounds, tautomers, and deuterated derivatives that can be employed in the treatment of AATD. Formula Ib-i encompasses Compounds 6 to 21, 33 to 42, 44 to 50, 52, 53, and 57 and tautomers of those compounds, deuterated derivatives of those compounds and tautomers, and pharmaceutically acceptable salts of those compounds, tautomers, and deuterated derivatives. Formula Ib-ii encompasses Compounds 22 to 32, 43, 51, and 54 to 56 and tautomers of those compounds, deuterated derivatives of those compounds and tautomers, and pharmaceutically acceptable salts of those compounds, tautomers, and deuterated derivatives.

Another aspect of the disclosure provides compounds of Formula Ic:

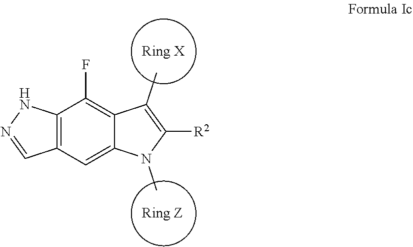

as well as tautomers of those compounds, deuterated derivatives of those compounds and tautomers, and pharmaceutically acceptable salts of those compounds, tautomers, and deuterated derivatives that can be employed in the treatment of AATD, wherein:
Ring X is selected from:

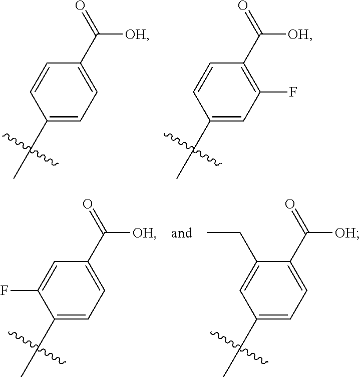 [SMILES: *c1ccc(C(=O)O)cc1 ; *c1ccc(C(=O)O)c(F)c1 ; *c1ccc(C(=O)O)cc1F ; "and" ; *c1ccc(C(=O)O)c(CC)c1]

R² is selected from;

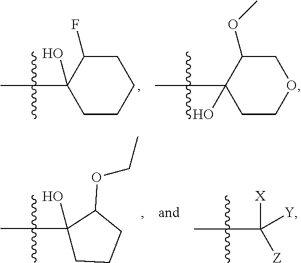

wherein
   one of X, Y, and Z is —OH;
   one of X, Y, and Z is —CH₂CH₃;
   and one of X, Y, and Z is chosen from —CH₂OCH₃, and —CH₂OCH₂CH₃; and
Ring Z is selected from

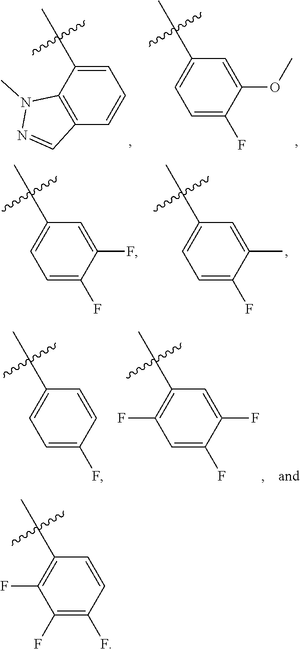

The compounds of Formula Ic have unexpectedly and significantly lower anticipated efficacious dose in humans (Compound Quality) than prior art compounds sharing this same scaffold. In one embodiment, the compounds of Formula Ic are selected from Compounds 58 to 67:

| | |
|---|---|
| 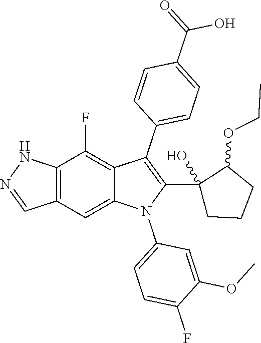 [SMILES: CCOC1CCCC1(O)c1c(-c2ccc(C(=O)O)cc2)c2c(F)c3[nH]ncc3cc2n1-c1ccc(F)c(OC)c1] | Compound 58 |
| 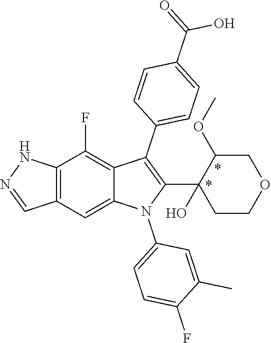 [SMILES: COC1COCCC1(O)c1c(-c2ccc(C(=O)O)cc2)c2c(F)c3[nH]ncc3cc2n1-c1ccc(F)c(C)c1] | Compound 59 |
| 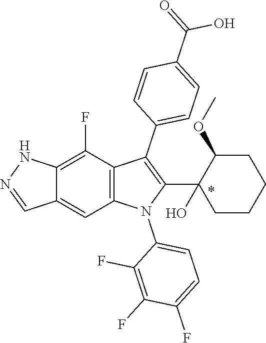 [SMILES: CO[C@H]1CCCCC1(O)c1c(-c2ccc(C(=O)O)cc2)c2c(F)c3[nH]ncc3cc2n1-c1ccc(F)c(F)c1F] | Compound 60 |
| 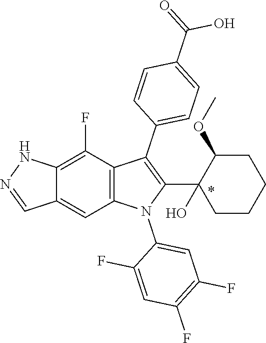 [SMILES: CO[C@H]1CCCCC1(O)c1c(-c2ccc(C(=O)O)cc2)c2c(F)c3[nH]ncc3cc2n1-c1cc(F)c(F)cc1F] | Compound 61 |
| 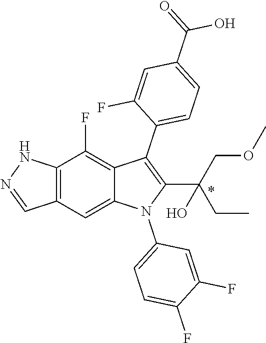 [SMILES: CCC(O)(COC)c1c(-c2ccc(C(=O)O)cc2F)c2c(F)c3[nH]ncc3cc2n1-c1ccc(F)c(F)c1] | Compound 62 |
| 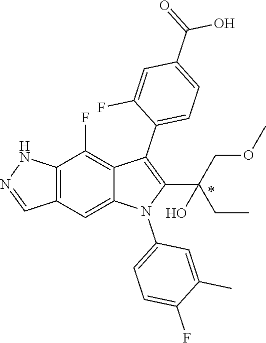 [SMILES: CCC(O)(COC)c1c(-c2ccc(C(=O)O)cc2F)c2c(F)c3[nH]ncc3cc2n1-c1ccc(F)c(C)c1] | Compound 63 |
| 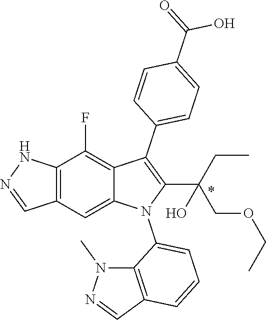 [SMILES: CCOCC(O)(CC)c1c(-c2ccc(C(=O)O)cc2)c2c(F)c3[nH]ncc3cc2n1-c1cccc2cnn(C)c12] | Compound 64 |
| 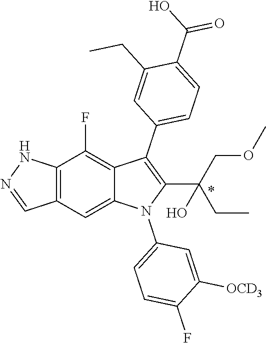 | Compound 65 |
| 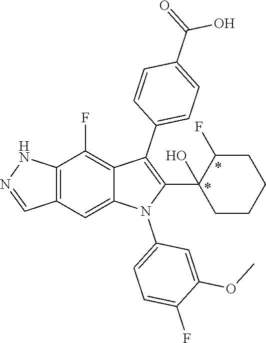 [SMILES: COc1cc(-n2c(C3(O)CCCCC3F)c(-c3ccc(C(=O)O)cc3)c3c(F)c4[nH]ncc4cc32)ccc1F] | Compound 66 |
| 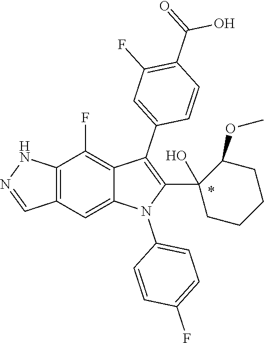 [SMILES: CO[C@H]1CCCCC1(O)c1c(-c2ccc(C(=O)O)c(F)c2)c2c(F)c3[nH]ncc3cc2n1-c1ccc(F)cc1] | Compound 67 |

and tautomers of those compounds, deuterated derivatives of those compounds and tautomers, and pharmaceutically acceptable salts of those compounds, tautomers, and deuterated derivatives that can be employed in the treatment of AATD.

The compounds of Formula I, including compounds of Formulae Ia, Ib, Ib-i, Ib-ii, and Ic, are modulators of AAT activity. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have an EC₅₀ of 0.10 μM or less when tested in an AAT Function Assay, such as, e.g., the MSD Assay NL20-SI Cell Line described in Example 6. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have an EC₅₀ of 0.06 μM or less when tested in an AAT Function Assay. In some embodiments, the compounds of Formula I, including compounds of Formulae Ia, Ib, Ib-i Ib-ii, and Ic, as well as tautomers of those compounds, deuterated derivatives of those tautomers and compounds, and pharmaceutically acceptable salts of those compounds, tautomers, and deuterated derivatives have an EC₅₀ of 0.04 μM or less when tested in an AAT Function Assay. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have an EC₅₀ of 0.02 μM or less when tested in an AAT Function Assay.

In some embodiments, the compounds of Formulae I and II, including compounds of Formulae IIa and IIb, as well as tautomers of those compounds, deuterated derivatives of those tautomers and compounds, and pharmaceutically acceptable salts of those compounds, tautomers, and deuterated derivatives have an unbound hepatocyte clearance value of 27 μL/min/million cells or less when tested in a human hepatocyte clearance assay, such as, e.g., the hepatocyte clearance assay described in Example 6 below. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have an unbound hepatocyte clearance of 16 μL/min/million cells or less or less when tested a human hepatocyte clearance assay. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have an unbound hepatocyte clearance of 12 μL/min/million cells or less or less when tested a human hepatocyte clearance assay.

In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have a Compound Quality (potency in an AAT functional assay multiplied by unbound clearance) score of less than 0.40. In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives have a Compound Quality score of less than 0.30.

In some embodiments, the compounds of Formula I, including compounds of Formulae Ia, Ib, Ib-i, Ib-ii, and Ic, as well as tautomers of those compounds, deuterated derivatives of those tautomers and compounds, and pharmaceutically acceptable salts of those compounds, tautomers, and deuterated derivatives are provided for use in the treatment of AATD.

In one aspect of the disclosure, the compounds of Formula Ia are selected from Compounds 1 to 5, tautomers of those compounds, deuterated derivatives of those compounds or tautomers, and pharmaceutically acceptable salts of any of the foregoing for use in the treatment of AATD. The compounds of Formula Ib are selected from Compounds 6 to 57 tautomers of those compounds, deuterated derivatives of those compounds or tautomers, and pharmaceutically acceptable salts of any of the foregoing for use in the treatment of AATD. The compounds of Formula Ib-i are selected from Compounds 6 to 21, 33 to 42, 44 to 50, 52, 53, and 57, tautomers of those compounds, deuterated derivatives of those compounds or tautomers, and pharmaceutically acceptable salts of any of the foregoing for use in the treatment of AATD. The compounds of Formula Ib-ii are selected from Compounds 22 to 32, 43, 51, and 54 to 56, tautomers of those compounds, deuterated derivatives of those compounds or tautomers, and pharmaceutically acceptable salts of any of the foregoing for use in the treatment of AATD. The compounds of Formula Ic are selected from Compounds 58 to 67, tautomers of those compounds, deuterated derivatives of those compounds or tautomers, and pharmaceutically acceptable salts of any of the foregoing for use in the treatment of AATD.

In some embodiments, the disclosure provides pharmaceutical compositions comprising at least one compound selected from compounds of Formula Ia, tautomers of those compounds, deuterated derivatives of those compounds and tautomers, and pharmaceutically acceptable salts of any of the foregoing. In some embodiments, the pharmaceutical compositions may comprise a compound selected from Compounds 1 to 5, tautomers of those compounds, deuterated derivatives of those compounds and tautomers, and pharmaceutically acceptable salts of any of the foregoing. These compositions may further include at least one additional active pharmaceutical ingredient and/or at least one carrier.

In some embodiments, the disclosure provides pharmaceutical compositions comprising at least one compound selected from compounds of Formula Ib, tautomers of those compounds, deuterated derivatives of those compounds and tautomers, and pharmaceutically acceptable salts of any of the foregoing. In some embodiments, the pharmaceutical compositions may comprise a compound selected from Compounds 6 to 57 (e.g., Compounds 6 to 21, 33 to 42, 44 to 50, 52, 53, and 57 and Compounds 22 to 32, 43, 51, and 54 to 56), tautomers of those compounds, deuterated derivatives of those compounds and tautomers, and pharmaceutically acceptable salts of any of the foregoing. These compositions may further include at least one additional active pharmaceutical ingredient and/or at least one carrier.

In some embodiments, the disclosure provides pharmaceutical compositions comprising at least one compound selected from compounds of Formula Ib-i, tautomers of those compounds, deuterated derivatives of those compounds and tautomers, and pharmaceutically acceptable salts of any of the foregoing. In some embodiments, the pharmaceutical compositions may comprise a compound selected from Compounds 6 to 21, 33 to 42, 44 to 50, 52, 53, and 57, tautomers of those compounds, deuterated derivatives of those compounds and tautomers, and pharmaceutically acceptable salts of any of the foregoing. In some embodiments, the disclosure provides pharmaceutical compositions comprising at least one compound selected from compounds of Formula Ib-ii, tautomers of those compounds, deuterated derivatives of those compounds and tautomers, and pharmaceutically acceptable salts of any of the foregoing. In some embodiments, the pharmaceutical compositions may comprise a compound selected from Compounds 22 to 32, 43, 51, and 54 to 56, tautomers of those compounds, deuterated derivatives of those compounds and tautomers, and pharmaceutically acceptable salts of any of the foregoing. These compositions may further include at least one additional active pharmaceutical ingredient and/or at least one carrier.

In some embodiments, the disclosure provides pharmaceutical compositions comprising at least one compound selected from compounds of Formula Ic, tautomers of those compounds, deuterated derivatives of those compounds and tautomers, and pharmaceutically acceptable salts of any of the foregoing. In some embodiments, the pharmaceutical compositions may comprise a compound selected from Compounds 58 to 67, tautomers of those compounds, deuterated derivatives of those compounds and tautomers, and pharmaceutically acceptable salts of any of the foregoing. These compositions may further include at least one additional active pharmaceutical ingredient and/or at least one carrier.

Another aspect of the disclosure provides methods of treating AATD comprising administering to a subject in need thereof at least one compound selected from compounds of Formula Ia, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 1 to 5, tautomers of those compounds, deuterated derivatives of those compounds and tautomers, and pharmaceutically acceptable salts of any of the foregoing.

Another aspect of the disclosure provides methods of treating AATD comprising administering to a subject in need thereof, at least one compound selected from compounds of Formula Ib, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 6 to 57, tautomers of those compounds, deuterated derivatives of those compounds and tautomers, and pharmaceutically acceptable salts of any of the foregoing.

Another aspect of the disclosure provides methods of treating AATD comprising administering to a subject in need thereof, at least one compound selected from compounds of Formula Ib-i, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 6 to 21, 33 to 42, 44 to 50, 52, 53, and 57, tautomers of those compounds, deuterated derivatives of those compounds and tautomers, and pharmaceutically acceptable salts of any of the foregoing.

Another aspect of the disclosure provides methods of treating AATD comprising administering to a subject in need thereof, at least one compound selected from compounds of Formula Ib-ii, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 22 to 32, 43, 51, and 54 to 56, tautomers of those compounds, deuterated derivatives of those compounds and tautomers, and pharmaceutically acceptable salts of any of the foregoing.

Another aspect of the disclosure provides methods of treating AATD comprising administering to a subject in need thereof, at least one compound selected from compounds of Formula Ic, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 58 to 67, tautomers of those compounds, deuterated derivatives of those compounds and tautomers, and pharmaceutically acceptable salts of any of the foregoing.

In some embodiments, the methods of treatment include administration of at least one additional active agent to the subject in need thereof, either in the same pharmaceutical composition as the at least one compound selected from compounds of Formula Ia, tautomers of those compounds, deuterated derivatives of those compounds and tautomers, and pharmaceutically acceptable salts of any of the foregoing, or as separate compositions. In some embodiments, the methods comprise administering a compound selected from Compounds 1 to 5, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formula Ib, tautomers of those compounds, deuterated derivatives of those compounds and tautomers, and pharmaceutically acceptable salts of any of the foregoing, or as separate compositions. In some embodiments, the methods comprise administering a compound selected from Compounds 6 to 57, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formula Ib-i, tautomers of those compounds, deuterated derivatives of those compounds and tautomers, and pharmaceutically acceptable salts of any of the foregoing, or as separate compositions. In some embodiments, the methods comprise administering a compound selected from Compounds 6 to 21, 33 to 42, 44 to 50, 52, 53, and 57, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formula Ib-ii, tautomers of those compounds, deuterated derivatives of those compounds and tautomers, and pharmaceutically acceptable salts of any of the foregoing, or as separate compositions. In some embodiments, the methods comprise administering a compound selected from Compounds 22 to 32, 43, 51, and 54 to 56, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formula Ic, tautomers of those compounds, deuterated derivatives of those compounds and tautomers, and pharmaceutically acceptable salts of any of the foregoing, or as separate compositions. In some embodiments, the methods comprise administering a compound selected from Compounds 58 to 67, tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formula I (including Formulae Ia, Ib, Ib-i, Ib-ii, and Ic), tautomers of those compounds, deuterated derivatives of those compounds and tautomers, and pharmaceutically acceptable salts of any of the foregoing, or as separate compositions, wherein the additional active agent is alpha-1 antitrypsin protein (AAT) from the blood plasma of healthy human donors. In some embodiments, the methods comprise administering a compound selected from Compounds 1 to 5; Compounds 6 to 21, 33 to 42, 44 to 50, 52, 53 and 57; Compounds 22 to 32, 43, 51, and 54 to 56; and Compounds 58 to 67, tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is alpha-1 antitrypsin protein (AAT) from the blood plasma of healthy human donors.

In some embodiments, the methods of treatment include administration of at least one additional active agent to the subject in need thereof, either in the same pharmaceutical composition as the at least one compound selected from compounds of Formula I (including Formulae Ia, Ib, Ib-i, Ib-ii, and Ic), tautomers of those compounds, deuterated derivatives of those compounds and tautomers, and pharmaceutically acceptable salts of any of the foregoing, or as separate compositions, wherein the additional active agent is recombinant AAT. In some embodiments, the methods comprise administering a compound selected from Compounds 1 to 5; Compounds 6 to 21, 33 to 42, 44 to 50, 52, 53, and 57; Compounds 22 to 32, 43, 51, and 54 to 56; and Compounds 58 to 67, tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is recombinant AAT.

Also provided are methods of modulating AAT, comprising administering to a subject in need thereof, at least one compound selected from compounds of Formula I (including Formulae Ia, Ib, Ib-i, Ib-ii, and Ic), and tautomers of those compounds, deuterated derivatives of those compounds and tautomers, and pharmaceutically acceptable salts of any of the foregoing or a pharmaceutical composition comprising the at least one compound, tautomer, deuterated derivative, or pharmaceutically acceptable salt. In some embodiments, the methods of modulating AAT comprise administering at least one compound selected from Compounds 1 to 5; Compounds 6 to 21, 33 to 42, 44 to 50, 52, 53, and 57; Compounds 22 to 32, 43, 51, and 54 to 56; and Compounds 58 to 67, tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt.

Also provided is a compound of Formula I (including Formulae Ia, Ib, Ib-i, Ib-ii, and Ic, and tautomers of those compounds, deuterated derivatives of those compounds and tautomers, and pharmaceutically acceptable salts of any of the foregoing, for use in therapy. In some embodiments, there is provided a compound selected from Compounds 1 to 5; Compounds 6 to 21, 33 to 42, 44 to 50, 52, 53, and 57; Compounds 22 to 32, 43, 51, and 54 to 56; and Compounds 58 to 67, tautomers of those compounds, deuterated derivatives of those compounds and tautomers, and pharmaceutically acceptable salts of any of the foregoing, for use in therapy.

Also provided is a pharmaceutical composition comprising a compound of Formula I (including Formulae Ia, Ib, Ib-i, Ib-ii, and Ic, and tautomers of those compounds, deuterated derivatives of those compounds and tautomers, and pharmaceutically acceptable salts of any of the foregoing) for use in therapy. In some embodiments, there is provided a pharmaceutical composition comprising a compound selected from Compounds 1 to 5; Compounds 6 to 21, 33 to 42, 44 to 50, 52, 53, and 57; Compounds 22 to 32, 43, 51, and 54 to 56; and Compounds 58 to 67, tautomers of those compounds, deuterated derivatives of those compounds and tautomers, and pharmaceutically acceptable salts of any of the foregoing, for use in therapy.

### BRIEF DESCRIPTION OF THE DRAWINGS

FIG. 1 shows an XRPD diffractogram of Compound 5 free form Monohydrate Form A.

FIG. 2 shows a TGA thermogram of Compound 5 free form Monohydrate Form A.

FIG. 3 shows DSC thermogram of Compound 5 free form Monohydrate Form A.

FIG. 4 shows a solid state ¹³C NMR spectrum of Compound 5 free form Monohydrate Form A.

FIG. 5 shows solid state ¹⁹F NMR spectrum of Compound 5 free form Monohydrate Form A.

FIG. 6 shows an XRPD diffractogram of Compound 5 free form Form A.

FIG. 7 shows a TGA thermogram of Compound 5 free form Form A.

FIG. 8 shows a DSC thermogram of Compound 5 free form Form A.

FIG. 9 shows a solid state ¹³C NMR spectrum of Compound 5 free form Form A.

FIG. 10 shows solid state ¹⁹F NMR spectrum of Compound 5 free form Form A.

FIG. 11 shows an XRPD diffractogram of Compound 5 free form Form B.

FIG. 12 shows a TGA thermogram of Compound 5 free form Form B.

FIG. 13 shows a DSC thermogram of Compound 5 free form Form B.

FIG. 14 shows an XRPD diffractogram of Compound 5 free form NPA Solvate Form A.

FIG. 15 shows a TGA thermogram of Compound 5 free form NPA Solvate Form A.

FIG. 16 shows a DSC thermogram of Compound 5 free form NPA Solvate Form A.

FIG. 17 shows an XRPD diffractogram of Compound 5 free form EtOH Solvate Form A.

FIG. 18 shows a TGA thermogram of Compound 5 free form EtOH Solvate Form A.

FIG. 19 shows a DSC thermogram of Compound 5 free form EtOH Solvate Form A.

FIG. 20 shows a solid state ¹³C NMR spectrum of Compound 5 free form EtOH Solvate Form A.

FIG. 21 shows solid state ¹⁹F NMR spectrum of Compound 5 free form EtOH Solvate Form A.

FIG. 22 shows an XRPD diffractogram of Compound 5 free form MeOH Solvate Hydrate Form A.

FIG. 23 shows a TGA thermogram of Compound 5 free form MeOH Solvate Hydrate Form A.

FIG. 24 shows a DSC thermogram of Compound 5 free form MeOH Solvate Hydrate Form A.

FIG. 25 shows a solid state ¹³C NMR spectrum of Compound 5 free form MeOH Solvate Hydrate Form A.

FIG. 26 shows solid state ¹⁹F NMR spectrum of Compound 5 free form MeOH Solvate Hydrate Form A.

FIG. 27 shows an XRPD diffractogram of Compound 5 free form DCM Solvate Form A.

FIG. 28 shows a TGA thermogram of Compound 5 free form DCM Solvate Form A.

FIG. 29 shows a solid state ¹³C NMR spectrum of Compound 5 free form DCM Solvate Form A.

FIG. 30 shows solid state ¹⁹F NMR spectrum of Compound 5 free form DCM Solvate Form A.

FIG. 31 shows an XRPD diffractogram of Compound 5 free form EtOAc Heptane Solvate Form A.

FIG. 32 shows a TGA thermogram of Compound 5 free form EtOAc Heptane Solvate Form A.

FIG. 33 shows a DSC thermogram of Compound 5 free form EtOAc Heptane Solvate Form A.

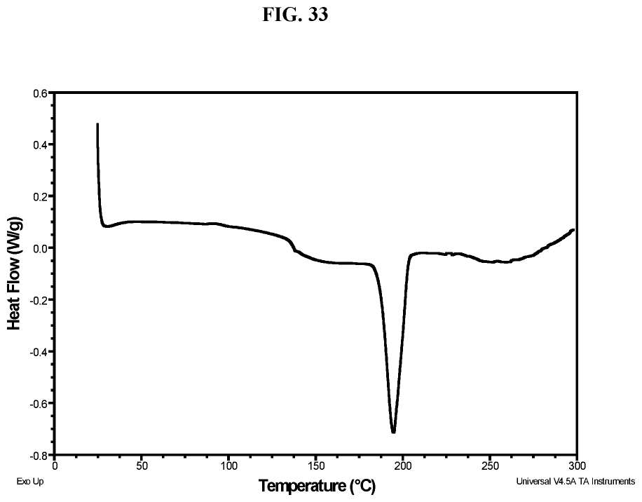

FIG. 34 shows a solid state ¹³C NMR spectrum of Compound 5 free form EtOAc Heptane Solvate Form A.

FIG. 35 shows solid state ¹⁹F NMR spectrum of Compound 5 free form EtOAc Heptane Solvate Form A.

FIG. 36 shows an XRPD diffractogram of Compound 3 free form Form A.

FIG. 37 shows a TGA thermogram of Compound 3 free form Form A.

FIG. 38 shows a DSC thermogram of Compound 3 free form Form A.

FIG. 39 shows a solid state ¹³C NMR spectrum of Compound 3 free form Form A.

FIG. 40 shows a solid state ¹⁹F NMR spectrum of Compound 3 free form Form A.

FIG. 41 shows an XRPD diffractogram of Compound 3 free form Form B.

FIG. 42 shows a TGA thermogram of Compound 3 free form Form B.

FIG. 43 shows a DSC thermogram of Compound 3 free form Form B.

FIG. 44 shows a solid state ¹³C NMR spectrum of Compound 3 free form Form B.

FIG. 45 shows a solid state ¹⁹F NMR spectrum of Compound 3 free form Form B.

FIG. 46 shows an XRPD diffractogram of Compound 3 free form Hydrate Form A.

FIG. 47 shows a TGA thermogram of Compound 3 free form Hydrate Form A.

FIG. 48 shows a DSC thermogram of Compound 3 free form Hydrate Form A.

FIG. 49 shows an XRPD diffractogram of Compound 3 free form Hydrate Form B.

FIG. 50 shows a TGA thermogram of Compound 3 free form Hydrate Form B.

FIG. 51 shows a DSC thermogram of Compound 3 free form Hydrate Form B.

FIG. 52 shows an XRPD diffractogram of Compound 3 free form Hydrate Form C.

FIG. 53 shows a TGA thermogram of Compound 3 free form Hydrate Form C.

FIG. 54 shows a DSC thermogram of Compound 3 free form Hydrate Form C.

FIG. 55 shows an XRPD diffractogram of Compound 3 free form MTBE Solvate Form A.

FIG. 56 shows a TGA thermogram Compound 3 free form MTBE Solvate Form A.

FIG. 57 shows a DSC thermogram of Compound 3 free form MTBE Solvate Form A.

FIG. 58 shows a solid state ¹³C NMR spectrum of Compound 3 free form MTBE Solvate Form A.

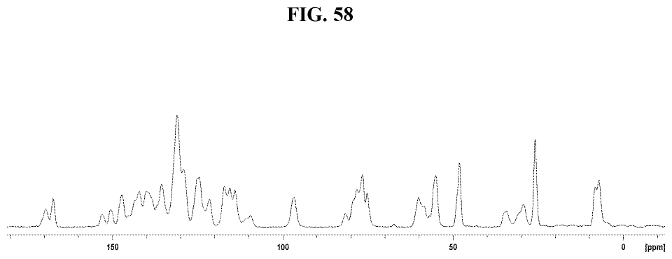

FIG. 59 shows a solid state ¹⁹F NMR spectrum of Compound 3 free form MTBE Solvate Form A.

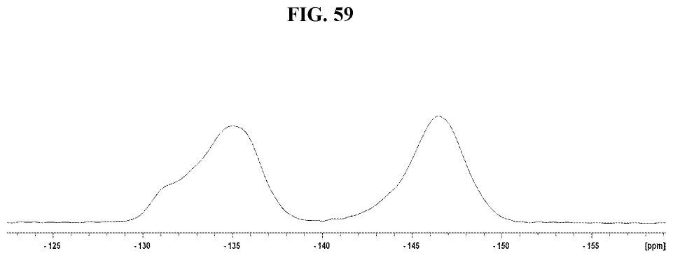

FIG. 60 shows an XRPD diffractogram of Compound 4 free form Form A.

FIG. 61 shows a TGA thermogram of Compound 4 free form Form A.

FIG. 62 shows a DSC thermogram of Compound 4 free form Form A.

FIG. 63 shows a solid state ¹³C NMR spectrum of Compound 4 free form Form A.

FIG. 64 shows solid state ¹⁹F NMR spectrum of Compound 4 free form Form A.

FIG. 65 shows an XRPD diffractogram of Compound 4 free form Form B.

FIG. 66 shows a TGA thermogram of Compound 4 free form Form B.

FIG. 67 shows a DSC thermogram of Compound 4 free form Form B.

FIG. 68 shows a solid state ¹³C NMR spectrum of Compound 4 free form Form B.

FIG. 69 shows solid state ¹⁹F NMR spectrum of Compound 4 free form Form B.

FIG. 70 shows an XRPD diffractogram of Compound 4 free form Form C.

FIG. 71 shows a TGA thermogram of Compound 4 free form Form C.

FIG. 72 shows a DSC thermogram of Compound 4 free form Form C.

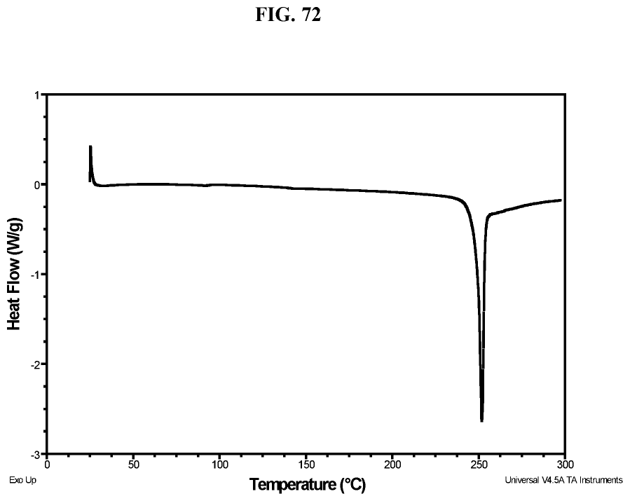

FIG. 73 shows an XRPD diffractogram of Compound 4 free form Form D.

FIG. 74 shows a TGA thermogram of Compound 4 free form Form D.

FIG. 75 shows a DSC thermogram of Compound 4 free form Form D.

FIG. 76 shows a solid state ¹³C NMR spectrum of Compound 4 free form Form D.

FIG. 77 shows solid state ¹⁹F NMR spectrum of Compound 4 free form Form D.

FIG. 78 shows an XRPD diffractogram of Compound 4 free form Hydrate Form A.

FIG. 79 shows a TGA thermogram of Compound 4 free form Hydrate Form A.

FIG. 80 shows a DSC thermogram of Compound 4 free form Hydrate Form A.

FIG. 81 shows a solid state ¹³C NMR spectrum of Compound 4 free form Hydrate Form A.

FIG. 82 shows solid state ¹⁹F NMR spectrum of Compound 4 free form Hydrate Form A.

FIG. 83 shows an XRPD diffractogram of Compound 4 free form Hydrate Form B.

FIG. 84 shows a TGA thermogram of Compound 4 free form Hydrate Form B.

FIG. 85 shows a DSC thermogram of Compound 4 free form Hydrate Form B.

FIG. 86 shows an XRPD diffractogram of Compound 4 free form Hydrate Form C.

FIG. 87 shows a DSC thermogram of Compound 4 free form Hydrate Form C.

### I. DEFINITIONS

The term “AAT” as used herein means alpha-1 antitrypsin or a mutation thereof, including, but not limited to, the AAT gene mutations such as Z mutations. As used herein, “Z-AAT” means AAT mutants which have the Z mutation.

As used herein, “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

As used herein, a patient who is “homozygous” for a particular gene mutation has the same mutation on each allele.

As used herein, a patient who has the PiZZ genotype is a patient who is homozygous for the Z mutation in the AAT protein.

The term “AATD” as used herein means alpha-1 antitrypsin deficiency, which is a genetic disorder characterized by low circulating levels of AAT.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

Compounds of the disclosure may optionally be substituted with one or more substituents. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a specific compound of this disclosure only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of a compound that exist together in equilibrium, and are readily interchanged by migration of an atom or group within the molecule.

“Stereoisomer” refers to both enantiomers and diastereomers.

It will be appreciated that certain compounds of this invention may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e., one stereoisomer). As used in the chemical structures disclosed herein, a “wavy” bond ( [symbol] ) to a stereogenic atom indicates that the compound was isolated as a mixture of isomers (e.g., a mixture of syn and/or anti isomers or a racemic mixture).

As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

As used in the chemical structures disclosed herein, a

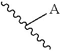

(a “wavy” line perpendicular to a “straight” bond to group “A”) indicates that group “A” is a substituent whose point of attachment is at the end of the bond that terminates at the “wavy” line.

As used herein, the prefix “rac-,” when used in connection with a chiral compound, refers to a racemic mixture of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

Certain compounds disclosed herein may exist as tautomers and both tautomeric forms are intended, even though only a single tautomeric structure is depicted. For example, a description of Compound A is understood to include its tautomer Compound B and vice versa, as well as mixtures thereof:

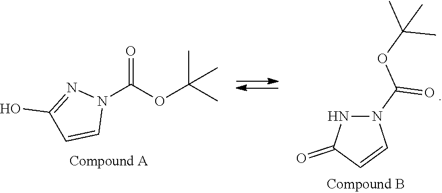

Unless otherwise stated, all tautomeric forms of the compounds of the disclosure are within the scope of the disclosure.

Unless otherwise stated, structures depicted herein are also meant to include all isomeric forms of the structure, e.g., geometric (or conformational), such as (Z) and (E) double bond isomers, and (Z) and (E) conformational isomers. Therefore, geometric and conformational mixtures of the compounds of the disclosure are within the scope of the disclosure.

As used herein, “deuterated derivative” refers to a compound having the same chemical structure as a reference compound, but with one or more hydrogen atoms replaced by a deuterium atom, depicted as “H²” or “D”. For example, a deuterated methyl group may be depicted as —CD₃,

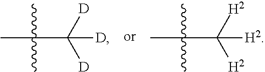

It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

The term “isotopic enrichment factor” as used herein means the ratio between the isotopic abundance and the natural abundance of a specified isotope.

The term “alkyl” as used herein, means a straight-chain (i.e., linear or unbranched) or branched, substituted or unsubstituted hydrocarbon chain that is completely saturated or may contain one or more units of saturation, without being fully aromatic. Unless otherwise specified, alkyl groups contain 1-12 alkyl carbon atoms. In some embodiments, alkyl groups contain 1-10 aliphatic carbon atoms. In other embodiments, alkyl groups contain 1-8 aliphatic carbon atoms. In still other embodiments, alkyl groups contain 1-6 alkyl carbon atoms, in other embodiments alkyl groups contain 1-4 alkyl carbon atoms, and in yet other embodiments alkyl groups contain 1-3 alkyl carbon atoms and 1-2 alkyl carbon atoms.

The term “heteroalkyl” as used herein, refers to aliphatic groups wherein one or two carbon atoms are independently replaced by one or more of oxygen, sulfur, nitrogen, phosphorus, or silicon. Heteroalkyl groups may be substituted or unsubstituted, branched or unbranched.

The term “alkenyl” as used herein, means a straight-chain (i.e., linear or unbranched), branched, substituted or unsubstituted hydrocarbon chain that contains one or more carbon-to-carbon double bonds.

The terms “cycloalkyl,” “cyclic alkyl,” “carbocyclyl,” and “carbocycle” refer to a fused, spirocyclic, or bridged monocyclic C₃₋₉ hydrocarbon or a fused, spirocyclic, or bridged bicyclic or tricyclic, C₈₋₁₄ hydrocarbon that is completely saturated or that contains one or more units of unsaturation, but which is not fully aromatic, wherein any individual ring in said bicyclic ring system has 3-9 members. Typically, a cycloalkyl is completely saturated, while a carbocyclyl may contain one or more units of unsaturation but is not aromatic. In some embodiments, the cycloalkyl or carbocycle group contains 3 to 12 carbon atoms. In some embodiments, the cycloalkyl or carbocycle group contains 3 to 8 carbon atoms. In some embodiments, the cycloalkyl or carbocycle group contains 3 to 6 carbon atoms.

The term “heterocycle,” “heterocyclyl,” or “heterocyclic” as used herein refers to fused, spirocyclic, or bridged non-aromatic, monocyclic, bicyclic, or tricyclic ring systems in which one or more ring members is a heteroatom. In some embodiments, “heterocycle,” “heterocyclyl,” or “heterocyclic” group has 3 to 14 ring members in which one or more ring members is a heteroatom independently selected from oxygen, sulfur, nitrogen, phosphorus, and silicon and each ring in the system contains 3 to 9 ring members. In some embodiments, the heterocyclyl contains 3 to 12 ring member atoms. In some embodiments, the heterocyclyl contains 3 to 8 ring member atoms. In some embodiments, the heterocyclyl contains 3 to 6 ring member atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “alkoxy” as used herein, refers to an alkyl group, as previously defined, wherein one carbon of the alkyl group is replaced by an oxygen (“alkoxy”) atom, respectively, provided that the oxygen atom is linked between two carbon atoms. A “cyclic alkoxy” refers to a monocyclic, fused, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl” and “haloalkoxy” means an alkyl or alkoxy, as the case may be, which is substituted with one or more halogen atoms. The term “halogen” or means F, Cl, Br, or I. In some embodiments, the halogen is selected from F, Cl, and Br. Examples of haloalkyls include —CHF₂, —CH₂F, —CF₃, —CF₂—, or perhaloalkyl, such as, —CF₂CF₃.

As used herein, “═O” refers to an oxo group.

As used herein, a “cyano” or “nitrile” groups refers to —C≡N.

As used herein, a “hydroxy” group refers to —OH.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Nonlimiting examples of aromatic groups include aryl and heteroaryl groups.

The term “aryl” refers to monocyclic, bicyclic, and tricyclic ring systems having a total of 5 to 14 ring members, wherein at least one ring in the system is aromatic and wherein each ring in the system contains 3 to 7 ring members. In some embodiments, an aryl contains 6 or 10 carbon atoms. A nonlimiting example of an aryl group is a phenyl ring.

The term “heteroaryl” refers to monocyclic, bicyclic, and tricyclic ring systems having a total of 5 to 10 ring members, wherein at least one ring in the system is aromatic, at least one ring in the system contains one or more heteroatoms, and wherein each ring in the system contains 3 to 7 ring members. In some embodiments, a heteroaryl contains 6 or 10 ring atoms.

Examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999).

Examples of suitable solvents that may be used in this disclosure include, but not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Examples of suitable bases that may be used in this disclosure include, but not limited to, 1,8-diazabicyclo[5.4.0]undec-7-ene (DBU), potassium tert-butoxide (KOtBu), potassium carbonate (K₂CO₃), N-methylmorpholine (NMN), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound of the disclosure is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J Pharmaceutical Sciences, 1977, 66, 1-19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, the term “pharmaceutically acceptable solid form” refers to a solid form of the referenced compound of this disclosure wherein the solid form (e.g., crystalline free form, crystalline salt, crystalline salt solvate, crystalline salt hydrate, and amorphous form) of the referenced compound of the disclosure is nontoxic and suitable for use in pharmaceutical compositions.

As used herein, the term “amorphous” refers to a solid material having no long-range order in the position of its molecules. Amorphous solids are generally glasses or supercooled liquids in which the molecules are arranged in a random manner so that there is no well-defined arrangement, e.g., molecular packing, and no long-range order. Amorphous solids are generally rather isotropic, i.e., exhibit similar properties in all directions and do not have definite melting points. Instead, they typically exhibit a glass transition temperature which marks a transition from glassy amorphous state to supercooled liquid amorphous state upon heating. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a representative comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, and solid state nuclear magnetic resonance (e.g., ¹³C, ¹⁹F, ¹⁵N, and ³¹P SSNMR). Accordingly, as used herein, the terms “crystalline Compound 5 free form Form A” and “crystalline Compound 5 free form NPA Solvate Form A” refer to unique crystalline forms that can be identified and distinguished from each other by one or more characterization techniques including, for example, XRPD, single crystal X-ray diffraction, and ¹³C SSNMR. In some embodiments, the novel crystalline forms are characterized by an X-ray powder diffractogram having one or more signals at one or more specified degree two-theta (° 20) values.

As used herein, the term “free form” refers to a non-ionized version of the compound in the solid state. Examples of free forms include free bases and free acids.

As used herein, the term “neat form” refers to an unsolvated and unhydrated free form version of a compound in the solid state.

As used herein, the term “solvate” refers to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate.” A “solvate hydrate” refers to a solid form that has been scientifically determined to contain both solvent and water, but may not yet be confirmed as to whether the solvent and water are within one crystal lattice or many.

In some embodiments, a solid material may comprise a mixture of crystalline solids and amorphous solids. A solid material comprising an amorphous compound may also, for example, contain up to 30% of a crystalline solid. In some embodiments, a solid material prepared to comprise an amorphous compound may also, for example, contain up to 25%, 20%, 15%, 10%, 5%, or 2% of a crystalline solid. In embodiments wherein the solid material contains a mixture of crystalline solids and amorphous solids, the characterizing data, such as XRPD, may contain indicators of both crystalline and amorphous solids. In some embodiments, a crystalline form of this disclosure may contain up to 30% amorphous compound. In some embodiments, a crystalline preparation of a referenced compound of the disclosure may contain up to 25%, 20%, 15%, 10%, 5%, or 2% of an amorphous solid.

As used herein, the term “substantially amorphous” refers to a solid material having little or no long-range order in the position of its molecules. For example, substantially amorphous materials have less than 15% crystallinity (e.g., less than 10% crystallinity, less than 5% crystallinity, or less than 2% crystallinity). It is also noted that the term “substantially amorphous” includes the descriptor, “amorphous,” which refers to materials having no (0%) crystallinity.

As used herein, the term “substantially crystalline” refers to a solid material having little or no amorphous molecules. For example, substantially crystalline materials have less than 15% amorphous molecules (e.g., less than 10% amorphous molecules, less than 5% amorphous molecules, or less than 2% amorphous molecules). It is also noted that the term “substantially crystalline” includes the descriptor “crystalline,” which refers to materials that are 100% crystalline form.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the term “ambient conditions” means room temperature, open air condition and uncontrolled humidity condition. As used herein, the terms “room temperature” and “ambient temperature” mean 15° C. to 30° C.

As used herein, the terms “X-ray powder diffractogram,” “X-ray powder diffraction pattern,” “XRPD pattern,” “XRPD spectrum” interchangeably refer to an experimentally obtained pattern plotting signal positions (on the abscissa) versus signal intensities (on the ordinate).

A “signal” or “peak” as used herein refers to a point in the XRPD pattern where the intensity as measured in counts is at a local maximum. An XRPD peak is identified by its angular value as measured in degrees 2θ (° 20), depicted on the abscissa of an X-ray powder diffractogram, which may be expressed, for example, as “a signal at . . . degrees two-theta,” “a signal at [a] two-theta value(s) of . . . ” and/or “a signal at at least . . . two-theta value(s) selected from . . . .”

The repeatability of the measured angular values is in the range of 0.2° 20, i.e., the angular value can be at the recited angular value+0.2 degrees two-theta, the angular value −0.2 degrees two-theta, or any value between those two end points (angular value+0.2 degrees two-theta and angular value −0.2 degrees two-theta).

One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta) generally mean that value is identified as ±0.2 degrees two-theta of the reported value, an art-recognized variance.

As used herein, the term “TGA” refers to thermogravimetric analysis and “TGA/DSC” refers to thermogravimetric analysis and differential scanning calorimetry.

As used herein, the term “DSC” refers to the analytical method of differential scanning calorimetry.

As used herein, the term “solvent” refers to any liquid in which the product is at least partially soluble (solubility of product >1 g/L).

As used herein, the term “glass transition temperature” or “Tg” refers to the temperature above which a hard and brittle “glassy” amorphous solid becomes viscous or rubbery.

As used herein, the terms “melting temperature”, “melting point”, and “Tm” refer to a temperature at which the solid and liquid state are at equilibrium.

As used herein, the term “Compound Quality” refers to the potency of a compound multiplied (x) by the compound's unbound clearance as measured using the assays described in Example 6.

The terms “unbound clearance” refer to unbound intrinsic clearance (unbound CLint) in hepatocytes—the intrinsic clearance a drug would have in the absence of protein binding. Unbound CLint=CLint,hep/fu,hep, where CLint,hep is intrinsic clearance in hepatocytes and fu,hep is unbound fraction in hepatocytes.

“Exposure multiple” as used herein, refers to an assessment of relative compound exposure in toxicology studies. The calculation of an exposure multiple is accomplished by comparing the exposure (AUC) achieved in a toxicology species relative to the target efficacious exposure at steady state (AUCss). The larger exposure multiple provides the opportunity to explore higher doses relative to efficacious concentration in clinical development. However, the exposure that results in an adverse toxicological outcome is unpredictable.

The terms “patient” and “subject” are used interchangeably and refer to an animal including a human.

The terms “effective dose,” “effective amount,” “therapeutically effective dose,” and “therapeutically effective amount” are used interchangeably herein and refer to that amount of a compound that produces the desired effect for which it is administered (e.g., improvement in AATD or a symptom of AATD, lessening the severity of AATD or a symptom of AATD, and/or reducing the rate of onset or incidence of AATD or a symptom of AATD). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the term “treatment and its cognates (e.g., “treat,” “treating”) refer to improving AATD or its symptoms in a subject, delaying the onset of AATD or its symptoms in a subject, or lessening the severity of AATD or its symptoms in a subject. “Treatment” and its cognates as used herein, include, but are not limited to the following: improved liver and/or spleen function, lessened jaundice, improved lung function, lessened lung diseases and/or pulmonary exacerbations (e.g., emphysema), lessened skin disease (e.g., necrotizing panniculitis), increased growth in children, improved appetite, and reduced fatigue. Improvements in or lessening the severity of any of these symptoms can be readily assessed according to methods and techniques known in the art or subsequently developed.

The terms “about” and “approximately”, when used in connection with temperatures, peaks, signals, doses, amounts, or weight percent of ingredients of a composition or a dosage form, include the value of a specified temperature, peak, signal, dose, amount, or weight percent or a range of the dose, amount, or weight percent that is recognized by one of ordinary skill in the art to provide a pharmacological effect equivalent to that obtained from the specified temperature, peak, signal, dose, amount, or weight percent. Typically, the term “about” refers to a variation of up to 10%, up to 5%, or up to 2% of a stated value.

The compounds of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing may be administered once daily, twice daily, or three times daily for the treatment of AATD. In some embodiments, the any one or more compounds are selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67, tautomers of those compounds, deuterated derivatives of those compounds or tautomers, and pharmaceutically acceptable salts of any of the foregoing. In some embodiments, at least one compound chosen from compounds of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing is administered once daily. In some embodiments, a compound selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67), tautomers of those compounds, deuterated derivatives of those compounds or tautomers, and pharmaceutically acceptable salts of any of the foregoing is administered once daily. In some embodiments, at least one compound selected from compounds of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing are administered twice daily. In some embodiments, a compound selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67), tautomers of those compounds, deuterated derivatives of those compounds or tautomers, and pharmaceutically acceptable salts of any of the foregoing is administered twice daily. In some embodiments, at least one compound chosen from compounds of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing are administered three times daily. In some embodiments, a compound selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67), tautomers of those compounds, deuterated derivatives of those compounds or tautomers, and pharmaceutically acceptable salts of any of the foregoing is administered three times daily.

Any one or more of the compounds of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing may be administered in combination with AAT augmentation therapy or AAT replacement therapy for the treatment of AATD. In some embodiments, the any one or more compounds are selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67), tautomers of those compounds, deuterated derivatives of those compounds or tautomers, and pharmaceutically acceptable salts of any of the foregoing.

As used herein, “AAT augmentation therapy” refers to the use of alpha-1 antitrypsin protein (AAT) from the blood plasma of healthy human donors to augment (increase) the alpha-1 antitrypsin levels circulating in the blood. “AAT replacement therapy” refers to administration of recombinant AAT.

In some embodiments, 5 mg to 1,000 mg, 10 mg to 1,500 mg, 100 mg to 1,800 mg, 100 mg to 500 mg, 200 mg to 600 mg, 200 mg to 800 mg, 400 mg to 2,000 mg, 400 mg to 2,500 mg or 400 mg to 600 mg of a compound of Formula I, including compounds of Formula Ia, Ib, Ib-i, Ib-ii, and Ic, tautomers of those compounds, deuterated derivatives of those compounds or tautomers, and pharmaceutically acceptable salts of any of the foregoing is administered once daily, twice daily, or three times daily. In some embodiments, 5 mg to 1,000 mg, 10 mg to 1,500 mg, 100 mg to 1,800 mg, 100 mg to 500 mg, 200 mg to 600 mg, 200 mg to 800 mg, 400 mg to 2000 mg, or 400 mg to 600 mg of a compound selected from Compounds 1 to 67 (e.g., Compounds 1 to 5; Compounds 6 to 21, 33 to 42, 44 to 50, 52, 53, and 57; Compounds 22 to 32, 43, 51, and 54 to 56; and Compounds 58 to 67), is administered once daily, twice daily, or three times daily.

One of ordinary skill in the art would recognize that, when an amount of a compound is disclosed, the relevant amount of a pharmaceutically acceptable salt form of the compound is an amount equivalent to the concentration of the free base of the compound. It is noted that the disclosed amounts of the compounds, tautomers, deuterated derivatives, and pharmaceutically acceptable salts are based upon the free base form of the reference compound. For example, “10 mg of at least one compound chosen from compounds of Formulae Ia, Ib, or Ic and pharmaceutically acceptable salts thereof” includes 10 mg of a compound of Formulae Ia, Ib, or Ic and a concentration of a pharmaceutically acceptable salt of compounds of Formulae Ia, Ib, or Ic equivalent to 10 mg of compounds of Formulae Ia, Ib, or Ic.

It should be understood that references herein to methods of treatment (e.g., methods of treating AATD) using one or more compounds (e.g., compounds of Formula I, including compounds of Formula Ia, Ib, Ib-i, Ib-ii, and Ic, as well as tautomers of those compounds, deuterated derivatives of those compounds and tautomers, and pharmaceutically acceptable salts of those compounds) should also be interpreted as references to:
one or more compounds (e.g., compounds of Formula I, including compounds of Formula Ia, Ib, Ib-i, Ib-ii, and Ic, as well as tautomers of those compounds, deuterated derivatives of those compounds and tautomers, and pharmaceutically acceptable salts of those compounds) for use in methods of treating, e.g., AATD; and/or
the use of one or more compounds (e.g., compounds of Formula I, including compounds of Formula Ia, Ib, Ib-i, Ib-ii, and Ic, as well as tautomers of those compounds, deuterated derivatives of those compounds and tautomers, and pharmaceutically acceptable salts of those compounds) in the manufacture of a medicament for treating, e.g., AATD.

### II. SOLID FORMS OF COMPOUNDS WITH IMPROVED COMPOUND QUALITY VALUES

Non-limiting exemplary embodiments of the disclosure include:
1. Substantially crystalline Compound 5 free form Monohydrate Form A:

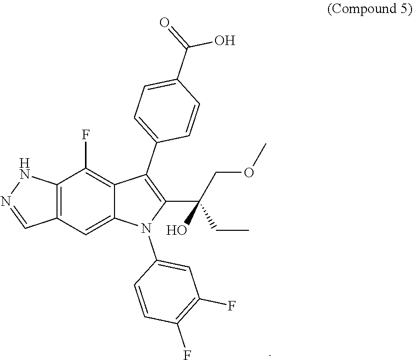 [SMILES: CC[C@@](O)(COC)c1c(-c2ccc(C(=O)O)cc2)c2c(F)c3[nH]ncc3cc2n1-c1ccc(F)c(F)c1]

2. The substantially crystalline Compound 5 free form Monohydrate Form A according to Embodiment 1, characterized by one or more XRPD signals selected from 12.1±0.2 degrees two theta, 19.5±0.2 degrees two theta, and 19.8±0.2 degrees two theta.
3. The substantially crystalline Compound 5 free form Monohydrate Form A according to Embodiment 1, characterized by two or more XRPD signals selected from 12.1±0.2 degrees two theta, 19.5±0.2 degrees two theta, and 19.8±0.2 degrees two theta.
4. The substantially crystalline Compound 5 free form Monohydrate Form A according to Embodiment 1, characterized by XRPD signals at 12.1±0.2 degrees two theta, 19.5±0.2 degrees two theta, and 19.8±0.2 degrees two theta.
5. The substantially crystalline Compound 5 free form Monohydrate Form A according to Embodiment 1, characterized by (a) one or more XRPD signals selected from 12.1±0.2 degrees two theta, 19.5±0.2 degrees two theta, and 19.8±0.2 degrees two theta; and (b) one or more XRPD signals selected from 8.2±0.2 degrees two theta, 10.3±0.2 degrees two theta, 23.2±0.2 degrees two theta, 17.1±0.2 degrees two theta, and 21.0±0.2 degrees two theta.
6. The substantially crystalline Compound 5 free form Monohydrate Form A according to Embodiment 1, characterized by (a) one or more XRPD signals selected from 12.1±0.2 degrees two theta, 19.5±0.2 degrees two theta, and 19.8±0.2 degrees two theta; and (b) two or more XRPD signals selected from 8.2±0.2 degrees two theta, 10.3±0.2 degrees two theta, 23.2±0.2 degrees two theta, 17.1±0.2 degrees two theta, and 21.0±0.2 degrees two theta.
7. The substantially crystalline Compound 5 free form Monohydrate Form A according to Embodiment 1, characterized by (a) one or more XRPD signals selected from 12.1±0.2 degrees two theta, 19.5±0.2 degrees two theta, and 19.8±0.2 degrees two theta; and (b) three or more XRPD signals selected from 8.2±0.2 degrees two theta, 10.3±0.2 degrees two theta, 23.2±0.2 degrees two theta, 17.1±0.2 degrees two theta, and 21.0±0.2 degrees two theta.
8. The substantially crystalline Compound 5 free form Monohydrate Form A according to Embodiment 1, characterized by (a) one or more XRPD signals selected from 12.1±0.2 degrees two theta, 19.5±0.2 degrees two theta, and 19.8±0.2 degrees two theta; and (b) four or more XRPD signals selected from 8.2±0.2 degrees two theta, 10.3±0.2 degrees two theta, 23.2±0.2 degrees two theta, 17.1±0.2 degrees two theta, and 21.0±0.2 degrees two theta.
9. The substantially crystalline Compound 5 free form Monohydrate Form A according to Embodiment 1, characterized by (a) one or more XRPD signals selected from 12.1±0.2 degrees two theta, 19.5±0.2 degrees two theta, and 19.8±0.2 degrees two theta; and (b) XRPD signals at 8.2±0.2 degrees two theta, 10.3±0.2 degrees two theta, 23.2±0.2 degrees two theta, 17.1±0.2 degrees two theta, and 21.0±0.2 degrees two theta.
10. The substantially crystalline Compound 5 free form Monohydrate Form A according to Embodiment 1, characterized by (a) two or more XRPD signals selected from 12.1±0.2 degrees two theta, 19.5±0.2 degrees two theta, and 19.8±0.2 degrees two theta; and (b) XRPD signals at 8.2±0.2 degrees two theta, 10.3±0.2 degrees two theta, 23.2±0.2 degrees two theta, 17.1±0.2 degrees two theta, and 21.0±0.2 degrees two theta.
11. The substantially crystalline Compound 5 free form Monohydrate Form A according to Embodiment 1, characterized by (a) XRPD signals at 12.1±0.2 degrees two theta, 19.5±0.2 degrees two theta, and 19.8±0.2 degrees two theta; and (b) XRPD signals at 8.2±0.2 degrees two theta, 10.3±0.2 degrees two theta, 23.2±0.2 degrees two theta, 17.1±0.2 degrees two theta, and 21.0±0.2 degrees two theta.
12. The substantially crystalline Compound 5 free form Monohydrate Form A according to Embodiment 1, characterized by a thermogravimetric analysis (TGA) showing about 3.3% weight loss from ambient temperature up to about 160° C.
13. The substantially crystalline Compound 5 free form Monohydrate Form A according to Embodiment 1, characterized by a differential scanning calorimetry (DSC) analysis showing endothermic peaks at about 141° C. and 163° C.
14. The substantially crystalline Compound 5 free form Monohydrate Form A according to Embodiment 1, characterized by a ¹³C CPMAS having one or more signals selected from 140.9±0.2 ppm, 131.0±0.2 ppm, 128.1±0.2 ppm, 124.8±0.2 ppm, 59.6±0.2 ppm, and 7.6±0.2 ppm.
15. The substantially crystalline Compound 5 free form Monohydrate Form A according to Embodiment 1, characterized by a ¹³C CPMAS having two or more signals selected from 140.9±0.2 ppm, 131.0±0.2 ppm, 128.1±0.2 ppm, 124.8±0.2 ppm, 59.6±0.2 ppm, and 7.6±0.2 ppm.
16. The substantially crystalline Compound 5 free form Monohydrate Form A according to Embodiment 1, characterized by a ¹³C CPMAS having three or more signals selected from 140.9±0.2 ppm, 131.0±0.2 ppm, 128.1±0.2 ppm, 124.8±0.2 ppm, 59.6±0.2 ppm, and 7.6±0.2 ppm.
17. The substantially crystalline Compound 5 free form Monohydrate Form A according to Embodiment 1, characterized by a ¹³C CPMAS having four or more signals selected from 140.9±0.2 ppm, 131.0±0.2 ppm, 128.1±0.2 ppm, 124.8±0.2 ppm, 59.6±0.2 ppm, and 7.6±0.2 ppm.
18. The substantially crystalline Compound 5 free form Monohydrate Form A according to Embodiment 1, characterized by a ¹³C CPMAS having five or more signals selected from 140.9±0.2 ppm, 131.0±0.2 ppm, 128.1±0.2 ppm, 124.8±0.2 ppm, 59.6±0.2 ppm, and 7.6±0.2 ppm.
19. The substantially crystalline Compound 5 free form Monohydrate Form A according to Embodiment 1, characterized by a ¹³C CPMAS having signals at 140.9±0.2 ppm, 131.0±0.2 ppm, 128.1±0.2 ppm, 124.8±0.2 ppm, 59.6±0.2 ppm, and 7.6±0.2 ppm.
20. The substantially crystalline Compound 5 free form Monohydrate Form A according to Embodiment 1, characterized by a ¹⁹F MAS having one or more signals selected from −134.4±0.2 ppm, −136.9±0.2 ppm, −143.5±0.2 ppm, and −146.8±0.2 ppm.
21. The substantially crystalline Compound 5 free form Monohydrate Form A according to Embodiment 1, characterized by a ¹⁹F MAS having two or more signals selected from −134.4±0.2 ppm, −136.9±0.2 ppm, −143.5±0.2 ppm, and −146.8±0.2 ppm.
22. The substantially crystalline Compound 5 free form Monohydrate Form A according to Embodiment 1, characterized by a ¹⁹F MAS having three or more signals selected from −134.4±0.2 ppm, −136.9±0.2 ppm, −143.5±0.2 ppm, and −146.8±0.2 ppm.
23. The substantially crystalline Compound 5 free form Monohydrate Form A according to Embodiment 1, characterized by a ¹⁹F MAS having signals at −134.4±0.2 ppm, −136.9±0.2 ppm, −143.5±0.2 ppm, and −146.8±0.2 ppm.
24. The substantially crystalline Compound 5 free form Monohydrate Form A according to Embodiment 1, characterized by a monoclinic crystal system, a P2₁ space group, and unit cell dimensions measured at 100 K on a Bruker diffractometer equipped with Cu K, radiation (λ=1.54178 Å) and a CMOS detector of:

| | | | |
|---|---|---|---|
| a | 9.7 ± 0.1 Å | α | 90° |
| b | 12.7 ± 0.1 Å | β | 92.4° ± 0.1º |
| c | 20.1 ± 0.1 Å | γ | 90°. |

25. The substantially crystalline Compound 5 free form Monohydrate Form A according to Embodiment 1, characterized by a monoclinic crystal system, a P2₁ space group, and unit cell dimensions measured at 293 K on a Bruker diffractometer equipped with Cu K, radiation (λ=1.54178 Å) and a CMOS detector of:

| | | | |
|---|---|---|---|
| a | 9.8 ± 0.1 Å | α | 90° |
| b | 12.7 ± 0.1 Å | β | 93.0° ± 0.1º |
| c | 20.3 ± 0.1 Å | γ | 90°. |

26. Substantially crystalline Compound 5 free form Form A:

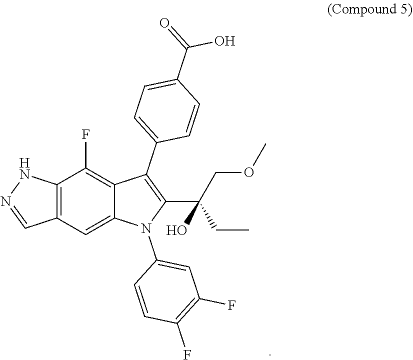 [SMILES: CC[C@@](O)(COC)c1c(-c2ccc(C(=O)O)cc2)c2c(F)c3[nH]ncc3cc2n1-c1ccc(F)c(F)c1]

27. The substantially crystalline Compound 5 free form Form A according to Embodiment 26, characterized by an XRPD signal at 3.3±0.2 degrees two theta or 8.1±0.2 degrees two theta.
28. The substantially crystalline Compound 5 free form Form A according to Embodiment 26, characterized by XRPD signals at 3.3±0.2 degrees two theta and 8.1±0.2 degrees two theta.
29. The substantially crystalline Compound 5 free form Form A according to Embodiment 26, characterized XRPD signals at 3.3±0.2 degrees two theta, 8.1±0.2 degrees two theta, and 10.7±0.2 degrees two theta.
30. The substantially crystalline Compound 5 free form Form A according to Embodiment 26, characterized by (a) an XRPD signal at 3.3±0.2 degrees two theta or 8.1±0.2 degrees two theta; and (b) one or more XRPD signals selected from 3.0±0.2 degrees two theta, 7.1±0.2 degrees two theta, 8.6±0.2 degrees two theta, 15.5±0.2 degrees two theta, and 22.1±0.2 degrees two theta.
31. The substantially crystalline Compound 5 free form Form A according to Embodiment 26, characterized by (a) an XRPD signal at 3.3±0.2 degrees two theta or 8.1±0.2 degrees two theta; and (b) two or more XRPD signals selected from 3.0±0.2 degrees two theta, 7.1±0.2 degrees two theta, 8.6±0.2 degrees two theta, 15.5±0.2 degrees two theta, and 22.1±0.2 degrees two theta.
32. The substantially crystalline Compound 5 free form Form A according to Embodiment 26, characterized by (a) an XRPD signal at 3.3±0.2 degrees two theta or 8.1±0.2 degrees two theta; and (b) three or more XRPD signals selected from 3.0±0.2 degrees two theta, 7.1±0.2 degrees two theta, 8.6±0.2 degrees two theta, 15.5±0.2 degrees two theta, and 22.1±0.2 degrees two theta.
33. The substantially crystalline Compound 5 free form Form A according to Embodiment 26, characterized by (a) an XRPD signal at 3.3±0.2 degrees two theta or 8.1±0.2 degrees two theta; and (b) four or more XRPD signals selected from 3.0±0.2 degrees two theta, 7.1±0.2 degrees two theta, 8.6±0.2 degrees two theta, 15.5±0.2 degrees two theta, and 22.1±0.2 degrees two theta.
34. The substantially crystalline Compound 5 free form Form A according to Embodiment 26, characterized by (a) an XRPD signal at 3.3±0.2 degrees two theta or 8.1±0.2 degrees two theta; and (b) XRPD signals at 3.0±0.2 degrees two theta, 7.1±0.2 degrees two theta, 8.6±0.2 degrees two theta, 15.5±0.2 degrees two theta, and 22.1±0.2 degrees two theta.
35. The substantially crystalline Compound 5 free form Form A according to Embodiment 26, characterized by (a) XRPD signals at 3.3±0.2 degrees two theta and 8.1±0.2 degrees two theta; and (b) XRPD signals at 3.0±0.2 degrees two theta, 7.1±0.2 degrees two theta, 8.6±0.2 degrees two theta, 15.5±0.2 degrees two theta, and 22.1±0.2 degrees two theta.
36. The substantially crystalline Compound 5 free form Form A according to Embodiment 26, characterized by (a) XRPD signals at 3.3±0.2 degrees two theta, 8.1±0.2 degrees two theta, and 10.7±0.2 degrees two theta; and (b) 3.0±0.2 degrees two theta, 7.1±0.2 degrees two theta, 8.6±0.2 degrees two theta, 15.5±0.2 degrees two theta, and 22.1±0.2 degrees two theta.
37. The substantially crystalline Compound 5 free form Form A according to Embodiment 26, characterized by a thermogravimetric analysis showing about 0.2% weight loss from ambient temperature up to about 180° C.
38. The substantially crystalline Compound 5 free form Form A according to Embodiment 26, characterized by a differential scanning calorimetry analysis showing an endothermic peak at about 193° C.
39. The substantially crystalline Compound 5 free form Form A according to Embodiment 26, characterized by a ¹³C CPMAS having one or more signals selected from 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
40. The substantially crystalline Compound 5 free form Form A according to Embodiment 26, characterized by a ¹³C CPMAS having two or more signals selected from 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
41. The substantially crystalline Compound 5 free form Form A according to Embodiment 26, characterized by a ¹³C CPMAS having three or more signals selected from 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
42. The substantially crystalline Compound 5 free form Form A according to Embodiment 26, characterized by a ¹³C CPMAS having four or more signals selected from 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
43. The substantially crystalline Compound 5 free form Form A according to Embodiment 26, characterized by a ¹³C CPMAS having five or more signals selected from 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
44. The substantially crystalline Compound 5 free form Form A according to Embodiment 26, characterized by a ¹³C CPMAS having signals at 77.7±0.2 ppm, 117.9±0.2 ppm, 125.8±0.2 ppm, 128.5±0.2 ppm, 130.1±0.2 ppm, 132.0±0.2 ppm, and 138.4±0.2 ppm.
45. The substantially crystalline Compound 5 free form Form A according to Embodiment 26, characterized by a ¹⁹F MAS having one or more signals selected from −134.3±0.2 ppm, −136.4±0.2 ppm, −137.4±0.2 ppm, −138.6±0.2 ppm, −140.1±0.2 ppm, and −141.6±0.2 ppm.
46. The substantially crystalline Compound 5 free form Form A according to Embodiment 26, characterized by a ¹⁹F MAS having two or more signals selected from −134.3±0.2 ppm, −136.4±0.2 ppm, −137.4±0.2 ppm, −138.6±0.2 ppm, −140.1±0.2 ppm, and −141.6±0.2 ppm.
47. The substantially crystalline Compound 5 free form Form A according to Embodiment 26, characterized by a ¹⁹F MAS having three or more signals selected from −134.3±0.2 ppm, −136.4±0.2 ppm, −137.4±0.2 ppm, −138.6±0.2 ppm, −140.1±0.2 ppm, and −141.6±0.2 ppm.
48. The substantially crystalline Compound 5 free form Form A according to Embodiment 26, characterized by a ¹⁹F MAS having four or more signals selected from −134.3±0.2 ppm, −136.4±0.2 ppm, −137.4±0.2 ppm, −138.6±0.2 ppm, −140.1±0.2 ppm, and −141.6±0.2 ppm.
49. The substantially crystalline Compound 5 free form Form A according to Embodiment 26, characterized by a ¹⁹F MAS having signals at −134.3±0.2 ppm, −136.4±0.2 ppm, −137.4±0.2 ppm, −138.6±0.2 ppm, −140.1±0.2 ppm, and −141.6±0.2 ppm.
50. The substantially crystalline Compound 5 free form Form A according to Embodiment 26, characterized by a triclinic crystal system, a P1 space group, and unit cell dimensions measured at 100 K on a Bruker diffractometer equipped with Cu K_{α} radiation (λ=1.54178 Å) and a CMOS detector of:

| | | | |
|---|---|---|---|
| a | 12.9 ± 0.1 Å | α | 93.8° ± 0.1° |
| b | 15.6 ± 0.1 Å | β | 99.7° ± 0.1° |
| c | 26.6 ± 0.1 Å | γ | 108.8° ± 0.1°. |

51. Substantially crystalline Compound 5 free form Form B:

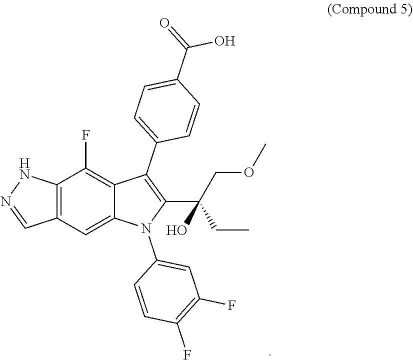 [SMILES: CC[C@@](O)(COC)c1c(-c2ccc(C(=O)O)cc2)c2c(F)c3[nH]ncc3cc2n1-c1ccc(F)c(F)c1]

52. The substantially crystalline Compound 5 free form Form B according to Embodiment 51, characterized by one or more XRPD signals selected from 9.8±0.2 degrees two theta, 11.2±0.2 degrees two theta, 20.3±0.2 degrees two theta, and 28.1±0.2 degrees two theta.
53. The substantially crystalline Compound 5 free form Form B according to Embodiment 51, characterized by two or more XRPD signals selected from 9.8±0.2 degrees two theta, 11.2±0.2 degrees two theta, 20.3±0.2 degrees two theta, and 28.1±0.2 degrees two theta.
54. The substantially crystalline Compound 5 free form Form B according to Embodiment 51, characterized by three or more XRPD signals selected from 9.8±0.2 degrees two theta, 11.2±0.2 degrees two theta, 20.3±0.2 degrees two theta, and 28.1±0.2 degrees two theta.
54. The substantially crystalline Compound 5 free form Form B according to Embodiment 51, characterized by XRPD signals at 9.8±0.2 degrees two theta, 11.2±0.2 degrees two theta, 20.3±0.2 degrees two theta, and 28.1±0.2 degrees two theta.
55. The substantially crystalline Compound 5 free form Form B according to Embodiment 51, characterized by (a) one or more XRPD signals selected from 9.8±0.2 degrees two theta, 11.2±0.2 degrees two theta, 20.3±0.2 degrees two theta, and 28.1±0.2 degrees two theta; and (b) one or more XRPD signals selected from 9.4±0.2 degrees two theta, 11.8±0.2 degrees two theta, 17.1±0.2 degrees two theta, 18.4±0.2 degrees two theta, and 23.9±0.2 degrees two theta.
56. The substantially crystalline Compound 5 free form Form B according to Embodiment 51, characterized by (a) one or more XRPD signals selected from 9.8±0.2 degrees two theta, 11.2±0.2 degrees two theta, 20.3±0.2 degrees two theta, and 28.1±0.2 degrees two theta; and (b) two or more XRPD signals selected from 9.4±0.2 degrees two theta, 11.8±0.2 degrees two theta, 17.1±0.2 degrees two theta, 18.4±0.2 degrees two theta, and 23.9±0.2 degrees two theta.
57. The substantially crystalline Compound 5 free form Form B according to Embodiment 51, characterized by (a) one or more XRPD signals selected from 9.8±0.2 degrees two theta, 11.2±0.2 degrees two theta, 20.3±0.2 degrees two theta, and 28.1±0.2 degrees two theta; and (b) three or more XRPD signals selected from 9.4±0.2 degrees two theta, 11.8±0.2 degrees two theta, 17.1±0.2 degrees two theta, 18.4±0.2 degrees two theta, and 23.9±0.2 degrees two theta.
58. The substantially crystalline Compound 5 free form Form B according to Embodiment 51, characterized by (a) one or more XRPD signals selected from 9.8±0.2 degrees two theta, 11.2±0.2 degrees two theta, 20.3±0.2 degrees two theta, and 28.1±0.2 degrees two theta; and (b) four or more XRPD signals selected from 9.4±0.2 degrees two theta, 11.8±0.2 degrees two theta, 17.1±0.2 degrees two theta, 18.4±0.2 degrees two theta, and 23.9±0.2 degrees two theta.
59. The substantially crystalline Compound 5 free form Form B according to Embodiment 51, characterized by (a) one or more XRPD signals selected from 9.8±0.2 degrees two theta, 11.2±0.2 degrees two theta, 20.3±0.2 degrees two theta, and 28.1±0.2 degrees two theta; and (b) XRPD signals at 9.4±0.2 degrees two theta, 11.8±0.2 degrees two theta, 17.1±0.2 degrees two theta, 18.4±0.2 degrees two theta, and 23.9±0.2 degrees two theta.
60. The substantially crystalline Compound 5 free form Form B according to Embodiment 51, characterized by (a) two or more XRPD signals selected from 9.8±0.2 degrees two theta, 11.2±0.2 degrees two theta, 20.3±0.2 degrees two theta, and 28.1±0.2 degrees two theta; and (b) XRPD signals at 9.4±0.2 degrees two theta, 11.8±0.2 degrees two theta, 17.1±0.2 degrees two theta, 18.4±0.2 degrees two theta, and 23.9±0.2 degrees two theta.
61. The substantially crystalline Compound 5 free form Form B according to Embodiment 51, characterized by (a) three or more XRPD signals selected from 9.8±0.2 degrees two theta, 11.2±0.2 degrees two theta, 20.3±0.2 degrees two theta, and 28.1±0.2 degrees two theta; and (b) XRPD signals at 9.4±0.2 degrees two theta, 11.8±0.2 degrees two theta, 17.1±0.2 degrees two theta, 18.4±0.2 degrees two theta, and 23.9±0.2 degrees two theta.
62. The substantially crystalline Compound 5 free form Form B according to Embodiment 51, characterized by (a) XRPD signals at 9.8±0.2 degrees two theta, 11.2±0.2 degrees two theta, 20.3±0.2 degrees two theta, and 28.1±0.2 degrees two theta; and (b) signals at 9.4±0.2 degrees two theta, 11.8±0.2 degrees two theta, 17.1±0.2 degrees two theta, 18.4±0.2 degrees two theta, and 23.9±0.2 degrees two theta.
63. The substantially crystalline Compound 5 free form Form B according to Embodiment 51, characterized by a thermogravimetric analysis (TGA) showing about 1.0% weight loss from ambient temperature up to about 200° C.
64. The substantially crystalline Compound 5 free form Form B according to Embodiment 51, characterized by a differential scanning calorimetry (DSC) analysis showing an endothermic peak at about 240° C.
65. Substantially crystalline Compound 5 free form NPA Solvate Form A:

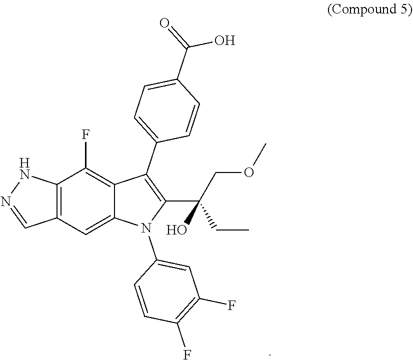 [SMILES: CC[C@@](O)(COC)c1c(-c2ccc(C(=O)O)cc2)c2c(F)c3[nH]ncc3cc2n1-c1ccc(F)c(F)c1]

66. The substantially crystalline Compound 5 free form NPA Solvate Form A according to Embodiment 65, characterized by one or more XRPD signals selected from 7.4±0.2 degrees two theta, 10.1±0.2 degrees two theta, and 22.1±0.2 degrees two theta.
67. The substantially crystalline Compound 5 free form NPA Solvate Form A according to Embodiment 65, characterized by two or more XRPD signals selected from 7.4±0.2 degrees two theta, 10.1±0.2 degrees two theta, and 22.1±0.2 degrees two theta.
68. The substantially crystalline Compound 5 free form NPA Solvate Form A according to Embodiment 65, characterized by XRPD signals at 7.4±0.2 degrees two theta, 10.1±0.2 degrees two theta, and 22.1±0.2 degrees two theta.
69. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) one or more XRPD signals selected from 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
70. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) two or more XRPD signals selected from 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
71. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) three or more XRPD signals selected from 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
72. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) four or more XRPD signals selected from 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
73. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) five or more XRPD signals selected from 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
74. The substantially crystalline Compound 5 free form NPA Solvate Form A according to Embodiment 65, characterized by (a) one or more XRPD signals selected from 7.4±0.2 degrees two theta, 10.1±0.2 degrees two theta, and 22.1±0.2 degrees two theta; and (b) XRPD signals at 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
75. The substantially crystalline Compound 5 free form NPA Solvate Form A according to Embodiment 65, characterized by (a) two or more XRPD signals selected from 7.4±0.2 degrees two theta, 10.1±0.2 degrees two theta, and 22.1±0.2 degrees two theta; and (b) XRPD signals at 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
76. The substantially crystalline Compound 5 free form NPA Solvate Form A according to Embodiment 65, characterized by (a) XRPD signals at 7.4±0.2 degrees two theta, 10.1±0.2 degrees two theta, and 22.1±0.2 degrees two theta; and (b) XRPD signals at 9.6±0.2 degrees two theta, 10.2±0.2 degrees two theta, 18.0±0.2 degrees two theta, 17.2±0.2 degrees two theta, 21.0±0.2 degrees two theta, and 21.4±0.2 degrees two theta.
77. The substantially crystalline Compound 5 free form NPA Solvate Form A according to Embodiment 65, characterized by a thermogravimetric analysis (TGA) showing about 8.7% weight loss from ambient temperature up to about 160° C.
78. The substantially crystalline Compound 5 free form NPA Solvate Form A according to Embodiment 65, characterized by a differential scanning calorimetry analysis showing endothermic peaks at about 133° C., 166° C., and 219° C.
79. Substantially crystalline Compound 5 free form EtOH Solvate Form A:

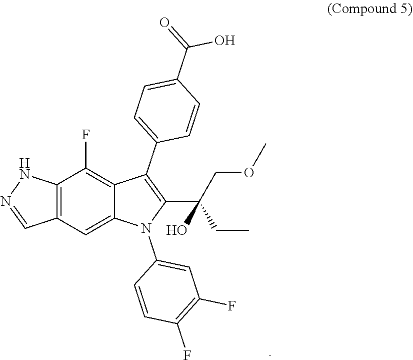 [SMILES: CC[C@@](O)(COC)c1c(-c2ccc(C(=O)O)cc2)c2c(F)c3[nH]ncc3cc2n1-c1ccc(F)c(F)c1]

80. The substantially crystalline Compound 5 free form EtOH Solvate Form A according to Embodiment 79, characterized by one or more XRPD signals selected from 7.7±0.2 degrees two theta, 10.2±0.2 degrees two theta, and 18.7±0.2 degrees two theta.
81. The substantially crystalline Compound 5 free form EtOH Solvate Form A according to Embodiment 79, characterized by two or more XRPD signals selected from 7.7±0.2 degrees two theta, 10.2±0.2 degrees two theta, and 18.7±0.2 degrees two theta.
82. The substantially crystalline Compound 5 free form EtOH Solvate Form A according to Embodiment 79, characterized by XRPD signals at 7.7±0.2 degrees two theta, 10.2±0.2 degrees two theta, and 18.7±0.2 degrees two theta.
83. The substantially crystalline Compound 5 free form EtOH Solvate Form A according to Embodiment 79, characterized by (a) one or more XRPD signals selected from 7.7±0.2 degrees two theta, 10.2±0.2 degrees two theta, and 18.7±0.2 degrees two theta; and (b) one or more XRPD signals selected from 10.4±0.2 degrees two theta, 12.4±0.2 degrees two theta, 17.2±0.2 degrees two theta, 23.1±0.2 degrees two theta, and 25.1±0.2 degrees two theta.
84. The substantially crystalline Compound 5 free form EtOH Solvate Form A according to Embodiment 79, characterized by (a) one or more XRPD signals selected from 7.7±0.2 degrees two theta, 10.2±0.2 degrees two theta, and 18.7±0.2 degrees two theta; and (b) two or more XRPD signals selected from 10.4±0.2 degrees two theta, 12.4±0.2 degrees two theta, 17.2±0.2 degrees two theta, 23.1±0.2 degrees two theta, and 25.1±0.2 degrees two theta.
85. The substantially crystalline Compound 5 free form EtOH Solvate Form A according to Embodiment 79, characterized by (a) one or more XRPD signals selected from 7.7±0.2 degrees two theta, 10.2±0.2 degrees two theta, and 18.7±0.2 degrees two theta; and (b) three or more XRPD signals selected from 10.4±0.2 degrees two theta, 12.4±0.2 degrees two theta, 17.2±0.2 degrees two theta, 23.1±0.2 degrees two theta, and 25.1±0.2 degrees two theta.
86. The substantially crystalline Compound 5 free form EtOH Solvate Form A according to Embodiment 79, characterized by (a) one or more XRPD signals selected from 7.7±0.2 degrees two theta, 10.2±0.2 degrees two theta, and 18.7±0.2 degrees two theta; and (b) four or more XRPD signals selected from 10.4±0.2 degrees two theta, 12.4±0.2 degrees two theta, 17.2±0.2 degrees two theta, 23.1±0.2 degrees two theta, and 25.1±0.2 degrees two theta.
87. The substantially crystalline Compound 5 free form EtOH Solvate Form A according to Embodiment 79, characterized by (a) one or more XRPD signals selected from 7.7±0.2 degrees two theta, 10.2±0.2 degrees two theta, and 18.7±0.2 degrees two theta; and (b) XRPD signals at 10.4±0.2 degrees two theta, 12.4±0.2 degrees two theta, 17.2±0.2 degrees two theta, 23.1±0.2 degrees two theta, and 25.1±0.2 degrees two theta.
88. The substantially crystalline Compound 5 free form EtOH Solvate Form A according to Embodiment 79, characterized by (a) two or more XRPD signals selected from 7.7±0.2 degrees two theta, 10.2±0.2 degrees two theta, and 18.7±0.2 degrees two theta; and (b) XRPD signals at 10.4±0.2 degrees two theta, 12.4±0.2 degrees two theta, 17.2±0.2 degrees two theta, 23.1±0.2 degrees two theta, and 25.1±0.2 degrees two theta.
89. The substantially crystalline Compound 5 free form EtOH Solvate Form A according to Embodiment 79, characterized by (a) XRPD signals at 7.7±0.2 degrees two theta, 10.2±0.2 degrees two theta, and 18.7±0.2 degrees two theta; and (b) XRPD signals at 10.4±0.2 degrees two theta, 12.4±0.2 degrees two theta, 17.2±0.2 degrees two theta, 23.1±0.2 degrees two theta, and 25.1±0.2 degrees two theta.
90. The substantially crystalline Compound 5 free form EtOH Solvate Form A according to Embodiment 79, characterized by a thermogravimetric analysis (TGA) showing about 7.0% weight loss from ambient temperature up to about 180° C.
91. The substantially crystalline Compound 5 free form EtOH Solvate Form A according to Embodiment 79, characterized by a differential scanning calorimetry (DSC) analysis showing an endothermic peak at about 163° C.
92. The substantially crystalline Compound 5 free form EtOH Solvate Form A according to Embodiment 79, characterized by a ¹³C CPMAS having one or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
93. The substantially crystalline Compound 5 free form EtOH Solvate Form A according to Embodiment 79, characterized by a ¹³C CPMAS having two or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
94. The substantially crystalline Compound 5 free form EtOH Solvate Form A according to Embodiment 79, characterized by a ¹³C CPMAS having three or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
95. The substantially crystalline Compound 5 free form EtOH Solvate Form A according to Embodiment 79, characterized by a ¹³C CPMAS having four or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
96. The substantially crystalline Compound 5 free form EtOH Solvate Form A according to Embodiment 79, characterized by a ¹³C CPMAS having five or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
97. The substantially crystalline Compound 5 free form EtOH Solvate Form A according to Embodiment 79, characterized by a ¹³C CPMAS having six or more signals selected from 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
98. The substantially crystalline Compound 5 free form EtOH Solvate Form A according to Embodiment 79, characterized by a ¹³C CPMAS having signals at 59.0±0.2 ppm, 76.2±0.2 ppm, 117.4±0.2 ppm, 123.6±0.2 ppm, 126.7±0.2 ppm, 127.6±0.2 ppm, 129.6±0.2 ppm, 132.1±0.2 ppm, and 140.2±0.2 ppm.
99. The substantially crystalline Compound 5 free form EtOH Solvate Form A according to Embodiment 79, characterized by a ¹⁹F MAS having one or more signals selected from −130.0±0.2 ppm, −131.7±0.2 ppm, −135.6±0.2 ppm, and −139.0±0.2 ppm.
100. The substantially crystalline Compound 5 free form EtOH Solvate Form A according to Embodiment 79, characterized by a ¹⁹F MAS having two or more signals selected from −130.0±0.2 ppm, −131.7±0.2 ppm, −135.6±0.2 ppm, and −139.0±0.2 ppm.
101. The substantially crystalline Compound 5 free form EtOH Solvate Form A according to Embodiment 79, characterized by a ¹⁹F MAS having three or more signals selected from −130.0±0.2 ppm, −131.7±0.2 ppm, −135.6±0.2 ppm, and −139.0±0.2 ppm.
102. The substantially crystalline Compound 5 free form EtOH Solvate Form A according to Embodiment 79, characterized by a ¹⁹F MAS having signals at −130.0±0.2 ppm, −131.7±0.2 ppm, −135.6±0.2 ppm, and −139.0±0.2 ppm.
103. The substantially crystalline Compound 5 free form EtOH Solvate Form A according to Embodiment 79, characterized by a triclinic crystal system, a P1 space group, and unit cell dimensions measured at 100 K on a Bruker diffractometer equipped with Cu K, radiation (λ=1.54178 Å) and a CMOS detector of:

| a | 10.5 ± 0.1 Å | α | 98.3° ± 0.1º |
|---|---|---|---|
| b | 11.1 ± 0.1 Å | β | 92.4° ± 0.1º |
| c | 11.4 ± 0.1 Å | γ | 104.2° ± 0.1°. |

104. Substantially crystalline Compound 5 free form MeOH Solvate Hydrate Form A:

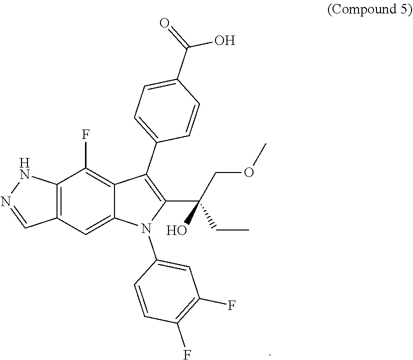 [SMILES: CC[C@@](O)(COC)c1c(-c2ccc(C(=O)O)cc2)c2c(F)c3[nH]ncc3cc2n1-c1ccc(F)c(F)c1]

105. The substantially crystalline Compound 5 free form MeOH Solvate Hydrate Form A according to Embodiment 104, characterized by one or more XRPD signals selected from 7.7±0.2 degrees two theta, 10.4±0.2 degrees two theta, and 19.0±0.2 degrees two theta.
106. The substantially crystalline Compound 5 free form MeOH Solvate Hydrate Form A according to Embodiment 104, characterized by two or more XRPD signals selected from 7.7±0.2 degrees two theta, 10.4±0.2 degrees two theta, and 19.0±0.2 degrees two theta.
107. The substantially crystalline Compound 5 free form MeOH Solvate Hydrate Form A according to Embodiment 104, characterized by XRPD signals at 7.7±0.2 degrees two theta, 10.4±0.2 degrees two theta, and 19.0±0.2 degrees two theta.
108. The substantially crystalline Compound 5 free form MeOH Solvate Hydrate Form A according to Embodiment 104, characterized by (a) one or more XRPD signals selected from 7.7±0.2 degrees two theta, 10.4±0.2 degrees two theta, and 19.0±0.2 degrees two theta; and (b) one or more XRPD signals selected from 12.6±0.2 degrees two theta, 17.3±0.2 degrees two theta, 22.1±0.2 degrees two theta, 23.4±0.2 degrees two theta, and 24.4±0.2 degrees two theta.
109. The substantially crystalline Compound 5 free form MeOH Solvate Hydrate Form A according to Embodiment 104, characterized by (a) one or more XRPD signals selected from 7.7±0.2 degrees two theta, 10.4±0.2 degrees two theta, and 19.0±0.2 degrees two theta; and (b) two or more XRPD signals selected from 12.6±0.2 degrees two theta, 17.3±0.2 degrees two theta, 22.1±0.2 degrees two theta, 23.4±0.2 degrees two theta, and 24.4±0.2 degrees two theta.
110. The substantially crystalline Compound 5 free form MeOH Solvate Hydrate Form A according to Embodiment 104, characterized by (a) one or more XRPD signals selected from 7.7±0.2 degrees two theta, 10.4±0.2 degrees two theta, and 19.0±0.2 degrees two theta; and (b) three or more XRPD signals selected from 12.6±0.2 degrees two theta, 17.3±0.2 degrees two theta, 22.1±0.2 degrees two theta, 23.4±0.2 degrees two theta, and 24.4±0.2 degrees two theta.
111. The substantially crystalline Compound 5 free form MeOH Solvate Hydrate Form A according to Embodiment 104, characterized by (a) one or more XRPD signals selected from 7.7±0.2 degrees two theta, 10.4±0.2 degrees two theta, and 19.0±0.2 degrees two theta; and (b) four or more XRPD signals selected from 12.6±0.2 degrees two theta, 17.3±0.2 degrees two theta, 22.1±0.2 degrees two theta, 23.4±0.2 degrees two theta, and 24.4±0.2 degrees two theta.
112. The substantially crystalline Compound 5 free form MeOH Solvate Hydrate Form A according to Embodiment 104, characterized by (a) one or more XRPD signals selected from 7.7±0.2 degrees two theta, 10.4±0.2 degrees two theta, and 19.0±0.2 degrees two theta; and (b) XRPD signals at 12.6±0.2 degrees two theta, 17.3±0.2 degrees two theta, 22.1±0.2 degrees two theta, 23.4±0.2 degrees two theta, and 24.4±0.2 degrees two theta.
113. The substantially crystalline Compound 5 free form MeOH Solvate Hydrate Form A according to Embodiment 104, characterized by (a) two or more XRPD signals selected from 7.7±0.2 degrees two theta, 10.4±0.2 degrees two theta, and 19.0±0.2 degrees two theta; and (b) XRPD signals at 12.6±0.2 degrees two theta, 17.3±0.2 degrees two theta, 22.1±0.2 degrees two theta, 23.4±0.2 degrees two theta, and 24.4±0.2 degrees two theta.
114. The substantially crystalline Compound 5 free form MeOH Solvate Hydrate Form A according to Embodiment 104, characterized by (a) XRPD signals at 7.7±0.2 degrees two theta, 10.4±0.2 degrees two theta, and 19.0±0.2 degrees two theta; and (b) XRPD signals at 12.6±0.2 degrees two theta, 17.3±0.2 degrees two theta, 22.1±0.2 degrees two theta, 23.4±0.2 degrees two theta, and 24.4±0.2 degrees two theta.
115. The substantially crystalline Compound 5 free form MeOH Solvate Hydrate Form A according to Embodiment 104, characterized by a thermogravimetric analysis (TGA) showing about 5.0% weight loss from ambient temperature up to about 150° C.
116. The substantially crystalline Compound 5 free form MeOH Solvate Hydrate Form A according to Embodiment 104, characterized by a differential scanning calorimetry (DSC) analysis showing endothermic peaks at about 31° C. and 174° C., and an exothermic peak at about 246° C.
117. The substantially crystalline Compound 5 free form MeOH Solvate Hydrate Form A according to Embodiment 104, characterized by a ¹³C CPMAS having one or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
118. The substantially crystalline Compound 5 free form MeOH Solvate Hydrate Form A according to Embodiment 104, characterized by a ¹³C CPMAS having two or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
119. The substantially crystalline Compound 5 free form MeOH Solvate Hydrate Form A according to Embodiment 104, characterized by a ¹³C CPMAS having three or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
120. The substantially crystalline Compound 5 free form MeOH Solvate Hydrate Form A according to Embodiment 104, characterized by a ¹³C CPMAS having four or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
121. The substantially crystalline Compound 5 free form MeOH Solvate Hydrate Form A according to Embodiment 104, characterized by a ¹³C CPMAS having five or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
122. The substantially crystalline Compound 5 free form MeOH Solvate Hydrate Form A according to Embodiment 104, characterized by a ¹³C CPMAS having six or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
123. The substantially crystalline Compound 5 free form MeOH Solvate Hydrate Form A according to Embodiment 104, characterized by a ¹³C CPMAS having seven or more signals selected from 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
124. The substantially crystalline Compound 5 free form MeOH Solvate Hydrate Form A according to Embodiment 104, characterized by a ¹³C CPMAS having signals at 48.3±0.2 ppm, 116.5±0.2 ppm, 117.4±0.2 ppm, 123.5±0.2 ppm, 126.5±0.2 ppm, 127.5±0.2 ppm, 129.5±0.2 ppm, 132.1±0.2 ppm, and 140.1±0.2 ppm.
125. The substantially crystalline Compound 5 free form MeOH Solvate Hydrate Form A according to Embodiment 104, characterized by a ¹⁹F MAS having one or more signals selected from −129.4±0.2 ppm, −129.9±0.2 ppm, −131.5±0.2 ppm, −135.6±0.2 ppm, and −139.0±0.2 ppm.
126. The substantially crystalline Compound 5 free form MeOH Solvate Hydrate Form A according to Embodiment 104, characterized by a ¹⁹F MAS having two or more signals selected from −129.4±0.2 ppm, −129.9±0.2 ppm, −131.5±0.2 ppm, −135.6±0.2 ppm, and −139.0±0.2 ppm.
127. The substantially crystalline Compound 5 free form MeOH Solvate Hydrate Form A according to Embodiment 104, characterized by a ¹⁹F MAS having three or more signals selected from −129.4±0.2 ppm, −129.9±0.2 ppm, −131.5±0.2 ppm, −135.6±0.2 ppm, and −139.0±0.2 ppm.
128. The substantially crystalline Compound 5 free form MeOH Solvate Hydrate Form A according to Embodiment 104, characterized by a ¹⁹F MAS having signals at −129.4±0.2 ppm, −129.9±0.2 ppm, −131.5±0.2 ppm, −135.6±0.2 ppm, and −139.0±0.2 ppm.
129. Substantially crystalline Compound 5 free form DCM Solvate Form A:

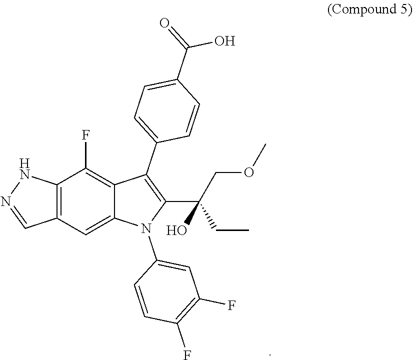 [SMILES: CC[C@@](O)(COC)c1c(-c2ccc(C(=O)O)cc2)c2c(F)c3[nH]ncc3cc2n1-c1ccc(F)c(F)c1]

130. The substantially crystalline Compound 5 free form DCM Solvate Form A according to Embodiment 129, characterized by one or more XRPD signals selected from 8.1±0.2 degrees two theta, 10.3±0.2 degrees two theta, and 17.4±0.2 degrees two theta.
131. The substantially crystalline Compound 5 free form DCM Solvate Form A according to Embodiment 129, characterized by two or more XRPD signals selected from 8.1±0.2 degrees two theta, 10.3±0.2 degrees two theta, and 17.4±0.2 degrees two theta.
132. The substantially crystalline Compound 5 free form DCM Solvate Form A according to Embodiment 129, characterized by XRPD signals at 8.1±0.2 degrees two theta, 10.3±0.2 degrees two theta, and 17.4±0.2 degrees two theta.
133. The substantially crystalline Compound 5 free form DCM Solvate Form A according to Embodiment 129, characterized by (a) one or more XRPD signals selected from 8.1±0.2 degrees two theta, 10.3±0.2 degrees two theta, and 17.4±0.2 degrees two theta; and (b) one or more XRPD signals selected from 8.6±0.2 degrees two theta, 15.5±0.2 degrees two theta, 18.1±0.2 degrees two theta, 18.3±0.2 degrees two theta, and 24.3±0.2 degrees two theta.
134. The substantially crystalline Compound 5 free form DCM Solvate Form A according to Embodiment 129, characterized by (a) two or more XRPD signals selected from 8.1±0.2 degrees two theta, 10.3±0.2 degrees two theta, and 17.4±0.2 degrees two theta; and (b) one or more XRPD signals selected from 8.6±0.2 degrees two theta, 15.5±0.2 degrees two theta, 18.1±0.2 degrees two theta, 18.3±0.2 degrees two theta, and 24.3±0.2 degrees two theta.
135. The substantially crystalline Compound 5 free form DCM Solvate Form A according to Embodiment 129, characterized by (a) three or more XRPD signals selected from 8.1±0.2 degrees two theta, 10.3±0.2 degrees two theta, and 17.4±0.2 degrees two theta; and (b) one or more XRPD signals selected from 8.6±0.2 degrees two theta, 15.5±0.2 degrees two theta, 18.1±0.2 degrees two theta, 18.3±0.2 degrees two theta, and 24.3±0.2 degrees two theta.
136. The substantially crystalline Compound 5 free form DCM Solvate Form A according to Embodiment 129, characterized by (a) four or more XRPD signals selected from 8.1±0.2 degrees two theta, 10.3±0.2 degrees two theta, and 17.4±0.2 degrees two theta; and (b) one or more XRPD signals selected from 8.6±0.2 degrees two theta, 15.5±0.2 degrees two theta, 18.1±0.2 degrees two theta, 18.3±0.2 degrees two theta, and 24.3±0.2 degrees two theta.
137. The substantially crystalline Compound 5 free form DCM Solvate Form A according to Embodiment 129, characterized by (a) one or more XRPD signals selected from 8.1±0.2 degrees two theta, 10.3±0.2 degrees two theta, and 17.4±0.2 degrees two theta; and (b) XRPD signals at 8.6±0.2 degrees two theta, 15.5±0.2 degrees two theta, 18.1±0.2 degrees two theta, 18.3±0.2 degrees two theta, and 24.3±0.2 degrees two theta.
138. The substantially crystalline Compound 5 free form DCM Solvate Form A according to Embodiment 129, characterized by (a) two or more XRPD signals selected from 8.1±0.2 degrees two theta, 10.3±0.2 degrees two theta, and 17.4±0.2 degrees two theta; and (b) XRPD signals at 8.6±0.2 degrees two theta, 15.5±0.2 degrees two theta, 18.1±0.2 degrees two theta, 18.3±0.2 degrees two theta, and 24.3±0.2 degrees two theta.
139. The substantially crystalline Compound 5 free form DCM Solvate Form A according to Embodiment 129, characterized by (a) XRPD signals at 8.1±0.2 degrees two theta, 10.3±0.2 degrees two theta, and 17.4±0.2 degrees two theta; and (b) XRPD signals at 8.6±0.2 degrees two theta, 15.5±0.2 degrees two theta, 18.1±0.2 degrees two theta, 18.3±0.2 degrees two theta, and 24.3±0.2 degrees two theta.
140. The substantially crystalline Compound 5 free form DCM Solvate Form A according to Embodiment 129, characterized by a thermogravimetric analysis (TGA) showing about 8.7% weight loss from ambient temperature up to about 180° C.
141. The substantially crystalline Compound 5 free form DCM Solvate Form A according to Embodiment 129, characterized by a ¹³C CPMAS having one or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
142. The substantially crystalline Compound 5 free form DCM Solvate Form A according to Embodiment 129, characterized by a ¹³C CPMAS having two or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
143. The substantially crystalline Compound 5 free form DCM Solvate Form A according to Embodiment 129, characterized by a ¹³C CPMAS having three or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
144. The substantially crystalline Compound 5 free form DCM Solvate Form A according to Embodiment 129, characterized by a ¹³C CPMAS having four or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
145. The substantially crystalline Compound 5 free form DCM Solvate Form A according to Embodiment 129, characterized by a ¹³C CPMAS having five or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
146. The substantially crystalline Compound 5 free form DCM Solvate Form A according to Embodiment 129, characterized by a ¹³C CPMAS having six or more signals selected from 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
147. The substantially crystalline Compound 5 free form DCM Solvate Form A according to Embodiment 129, characterized by a ¹³C CPMAS having signals at 6.5±0.2 ppm, 77.1±0.2 ppm, 78.4±0.2 ppm, 117.7±0.2 ppm, 125.8±0.2 ppm, 128.8±0.2 ppm, 129.9±0.2 ppm, and 131.2±0.2 ppm.
148. The substantially crystalline Compound 5 free form DCM Solvate Form A according to Embodiment 129, characterized by a ¹⁹F MAS having one or more signals selected from −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
149. The substantially crystalline Compound 5 free form DCM Solvate Form A according to Embodiment 129, characterized by a ¹⁹F MAS having two or more signals selected from −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
150. The substantially crystalline Compound 5 free form DCM Solvate Form A according to Embodiment 129, characterized by a ¹⁹F MAS having three or more signals selected from −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
151. The substantially crystalline Compound 5 free form DCM Solvate Form A according to Embodiment 129, characterized by a ¹⁹F MAS having four or more signals selected from −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
152. The substantially crystalline Compound 5 free form DCM Solvate Form A according to Embodiment 129, characterized by a ¹⁹F MAS having five or more signals selected from −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
153. The substantially crystalline Compound 5 free form DCM Solvate Form A according to Embodiment 129, characterized by a ¹⁹F MAS having signals at −129.4±0.2 ppm, −131.8±0.2 ppm, −133.8±0.2 ppm, −136.8±0.2 ppm, 139.7±0.2 ppm, −141.5±0.2 ppm, −142.6±0.2 ppm, and −146.5±0.2 ppm.
154. Substantially crystalline Compound 5 free form EtOAc Heptane Solvate Form

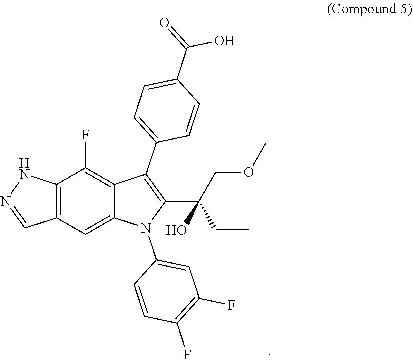 [SMILES: CC[C@@](O)(COC)c1c(-c2ccc(C(=O)O)cc2)c2c(F)c3[nH]ncc3cc2n1-c1ccc(F)c(F)c1]

155. The substantially crystalline Compound 5 free form EtOAc Heptane Solvate Form A according to Embodiment 154, characterized by one or more XRPD signals selected from 17.7±0.2 degrees two theta, 17.2±0.2 degrees two theta, and 8.1±0.2 degrees two theta.
156. The substantially crystalline Compound 5 free form EtOAc Heptane Solvate Form A according to Embodiment 154, characterized by two or more XRPD signals selected from 17.7±0.2 degrees two theta, 17.2±0.2 degrees two theta, and 8.1±0.2 degrees two theta.
157. The substantially crystalline Compound 5 free form EtOAc Heptane Solvate Form A according to Embodiment 154, characterized by XRPD signals at 17.7±0.2 degrees two theta, 17.2±0.2 degrees two theta, and 8.1±0.2 degrees two theta.
158. The substantially crystalline Compound 5 free form EtOAc Heptane Solvate Form A according to Embodiment 154, characterized by (a) one or more XRPD signals selected from 17.7±0.2 degrees two theta, 17.2±0.2 degrees two theta, and 8.1±0.2 degrees two theta; and (b) one or more XRPD signals selected from 23.8±0.2 degrees two theta, 18.8±0.2 degrees two theta, 15.5±0.2 degrees two theta, 12.6±0.2 degrees two theta, and 10.0±0.2 degrees two theta.
159. The substantially crystalline Compound 5 free form EtOAc Heptane Solvate Form A according to Embodiment 154, characterized by (a) one or more XRPD signals selected from 17.7±0.2 degrees two theta, 17.2±0.2 degrees two theta, and 8.1±0.2 degrees two theta; and (b) two or more XRPD signals selected from 23.8±0.2 degrees two theta, 18.8±0.2 degrees two theta, 15.5±0.2 degrees two theta, 12.6±0.2 degrees two theta, and 10.0±0.2 degrees two theta.
160. The substantially crystalline Compound 5 free form EtOAc Heptane Solvate Form A according to Embodiment 154, characterized by (a) one or more XRPD signals selected from 17.7±0.2 degrees two theta, 17.2±0.2 degrees two theta, and 8.1±0.2 degrees two theta; and (b) three or more XRPD signals selected from 23.8±0.2 degrees two theta, 18.8±0.2 degrees two theta, 15.5±0.2 degrees two theta, 12.6±0.2 degrees two theta, and 10.0±0.2 degrees two theta.
161. The substantially crystalline Compound 5 free form EtOAc Heptane Solvate Form A according to Embodiment 154, characterized by (a) one or more XRPD signals selected from 17.7±0.2 degrees two theta, 17.2±0.2 degrees two theta, and 8.1±0.2 degrees two theta; and (b) four or more XRPD signals selected from 23.8±0.2 degrees two theta, 18.8±0.2 degrees two theta, 15.5±0.2 degrees two theta, 12.6±0.2 degrees two theta, and 10.0±0.2 degrees two theta.
162. The substantially crystalline Compound 5 free form EtOAc Heptane Solvate Form A according to Embodiment 154, characterized by (a) one or more XRPD signals selected from 17.7±0.2 degrees two theta, 17.2±0.2 degrees two theta, and 8.1±0.2 degrees two theta; and (b) XRPD signals at 23.8±0.2 degrees two theta, 18.8±0.2 degrees two theta, 15.5±0.2 degrees two theta, 12.6±0.2 degrees two theta, and 10.0±0.2 degrees two theta.
163. The substantially crystalline Compound 5 free form EtOAc Heptane Solvate Form A according to Embodiment 154, characterized by (a) two or more XRPD signals selected from 17.7±0.2 degrees two theta, 17.2±0.2 degrees two theta, and 8.1±0.2 degrees two theta; and (b) XRPD signals at 23.8±0.2 degrees two theta, 18.8±0.2 degrees two theta, 15.5±0.2 degrees two theta, 12.6±0.2 degrees two theta, and 10.0±0.2 degrees two theta.
164. The substantially crystalline Compound 5 free form EtOAc Heptane Solvate Form A according to Embodiment 154, characterized by (a) XRPD signals at 17.7±0.2 degrees two theta, 17.2±0.2 degrees two theta, and 8.1±0.2 degrees two theta; and (b) XRPD signals at 23.8±0.2 degrees two theta, 18.8±0.2 degrees two theta, 15.5±0.2 degrees two theta, 12.6±0.2 degrees two theta, and 10.0±0.2 degrees two theta.
165. The substantially crystalline Compound 5 free form EtOAc Heptane Solvate Form A according to Embodiment 154, characterized by a ¹³C CPMAS having one or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.00±0.2 ppm, and 6.6±0.2 ppm.
166. The substantially crystalline Compound 5 free form EtOAc Heptane Solvate Form A according to Embodiment 154, characterized by a ¹³C CPMAS having two or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
167. The substantially crystalline Compound 5 free form EtOAc Heptane Solvate Form A according to Embodiment 154, characterized by a ¹³C CPMAS having three or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
168. The substantially crystalline Compound 5 free form EtOAc Heptane Solvate Form A according to Embodiment 154, characterized by a ¹³C CPMAS having four or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
169. The substantially crystalline Compound 5 free form EtOAc Heptane Solvate Form A according to Embodiment 154, characterized by a ¹³C CPMAS having five or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
170. The substantially crystalline Compound 5 free form EtOAc Heptane Solvate Form A according to Embodiment 154, characterized by a ¹³C CPMAS having six or more signals selected from 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
171. The substantially crystalline Compound 5 free form EtOAc Heptane Solvate Form A according to Embodiment 154, characterized by a ¹³C CPMAS having signals at 142.7±0.2 ppm, 139.0±0.2 ppm, 132.3±0.2 ppm, 130.4±0.2 ppm, 128.6±0.2 ppm, 125.9±0.2 ppm, 124.3±0.2 ppm, 118.0±0.2 ppm, and 6.6±0.2 ppm.
172. The substantially crystalline Compound 5 free form EtOAc Heptane Solvate Form A according to Embodiment 154, characterized by a ¹⁹F MAS having one or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
173. The substantially crystalline Compound 5 free form EtOAc Heptane Solvate Form A according to Embodiment 154, characterized by a ¹⁹F MAS having two or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
174. The substantially crystalline Compound 5 free form EtOAc Heptane Solvate Form A according to Embodiment 154, characterized by a ¹⁹F MAS having three or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
175. The substantially crystalline Compound 5 free form EtOAc Heptane Solvate Form A according to Embodiment 154, characterized by a ¹⁹F MAS having four or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
176. The substantially crystalline Compound 5 free form EtOAc Heptane Solvate Form A according to Embodiment 154, characterized by a ¹⁹F MAS having five or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
177. The substantially crystalline Compound 5 free form EtOAc Heptane Solvate Form A according to Embodiment 154, characterized by a ¹⁹F MAS having six or more signals selected from −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
178. The substantially crystalline Compound 5 free form EtOAc Heptane Solvate Form A according to Embodiment 154, characterized by a ¹⁹F MAS having signals at −129.6±0.2 ppm, −132.2±0.2 ppm, −135.3±0.2 ppm, −136.9±0.2 ppm, ±0.2 ppm, −139.2±0.2 ppm, −141.0±0.2 ppm, −142.2±0.2 ppm, and −143.7±0.2 ppm.
179. A pharmaceutical composition comprising the substantially crystalline Compound 5 according to any one of Embodiments 1 to 178, and a pharmaceutically acceptable carrier.
180. A method of treating alpha-1 antitrypsin deficiency (AATD) comprising administering to a patient in need thereof a therapeutically effective amount of the substantially crystalline form of Compound 5 according to any one of Embodiments 1 to 178, or a therapeutically effective amount of a pharmaceutical composition according to Embodiment 179.
181. Use of the substantially crystalline form of Compound 5 according to any one of Embodiments 1 to 178, or the pharmaceutical composition according to Embodiment 179 in the manufacture of a medicament for treating AATD.
182. The substantially crystalline form of Compound 5 according to any one of Embodiments 1 to 178, or a therapeutically effective amount of a pharmaceutical composition according to Embodiment 179 for use in treating AATD.
183. A method of preparing substantially crystalline Compound 5 free form Monohydrate Form A comprising the steps of (a) stirring a mixture of Compound 5 free form Form A and 1-propanol while heating to obtain a clear solution, (b) adding water slowly while stirring until a slurry is formed; (c) cooling the slurry under continued agitation (d) washing filtered solids with mixture of 1-propanol and water and drying in a vacuum oven with nitrogen bleed at 50° C.; and (e) collecting the substantially crystalline Compound 5 free form Monohydrate Form A by centrifuge filtration.
184. A method of preparing substantially crystalline Compound 5 free form Form A comprising the steps of (a) dissolving Compound 5 free form EtOH solvate Form A in 4:1 DCM:MeOH and evaporating to dryness; (b) dissolving the residue from step (a) in DCM and refluxing to provide a uniform suspension; (c) allowing to cool slowly at room temperature, and filtering and washing with DCM; and (d) collecting solid and drying under suction and on rotovap, followed by vacuum oven drying at 75-90° C. to provide substantially crystalline Compound 5 free form Form A.
185. A method of preparing substantially crystalline Compound 5 free form Form B comprising the steps of (a) adding Compound 5 free form Form A and octanol to a vial with a stir bar; (b) stirring at 80° C. for 3 days while protected from exposure to light, and (c) collecting the substantially crystalline Compound 5 free form Form B by centrifuge filtration.
186. A method of preparing substantially crystalline Compound 5 free form NPA Solvate Form A, comprising the steps of (a) adding Compound 5 free form Form A and 1-propanol to a vial with a stir bar; (b) stirring for 7 days at 20° C. while protected from exposure to light; and (c) collecting the substantially crystalline Compound 5 free form NPA Solvate Form A by centrifuge filtration.
187. A method of preparing substantially crystalline Compound 5 free form EtOH Solvate Form A, comprising the steps of (a) diluting raw Compound 5 in EtOAc and treating with activated charcoal; (b) refluxing and then cooling to room temperature before filtering and washing with EtOAc to provide a yellow foam; (c) treating the yellow foam with EtOH and heating to reflux in a heating bath; (d) refluxing to provide a uniform suspension and allowing to cool at room temperature and (e) filtering the suspension to collect solid, washing with EtOH, and drying under suction and rotovap to yield substantially crystalline Compound 5 free form EtOH Solvate Form A.
188. A method of preparing substantially crystalline Compound 5 free form MeOH Solvate Hydrate Form A, comprising the steps of (a) adding EtOH to Compound 5 followed by water-bath sonication; (b) stirring at room temperature and isolating solids; (c) adding solids and 9:1 (v/v) MeOH:water followed by sonication; and (d) stirring at ambient temperature for 5 days and isolate substantially crystalline Compound 5 free form MeOH Solvate Hydrate Form A.
189. A method of preparing substantially crystalline Compound 5 free form MeOH Solvate Hydrate Form A, comprising the steps of (a) adding amorphous Compound 5 to 9:1 (v/v) MeOH:water followed by sonication; and (b) stirring at ambient temperature for 5 days and isolate substantially crystalline Compound 5 free form MeOH Solvate Hydrate Form A.
190. A method of preparing substantially crystalline Compound 5 free form DCM Solvate Form A, comprising the steps of (a) combining Compound 5 with water acidified to pH 3 with 2N HCl and extracting with EtOAc; (b) drying filtering, and evaporating the extract in vacuo to provide an orange-red oil; (c) dissolving the oil in DCM to form a clear red solution; and (d) swirling the solution to crystallize product; (e) filtering the suspension and washing the resulting cake with DCM to provide substantially crystalline Compound 5 free form DCM Solvate Form A.
191. A method of preparing substantially crystalline Compound 5 free form EtOH Heptane Solvate Form A, comprising the steps of (a) heating raw Compound 5 in EtOAc to 55° C. until dissolved; (b) cooling the slurry to room temperature and stirring overnight; and (c) filtering the solids to provide substantially crystalline Compound 5 free form EtOH Heptane Solvate Form A.
192. Substantially crystalline Compound 3 free form Form A:

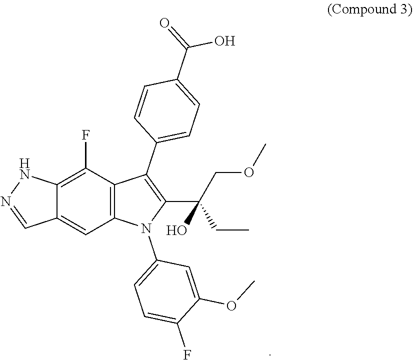 [SMILES: CC[C@@](O)(COC)c1c(-c2ccc(C(=O)O)cc2)c2c(F)c3[nH]ncc3cc2n1-c1ccc(F)c(OC)c1]

193. The substantially crystalline Compound 3 free form Form A according to Embodiment 192, characterized by one or more XRPD signals selected from 5.7±0.2 degrees two theta, 12.3±0.2 degrees two theta, 15.9±0.2 degrees two theta, and 20.2±0.2 degrees two theta.
194. The substantially crystalline Compound 3 free form Form A according to Embodiment 192, characterized by two or more XRPD signals selected from 5.7±0.2 degrees two theta, 12.3±0.2 degrees two theta, 15.9±0.2 degrees two theta, and 20.2±0.2 degrees two theta.
195. The substantially crystalline Compound 3 free form Form A according to Embodiment 192, characterized by three or more XRPD signals at 5.7±0.2 degrees two theta, 12.3±0.2 degrees two theta, 15.9±0.2 degrees two theta, and 20.2±0.2 degrees two theta.
196. The substantially crystalline Compound 3 free form Form A according to Embodiment 192, characterized by XRPD signals at 5.7±0.2 degrees two theta, 12.3±0.2 degrees two theta, 15.9±0.2 degrees two theta, and 20.2±0.2 degrees two theta.
197. The substantially crystalline Compound 3 free form Form A according to Embodiment 192, characterized by XRPD signals at 12.3±0.2 degrees two theta, 18.2±0.2 degrees two theta, and 22.4±0.2 degrees two theta.
198. The substantially crystalline Compound 3 free form Form A according to Embodiment 192, characterized by (a) XRPD signals at 12.3±0.2 degrees two theta, 18.2±0.2 degrees two theta, and 22.4±0.2 degrees two theta; and (b) one or more XRPD signals selected from 5.7±0.2 degrees two theta, 11.0±0.2 degrees two theta, 15.9±0.2 degrees two theta, 19.5±0.2 degrees two theta, and 20.2±0.2 degrees two theta.
199. The substantially crystalline Compound 3 free form Form A according to Embodiment 192, characterized by (a) XRPD signals at 12.3±0.2 degrees two theta, 18.2±0.2 degrees two theta, and 22.4±0.2 degrees two theta; and (b) two or more XRPD signals selected from 5.7±0.2 degrees two theta, 11.0±0.2 degrees two theta, 15.9±0.2 degrees two theta, 19.5±0.2 degrees two theta, and 20.2±0.2 degrees two theta.
200. The substantially crystalline Compound 3 free form Form A according to Embodiment 192, characterized by (a) XRPD signals at 12.3±0.2 degrees two theta, 18.2±0.2 degrees two theta, and 22.4±0.2 degrees two theta; and (b) three or more XRPD signals selected from 5.7±0.2 degrees two theta, 11.0±0.2 degrees two theta, 15.9±0.2 degrees two theta, 19.5±0.2 degrees two theta, and 20.2±0.2 degrees two theta.
201. The substantially crystalline Compound 3 free form Form A according to Embodiment 192, characterized by (a) XRPD signals at 12.3±0.2 degrees two theta, 18.2±0.2 degrees two theta, and 22.4±0.2 degrees two theta; and (b) four or more XRPD signals selected from 5.7±0.2 degrees two theta, 11.0±0.2 degrees two theta, 15.9±0.2 degrees two theta, 19.5±0.2 degrees two theta, and 20.2±0.2 degrees two theta.
202. The substantially crystalline Compound 3 free form Form A according to Embodiment 192, characterized by a ¹³C CPMAS having signals at 170.6±0.2 ppm, 128.1±0.2 ppm, and 116.8±0.2 ppm.
203. The substantially crystalline Compound 3 free form Form A according to Embodiment 192, characterized by a ¹³C CPMAS having (a) signals at 170.6±0.2 ppm, 128.1±0.2 ppm, and 116.8±0.2 ppm; and (b) one or more signals selected from 141.1±0.2 ppm, 133.5±0.2 ppm, 126.7±0.2 ppm, 124.9±0.2 ppm, 80.0±0.2 ppm, and 54.6±0.2 ppm.
204. The substantially crystalline Compound 3 free form Form A according to Embodiment 192, characterized by a ¹³C CPMAS having (a) signals at 170.6±0.2 ppm, 128.1±0.2 ppm, and 116.8±0.2 ppm; and (b) two or more signals selected from 141.1±0.2 ppm, 133.5±0.2 ppm, 126.7±0.2 ppm, 124.9±0.2 ppm, 80.0±0.2 ppm, and 54.6±0.2 ppm.
205. The substantially crystalline Compound 3 free form Form A according to Embodiment 192, characterized by a ¹³C CPMAS having (a) signals at 170.6±0.2 ppm, 128.1±0.2 ppm, and 116.8±0.2 ppm; and (b) three or more signals selected from 141.1±0.2 ppm, 133.5±0.2 ppm, 126.7±0.2 ppm, 124.9±0.2 ppm, 80.0±0.2 ppm, and 54.6±0.2 ppm.
206. The substantially crystalline Compound 3 free form Form A according to Embodiment 192, characterized by a ¹³C CPMAS having (a) signals at 170.6±0.2 ppm, 128.1±0.2 ppm, and 116.8±0.2 ppm; and (b) four or more signals selected from 141.1±0.2 ppm, 133.5±0.2 ppm, 126.7±0.2 ppm, 124.9±0.2 ppm, 80.0±0.2 ppm, and 54.6±0.2 ppm.
207. The substantially crystalline Compound 3 free form Form A according to Embodiment 192, characterized by a ¹³C CPMAS having signals at 170.6±0.2 ppm, 141.1±0.2 ppm, 133.5±0.2 ppm, 128.1±0.2 ppm, 126.7±0.2 ppm, 124.9±0.2 ppm, 116.8±0.2 ppm, 80.0±0.2 ppm, and 54.6±0.2 ppm.
208. The substantially crystalline Compound 3 free form Form A according to Embodiment 192, characterized by a ¹⁹F MAS having one or more signals selected from −132.8±0.2 ppm, −134.8±0.2 ppm, and −138.3±0.2 ppm.
209. The substantially crystalline Compound 3 free form Form A according to Embodiment 192, characterized by a ¹⁹F MAS having two or more signals selected from −132.8±0.2 ppm, −134.8±0.2 ppm, and −138.3±0.2 ppm.
210. The substantially crystalline Compound 3 free form Form A according to Embodiment 192, characterized by a ¹⁹F MAS having signals at −132.8±0.2 ppm, −134.8±0.2 ppm, and −138.3±0.2 ppm.
211. The substantially crystalline Compound 3 free form Form A according to Embodiment 192, characterized by a thermogravimetric analysis (TGA) showing about 0.4% weight loss from ambient temperature up to about 230° C.
212. The substantially crystalline Compound 3 free form Form A according to Embodiment 192, characterized by a differential scanning calorimetry (DSC) analysis showing an endothermic peak at about 244° C.
213. Substantially crystalline Compound 3 free form Form B:

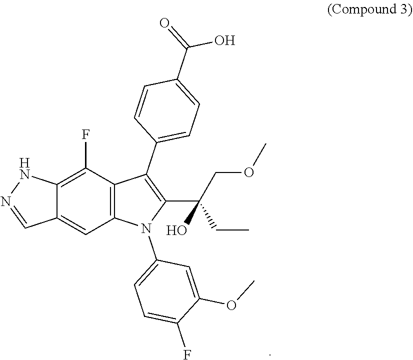 [SMILES: CC[C@@](O)(COC)c1c(-c2ccc(C(=O)O)cc2)c2c(F)c3[nH]ncc3cc2n1-c1ccc(F)c(OC)c1]

214. The substantially crystalline Compound 3 free form Form B according to Embodiment 213, characterized by one or more XRPD signals selected from 8.2±0.2 degrees two theta, 13.5±0.2 degrees two theta, and 16.6±0.2 degrees two theta.
215. The substantially crystalline Compound 3 free form Form B according to Embodiment 213, characterized by two or more XRPD signals selected from 8.2±0.2 degrees two theta, 13.5±0.2 degrees two theta, and 16.6±0.2 degrees two theta.
216. The substantially crystalline Compound 3 free form Form B according to Embodiment 213, characterized by XRPD signals at 8.2±0.2 degrees two theta, 13.5±0.2 degrees two theta, and 16.6±0.2 degrees two theta.
217. The substantially crystalline Compound 3 free form Form B according to Embodiment 213, characterized by XRPD signals at 10.5±0.2 degrees two theta, 11.6±0.2 degrees two theta, and 16.6±0.2 degrees two theta.
218. The substantially crystalline Compound 3 free form Form B according to Embodiment 213, characterized by (a) XRPD signals at 10.5±0.2 degrees two theta, 11.6±0.2 degrees two theta, and 16.6±0.2 degrees two theta; and (b) one or more XRPD signals selected from 8.2±0.2 degrees two theta, 8.5±0.2 degrees two theta, 13.5±0.2 degrees two theta, 17.1±0.2 degrees two theta, and 21.8±0.2 degrees two theta.
219. The substantially crystalline Compound 3 free form Form B according to Embodiment 213, characterized by (a) XRPD signals at 10.5±0.2 degrees two theta, 11.6±0.2 degrees two theta, and 16.6±0.2 degrees two theta; and (b) two or more XRPD signals selected from 8.2±0.2 degrees two theta, 8.5±0.2 degrees two theta, 13.5±0.2 degrees two theta, 17.1±0.2 degrees two theta, and 21.8±0.2 degrees two theta.
220. The substantially crystalline Compound 3 free form Form B according to Embodiment 213, characterized by (a) XRPD signals at 10.5±0.2 degrees two theta, 11.6±0.2 degrees two theta, and 16.6±0.2 degrees two theta; and (b) three or more XRPD signals selected from 8.2±0.2 degrees two theta, 8.5±0.2 degrees two theta, 13.5±0.2 degrees two theta, 17.1±0.2 degrees two theta, and 21.8±0.2 degrees two theta.
221. The substantially crystalline Compound 3 free form Form B according to Embodiment 213, characterized by (a) XRPD signals at 10.5±0.2 degrees two theta, 11.6±0.2 degrees two theta, and 16.6±0.2 degrees two theta; and (b) four or more XRPD signals selected from 8.2±0.2 degrees two theta, 8.5±0.2 degrees two theta, 13.5±0.2 degrees two theta, 17.1±0.2 degrees two theta, and 21.8±0.2 degrees two theta.
222. The substantially crystalline Compound 3 free form Form B according to Embodiment 213, characterized by XRPD signals at 8.2±0.2 degrees two theta, 8.5±0.2 degrees two theta, 10.5±0.2 degrees two theta, 11.6±0.2 degrees two theta, 13.5±0.2 degrees two theta, 16.6±0.2 degrees two theta, 17.1±0.2 degrees two theta, and 21.8±0.2 degrees two theta.
223. The substantially crystalline Compound 3 free form Form B according to Embodiment 213, characterized by a ¹³C CPMAS having one or more signals selected from 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
224. The substantially crystalline Compound 3 free form Form B according to Embodiment 213, characterized by a ¹³C CPMAS having two or more signals selected from 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
225. The substantially crystalline Compound 3 free form Form B according to Embodiment 213, characterized by a ¹³C CPMAS having three or more signals selected from 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
226. The substantially crystalline Compound 3 free form Form B according to Embodiment 213, characterized by a ¹³C CPMAS having four or more signals selected from 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
227. The substantially crystalline Compound 3 free form Form B according to Embodiment 213, characterized by a ¹³C CPMAS having five or more signals selected from 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
228. The substantially crystalline Compound 3 free form Form B according to Embodiment 213, characterized by a ¹³C CPMAS having signals at 143.4±0.2 ppm, 142.5±0.2 ppm, 115.2±0.2 ppm, 76.9±0.2 ppm, 58.0±0.2 ppm, 7.2±0.2 ppm, and 6.7±0.2 ppm.
229. The substantially crystalline Compound 3 free form Form B according to Embodiment 213, characterized by a ¹⁹F MAS having one or more signals selected from −129.4±0.2 ppm, −132.3±0.2 ppm, −137.6±0.2 ppm, −142.0±0.2 ppm, −145.1±0.2 ppm, and −146.9±0.2 ppm.
230. The substantially crystalline Compound 3 free form Form B according to Embodiment 213, characterized by a ¹⁹F MAS having two or more signals selected from −129.4±0.2 ppm, −132.3±0.2 ppm, −137.6±0.2 ppm, −142.0±0.2 ppm, −145.1±0.2 ppm, and −146.9±0.2 ppm.
231. The substantially crystalline Compound 3 free form Form B according to Embodiment 213, characterized by a ¹⁹F MAS having three or more signals selected from −129.4±0.2 ppm, −132.3±0.2 ppm, −137.6±0.2 ppm, −142.0±0.2 ppm, −145.1±0.2 ppm, and −146.9±0.2 ppm.
232. The substantially crystalline Compound 3 free form Form B according to Embodiment 213, characterized by a ¹⁹F MAS having four or more signals selected from −129.4±0.2 ppm, −132.3±0.2 ppm, −137.6±0.2 ppm, −142.0±0.2 ppm, −145.1±0.2 ppm, and −146.9±0.2 ppm.
233. The substantially crystalline Compound 3 free form Form B according to Embodiment 213, characterized by a ¹⁹F MAS having signals at −129.4±0.2 ppm, −132.3±0.2 ppm, −137.6±0.2 ppm, −142.0±0.2 ppm, −145.1±0.2 ppm, and −146.9±0.2 ppm.
234. The substantially crystalline Compound 3 free form Form B according to Embodiment 213, characterized by a thermogravimetric analysis (TGA) showing about 0.6% weight loss from ambient temperature up to about 260° C.
235. The substantially crystalline Compound 3 free form Form B according to Embodiment 213, characterized by a differential scanning calorimetry (DSC) analysis showing endothermic peaks at about 231 and 255° C. 236. Substantially crystalline Compound 3 free form Hydrate Form A:

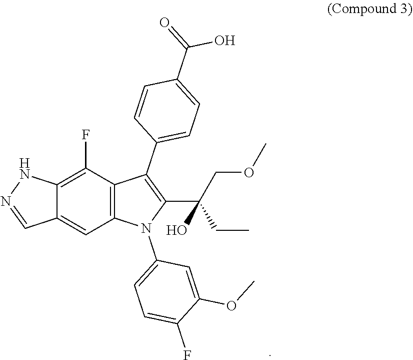 [SMILES: CC[C@@](O)(COC)c1c(-c2ccc(C(=O)O)cc2)c2c(F)c3[nH]ncc3cc2n1-c1ccc(F)c(OC)c1]

237. The substantially crystalline Compound 3 free form Hydrate Form A according to Embodiment 236, characterized by an XRPD signal at 10.0±0.2 degrees two theta, and one or more XRPD signals selected from 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
238. The substantially crystalline Compound 3 free form Hydrate Form A according to Embodiment 236, characterized by an XRPD signal at 10.0±0.2 degrees two theta, and two or more XRPD signals selected from 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
239. The substantially crystalline Compound 3 free form Hydrate Form A according to Embodiment 236, characterized by an XRPD signal at 10.0±0.2 degrees two theta, and three or more XRPD signals selected from 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
240. The substantially crystalline Compound 3 free form Hydrate Form A according to Embodiment 236, characterized by an XRPD signal at 10.0±0.2 degrees two theta, and four or more XRPD signals selected from 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
241. The substantially crystalline Compound 3 free form Hydrate Form A according to Embodiment 236, characterized by an XRPD signal at 10.0±0.2 degrees two theta, and five or more XRPD signals selected from 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
242. The substantially crystalline Compound 3 free form Hydrate Form A according to Embodiment 236, characterized by XRPD signals at 10.0±0.2 degrees two theta, 11.6±0.2 degrees two theta, 12.3±0.2 degrees two theta, 15.3±0.2 degrees two theta, 17.9±0.2 degrees two theta, 18.2±0.2 degrees two theta, 22.2±0.2 degrees two theta, and 24.4±0.2 degrees two theta.
243. The substantially crystalline Compound 3 free form Hydrate Form A according to Embodiment 236, characterized by a thermogravimetric analysis (TGA) showing about 1.6% weight loss from ambient temperature up to about 170° C.
244. The substantially crystalline Compound 3 free form Hydrate Form A according to Embodiment 236, characterized by a differential scanning calorimetry (DSC) analysis showing endothermic peaks at about 155° C. and 242° C. and an exothermic peak at about 172° C.
245. Substantially crystalline Compound 3 free form Hydrate Form B:

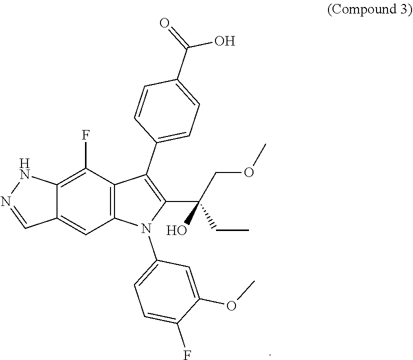 [SMILES: CC[C@@](O)(COC)c1c(-c2ccc(C(=O)O)cc2)c2c(F)c3[nH]ncc3cc2n1-c1ccc(F)c(OC)c1]

246. The substantially crystalline Compound 3 free form Hydrate Form B according to Embodiment 245, characterized by one or more XRPD signals selected from 19.0±0.2 degrees two theta, 21.1±0.2 degrees two theta, 26.4±0.2 degrees two theta, and 27.4±0.2 degrees two theta.
247. The substantially crystalline Compound 3 free form Hydrate Form B according to Embodiment 245, characterized by two or more XRPD signals selected from 19.0±0.2 degrees two theta, 21.1±0.2 degrees two theta, 26.4±0.2 degrees two theta, and 27.4±0.2 degrees two theta.
248. The substantially crystalline Compound 3 free form Hydrate Form B according to Embodiment 245, characterized by three or more XRPD signals selected from 19.0±0.2 degrees two theta, 21.1±0.2 degrees two theta, 26.4±0.2 degrees two theta, and 27.4±0.2 degrees two theta.
249. The substantially crystalline Compound 3 free form Hydrate Form B according to Embodiment 245, characterized by XRPD signals at 19.0±0.2 degrees two theta, 21.1±0.2 degrees two theta, 26.4±0.2 degrees two theta, and 27.4±0.2 degrees two theta.
250. The substantially crystalline Compound 3 free form Hydrate Form B according to Embodiment 245, characterized by (a) one or more XRPD signals selected from 19.0±0.2 degrees two theta, 21.1±0.2 degrees two theta, 26.4±0.2 degrees two theta, and 27.4±0.2 degrees two theta; and (b) one or more XRPD signals selected from 11.5±0.2 degrees two theta, 15.3±0.2 degrees two theta, 15.8±0.2 degrees two theta, 17.6±0.2 degrees two theta, and 17.9±0.2 degrees two theta.
251. The substantially crystalline Compound 3 free form Hydrate Form B according to Embodiment 245, characterized by (a) one or more XRPD signals selected from 19.0±0.2 degrees two theta, 21.1±0.2 degrees two theta, 26.4±0.2 degrees two theta, and 27.4±0.2 degrees two theta; and (b) two or more XRPD signals selected from 11.5±0.2 degrees two theta, 15.3±0.2 degrees two theta, 15.8±0.2 degrees two theta, 17.6±0.2 degrees two theta, and 17.9±0.2 degrees two theta.
252. The substantially crystalline Compound 3 free form Hydrate Form B according to Embodiment 245, characterized by (a) one or more XRPD signals selected from 19.0±0.2 degrees two theta, 21.1±0.2 degrees two theta, 26.4±0.2 degrees two theta, and 27.4±0.2 degrees two theta; and (b) three or more XRPD signals selected from 11.5±0.2 degrees two theta, 15.3±0.2 degrees two theta, 15.8±0.2 degrees two theta, 17.6±0.2 degrees two theta, and 17.9±0.2 degrees two theta.
253. The substantially crystalline Compound 3 free form Hydrate Form B according to Embodiment 245, characterized by (a) one or more XRPD signals selected from 19.0±0.2 degrees two theta, 21.1±0.2 degrees two theta, 26.4±0.2 degrees two theta, and 27.4±0.2 degrees two theta; and (b) four or more XRPD signals selected from 11.5±0.2 degrees two theta, 15.3±0.2 degrees two theta, 15.8±0.2 degrees two theta, 17.6±0.2 degrees two theta, and 17.9±0.2 degrees two theta.
254. The substantially crystalline Compound 3 free form Hydrate Form B according to Embodiment 245, characterized by a thermogravimetric analysis (TGA) showing about 4.8% weight loss from ambient temperature up to about 200° C.
255. The substantially crystalline Compound 3 free form Hydrate Form B according to Embodiment 245, characterized by a differential scanning calorimetry (DSC) analysis showing endothermic peaks at about 79° C. and 244° C., and an exothermic peak at about 184° C.
256. Substantially crystalline Compound 3 free form Hydrate Form C:

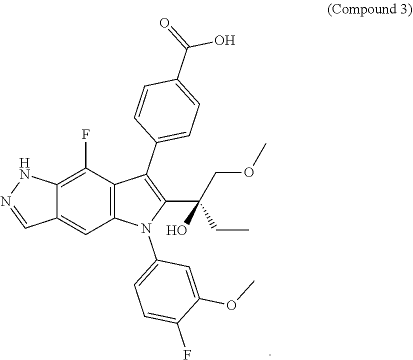 [SMILES: CC[C@@](O)(COC)c1c(-c2ccc(C(=O)O)cc2)c2c(F)c3[nH]ncc3cc2n1-c1ccc(F)c(OC)c1]

257. The substantially crystalline Compound 3 free form Hydrate Form C according to Embodiment 256, characterized by XRPD signals selected from 5.7±0.2 degrees two theta and 19.7±0.2 degrees two theta.
258. The substantially crystalline Compound 3 free form Hydrate Form C according to Embodiment 256, characterized by XRPD signals at 5.7±0.2 degrees two theta and 19.7±0.2 degrees two theta.
259. The substantially crystalline Compound 3 free form Hydrate Form C according to Embodiment 256, characterized by (a) an XRPD signal at 5.7±0.2 degrees two theta and/or at 19.7±0.2 degrees two theta; and (b) one or more XRPD signals selected from 11.4±0.2 degrees two theta, 15.2±0.2 degrees two theta, 18.5±0.2 degrees two theta, 22.0±0.2 degrees two theta, and 22.9±0.2 degrees two theta.
260. The substantially crystalline Compound 3 free form Hydrate Form C according to Embodiment 256, characterized by (a) an XRPD signal at 5.7±0.2 degrees two theta and/or at 19.7±0.2 degrees two theta; and (b) two or more XRPD signals selected from 11.4±0.2 degrees two theta, 15.2±0.2 degrees two theta, 18.5±0.2 degrees two theta, 22.0±0.2 degrees two theta, and 22.9±0.2 degrees two theta.
261. The substantially crystalline Compound 3 free form Hydrate Form C according to Embodiment 256, characterized by (a) an XRPD signal at 5.7±0.2 degrees two theta and/or at 19.7±0.2 degrees two theta; and (b) three or more XRPD signals selected from 11.4±0.2 degrees two theta, 15.2±0.2 degrees two theta, 18.5±0.2 degrees two theta, 22.0±0.2 degrees two theta, and 22.9±0.2 degrees two theta.
262. The substantially crystalline Compound 3 free form Hydrate Form C according to Embodiment 256, characterized by (a) an XRPD signal at 5.7±0.2 degrees two theta and/or at 19.7±0.2 degrees two theta; and (b) four or more XRPD signals selected from 11.4±0.2 degrees two theta, 15.2±0.2 degrees two theta, 18.5±0.2 degrees two theta, 22.0±0.2 degrees two theta, and 22.9±0.2 degrees two theta.
263. The substantially crystalline Compound 3 free form Hydrate Form C according to Embodiment 256, characterized by (a) an XRPD signal at 5.7±0.2 degrees two theta and/or at 19.7±0.2 degrees two theta; and (b) XRPD signals at 11.4±0.2 degrees two theta, 15.2±0.2 degrees two theta, 18.5±0.2 degrees two theta, 22.0±0.2 degrees two theta, and 22.9±0.2 degrees two theta.
264. The substantially crystalline Compound 3 free form Hydrate Form C according to Embodiment 256, characterized by a thermogravimetric analysis (TGA) showing about 4.2% weight loss from ambient temperature up to about 180° C.
265. The substantially crystalline Compound 3 free form Hydrate Form C according to Embodiment 256, characterized by a differential scanning calorimetry (DSC) analysis showing an endothermic peak at about 241° C.
266. Substantially crystalline Compound 3 free form MTBE Solvate Form A:

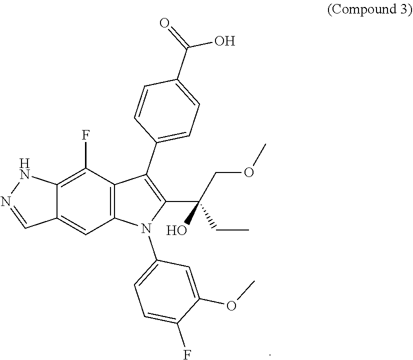 [SMILES: CC[C@@](O)(COC)c1c(-c2ccc(C(=O)O)cc2)c2c(F)c3[nH]ncc3cc2n1-c1ccc(F)c(OC)c1]

267. The substantially crystalline Compound 3 free form MTBE Solvate Form A according to Embodiment 266, characterized by one or more XRPD signals selected from 15.6±0.2 degrees two theta, 17.9±0.2 degrees two theta, and 22.1±0.2 degrees two theta.
268. The substantially crystalline Compound 3 free form MTBE Solvate Form A according to Embodiment 266, characterized by two or more XRPD signals selected from 15.6±0.2 degrees two theta, 17.9±0.2 degrees two theta, and 22.1±0.2 degrees two theta.
269. The substantially crystalline Compound 3 free form MTBE Solvate Form A according to Embodiment 266, characterized by XRPD signals at 15.6±0.2 degrees two theta, 17.9±0.2 degrees two theta, and 22.1±0.2 degrees two theta.
270. The substantially crystalline Compound 3 free form MTBE Solvate Form A according to Embodiment 266, characterized by (a) one or more XRPD signals selected from 15.6±0.2 degrees two theta, 17.9±0.2 degrees two theta, and 22.1±0.2 degrees two theta; and (b) one or more XRPD signals selected from 9.1±0.2 degrees two theta, 10.2±0.2 degrees two theta, 13.6±0.2 degrees two theta, 17.0±0.2 degrees two theta, and 19.7±0.2 degrees two theta.
271. The substantially crystalline Compound 3 free form MTBE Solvate Form A according to Embodiment 266, characterized by (a) one or more XRPD signals selected from 15.6±0.2 degrees two theta, 17.9±0.2 degrees two theta, and 22.1±0.2 degrees two theta; and (b) two or more XRPD signals selected from 9.1±0.2 degrees two theta, 10.2±0.2 degrees two theta, 13.6±0.2 degrees two theta, 17.0±0.2 degrees two theta, and 19.7±0.2 degrees two theta.
272. The substantially crystalline Compound 3 free form MTBE Solvate Form A according to Embodiment 266, characterized by (a) one or more XRPD signals selected from 15.6±0.2 degrees two theta, 17.9±0.2 degrees two theta, and 22.1±0.2 degrees two theta; and (b) three or more XRPD signals selected from 9.1±0.2 degrees two theta, 10.2±0.2 degrees two theta, 13.6±0.2 degrees two theta, 17.0±0.2 degrees two theta, and 19.7±0.2 degrees two theta.
273. The substantially crystalline Compound 3 free form MTBE Solvate Form A according to Embodiment 266, characterized by (a) one or more XRPD signals selected from 15.6±0.2 degrees two theta, 17.9±0.2 degrees two theta, and 22.1±0.2 degrees two theta; and (b) four or more XRPD signals selected from 9.1±0.2 degrees two theta, 10.2±0.2 degrees two theta, 13.6±0.2 degrees two theta, 17.0±0.2 degrees two theta, and 19.7±0.2 degrees two theta.
274. The substantially crystalline Compound 3 free form MTBE Solvate Form A according to Embodiment 266, characterized by (a) one or more XRPD signals selected from 15.6±0.2 degrees two theta, 17.9±0.2 degrees two theta, and 22.1±0.2 degrees two theta; and (b) XRPD signals at 9.1±0.2 degrees two theta, 10.2±0.2 degrees two theta, 13.6±0.2 degrees two theta, 17.0±0.2 degrees two theta, and 19.7±0.2 degrees two theta.
275. The substantially crystalline Compound 3 free form MTBE Solvate Form A according to Embodiment 266, characterized by a ¹³C CPMAS having one or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
276. The substantially crystalline Compound 3 free form MTBE Solvate Form A according to Embodiment 266, characterized by a ¹³C CPMAS having two or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
277. The substantially crystalline Compound 3 free form MTBE Solvate Form A according to Embodiment 266, characterized by a ¹³C CPMAS having three or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
278. The substantially crystalline Compound 3 free form MTBE Solvate Form A according to Embodiment 266, characterized by a ¹³C CPMAS having four or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
279. The substantially crystalline Compound 3 free form MTBE Solvate Form A according to Embodiment 266, characterized by a ¹³C CPMAS having five or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
280. The substantially crystalline Compound 3 free form MTBE Solvate Form A according to Embodiment 266, characterized by a ¹³C CPMAS having six or more signals selected from 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
281. The substantially crystalline Compound 3 free form MTBE Solvate Form A according to Embodiment 266, characterized by a ¹³C CPMAS having signals at 131.0±0.2 ppm, 129.1±0.2 ppm, 125.2±0.2 ppm, 124.5±0.2 ppm, 76.6±0.2 ppm, 55.1±0.2 ppm, 48.1±0.2 ppm, 25.9±0.2 ppm.
282. The substantially crystalline Compound 3 free form MTBE Solvate Form A according to Embodiment 266, characterized by a ¹⁹F MAS having one or more signals selected from −131.4±0.2 ppm, −135.0±0.2 ppm, and −146.5±0.2 ppm.
283. The substantially crystalline Compound 3 free form MTBE Solvate Form A according to Embodiment 266, characterized by a ¹⁹F MAS having two or more signals selected from −131.4±0.2 ppm, −135.0±0.2 ppm, and −146.5±0.2 ppm.
284. The substantially crystalline Compound 3 free form MTBE Solvate Form A according to Embodiment 266, characterized by a ¹⁹F MAS having signals at −131.4±0.2 ppm, −135.0±0.2 ppm, and −146.5±0.2 ppm.
285. The substantially crystalline Compound 3 free form MTBE Solvate Form A according to Embodiment 266, characterized by a thermogravimetric analysis (TGA) showing about 14.7% weight loss from ambient temperature up to about 175° C.
286. The substantially crystalline Compound 3 free MTBE Solvate Form A according to Embodiment 266, characterized by a differential scanning calorimetry (DSC) analysis showing endothermic peaks at about 159° C. and 251° C., and an exothermic peak at about 201° C.
287. A pharmaceutical composition comprising the substantially crystalline Compound 3 according to any one of Embodiments 192 to 286, and a pharmaceutically acceptable carrier.
288. A method of treating alpha-1 antitrypsin deficiency (AATD) comprising administering to a patient in need thereof a therapeutically effective amount of the substantially crystalline form of Compound 3 according to any one of Embodiments 192 to 286, or a therapeutically effective amount of a pharmaceutical composition according to Embodiment 287.
289. Use of the substantially crystalline form of Compound 3 according to any one of Embodiments 192 to 286, or the pharmaceutical composition according to Embodiment 287 in the manufacture of a medicament for treating AATD.
290. The substantially crystalline form of Compound 3 according to any one of Embodiments 192 to 286, or a therapeutically effective amount of a pharmaceutical composition according to Embodiment 287 for use in treating AATD.
291. A method of preparing substantially crystalline Compound 3 free form Form A comprising the steps of (a) concentrating reaction mixture in vacuo, (b) combining Compound 3 with water acidified to pH 3 with 2N HCl, cooling to 0° C., and extracting with EtOAc, (c) drying, filtering, and evaporating the extract in vacuo to provide a foam, (d) treating the foam with acetonitrile, reflux, water, further reflux, and then bringing to room temperature (e) filtering the suspension resulting from step (d) and washing the collected solid with acetonitrile:water (1:1), and (f) drying to provide substantially crystalline Compound 3 free form Form A.
292. A method of preparing substantially crystalline Compound 3 free form Form B comprising the steps of (a) slurring amorphous Compound 3 in chlorobenzene or toluene solution for about 2 weeks at room temperature, (b) isolating the solid to provide substantially crystalline Compound 3 free form Form B.
293. A method of preparing substantially crystalline Compound 3 free form Hydrate Form A comprising the steps of (a) dissolving Compound 3 free form Form A in 1,2-dimethoxyethane, (b) while stirring, slowly adding the resulting solution to a vial containing water, (c) stirring the resulting solution until the precipitate appears, and (d) isolating the precipitate to provide substantially crystalline Compound 3 free form Hydrate Form A.
294. A method of preparing substantially crystalline Compound 3 free form Hydrate Form B comprising the steps of (a) dissolving Compound 3 free form Form A in acetone, (b) while stirring, slowly adding the resulting solution to a vial containing water, (c) stirring the resulting solution until the precipitate appears, and (d) isolating the precipitate to provide substantially crystalline Compound 3 free form Hydrate Form B.
295. A method of preparing substantially crystalline Compound 3 free form Hydrate Form C comprising the steps of (a) dissolving Compound 3 free form Form A in THF, (b) while stirring, slowly adding methyl tert-butyl ether (MTBE) to the resulting solution until precipitate appears, (c) isolating the precipitate to provide substantially crystalline Compound 3 free form Hydrate Form C.
296. A method of preparing substantially crystalline Compound 3 free form MTBE Solvate Form A comprising the steps of (a) triturating raw Compound 3 with MTBE, (b) dissolving isolated solids in THF at 50° C. for resin treatment, (c) concentrating THF filtrate, (d) re-crystallizing from MTBE, and (e) isolating the solid to provide substantially crystalline Compound 3 free form MTBE Solvate Form A.
297. Substantially crystalline Compound 4 free form Form A:

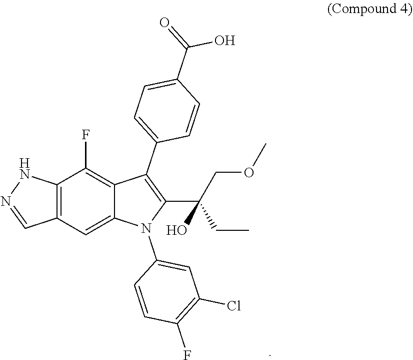 [SMILES: CC[C@@](O)(COC)c1c(-c2ccc(C(=O)O)cc2)c2c(F)c3[nH]ncc3cc2n1-c1ccc(F)c(Cl)c1]

298. The substantially crystalline Compound 4 free form Form A according to Embodiment 297, characterized by an XRPD signal at 16.2±0.2 degrees two theta or at 26.3±0.2 degrees two theta.
299. The substantially crystalline Compound 4 free form Form A according to Embodiment 297, characterized by XRPD signals at 16.2±0.2 degrees two theta and 26.3±0.2 degrees two theta.
300. The substantially crystalline Compound 4 free form Form A according to Embodiment 297, characterized by (a) XRPD signals at 16.2±0.2 degrees two theta and 26.3±0.2 degrees two theta; and (b) one or more signals selected from 11.6±0.2 degrees two theta, 12.0±0.2 degrees two theta, and 18.9±0.2 degrees two theta.
301. The substantially crystalline Compound 4 free form Form A according to Embodiment 297, characterized by (a) an XRPD signal at 16.2±0.2 degrees two theta or at 26.3±0.2 degrees two theta; and (b) XRPD signals at 11.6±0.2 degrees two theta, 12.0±0.2 degrees two theta, and 18.9±0.2 degrees two theta.
302. The substantially crystalline Compound 4 free form Form A according to Embodiment 297, characterized by XRPD signals at 11.6±0.2 degrees two theta, 12.0±0.2 degrees two theta, 16.2±0.2 degrees two theta, 18.9±0.2 degrees two theta, and 26.3±0.2 degrees two theta.
303. The substantially crystalline Compound 4 free form Form A according to Embodiment 297, characterized by (a) an XRPD signal at 16.2±0.2 degrees two theta and/or at 26.3±0.2 degrees two theta; and (b) two or more XRPD signals selected from 10.8±0.2 degrees two theta, 11.6±0.2 degrees two theta, 12.0±0.2 degrees two theta, 15.5±0.2 degrees two theta, 18.9±0.2 degrees two theta, and 23.3±0.2 degrees two theta.
304. The substantially crystalline Compound 4 free form Form A according to Embodiment 297, characterized by (a) an XRPD signal at 16.2±0.2 degrees two theta and/or at 26.3±0.2 degrees two theta; and (b) three or more XRPD signals selected from 10.8±0.2 degrees two theta, 11.6±0.2 degrees two theta, 12.0±0.2 degrees two theta, 15.5±0.2 degrees two theta, 18.9±0.2 degrees two theta, and 23.3±0.2 degrees two theta.
305. The substantially crystalline Compound 4 free form Form A according to Embodiment 297, characterized by (a) an XRPD signal at 16.2±0.2 degrees two theta and/or at 26.3±0.2 degrees two theta; and (b) four or more XRPD signals selected from 10.8±0.2 degrees two theta, 11.6±0.2 degrees two theta, 12.0±0.2 degrees two theta, 15.5±0.2 degrees two theta, 18.9±0.2 degrees two theta, and 23.3±0.2 degrees two theta.
306. The substantially crystalline Compound 4 free form Form A according to Embodiment 297, characterized by (a) an XRPD signal at 16.2±0.2 degrees two theta and/or at 26.3±0.2 degrees two theta; and (b) five or more XRPD signals selected from 10.8±0.2 degrees two theta, 11.6±0.2 degrees two theta, 12.0±0.2 degrees two theta, 15.5±0.2 degrees two theta, 18.9±0.2 degrees two theta, and 23.3±0.2 degrees two theta.
307. The substantially crystalline Compound 4 free form Form A according to Embodiment 297, characterized by XRPD signals at 10.8±0.2 degrees two theta, 11.6±0.2 degrees two theta, 12.0±0.2 degrees two theta, 15.5±0.2 degrees two theta 16.2±0.2 degrees two theta, 18.9±0.2 degrees two theta, 23.3±0.2 degrees two theta, and 26.3±0.2 degrees two theta.
308. The substantially crystalline Compound 4 free form Form A according to Embodiment 297, characterized by a ¹³C CPMAS having one or more signals selected from 171.0±0.2 ppm, 131.3±0.2 ppm, 126.8±0.2 ppm, and 124.4±0.2 ppm.
309. The substantially crystalline Compound 4 free form Form A according to Embodiment 297, characterized by a ¹³C CPMAS having two or more signals selected from 171.0±0.2 ppm, 131.3±0.2 ppm, 126.8±0.2 ppm, and 124.4±0.2 ppm.
310. The substantially crystalline Compound 4 free form Form A according to Embodiment 297, characterized by a ¹³C CPMAS having three or more signals selected from 171.0±0.2 ppm, 131.3±0.2 ppm, 126.8±0.2 ppm, and 124.4±0.2 ppm.
311. The substantially crystalline Compound 4 free form Form A according to Embodiment 297, characterized by a ¹³C CPMAS having signals at 171.0±0.2 ppm, 131.3±0.2 ppm, 126.8±0.2 ppm, and 124.4±0.2 ppm.
312. The substantially crystalline Compound 4 free form Form A according to Embodiment 297, characterized by a ¹³C CPMAS (a) having signals at 171.0±0.2 ppm, 131.3±0.2 ppm, 126.8±0.2 ppm, and/or 124.4±0.2 ppm; and (b) having one or more signals selected from 141.5±0.2 ppm, 138.7±0.2 ppm, 132.9±0.2 ppm, 130.5±0.2 ppm, 127.3±0.2 ppm, and 117.6±0.2 ppm.
313. The substantially crystalline Compound 4 free form Form A according to Embodiment 297, characterized by a ¹³C CPMAS (a) having signals at 171.0±0.2 ppm, 131.3±0.2 ppm, 126.8±0.2 ppm, and/or 124.4±0.2 ppm; and (b) having two or more signals selected from 141.5±0.2 ppm, 138.7±0.2 ppm, 132.9±0.2 ppm, 130.5±0.2 ppm, 127.3±0.2 ppm, and 117.4±0.2 ppm.
314. The substantially crystalline Compound 4 free form Form A according to Embodiment 297, characterized by a ¹³C CPMAS (a) having signals at 171.0±0.2 ppm, 131.3±0.2 ppm, 126.8±0.2 ppm, and/or 124.4±0.2 ppm; and (b) having three or more signals selected from 141.5±0.2 ppm, 138.7±0.2 ppm, 132.9±0.2 ppm, 130.5±0.2 ppm, 127.3±0.2 ppm, and 117.4±0.2 ppm.
315. The substantially crystalline Compound 4 free form Form A according to Embodiment 297, characterized by a ¹³C CPMAS (a) having signals at 171.0±0.2 ppm, 131.3±0.2 ppm, 126.8±0.2 ppm, and/or 124.4±0.2 ppm; and (b) having four or more signals selected from 141.5±0.2 ppm, 138.7±0.2 ppm, 132.9±0.2 ppm, 130.5±0.2 ppm, 127.3±0.2 ppm, and 117.4±0.2 ppm.
316. The substantially crystalline Compound 4 free form Form A according to Embodiment 297, characterized by a ¹³C CPMAS having signals at 171.0±0.2 ppm, 131.3±0.2 ppm, 126.8±0.2 ppm, and/or 124.4±0.2 ppm; and (b) signals at 141.5±0.2 ppm, 138.7±0.2 ppm, 132.9±0.2 ppm, 130.5±0.2 ppm, 127.3±0.2 ppm, and 117.4±0.2 ppm.
317. The substantially crystalline Compound 4 free form Form A according to Embodiment 297, characterized by a ¹⁹F MAS having a signal at −100.1±0.2 ppm and/or a signal at 116.5±0.2 ppm.
318a. The substantially crystalline Compound 4 free form Form A according to Embodiment 297, characterized by a ¹⁹F MAS having signals at −100.1±0.2 ppm, −105.0±0.2 ppm, −114.7±0.2 ppm, and 116.5±0.2 ppm.
318b. The substantially crystalline Compound 4 free form Form A according to Embodiment 297, characterized by a thermogravimetric analysis (TGA) showing about 0.6% weight loss from ambient temperature up to about 200° C.
318c. The substantially crystalline Compound 4 free form Form A according to Embodiment 297, characterized by a differential scanning calorimetry (DSC) analysis showing an endothermic peak at about 253° C.
318d. The substantially crystalline Compound 4 free form Form A according to Embodiment 297, characterized by a monoclinic crystal system, a P2₁ space group, and unit cell dimensions measured at 100 K on a Bruker diffractometer equipped with Cu K_{α} radiation (λ=1.54178 Å) and a CMOS detector of:

| | | | |
|---|---|---|---|
| a | 15.3 ± 0.1 Å | α | 90° |
| b | 9.2 ± 0.1 Å | β | 97.2° ± 0.1º |
| c | 17.1 ± 0.1 Å | γ | 90°. |

319. Substantially crystalline Compound 4 free form Form B:

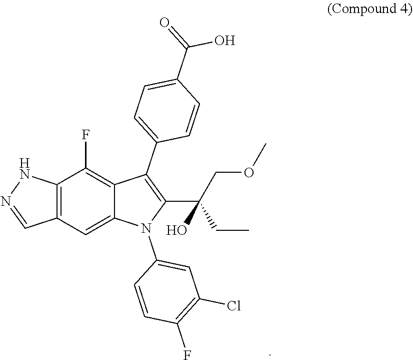 [SMILES: CC[C@@](O)(COC)c1c(-c2ccc(C(=O)O)cc2)c2c(F)c3[nH]ncc3cc2n1-c1ccc(F)c(Cl)c1]

320. The substantially crystalline Compound 4 free form Form B according to Embodiment 319, characterized by one or more XRPD signals selected from 3.2±0.2 degrees two theta, 6.4±0.2 degrees two theta, 8.3±0.2 degrees two theta, and 8.7±0.2 degrees two theta.
321. The substantially crystalline Compound 4 free form Form B according to Embodiment 319, characterized by two or more XRPD signals selected from 3.2±0.2 degrees two theta, 6.4±0.2 degrees two theta, 8.3±0.2 degrees two theta, and 8.7±0.2 degrees two theta.
322. The substantially crystalline Compound 4 free form Form B according to Embodiment 319, characterized by three or more XRPD signals selected from 3.2±0.2 degrees two theta, 6.4±0.2 degrees two theta, 8.3±0.2 degrees two theta, and 8.7±0.2 degrees two theta.
323. The substantially crystalline Compound 4 free form Form B according to Embodiment 319, characterized by XRPD signals at 3.2±0.2 degrees two theta, 6.4±0.2 degrees two theta, 8.3±0.2 degrees two theta, and 8.7±0.2 degrees two theta.
324. The substantially crystalline Compound 4 free form Form B according to Embodiment 319, characterized by (a) one or more XRPD signals selected from 3.2±0.2 degrees two theta, 6.4±0.2 degrees two theta, 8.3±0.2 degrees two theta, and 8.7±0.2 degrees two theta; and (b) one or more XRPD signals selected from 10.7±0.2 degrees two theta, 11.7±0.2 degrees two theta, 16.9±0.2 degrees two theta, and 18.9±0.2 degrees two theta.
325. The substantially crystalline Compound 4 free form Form B according to Embodiment 319, characterized by (a) two or more XRPD signals selected from 3.2±0.2 degrees two theta, 6.4±0.2 degrees two theta, 8.3±0.2 degrees two theta, and 8.7±0.2 degrees two theta; and (b) one or more XRPD signals selected from 10.7±0.2 degrees two theta, 11.7±0.2 degrees two theta, 16.9±0.2 degrees two theta, and 18.9±0.2 degrees two theta.
326. The substantially crystalline Compound 4 free form Form B according to Embodiment 319, characterized by (a) three or more XRPD signals selected from 3.2±0.2 degrees two theta, 6.4±0.2 degrees two theta, 8.3±0.2 degrees two theta, and 8.7±0.2 degrees two theta; and (b) one or more XRPD signals selected from 10.7±0.2 degrees two theta, 11.7±0.2 degrees two theta, 16.9±0.2 degrees two theta, and 18.9±0.2 degrees two theta.
327. The substantially crystalline Compound 4 free form Form B according to Embodiment 319, characterized by (a) XRPD signals at 3.2±0.2 degrees two theta, 6.4±0.2 degrees two theta, 8.3±0.2 degrees two theta, and 8.7±0.2 degrees two theta; and (b) one or more XRPD signals selected from 10.7±0.2 degrees two theta, 11.7±0.2 degrees two theta, 16.9±0.2 degrees two theta, and 18.9±0.2 degrees two theta.
328. The substantially crystalline Compound 4 free form Form B according to Embodiment 319, characterized by (a) XRPD signals at 3.2±0.2 degrees two theta, 6.4±0.2 degrees two theta, 8.3±0.2 degrees two theta, and 8.7±0.2 degrees two theta; and (b) two or more XRPD signals selected from 10.7±0.2 degrees two theta, 11.7±0.2 degrees two theta, 16.9±0.2 degrees two theta, and 18.9±0.2 degrees two theta.
329. The substantially crystalline Compound 4 free form Form B according to Embodiment 319, characterized by (a) XRPD signals at 3.2±0.2 degrees two theta, 6.4±0.2 degrees two theta, 8.3±0.2 degrees two theta, and 8.7±0.2 degrees two theta; and (b) three or more XRPD signals selected from 10.7±0.2 degrees two theta, 11.7±0.2 degrees two theta, 16.9±0.2 degrees two theta, and 18.9±0.2 degrees two theta.
330. The substantially crystalline Compound 4 free form Form B according to Embodiment 319, characterized by XRPD signals at 3.2±0.2 degrees two theta, 6.4±0.2 degrees two theta, 8.3±0.2 degrees two theta, 8.7±0.2 degrees two theta; 10.7±0.2 degrees two theta, 11.7±0.2 degrees two theta, 16.9±0.2 degrees two theta, and 18.9±0.2 degrees two theta.
331. The substantially crystalline Compound 4 free form Form B according to Embodiment 319, characterized by a ¹³C CPMAS having one or more signals selected from 134.0±0.2 ppm, 128.9±0.2 ppm, 125.9±0.2 ppm, and 123.6±0.2 ppm.
332. The substantially crystalline Compound 4 free form Form B according to Embodiment 319, characterized by a ¹³C CPMAS having two or more signals selected from 134.0±0.2 ppm, 128.9±0.2 ppm, 125.9±0.2 ppm, and 123.6±0.2 ppm.
333. The substantially crystalline Compound 4 free form Form B according to Embodiment 319, characterized by a ¹³C CPMAS having three or more signals selected from 134.0±0.2 ppm, 128.9±0.2 ppm, 125.9±0.2 ppm, and 123.6±0.2 ppm.
334. The substantially crystalline Compound 4 free form Form B according to Embodiment 319, characterized by a ¹³C CPMAS having signals at 134.0±0.2 ppm, 128.9±0.2 ppm, 125.9±0.2 ppm, and 123.6±0.2 ppm.
335a. The substantially crystalline Compound 4 free form Form B according to Embodiment 319, characterized by a ¹³C CPMAS (a) having signals at 134.0±0.2 ppm, 128.9±0.2 ppm, 125.9±0.2 ppm, and/or 123.6±0.2 ppm; and (b) having one or more signals selected from 143.3±0.2 ppm, 138.7±0.2 ppm, 130.7±0.2 ppm, 129.5±0.2 ppm, 117.6±0.2 ppm, and 6.7±0.2 ppm.
335b. The substantially crystalline Compound 4 free form Form B according to Embodiment 319, characterized by a ¹³C CPMAS (a) having signals at 134.0±0.2 ppm, 128.9±0.2 ppm, 125.9±0.2 ppm, and/or 123.6±0.2 ppm; and (b) having two or more signals selected from 143.3±0.2 ppm, 138.7±0.2 ppm, 130.7±0.2 ppm, 129.5±0.2 ppm, 117.6±0.2 ppm, and 6.7±0.2 ppm.
335c. The substantially crystalline Compound 4 free form Form B according to Embodiment 319, characterized by a ¹³C CPMAS (a) having signals at 134.0±0.2 ppm, 128.9±0.2 ppm, 125.9±0.2 ppm, and/or 123.6±0.2 ppm; and (b) having three or more signals selected from 143.3±0.2 ppm, 138.7±0.2 ppm, 130.7±0.2 ppm, 129.5±0.2 ppm, 117.6±0.2 ppm, and 6.7±0.2 ppm.
336. The substantially crystalline Compound 4 free form Form B according to Embodiment 319, characterized by a ¹³C CPMAS (a) having signals at 134.0±0.2 ppm, 128.9±0.2 ppm, 125.9±0.2 ppm, and/or 123.6±0.2 ppm; and (b) having signals at 143.3±0.2 ppm, 138.7±0.2 ppm, 130.7±0.2 ppm, 129.5±0.2 ppm, 117.6±0.2 ppm, and 6.7±0.2 ppm.
337. The substantially crystalline Compound 4 free form Form B according to Embodiment 319, characterized by a ¹⁹F MAS having a signal at −109.4±0.2 ppm, −110.5±0.2 ppm, and/or −113.6±0.2 ppm.
338a. The substantially crystalline Compound 4 free form Form B according to Embodiment 319, characterized by a ¹⁹F MAS having signals at −105.0±0.2 ppm, −109.4±0.2 ppm, −110.5±0.2 ppm, and −113.6±0.2 ppm.
338b. The substantially crystalline Compound 4 free form Form B according to Embodiment 319, characterized by a thermogravimetric analysis (TGA) showing minimal weight loss from ambient temperature up to about 200° C.
338c. The substantially crystalline Compound 4 free form Form A according to Embodiment 319, characterized by a differential scanning calorimetry (DSC) analysis showing an endothermic peak at about 246° C.
339. Substantially crystalline Compound 4 free form Form C:

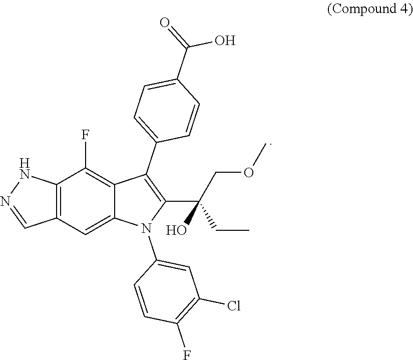 [SMILES: CC[C@@](O)(COC)c1c(-c2ccc(C(=O)O)cc2)c2c(F)c3[nH]ncc3cc2n1-c1ccc(F)c(Cl)c1]

340. The substantially crystalline Compound 4 free form Form C according to Embodiment 339, characterized by (a) an XRPD signal at 13.9±0.2 degrees two theta; and (b) one or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
341. The substantially crystalline Compound 4 free form Form C according to Embodiment 339, characterized by (a) an XRPD signal at 13.9±0.2 degrees two theta; and (b) two or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
342. The substantially crystalline Compound 4 free form Form C according to Embodiment 339, characterized by (a) an XRPD signal at 13.9±0.2 degrees two theta; and (b) three or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
343. The substantially crystalline Compound 4 free form Form C according to Embodiment 339, characterized by (a) an XRPD signal at 13.9±0.2 degrees two theta; and (b) four or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
345. The substantially crystalline Compound 4 free form Form C according to Embodiment 339, characterized by (a) an XRPD signal at 13.9±0.2 degrees two theta; and (b) five or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
346. The substantially crystalline Compound 4 free form Form C according to Embodiment 339, characterized by (a) an XRPD signal at 13.9±0.2 degrees two theta; and (b) six or more XRPD signals selected from 5.6±0.2 degrees two theta, 9.2±0.2 degrees two theta, 11.1±0.2 degrees two theta, 12.2±0.2 degrees two theta, 17.4±0.2 degrees two theta, 18.3±0.2 degrees two theta, and 25.3±0.2 degrees two theta.
347a. The substantially crystalline Compound 4 free form Form C according to Embodiment 339, characterized by XRPD signals at 5.6±0.2 degrees two theta, 9.2±0.2 degrees two theta, 11.1±0.2 degrees two theta, 12.2±0.2 degrees two theta, 13.9±0.2 degrees two theta; 17.4±0.2 degrees two theta, 18.3±0.2 degrees two theta, and 25.3±0.2 degrees two theta.
347b. The substantially crystalline Compound 4 free form Form C according to Embodiment 339, characterized by a thermogravimetric analysis (TGA) showing negligible weight loss from ambient temperature up to about 200° C.
347c. The substantially crystalline Compound 4 free form Form C according to Embodiment 339, characterized by a differential scanning calorimetry (DSC) analysis showing an endothermic peak at about 252° C.
348. Substantially crystalline Compound 4 free form Form D:

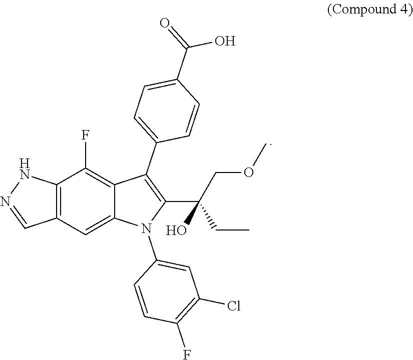 [SMILES: CC[C@@](O)(COC)c1c(-c2ccc(C(=O)O)cc2)c2c(F)c3[nH]ncc3cc2n1-c1ccc(F)c(Cl)c1]

349. The substantially crystalline Compound 4 free form Form D according to Embodiment 348, characterized by one or more XRPD signals selected from 9.9±0.2 degrees two theta, 23.9±0.2 degrees two theta, and 27.6±0.2 degrees two theta.
350. The substantially crystalline Compound 4 free form Form D according to Embodiment 348, characterized by two or more XRPD signals selected from 9.9±0.2 degrees two theta, 23.9±0.2 degrees two theta, and 27.6±0.2 degrees two theta.
350. The substantially crystalline Compound 4 free form Form D according to Embodiment 348, characterized by XRPD signals at 9.9±0.2 degrees two theta, 23.9±0.2 degrees two theta, and 27.6±0.2 degrees two theta.
351. The substantially crystalline Compound 4 free form Form D according to Embodiment 348, characterized by (a) one or more XRPD signals selected from 9.9±0.2 degrees two theta, 23.9±0.2 degrees two theta, and 27.6±0.2 degrees two theta; and (b) one or more XRPD signals selected from 11.1±0.2 degrees two theta, 11.8±0.2 degrees two theta, 17.1±0.2 degrees two theta, 19.0±0.2 degrees two theta, and 24.9±0.2 degrees two theta.
352. The substantially crystalline Compound 4 free form Form D according to Embodiment 348, characterized by (a) one or more XRPD signals selected from 9.9±0.2 degrees two theta, 23.9±0.2 degrees two theta, and 27.6±0.2 degrees two theta; and (b) two or more XRPD signals selected from 11.1±0.2 degrees two theta, 11.8±0.2 degrees two theta, 17.1±0.2 degrees two theta; 19.0±0.2 degrees two theta, and 24.9±0.2 degrees two theta.
353. The substantially crystalline Compound 4 free form Form D according to Embodiment 348, characterized by (a) one or more XRPD signals selected from 9.9±0.2 degrees two theta, 23.9±0.2 degrees two theta, and 27.6±0.2 degrees two theta; and (b) three or more XRPD signals selected from 11.1±0.2 degrees two theta, 11.8±0.2 degrees two theta, 17.1±0.2 degrees two theta; 19.0±0.2 degrees two theta, and 24.9±0.2 degrees two theta.
354. The substantially crystalline Compound 4 free form Form D according to Embodiment 348, characterized by (a) one or more XRPD signals selected from 9.9±0.2 degrees two theta, 23.9±0.2 degrees two theta, and 27.6±0.2 degrees two theta; and (b) four or more XRPD signals selected from 11.1±0.2 degrees two theta, 11.8±0.2 degrees two theta, 17.1±0.2 degrees two theta; 19.0±0.2 degrees two theta, and 24.9±0.2 degrees two theta.
355. The substantially crystalline Compound 4 free form Form D according to Embodiment 348, characterized by (a) one or more XRPD signals selected from 9.9±0.2 degrees two theta, 23.9±0.2 degrees two theta, and 27.6±0.2 degrees two theta; and (b) XRPD signals at 11.1±0.2 degrees two theta, 11.8±0.2 degrees two theta, 17.1±0.2 degrees two theta; 19.0±0.2 degrees two theta, and 24.9±0.2 degrees two theta.
356. The substantially crystalline Compound 4 free form Form D according to Embodiment 348, characterized by (a) two or more XRPD signals selected from 9.9±0.2 degrees two theta, 23.9±0.2 degrees two theta, and 27.6±0.2 degrees two theta; and (b) XRPD signals at 11.1±0.2 degrees two theta, 11.8±0.2 degrees two theta, 17.1±0.2 degrees two theta; 19.0±0.2 degrees two theta, and 24.9±0.2 degrees two theta.
357. The substantially crystalline Compound 4 free form Form D according to Embodiment 348, characterized by XRPD signals selected from 9.9±0.2 degrees two theta, 11.1±0.2 degrees two theta, 11.8±0.2 degrees two theta, 17.1±0.2 degrees two theta; 19.0±0.2 degrees two theta, 23.9±0.2 degrees two theta, 24.9±0.2 degrees two theta and 27.6±0.2 degrees two theta.
358. The substantially crystalline Compound 4 free form Form D according to Embodiment 348, characterized by a ¹³C CPMAS having one or more signals selected from 170.0±0.2 ppm, 140.6±0.2 ppm, 131.8±0.2 ppm, and 125.0±0.2 ppm.
359. The substantially crystalline Compound 4 free form Form D according to Embodiment 348, characterized by a ¹³C CPMAS having two or more signals selected from 170.0±0.2 ppm, 140.6±0.2 ppm, 131.8±0.2 ppm, and 125.0±0.2 ppm.
360. The substantially crystalline Compound 4 free form Form D according to Embodiment 348, characterized by a ¹³C CPMAS having three or more signals selected from 170.0±0.2 ppm, 140.6±0.2 ppm, 131.8±0.2 ppm, and 125.0±0.2 ppm.
361. The substantially crystalline Compound 4 free form Form D according to Embodiment 348, characterized by a ¹³C CPMAS having signals at 170.0±0.2 ppm, 140.6±0.2 ppm, 131.8±0.2 ppm, and 125.0±0.2 ppm.
362. The substantially crystalline Compound 4 free form Form D according to Embodiment 348, characterized by a ¹³C CPMAS (a) having signals at 170.0±0.2 ppm, 140.6±0.2 ppm, 131.8±0.2 ppm, and/or 125.0±0.2 ppm; and (b) having one or more signals selected from 141.9±0.2 ppm, 132.9±0.2 ppm, 129.8±0.2 ppm, 127.4±0.2 ppm, 116.0±0.2 ppm, and 7.3±0.2 ppm.
363. The substantially crystalline Compound 4 free form Form D according to Embodiment 348, characterized by a ¹³C CPMAS (a) having signals at 170.0±0.2 ppm, 140.6±0.2 ppm, 131.8±0.2 ppm, and/or 125.0±0.2 ppm; and (b) having two or more signals selected from 141.9±0.2 ppm, 132.9±0.2 ppm, 129.8±0.2 ppm, 127.4±0.2 ppm, 116.0±0.2 ppm, and 7.3±0.2 ppm.
364. The substantially crystalline Compound 4 free form Form D according to Embodiment 348, characterized by a ¹³C CPMAS (a) having signals at 170.0±0.2 ppm, 140.6±0.2 ppm, 131.8±0.2 ppm, and/or 125.0±0.2 ppm; and (b) having three or more signals selected from 141.9±0.2 ppm, 132.9±0.2 ppm, 129.8±0.2 ppm, 127.4±0.2 ppm, 116.0±0.2 ppm, and 7.3±0.2 ppm.
364. The substantially crystalline Compound 4 free form Form D according to Embodiment 348, characterized by a ¹³C CPMAS (a) having signals at 170.0±0.2 ppm, 140.6±0.2 ppm, 131.8±0.2 ppm, and/or 125.0±0.2 ppm; and (b) having signals at 141.9±0.2 ppm, 132.9±0.2 ppm, 129.8±0.2 ppm, 127.4±0.2 ppm, 116.0±0.2 ppm, and 7.3±0.2 ppm.
365. The substantially crystalline Compound 4 free form Form D according to Embodiment 348, characterized by a ¹³C CPMAS (a) having two or more signals selected from 170.0±0.2 ppm, 140.6±0.2 ppm, 131.8±0.2 ppm, and/or 125.0±0.2 ppm; and (b) having signals at 141.9±0.2 ppm, 132.9±0.2 ppm, 129.8±0.2 ppm, 127.4±0.2 ppm, 116.0±0.2 ppm, and 7.3±0.2 ppm.
366. The substantially crystalline Compound 4 free form Form D according to Embodiment 348, characterized by a ¹³C CPMAS (a) having three or more signals selected from 170.0±0.2 ppm, 140.6±0.2 ppm, 131.8±0.2 ppm, and/or 125.0±0.2 ppm; and (b) having signals at 141.9±0.2 ppm, 132.9±0.2 ppm, 129.8±0.2 ppm, 127.4±0.2 ppm, 116.0±0.2 ppm, and 7.3±0.2 ppm.
367. The substantially crystalline Compound 4 free form Form D according to Embodiment 348, characterized by a ¹³C CPMAS having signals at 170.0±0.2 ppm, 141.9±0.2 ppm, 140.6±0.2 ppm, 132.9±0.2 ppm, 131.8±0.2 ppm, 129.8±0.2 ppm, 127.4±0.2 ppm, 125.0±0.2 ppm, 116.0±0.2 ppm, and 7.3±0.2 ppm. 368. The substantially crystalline Compound 4 free form Form D according to Embodiment 348, characterized by a ¹⁹F MAS having a signal at −108.6±0.2 ppm or −115.9±0.2 ppm.
369. The substantially crystalline Compound 4 free form Form D according to Embodiment 348, characterized by a ¹⁹F MAS having signals at −108.6±0.2 ppm and −115.9±0.2 ppm.
370a. The substantially crystalline Compound 4 free form Form D according to Embodiment 348, characterized by a ¹⁹F MAS having signals at −105.0±0.2 ppm, −115.0±0.2 ppm, and −115.9±0.2 ppm.
370b. The substantially crystalline Compound 4 free form Form D according to Embodiment 348, characterized by a thermogravimetric analysis (TGA) showing negligible weight loss from ambient temperature up to about 200° C.
370c. The substantially crystalline Compound 4 free form Form D according to Embodiment 348, characterized by a differential scanning calorimetry (DSC) analysis showing an endothermic peak at about 268° C.
371. Substantially crystalline Compound 4 free form Hydrate Form A:

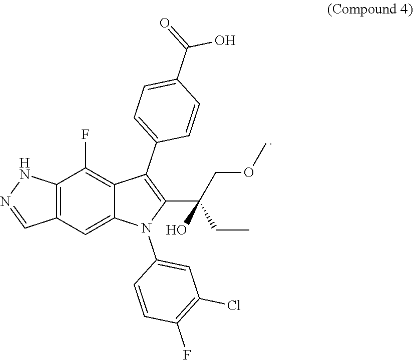 [SMILES: CC[C@@](O)(COC)c1c(-c2ccc(C(=O)O)cc2)c2c(F)c3[nH]ncc3cc2n1-c1ccc(F)c(Cl)c1]

372. The substantially crystalline Compound 4 free form Hydrate Form A according to Embodiment 371, characterized by an XRPD signal at 20.3±0.2 degrees two theta or at 23.3±0.2 degrees two theta.
373. The substantially crystalline Compound 4 free form Hydrate Form A according to Embodiment 371, characterized by an XRPD signal at 20.3±0.2 degrees two theta and a signal at 23.3±0.2 degrees two theta.
374. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) one or more XRPD signals selected from 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
375. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) two or more XRPD signals selected from 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
376. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) three or more XRPD signals selected from 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
377. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) four or more XRPD signals selected from 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
378. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) five or more XRPD signals selected from 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
379. The substantially crystalline Compound 4 free form Hydrate Form A according to Embodiment 371, characterized by (a) an XRPD signal at 20.3±0.2 degrees two theta and/or a signal at 23.3±0.2 degrees two theta; and (b) XRPD signals at 11.8±0.2 degrees two theta, 12.1±0.2 degrees two theta, 15.3±0.2 degrees two theta, 18.0±0.2 degrees two theta, 22.4±0.2 degrees two theta, and 23.6±0.2 degrees two theta.
380. The substantially crystalline Compound 4 free form Hydrate Form A according to Embodiment 371, characterized by XRPD signals at 11.8±0.2 degrees two theta, 12.1±0.2 degrees two theta, 15.3±0.2 degrees two theta, 18.0±0.2 degrees two theta, 20.3±0.2 degrees two theta, 22.4±0.2 degrees two theta, 23.3±0.2 degrees two theta, and 23.6±0.2 degrees two theta.
381. The substantially crystalline Compound 4 free form Hydrate Form A according to Embodiment 371, characterized by a ¹³C CPMAS having one or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
382. The substantially crystalline Compound 4 free form Hydrate Form A according to Embodiment 371, characterized by a ¹³C CPMAS having two or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
383. The substantially crystalline Compound 4 free form Hydrate Form A according to Embodiment 371, characterized by a ¹³C CPMAS having three or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
384. The substantially crystalline Compound 4 free form Hydrate Form A according to Embodiment 371, characterized by a ¹³C CPMAS having four or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
385. The substantially crystalline Compound 4 free form Hydrate Form A according to Embodiment 371, characterized by a ¹³C CPMAS having five or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
386. The substantially crystalline Compound 4 free form Hydrate Form A according to Embodiment 371, characterized by a ¹³C CPMAS having six or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
387. The substantially crystalline Compound 4 free form Hydrate Form A according to Embodiment 371, characterized by a ¹³C CPMAS having seven or more signals selected from 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
388. The substantially crystalline Compound 4 free form Hydrate Form A according to Embodiment 371, characterized by a ¹³C CPMAS having signals at 142.4±0.2 ppm, 136.2±0.2 ppm, 134.1±0.2 ppm, 133.2±0.2 ppm, 131.8±0.2 ppm, 128.8±0.2 ppm, 127.7±0.2 ppm, 124.2±0.2 ppm, 117.9±0.2 ppm, and 7.9±0.2 ppm.
389. The substantially crystalline Compound 4 free form Hydrate Form A according to Embodiment 371, characterized by a ¹⁹F MAS having one or more signals selected from −104.7±0.2 ppm, −105.5±0.2 ppm, −114.4±0.2 ppm, and −115.2±0.2 ppm.
390a. The substantially crystalline Compound 4 free form Hydrate Form A according to Embodiment 371, characterized by a ¹⁹F MAS having signals at −104.7±0.2 ppm, −105.5±0.2 ppm, −114.4±0.2 ppm, and −115.2±0.2 ppm.
390b. The substantially crystalline Compound 4 free form Hydrate Form A according to Embodiment 371, characterized by a thermogravimetric analysis (TGA) showing 3.3% weight loss from ambient temperature up to about 200° C.
390c. The substantially crystalline Compound 4 free form Hydrate Form A according to Embodiment 371, characterized by a differential scanning calorimetry (DSC) analysis showing endothermic peaks around 103, 178, and 257° C. and exothermic peaks around 126 and 204° C.
391. Substantially crystalline Compound 4 free form Hydrate Form B:

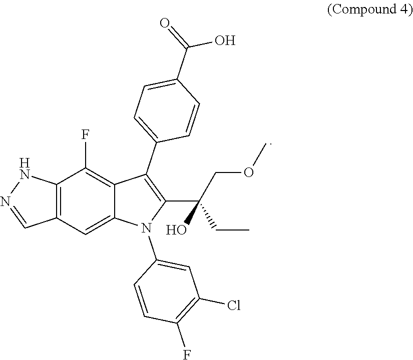 [SMILES: CC[C@@](O)(COC)c1c(-c2ccc(C(=O)O)cc2)c2c(F)c3[nH]ncc3cc2n1-c1ccc(F)c(Cl)c1]

392. The substantially crystalline Compound 4 free form Hydrate Form B according to Embodiment 391, characterized by an XRPD signal at 16.7±0.2 degrees two theta.
393. The substantially crystalline Compound 4 free form Hydrate Form B according to Embodiment 391, characterized by (a) an XRPD signal at 16.7±0.2 degrees two theta; and (b) one or more XRPD signals selected from 9.4±0.2 degrees two theta, 11.8±0.2 degrees two theta, 13.2±0.2 degrees two theta, 15.3±0.2 degrees two theta, 15.8±0.2 degrees two theta, and 19.1±0.2 degrees two theta.
394. The substantially crystalline Compound 4 free form Hydrate Form B according to Embodiment 391, characterized by (a) an XRPD signal at 16.7±0.2 degrees two theta; and (b) two or more XRPD signals selected from 9.4±0.2 degrees two theta, 11.8±0.2 degrees two theta, 13.2±0.2 degrees two theta, 15.3±0.2 degrees two theta, 15.8±0.2 degrees two theta, and 19.1±0.2 degrees two theta.
395. The substantially crystalline Compound 4 free form Hydrate Form B according to Embodiment 391, characterized by (a) an XRPD signal at 16.7±0.2 degrees two theta; and (b) three or more XRPD signals selected from 9.4±0.2 degrees two theta, 11.8±0.2 degrees two theta, 13.2±0.2 degrees two theta, 15.3±0.2 degrees two theta, 15.8±0.2 degrees two theta, and 19.1±0.2 degrees two theta.
396. The substantially crystalline Compound 4 free form Hydrate Form B according to Embodiment 391, characterized by (a) an XRPD signal at 16.7±0.2 degrees two theta; and (b) four or more XRPD signals selected from 9.4±0.2 degrees two theta, 11.8±0.2 degrees two theta, 13.2±0.2 degrees two theta, 15.3±0.2 degrees two theta, 15.8±0.2 degrees two theta, and 19.1±0.2 degrees two theta.
397. The substantially crystalline Compound 4 free form Hydrate Form B according to Embodiment 391, characterized by (a) an XRPD signal at 16.7±0.2 degrees two theta; and (b) five or more XRPD signals selected from 9.4±0.2 degrees two theta, 11.8±0.2 degrees two theta, 13.2±0.2 degrees two theta, 15.3±0.2 degrees two theta, 15.8±0.2 degrees two theta, and 19.1±0.2 degrees two theta.
398a. The substantially crystalline Compound 4 free form Hydrate Form B according to Embodiment 391, characterized by XRPD signals at 9.4±0.2 degrees two theta, 11.8±0.2 degrees two theta, 13.2±0.2 degrees two theta, 15.3±0.2 degrees two theta, 15.8±0.2 degrees two theta, 16.7±0.2 degrees two theta, and 19.1±0.2 degrees two theta.
398b. The substantially crystalline Compound 4 free form Hydrate Form B according to Embodiment 391, characterized by a thermogravimetric analysis (TGA) showing 5.2% weight loss from ambient temperature up to about 200° C.
398c. The substantially crystalline Compound 4 free form Hydrate Form B according to Embodiment 391, characterized by a differential scanning calorimetry (DSC) analysis showing endothermic peaks around 96, 179, and 244° C. and an exothermic peak at around 198° C.
399. Substantially crystalline Compound 4 free form Hydrate Form C:

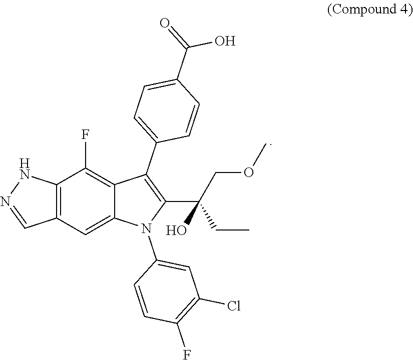 [SMILES: CC[C@@](O)(COC)c1c(-c2ccc(C(=O)O)cc2)c2c(F)c3[nH]ncc3cc2n1-c1ccc(F)c(Cl)c1]

400. The substantially crystalline Compound 4 free form Hydrate Form C according to Embodiment 399, characterized by one or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta.
401. The substantially crystalline Compound 4 free form Hydrate Form C according to Embodiment 399, characterized by two or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta.
402. The substantially crystalline Compound 4 free form Hydrate Form C according to Embodiment 399, characterized by three or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta.
403. The substantially crystalline Compound 4 free form Hydrate Form C according to Embodiment 399, characterized by four or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta.
404. The substantially crystalline Compound 4 free form Hydrate Form C according to Embodiment 399, characterized by XRPD signals at 3.0±0.2 degrees two theta, 6.6±0.2 degrees two theta, 10.5±0.2 degrees two theta, 21.0±0.2 degrees two theta, and 28.1±0.2 degrees two theta.
405. The substantially crystalline Compound 4 free form Hydrate Form C according to Embodiment 399, characterized by (a) one or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta; and (b) one or more XRPD signals selected from 11.6±0.2 degrees two theta, 15.5±0.2 degrees two theta, and 18.1±0.2 degrees two theta.
406. The substantially crystalline Compound 4 free form Hydrate Form C according to Embodiment 399, characterized by (a) one or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta; and (b) XRPD signals at 11.6±0.2 degrees two theta, 15.5±0.2 degrees two theta, and 18.1±0.2 degrees two theta.
407. The substantially crystalline Compound 4 free form Hydrate Form C according to Embodiment 399, characterized by (a) two or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta; and (b) XRPD signals at 11.6±0.2 degrees two theta, 15.5±0.2 degrees two theta, and 18.1±0.2 degrees two theta.
408. The substantially crystalline Compound 4 free form Hydrate Form C according to Embodiment 399, characterized by (a) three or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta; and (b) XRPD signals at 11.6±0.2 degrees two theta, 15.5±0.2 degrees two theta, and 18.1±0.2 degrees two theta.
409. The substantially crystalline Compound 4 free form Hydrate Form C according to Embodiment 399, characterized by (a) four or more XRPD signals selected from 3.0±0.2 degrees two theta, 6.6±0.2 degrees two theta, 10.5±0.2 degrees two theta, 21.0±0.2 degrees two theta, and 28.1±0.2 degrees two theta; and (b) XRPD signals at 11.6±0.2 degrees two theta, 15.5±0.2 degrees two theta, and 18.1±0.2 degrees two theta.
410. The substantially crystalline Compound 4 free form Hydrate Form C according to Embodiment 399, characterized by XRPD signals at 3.0±0.2 degrees two theta, 6.6±0.2 degrees two theta, 10.5±0.2 degrees two theta, 11.6±0.2 degrees two theta, 15.5±0.2 degrees two theta, 18.1±0.2 degrees two theta, 21.0±0.2 degrees two theta, and 28.1±0.2 degrees two theta.
411. The substantially crystalline Compound 4 free form Hydrate Form C according to Embodiment 399, characterized by a differential scanning calorimetry (DSC) analysis showing endothermic peaks around 234 and 249° C.
412. A pharmaceutical composition comprising the substantially crystalline Compound 4 according to any one of Embodiments 297 to 411, and a pharmaceutically acceptable carrier.
413. A method of treating alpha-1 antitrypsin deficiency (AATD) comprising administering to a patient in need thereof a therapeutically effective amount of the substantially crystalline form of Compound 4 according to any one of Embodiments 297 to 411, or a therapeutically effective amount of a pharmaceutical composition according to Embodiment 412.
414. Use of the substantially crystalline form of Compound 3 according to any one of Embodiments 297 to 411, or the pharmaceutical composition according to Embodiment 412 in the manufacture of a medicament for treating AATD.
415. The substantially crystalline form of Compound 3 according to any one of Embodiments 297 to 411, or a therapeutically effective amount of a pharmaceutical composition according to Embodiment 412 for use in treating AATD.
416. A method of preparing substantially crystalline Compound 4 free form Form A comprising the steps of (a) adding 1:2 EtOH:water (v/v) to amorphous Compound 4, (b) stirring at room temperature for 2 hours, (c) centrifuging to collect the solids as seeds, (d) again adding 1:2 EtOH:water (v/v) to amorphous Compound 4, (e) adding seeds from step (c) and stirring for approximately 2 days, and (f) centrifuging, collecting, and drying solids to provide substantially crystalline Compound 4 free form Form A.
417. A method of preparing substantially crystalline Compound 4 free form Form B comprising the steps of (a) adding water to crude Compound 4 and acidifying with HCl to about pH 4, (b) stirring for approximately 20 minutes, (c) washing the organic phase sequentially with water, brine, and drying over MgSO4, (d) filtering and concentrating under reduced pressure to provide Compound 4 as an oil, (e) treating the residue with methylene chloride and concentrating under reduced pressure to provide a tan colored crispy foam, and (f) treating the foam with methylene chloride, heating to reflux for 4 hours and stirring at room temperature for an hour, (f) filtering the resulting suspension and washing collected solids with DCM, (g) drying at 80° C. in vacuum oven to remove DCM and provide substantially crystalline Compound 4 free form Form B.
418. A method of preparing substantially crystalline Compound 4 free form Form C comprising the steps of (a) suspending a sample of Compound 4 free form Form B in MBTE, (b) stir for 3 days at 50° C., (c) isolating the solids and heat at 200° C. until all MBTE is removed to provide substantially crystalline Compound 4 free form Form C.
419. A method of preparing substantially crystalline Compound 4 free form Form D comprising the steps of (a) suspending a sample of amorphous Compound 4 in n-propyl acetate (b) stirring at 5° C. for 4 days, (c) isolating and drying solids at 150° C., until all n-propyl acetate is removed to provide crystalline Compound 4 free form Form D.
420. A method of preparing substantially crystalline Compound 4 free form Hydrate Form A comprising the steps of (a) adding water to a sample of amorphous Compound 4 in a container, (b) sealing the container and shaking at 800 rpms for about two weeks, (c) isolating the solids as seeds, (d) mixing water and Compound 4 free form Form B, (e) bath sonicating for 3 minutes followed by adding seed from step (c) and shaking at 30° C. overnight, (f) adding water and shaking at 30° C. for four days, (g) isolating the solids to provide substantially crystalline Compound 4 free form Hydrate Form A.
420. A method of preparing substantially crystalline Compound 4 free form Hydrate Form B comprising the steps of (a) suspending Compound 4 free form Form B in acetone/water, (b) stirring at room temperature for four days, (c) isolating solids to provide substantially crystalline Compound 4 free form Hydrate Form B.
421. A method of preparing substantially crystalline Compound 4 free form Hydrate Form C comprising the steps of (a) suspending Compound 4 free form Form B in chlorobenzene or methyl cyclohexane, (b) stirring at room temperature for 8 days, (c) isolating solids to provide substantially crystalline Compound 4 free form Hydrate Form C.

### III. PHARMACEUTICAL COMPOSITIONS

Another aspect of the disclosure provides a pharmaceutical composition comprising a compound of Formula Ia, a tautomer thereof, a deuterated derivative of the compound or tautomer, or a pharmaceutically acceptable salt of any of the foregoing. In some embodiments, the pharmaceutical composition comprises a compound of Formula Ia chosen from Compounds 1 to 5. In some embodiments, the pharmaceutical composition comprising a compound of Formula Ia, any of Compounds 1 to 5, a tautomer thereof, a deuterated derivative of the compound or tautomer, or a pharmaceutically acceptable salt of any of the foregoing, is administered to a patient in need thereof.

Some embodiments provide a pharmaceutical composition comprising a compound of Formula Ib, a tautomer thereof, a deuterated derivative of the compound or tautomer, or a pharmaceutically acceptable salt of any of the foregoing. In some embodiments, the pharmaceutical composition comprises a compound of Formula Ib chosen from Compounds 6 to 59. In some embodiments, the pharmaceutical composition comprising a compound of Formula Ib, any of Compounds 6 to 57 a tautomer thereof, a deuterated derivative of the compound or tautomer, or a pharmaceutically acceptable salt of any of the foregoing, is administered to a patient in need thereof.

Some embodiments provide a pharmaceutical composition comprising a compound of Formula Ib-i, a tautomer thereof, a deuterated derivative of the compound or tautomer, or a pharmaceutically acceptable salt of any of the foregoing. In some embodiments, the pharmaceutical composition comprises a compound of Formula Ib-i chosen from Compounds 6 to 21, 33 to 42, 44 to 50, 52, 53, and 57. In some embodiments, the pharmaceutical composition comprising a compound of Formula Ib-i, any of Compounds 6 to 21, 33 to 42, 44 to 50, 52, 53, and 57, a tautomer thereof, a deuterated derivative of the compound or tautomer, or a pharmaceutically acceptable salt of any of the foregoing, is administered to a patient in need thereof.

Some embodiments provide a pharmaceutical composition comprising a compound of Formula Ib-ii, a tautomer thereof, a deuterated derivative of the compound or tautomer, or a pharmaceutically acceptable salt of any of the foregoing. In some embodiments, the pharmaceutical composition comprises a compound of Formula Ib-ii chosen from Compounds 22 to 32, 43, 51, and 54 to 56. In some embodiments, the pharmaceutical composition comprising a compound of Formula Ib-ii, any of Compounds 22 to 32, 43, 51, and 54 to 56, a tautomer thereof, a deuterated derivative of the compound or tautomer, or a pharmaceutically acceptable salt of any of the foregoing, is administered to a patient in need thereof.

Some embodiments provide a pharmaceutical composition comprising a compound of Formula Ic, a tautomer thereof, a deuterated derivative of the compound or tautomer, or a pharmaceutically acceptable salt of any of the foregoing. In some embodiments, the pharmaceutical composition comprises a compound of Formula Ic chosen from Compounds 58 to 67. In some embodiments, the pharmaceutical composition comprising a compound of Formula Ic, any of Compounds 58 to 67, a tautomer thereof, a deuterated derivative of the compound or tautomer, or a pharmaceutically acceptable salt of any of the foregoing, is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure can be employed in combination therapies; that is, the pharmaceutical compositions described herein can further include at least one other active agent. Alternatively, a pharmaceutical composition comprising at least one compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent. In some embodiments, a pharmaceutical composition comprising at least one compound selected from Compounds 1 to 5, Compounds 6 to 21, Compounds 22 to 27, and compounds 28-67, tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent.

In some embodiments, a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, is combined with at least one additional active agent for simultaneous, separate, or sequential use in the treatment of AATD. In some embodiments, when the use is simultaneous, the compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, and the at least one additional active agent are in separate pharmaceutical compositions. In some embodiments, when the use is simultaneous, the compound of Formula I, including compounds of Ia, Ib, Ib-i, Ib-ii, and Ic, tautomers of those compounds, deuterated derivatives of those compounds and tautomers, and pharmaceutically acceptable salts of any of the foregoing, and the at least one additional active agent are together in the same pharmaceutical composition. In some embodiments, the compound is a compound selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, is provided for use in a method of treating AATD, wherein the method comprises co-administering the compound and an additional active agent. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, a combination of a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, and an additional active agent, is provided for use in a method of treating AATD. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, an additional active agent is provided for use in a method of treating AATD, wherein the method comprises co-administrating the additional active agent and a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, is provided for use in a method of treating AATD, wherein the compound is prepared for administration in combination with an additional active agent. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, a combination of a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing, and an additional active agent, is provided for use in a method of treating AATD. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, an additional active agent is provided for use in a method of treating AATD, wherein the additional active agent is prepared for administration in combination with a compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, the additional active agent is selected the group consisting of alpha-1 antitrypsin protein (AAT) from the blood plasma of healthy human donors and recombinant AAT. In some embodiments, the additional active agent is alpha-1 antitrypsin protein (AAT) from the blood plasma of healthy human donors. In some embodiments, the additional active agent is alpha-1 antitrypsin protein (AAT) from the blood plasma of healthy human donors.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants).

In another aspect of the disclosure, the compounds and the pharmaceutical compositions, described herein, are used to treat AATD. In some embodiments, the subject in need of treatment with the compounds and compositions of the disclosure carries the ZZ mutation. In some embodiments, the subject in need of treatment with the compounds and compositions of the disclosure carries the SZ mutation.

In some embodiments, the methods of the disclosure comprise administering to a patient in need thereof a compound chosen from any of the compounds of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing. In some embodiments, the compound are selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing. In some embodiments, said patient in need thereof has a Z mutation in the alpha-1 antitrypsin gene. In some embodiments said patient in need thereof is homozygous for the Z-mutation in the alpha-1 antitrypsin gene.

Another aspect of the disclosure provides methods of modulating alpha-1 antitrypsin activity comprising the step of contacting said alpha-1-antitrypsin with at least one compound of Formula I, including compounds of Formulae Ia, Ib, Ib-i, Ib-ii, and Ic, tautomers of those compounds, deuterated derivatives of those compounds and tautomers, and pharmaceutically acceptable salts of any of the foregoing. In some embodiments, the methods of modulating alpha-1 antitrypsin activity comprising the step of contacting said alpha-1-antitrypsin with at least one compound selected from Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing.

In some embodiments, the methods of modulating alpha-1 antitrypsin activity take place in vivo. In some embodiments, the methods of modulating alpha-1 antitrypsin activity take place ex vivo and said alpha-1-antitrypsin is from a biological sample obtained from a human subject. In some embodiments, the methods of modulating AAT take place in vitro and said alpha-1-antitrypsin is from a biological sample obtained from a human subject. In some embodiments, the biological sample is a blood sample. In some embodiments, the biological sample is a sample taken from a liver biopsy.

### IV. PREPARATION OF COMPOUNDS

All the generic, subgeneric, and specific compound formulae disclosed herein are considered part of the disclosure.

The compounds of the disclosure may be made according to standard chemical practices or as described herein. Throughout the following synthetic schemes and in the descriptions for preparing compounds of Formula I, including compounds of Formulae Ia, Ib, Ib-i, Ib-ii, and Ic, Compounds 1 to 5; Compounds 6 to 21; Compounds 22 to 27; Compounds 33-42, 44-50, 52, 53 and 57; Compounds 28-32, 43, 51 and 54 to 56; and Compounds 58 to 67, tautomers of those compounds, deuterated derivatives of those compounds and tautomers, and pharmaceutically acceptable salts of any of the foregoing, the following abbreviations are used:

### A. ABBREVIATIONS

Unless otherwise noted, or where the context dictates otherwise, the following abbreviations shall be understood to have the following meanings:

| | |
|---|---|
| NMR | Nuclear magnetic resonance |
| ESI-MS | Electrospray mass spectrometry |
| LC/MS | Liquid chromatography-mass spectrometry |
| SFC | Supercritical fluid chromatography |
| ESI | Electrospray ionization |
| cm | Centimeters |
| g | Grams |
| mg | Milligrams |
| L | Liter(s) |
| mL | Milliliters |
| μL | Microliters |
| mmol | Millimoles |
| h | Hours |
| min | Minutes |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/w | Weight-weight concentration |
| rac-BINAP | (±)-2,2′-Bis(diphenylphosphino)- |
| | 1,1′-binaphthalene |
| ^{t}buOK | Potassium tert-butoxide |
| Cbz-Cl | Benzyl chloroformate |
| Cy2NMe | N,N-dicyclohexylmethylamine |
| DCM | Dichloromethane |
| DIBAL | Diisobutylaluminium hydride |
| DIPA | Diisopropylamino |
| DMF | N,N-Dimethylformamide |
| DMSO | Dimethyl sulfoxide |
| Et₂O | Diethyl ether |
| EtOH | Ethanol |
| EtOAc | Ethyl acetate |
| LDA | Lithium diisopropylamide |
| LED | Light Emitting Diode |
| LiHMDS | Lithium bis(trimethylsilyl)amide |
| LiOh | Lithium Hydroxide |
| MeOH | Methanol |
| MeCN | Acetonitrile |
| (Me₂SiH)₂O | 1,1,3,3-Tetramethyldisiloxane |
| 2-MeTHF | 2-Methyltetrahydrofuran |
| MP-TMT | Macroporous polystyrene-bound |
| | 2,4,6-trimercaptotriazine |
| MTBE | Methyl tert-butyl ether |
| NaO^{t}Bu | Sodium tert-butoxide |
| N₂H₄•H₂O | Hydrazine monohydrate |
| NIS | N-Iodosuccinimide |
| PTSA | para-Toluenesulfonic acid |
| RT | Ambient temperature |
| rt | Retention time |
| THF | Tetrahydrofuran |
| TMSCI | Trimethylchlorosilane |

### B. STARTING MATERIALS

6-Bromo-7-fluoro-N-(4-fluoro-3-methoxyphenyl)-1H-indazol-5-amine (S1) and benzyl 6-bromo-7-fluoro-5-((4-fluoro-3-methoxyphenyl)amino)-1H-indazole-1-carboxylate (S2)

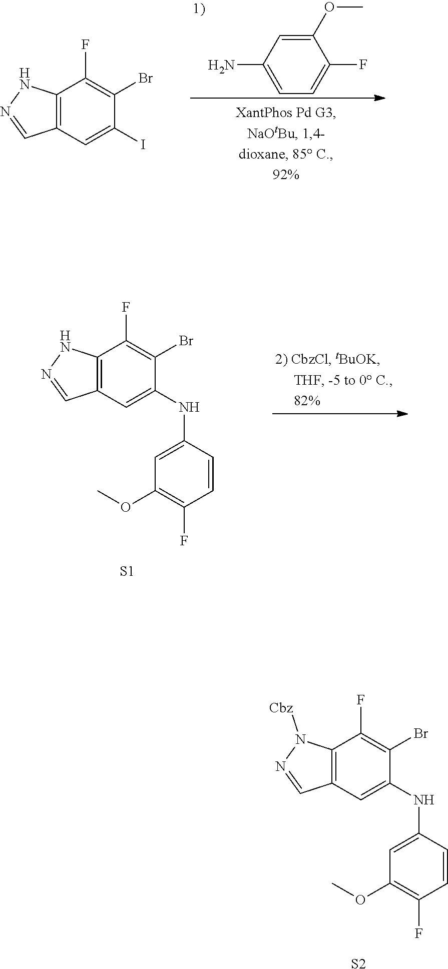

### Step 1

4-Fluoro-3-methoxyaniline (18 g, 126.25 mmol) was added to a stirred and degassed suspension of 6-bromo-7-fluoro-5-iodo-1H-indazole (33 g, 90.989 mmol), NaO^{t}Bu (26.5 g, 267.47 mmol) and XantPhos Pd G3 (4.5 g, 4.513 mmol) in anhydrous 1,4-dioxane (300 mL) at ambient temperature under a nitrogen atmosphere. The synthesis of 6-bromo-7-fluoro-5-iodo-1H-indazole is described in International Patent Publication WO 2021203010 (Compound 55). The mixture was stirred at 85° C. for 1.5 h, then cooled down to ambient temperature. A saturated aqueous NH₄Cl solution (10 vol), an aqueous KHCO₃ solution (20% w/w, 10 vol) and 2-MeTHF (10 vol) were added to the mixture. The organic layer was separated, dried (Na₂SO₄), filtered through a pad of Celite and concentrated in vacuo. The crude residue was taken up in DCM (10 vol) and the suspension was stirred for 3 days at ambient temperature. The suspension was filtered and the solids were dried under reduced pressure at 40° C. for 1 h to give 6-bromo-7-fluoro-N-(4-fluoro-3-methoxyphenyl)-1H-indazol-5-amine (S1, 30.094 g, 92%) as a beige solid. ¹H NMR (400 MHz, DMSO-d₆, 80° C.) δ 13.45 (s, 1H), 8.07 (s, 1H), 7.42 (s, 1H), 7.28 (s, 1H), 6.99 (dd, J=11.5, 8.8 Hz, 1H), 6.74 (dd, J=7.6, 2.7 Hz, 1H), 6.41 (dt, J=8.6, 3.1 Hz, 1H), 3.78 (s, 3H) ppm. ¹⁹F NMR (376 MHz, DMSO-d6, 80° C.) δ −119.43 (s, 1F), −145.92 (s, 1F) ppm. ESI-MS m/z calc. 352.998, found 354.0 (M+1)⁺.

### Step 2

In a 5 L 3-necked round bottom flask fitted with a magnetic stirrer, a J-Kem temperature probe, and a nitrogen inlet/outlet, ^{t}BuOK (40 g, 356.5 mmol) was added to a stirred solution of 6-bromo-7-fluoro-N-(4-fluoro-3-methoxyphenyl)-1H-indazol-5-amine (115 g, 324.7 mmol) in THF (1.2 L) cooled to −5° C. at such a rate to keep the temperature below 0° C. Cbz-Cl (50 mL, 350.2 mmol) was added dropwise via an addition funnel to the reaction mixture at such a rate to keep the temperature below 0° C. The reaction mixture was stirred at this temperature for 30 min. The reaction was quenched by addition of a saturated aqueous NH₄Cl solution (50 mL) and warmed up to ambient temperature over 30 min. The mixture was partitioned between a saturated aqueous NH₄Cl solution (600 mL), water (200 mL) and ethyl acetate (1.4 L) and stirred for 20 min. The organic phase was separated, dried (MgSO₄), filtered, and concentrated in vacuo. The crude residue was triturated from MTBE (1 L) by stirring the suspension for 12 h at ambient temperature. The solid was filtered, rinsed with MTBE (500 mL), dried with air suction for 1 h at ambient temperature and further dried in a vacuum oven at 80° C. for 2 h to give benzyl 6-bromo-7-fluoro-5-((4-fluoro-3-methoxyphenyl)amino)-1H-indazole-1-carboxylate (S2, 130 g, 82%) as a tan solid, which contained small amounts of MTBE and ethyl acetate. ¹H NMR (300 MHz, DMSO-d₆) δ 8.38 (d, J=2.0 Hz, 1H), 7.70 (s, 1H), 7.57-7.49 (m, 2H), 7.49-7.34 (m, 4H), 7.13 (dd, J=11.4, 8.7 Hz, 1H), 6.97 (dd, J=7.8, 2.6 Hz, 1H), 6.68 (ddd, J=8.7, 3.7, 2.6 Hz, 1H), 5.50 (s, 2H), 3.79 (s, 3H) ppm. ¹⁹F NMR (282 MHz, DMSO-d₆) δ −103.16, −143.49 ppm.

The following starting materials were made using the method described in Starting Material 1, except that, in Step 1, different anilines were used as the Buchwald coupling partner in place of 4-fluoro-3-methoxyaniline. In the case of S3 and S5, the reaction was carried out at 90° C. In the case of S4, Step 1 was carried out at 75° C. and Step 2 was carried out in 2-MeTHF as the solvent:

| Starting | | | | |
|---|---|---|---|---|
| Material | | | | |
| Number | Structure | Name | LC/MS | NMR (shifts in ppm) |
| S3 | 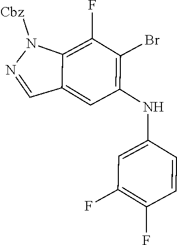 [SMILES: O=C(OCc1ccccc1)n1ncc2cc(Nc3ccc(F)c(F)c3)c(Br)c(F)c21] | Benzyl 6- bromo-5-((3,4- difluorophenyl) amino)-7- fluoro-1H- indazole-1- carboxylate | | 1H NMR (400 MHz, Chloroform-d) δ 8.04 (d, J = 1.9 Hz, 1H), 7.62- 7.52 (m, 2H), 7.50-7.34 (m, 3H), 7.24-7.11 (m, 2H), 7.07 (ddd, J = 11.5, 6.9, 2.7 Hz, 1H), 6.99- 6.85 (m, 1H), 6.11 (s, 1H), 5.56 (s, 2H) ppm. |
| S4 | 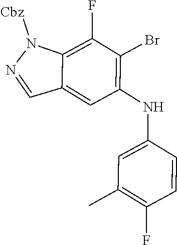 [SMILES: Cc1cc(Nc2cc3cnn(C(=O)OCc4ccccc4)c3c(F)c2Br)ccc1F] | Benzyl 6- bromo-7- fluoro-5-((4- fluoro-3- methylphenyl) amino)-1H- indazole-1- carboxylate | ESI-MS m/z calc. 471.039, found 469.8 (M − 1)⁻. | ¹H NMR (400 MHz, DMSO-d₆) δ 8.37 (d, J = 2.0 Hz, 1H), 7.61 (s, 1H), 7.56-7.48 (m, 2H), 7.48-7.35 (m, 3H), 7.29 (d, J = 1.1 Hz, 1H), 7.13-7.02 (m, 2H), 6.97 (ddd, J = 8.7, 4.5, 2.8 Hz, 1H), 5.49 (s, 2H), 2.21 (d, J = 2.0 Hz, 3H) ppm. |
| S5 | 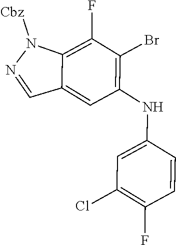 [SMILES: O=C(OCc1ccccc1)n1ncc2cc(Nc3ccc(F)c(Cl)c3)c(Br)c(F)c21] | Benzyl 6- bromo-5-((3- chloro-4- fluorophenyl) amino)-7- fluoro-1H- indazole-1- carboxylate | | ¹H NMR (400 MHz, DMSO-d₆) δ 8.41 (d, J = 1.9 Hz, 1H), 7.99 (s, 1H), 7.56-7.51 (m, 2H), 7.50 (d, J = 1.1 Hz, 1H), 7.47-7.36 (m, 3H), 7.31 (t, J = 9.0 Hz, 1H), 7.20 (dd, J = 6.5, 2.8 Hz, 1H), 7.04 (ddd, J = 8.9, 4.0, 2.7 Hz, 1H), 5.50 (s, 2H) ppm. |

The following starting materials were made using the method described in Starting Material 1, except that, in Step 1, different anilines were used as the Buchwald coupling partner in place of 4-fluoro-3-methoxyaniline. Step 2 was omitted. In the case of S6, Step 1 was carried out at 100° C. in the presence of rac-BINAP-Pd-G3 as the catalyst in place of XantPhos Pd G3. In the case of S7, S14, S15 and S16, Step 1 was carried out at 90° C. In the case of S8, Step 1 was carried out at 80° C. in the presence of ^{t}BuBrettPhos Pd G3 as the catalyst in place of XantPhos Pd G3. In the case of S21, Step 1 was carried out at 75° C. In the case of S35 and S36, Step 1 was carried out at 100° C.:

| Starting | | | | |
|---|---|---|---|---|
| Material | | | | |
| Number | Structure | Name | LC/MS | NMR (shifts in ppm) |
| S6 | 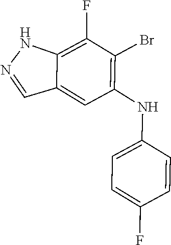 [SMILES: Fc1ccc(Nc2cc3cn[nH]c3c(F)c2Br)cc1] | 6-Bromo-7- fluoro-N-(4- fluorophenyl)- 1H-indazol-5- amine | ESI-MS m/z calc. 322.987, found 323.9 (M + 1)⁺. | ¹H NMR (400 MHz, DMSO-d₆) δ 13.64 (s, 1H), 8.08 (dd, J = 3.5, 1.6 Hz, 1H), 7.57 (s, 1H), 7.39 (s, 1H), 7.09- 6.96 (m, 2H), 6.96-6.81 (m, 2H) ppm. |
| S7 | 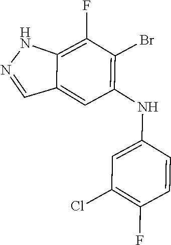 [SMILES: Fc1ccc(Nc2cc3cn[nH]c3c(F)c2Br)cc1Cl] | 6-Bromo-N-(3- chloro-4- fluorophenyl)-7- fluoro-1H- indazol-5-amine | | ¹H NMR (400 MHz, DMSO-d₆) δ 13.75 (s, 1H), 8.14 (dd, J = 3.6, 1.6 Hz, 1H), 7.91 (s, 1H), 7.53 (s, 1H), 7.21 (t, J = 9.1 Hz, 1H), 6.88 (dd, J = 6.4, 2.8 Hz, 1H), 6.76 (ddd, J = 9.0, 4.0, 2.8 Hz, 1H) ppm. |
| S8 | 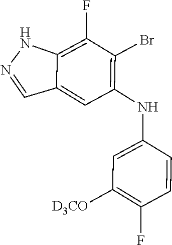 | 6-Bromo-7- fluoro-N-(4- fluoro-3- (methoxy- d₃)phenyl)-1H- indazol-5-amine | ESI-MS m/z calc. 356.016, found 357.0 (M + 1)⁺. | ¹H NMR (400 MHz, DMSO-d₆) δ 13.64 (s, 1H), 8.11-8.05 (m, 1H), 7.54 (s, 1H), 7.44 (s, 1H), 7.00 (dd, J = 11.4, 8.7 Hz, 1H), 6.72 (dd, J = 7.7, 2.6 Hz, 1H), 6.39-6.32 (m, 1H) ppm. |
| S13 | 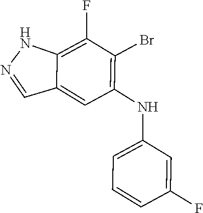 [SMILES: Fc1cccc(Nc2cc3cn[nH]c3c(F)c2Br)c1] | 6-Bromo-7- fluoro-N-(3- fluorophenyl)- 1H-indazol-5- amine | ESI-MS m/z calc. 322.987, found 324.0 (M + 1)⁺. | ¹H NMR (400 MHz, Chloroform-d) δ 10.30 (br d, J = 1.5 Hz, 1H), 8.02 (d, J = 3.4 Hz, 1H), 7.47 (s, 1H), 7.32-7.19 (m, 1H, overlapped with CHCl₃), 6.89-6.76 (m, 2H), 6.68 (br d, J = 2.2 Hz, 1H), 5.94 (s, 1H) ppm. |
| S14 | 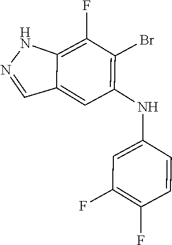 [SMILES: Fc1ccc(Nc2cc3cn[nH]c3c(F)c2Br)cc1F] | 6-Bromo-N-(3,4- difluorophenyl)- 7-fluoro-1H- indazol-5-amine | ESI-MS m/z calc. 340.978, found 341.8 (M + 1)⁺. | ¹H NMR (400 MHz, DMSO-d₆) δ 13.75 (s, 1H), 8.24-8.04 (m, 1H), 7.91 (s, 1H), 7.55 (s, 1H), 7.22 (dt, J = 10.7, 9.1 Hz, 1H), 6.76 (ddd, J = 13.2, 7.0, 2.7 Hz, 1H), 6.67-6.52 (m, 1H) ppm. |
| S15 | 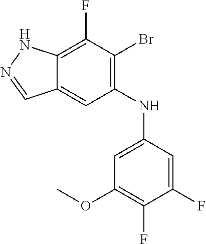 [SMILES: COc1cc(Nc2cc3cn[nH]c3c(F)c2Br)cc(F)c1F] | 6-Bromo-N-(3,4- difluoro-5- methoxyphenyl)- 7-fluoro-1H- indazol-5-amine | ESI-MS m/z calc. 370.988, found 372.0 (M + 1)⁺. | |
| S16 | 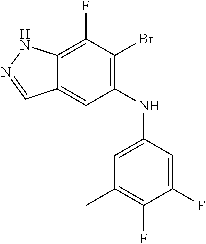 [SMILES: Cc1cc(Nc2cc3cn[nH]c3c(F)c2Br)cc(F)c1F] | 6-Bromo-N-(3,4- difluoro-5- methylphenyl)- 7-fluoro-1H- indazol-5-amine | ESI-MS m/z calc. 354.993, found 356.0 (M + 1)⁺. | |
| S21 | 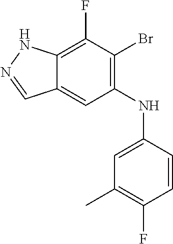 [SMILES: Cc1cc(Nc2cc3cn[nH]c3c(F)c2Br)ccc1F] | 6-Bromo-7- fluoro-N-(4- fluoro-3- methylphenyl)- 1H-indazol-5- amine | ESI-MS m/z calc. 337.003, found 339.0 (M + 1)⁺. | ¹H NMR (400 MHz, DMSO-d₆) δ 13.63 (s, 1H), 8.09 (dd, J = 3.5, 1.6 Hz, 1H), 7.46 (s, 1H), 7.38 (s, 1H), 6.97 (t, J = 9.2 Hz, 1H), 6.78 (dd, J = 6.7, 2.9 Hz, 1H), 6.70 (ddd, J = 7.8, 5.5, 3.3 Hz, 1H), 2.16 (d, J = 1.9 Hz, 3H) ppm. |
| S24 | 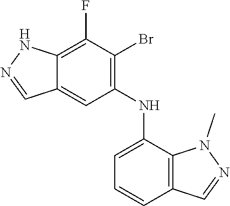 [SMILES: Cn1ncc2cccc(Nc3cc4cn[nH]c4c(F)c3Br)c21] | 6-Bromo-7- fluoro-N-(1- methyl-1H- indazol-7-yl)- 1H-indazol-5- amine | ESI-MS m/z calc. 359.018, found 360.0 (M + 1)⁺. | |
| S35 | 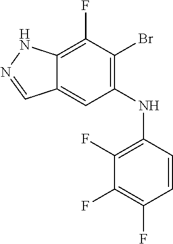 [SMILES: Fc1ccc(Nc2cc3cn[nH]c3c(F)c2Br)c(F)c1F] | 6-Bromo-7- fluoro-N-(2,3,4- trifluorophenyl)- 1H-indazol-5- amine | ESI-MS m/z calc. 358.968, found 360.1 (M + 1)⁺. | |
| S36 | 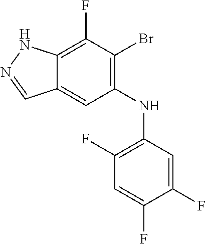 [SMILES: Fc1cc(F)c(Nc2cc3cn[nH]c3c(F)c2Br)cc1F] | 6-Bromo-7- fluoro-N-(2,4,5- trifluorophenyl)- 1H-indazol-5- amine | ESI-MS m/z calc. 358.968, found 360.1 (M + 1)⁺. | |
| S99 | 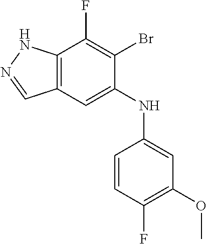 [SMILES: COc1cc(Nc2cc3cn[nH]c3c(F)c2Br)ccc1F] | 6-bromo-7- fluoro-N-(4- fluoro-3- methoxyphenyl)- 1H-indazol-5- amine | 353.94 | 1H NMR (400 MHz, DMSO-d6) δ 13.65 (s, 1H), 8.09 (d, J = 3.4 Hz, 1H), 7.56 (s, 1H), 7.44 (s, 1H), 7.01 (dd, J = 11.4, 8.7 Hz, 1H), 6.73 (dd, J = 7.7, 2.6 Hz, 1H), 6.36 (dt, J = 8.7, 3.3 Hz, 1H), 3.75 (s, 3H). |

The following starting materials were made using the method described in Starting Material 1, except that, in Step 1, different anilines were used as the Buchwald coupling partner in place of 4-fluoro-3-methoxyaniline. In Step 2, pivaloyl chloride was used in place of CbzCl and the reaction was carried out at 0° C. In the case of S25, S27, S29, and S37, Step 1 was carried out at 90° C. In the case of S26, Step 1 was carried out at 75° C. In the case of S28, Step 1 was carried out at 100° C.:

| Starting | | | | |
|---|---|---|---|---|
| Material | | | | |
| Number | Structure | Name | LC/MS | NMR (shifts in ppm) |
| S25 | 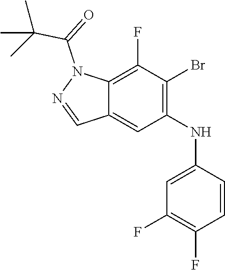 [SMILES: CC(C)(C)C(=O)n1ncc2cc(Nc3ccc(F)c(F)c3)c(Br)c(F)c21] | 1-(6-Bromo-5- ((3,4- difluorophenyl) amino)-7- fluoro-1H- indazol-1-yl)- 2,2- dimethylpropan- 1-one | ESI-MS m/z calc. 425.035, found 425.9 (M + 1)⁺. | ¹H NMR (400 MHz, Methanol-d₄) δ 8.17 (d, J = 1.7 Hz, 1H), 7.37 (d, J = 1.2 Hz, 1H), 7.22- 7.18 (m, 1H), 7.09-7.02 (m, 1H), 6.95-6.87 (m, 1H), 1.57 (s, 9H) ppm; amine NH not observed due to methanol-d₄ exchange. |
| S26 | 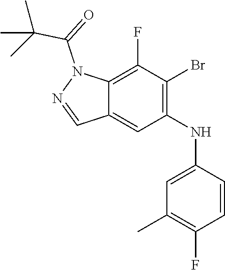 [SMILES: Cc1cc(Nc2cc3cnn(C(=O)C(C)(C)C)c3c(F)c2Br)ccc1F] | 1-(6-Bromo-7- fluoro-5-((4- fluoro-3- methylphenyl) amino)-1H- indazol-1-yl)- 2,2- dimethylpropan- 1-one | ESI-MS m/z calc. 421.060, found 422.1 (M + 1)⁺. | ¹H NMR (400 MHz, DMSO-d₆) δ 8.84 (d, J = 2.7 Hz, 1H), 7.50 (s, 1H), 7.10-7.04 (m, 2H), 7.02 (dd, J = 6.9, 2.8 Hz, 1H), 6.98-6.91 (m, 1H), 2.20 (m, 3H), 1.56 (s, 9H) ppm. |
| S28 | 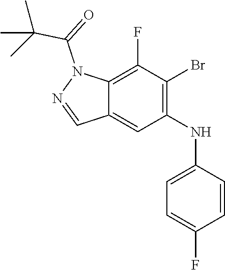 [SMILES: CC(C)(C)C(=O)n1ncc2cc(Nc3ccc(F)cc3)c(Br)c(F)c21] | 1-(6-Bromo-7- fluoro-5-((4- fluorophenyl) amino)-1H-indazol- 1-yl)-2,2- dimethylpropan- 1-one | ESI-MS m/z calc. 407.045, found 408.2 (M + 1)⁺. | ¹H NMR (400 MHz, DMSO-d₆) δ 8.38 (d, J = 1.7 Hz, 1H), 7.73 (s, 1H), 7.30 (s, 1H), 7.20-7.11 (m, 4H), 1.51-1.46 (m, 9H) ppm. |
| S37 | 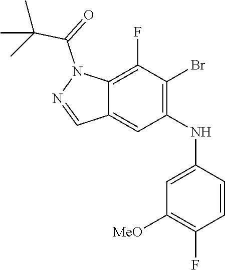 [SMILES: COc1cc(Nc2cc3cnn(C(=O)C(C)(C)C)c3c(F)c2Br)ccc1F] | 1-(6-Bromo-7- fluoro-5-((4- fluoro-3- methoxyphenyl) amino)-1H- indazol-1-yl)- 2,2- dimethylpropan- 1-one | ESI-MS m/z calc. 437.055, found 438.1 (M + 1)⁺. | ¹H NMR (400 MHz, DMSO-d₆) δ 8.40 (d, J = 1.7 Hz, 1H), 7.71 (s, 1H), 7.40 (d, J = 1.0 Hz, 1H), 7.13 (dd, J = 11.4, 8.7 Hz, 1H), 6.98 (dd, J = 7.7, 2.6 Hz, 1H), 6.69 (ddd, J = 8.7, 3.7, 2.6 Hz, 1H), 3.79 (s, 3H), 1.49 (s, 9H) ppm. |

### C. INTERMEDIATES

### Intermediate 1

### Methyl (rac)-4-((1-hydroxy-2-methoxycyclohexyl)ethynyl)benzoate (Peak A, N1), methyl (rac)-4-((1-hydroxy-2-methoxycyclohexyl)ethynyl)benzoate (Peak B, N2), methyl (rel)-4-((1-hydroxy-2-methoxycyclohexyl)ethynyl)benzoate (Peak AA, N3) and methyl (rel)-4-((1-hydroxy-2-methoxycyclohexyl)ethynyl)benzoate (Peak AB, N4)

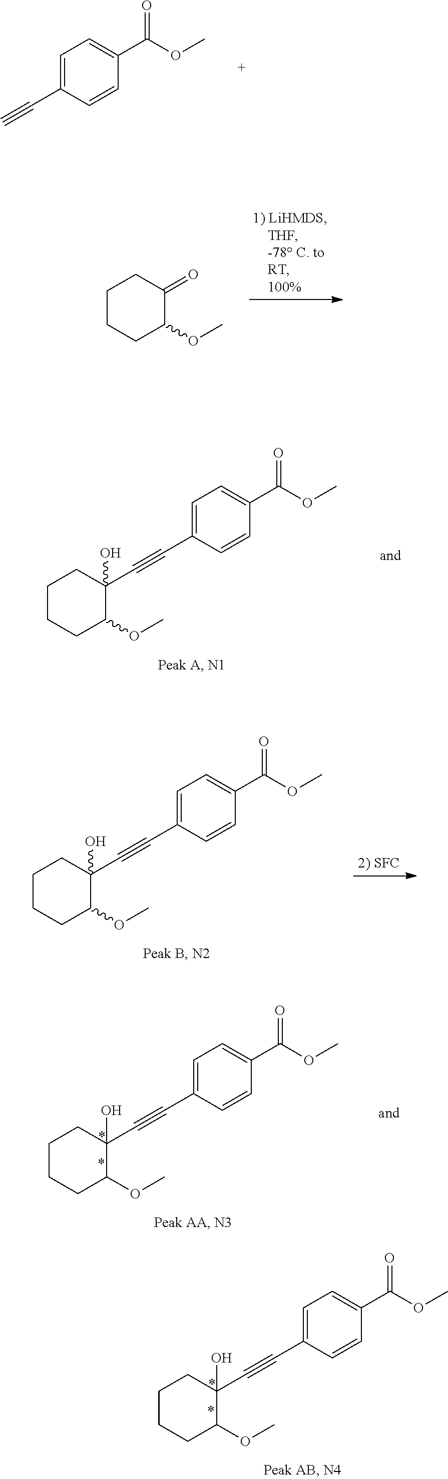

### Step 1

In a 100 mL round-bottom flask, LiHMDS (15 mL, 1 M solution in THF, 15.0 mmol) was added dropwise to a solution of methyl 4-ethynylbenzoate (2 g, 12.24 mmol) in THF (40 mL) at −78° C. under a nitrogen atmosphere and the reaction mixture was stirred at −78° C. for 30 min. (rac)-2-Methoxycyclohexan-1-one (1.9 mL, 15.12 mmol) was added dropwise to the solution via syringe. Upon complete addition, the cold bath was removed and the solution was stirred for 2 h at ambient temperature. The reaction was quenched by addition of an aqueous saturated NH₄Cl solution (20 mL of 1:1 solution) and stirred for 30 min. The mixture was extracted with EtOAc (3×). The combined organic extracts were dried (Na₂SO₄), filtered and concentrated in vacuo. Purification by flash chromatography (80 g SiO₂, 0 to 50% EtOAc in heptane) gave the syn and anti diastereoisomers of methyl 4-((1-hydroxy-2-methoxycyclohexyl)ethynyl)benzoate:

Peak A: methyl (rac)-4-((1-hydroxy-2-methoxycyclohexyl)ethynyl)benzoate (N1, 4.2 g, 90%) as a yellow oil. ¹H NMR (400 MHz, Chloroform-d) δ 8.03-7.89 (m, 2H), 7.53-7.43 (m, 2H), 3.91 (s, 3H), 3.51 (s, 3H), 3.46-3.37 (m, 1H), 2.93 (s, 1H), 2.12-1.96 (m, 1H), 1.90-1.69 (m, 3H), 1.69-1.59 (m, 2H), 1.57-1.47 (m, 1H), 1.43-1.30 (m, 1H) ppm.

Peak B: methyl (rac)-4-((1-hydroxy-2-methoxycyclohexyl)ethynyl)benzoate (N2, 482 mg, 12%) as a yellow oil. ¹H NMR (400 MHz, Chloroform-d) δ 8.03-7.86 (m, 2H), 7.67-7.42 (m, 2H), 3.91 (s, 3H), 3.47 (s, 3H), 3.25 (s, 1H), 3.11 (dd, J=11.3, 4.0 Hz, 1H), 2.22-2.03 (m, 2H), 1.88-1.77 (m, 1H), 1.77-1.58 (m, 3H), 1.56-1.44 (m, 1H), 1.35-1.23 (m, 1H) ppm.

### Step 2

The enantiomers of methyl (rac)-4-((1-hydroxy-2-methoxycyclohexyl)ethynyl)benzoate (Peak A) (N1, 3.33 g, 8.777 mmol) were separated by chiral SFC using a Chiralpak IC column, 5 m particle size, 15 cm×30 mm from Daicel Corporation (Mobile phase: 40% methanol (supplemented with 5 mM ammonia), 60% CO₂; Flow rate 100 mL/min):

Peak AA (rt=1.70 min): methyl rel-4-((1-hydroxy-2-methoxycyclohexyl)ethynyl)benzoate (N3, 1.211 g, 47%) as a yellow oil. ¹H NMR (400 MHz, Chloroform-d) δ 8.09-7.89 (m, 2H), 7.62-7.39 (m, 2H), 3.91 (s, 3H), 3.51 (s, 3H), 3.42 (dd, J=7.4, 3.7 Hz, 1H), 2.94 (s, 1H), 2.15-1.95 (m, 1H), 1.90-1.71 (m, 3H), 1.69-1.58 (m, 2H), 1.56-1.47 (m, 1H), 1.40-1.26 (m, 1H) ppm.

Peak AB (rt=2.61 min): methyl rel-4-((1-hydroxy-2-methoxycyclohexyl)ethynyl) benzoate (N4, 1.122 g, 43%) as a yellow oil. ¹H NMR (400 MHz, Chloroform-d) δ 8.04-7.92 (m, 2H), 7.54-7.42 (m, 2H), 3.91 (s, 3H), 3.51 (s, 3H), 3.47-3.37 (m, 1H), 2.93 (s, 1H), 2.12-1.94 (m, 1H), 1.89-1.69 (m, 3H), 1.68-1.58 (m, 2H), 1.55-1.46 (m, 1H), 1.40-1.27 (m, 1H) ppm.

The following reagents were made using the methods described in Intermediate 1 except that, in Step 1, different ketones were used as starting materials in place of (rac)-2-methoxycyclohexan-1-one:

| | | | | | NMR (shifts in |
|---|---|---|---|---|---|
| | Structure | Name | SFC conditions | LC/MS | ppm) |
| N5 | 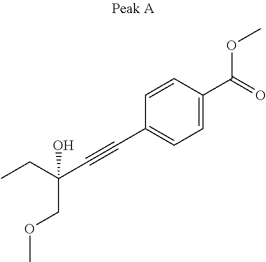 [SMILES: CC[C@@](O)(C#Cc1ccc(C(=O)OC)cc1)COC] | Methyl (R)- 4-(3- hydroxy-3- (methoxy- methyl)pent- 1-yn-1- yl)benzoate | Chiralpak IG column, 5 μm particle size, 25 cm × 20 mm from Daicel Corporation (Mobile phase: 10% methanol (supplemented with 5 mM ammonia), 90% CO₂; Flow rate 80 mL/min); rt = 1.84 min. | | ¹H NMR (400 MHz, DMSO-d₆) δ 7.98-7.90 (m, 2H), 7.57-7.49 (m, 2H), 5.52 (s, 1H), 3.86 (s, 3H), 3.45-3.37 (m, 2H), 3.35 (s, 3H), 1.75-1.58 (m, 2H), 0.99 (t, J = 7.4 Hz, 3H) ppm. |
| N6 | 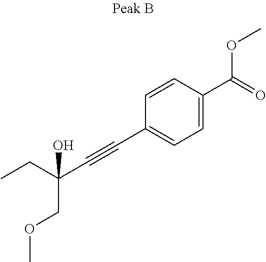 [SMILES: CC[C@](O)(C#Cc1ccc(C(=O)OC)cc1)COC] | Methyl (S)- 4-(3- hydroxy-3- (methoxy- methyl)pent- 1-yn-1- yl)benzoate | Chiralpak IG column, 5 μm particle size, 25 cm × 20 mm from Daicel Corporation (Mobile phase: 10% methanol (supplemented with 5 mM ammonia), 90% CO₂; Flow rate 80 mL/min); rt = 2.12 min. | | ¹H NMR (400 MHz, DMSO-d₆) δ 8.00-7.90 (m, 2H), 7.58-7.50 (m, 2H), 5.53 (s, 1H), 3.87 (s, 3H), 3.47-3.38 (m, 2H), 3.36 (s, 3H), 1.79-1.59 (m, 2H), 1.00 (t, J = 7.4 Hz, 3H) ppm. |
| N8 | 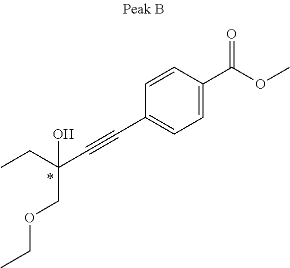 [SMILES: CCOCC(O)(C#Cc1ccc(C(=O)OC)cc1)CC] | Methyl (rel)-(S)-4- (3- (ethoxy- methyl)-3- hydroxypent- 1-yn-1- yl)benzoate | Chiralpak AS-H column, 5 μm particle size, 10 cm × 4.6 mm from Daicel Corporation (Mobile phase: 20% methanol, 80% CO₂; Flow rate 5 mL/min); rt = 0.81 min. | ESI-MS m/z calc. 276.136, found 277.2 (M + 1)⁺. | ¹H NMR (400 MHz, Methanol- d₄) δ 8.04-7.91 (m, 2H), 7.57- 7.42 (m, 2H), 3.90 (s, 3H), 3.69-3.57 (m, 2H), 3.55 (s, 2H), 1.90-1.67 (m, 2H), 1.22 (t, J = 7.0 Hz, 3H), 1.09 (t, J = 7.4 Hz, 3H) ppm; OH alcohol not observed. |
| N11 | 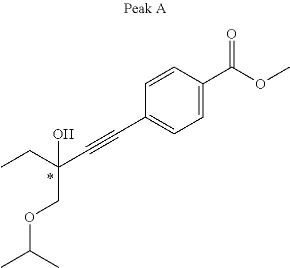 [SMILES: CCC(O)(C#Cc1ccc(C(=O)OC)cc1)COC(C)C] | Methyl (rel)-(R)-4- (3-hydroxy- 3- (isopropoxy- methyl)pent- 1-yn-1- yl)benzoate | Chiralpak AD-H column, 5 μm particle size, 25 cm × 20 mm from Daicel Corporation (Mobile phase: 10% isopropanol (supplemented with 5 mM ammonia), 90% CO₂; Flow rate 75 mL/min); rt = 0.47 min. | ESI-MS m/z calc. 290.152, found 291.1 (M + 1)⁺. | ¹H NMR (300 MHz, DMSO-d₆) δ 8.00-7.86 (m, 2H), 7.56-7.45 (m, 2H), 5.41 (s, 1H), 3.86 (s, 3H), 3.72-3.60 (m, 1H), 3.49- 3.39 (m, 2H), 1.79-1.56 (m, 2H), 1.11 (d, J = 6.1 Hz, 6H), 0.99 (t, J = 7.4 Hz, 3H) ppm. |
| N13 | 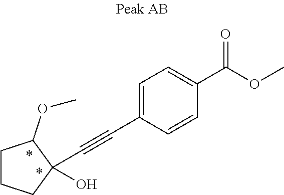 [SMILES: COC(=O)c1ccc(C#CC2(O)CCCC2OC)cc1] | Methyl (rel)-4-((1- hydroxy-2- methoxy- cyclopentyl) ethynyl) benzoate | Chiralpak AD-H column, 5 μm particle size, 25 cm × 20 mm from Daicel Corporation (Mobile phase: 30% methanol (supplemented with 5 mM ammonia), 70% CO₂; Flow rate 80 mL/min); rt = 1.99 min. | ESI-MS m/z calc. 274.121, found 275.2 (M + 1)⁺. | ¹H NMR (400 MHz, Chloroform-d) δ 8.07-7.88 (m, 2H), 7.54-7.40 (m, 2H), 3.91 (s, 3H), 3.86 (t, J = 7.3 Hz, 1H), 3.57 (s, 3H), 3.16 (d, J = 1.3 Hz, 1H), 2.22-2.10 (m, 1H), 2.10-1.99 (m, 2H), 1.96- |
| | | | | | 1.80 (m, 1H), |
| | | | | | 1.79-1.61 (m, |
| | | | | | 2H) ppm. |
| N80 | 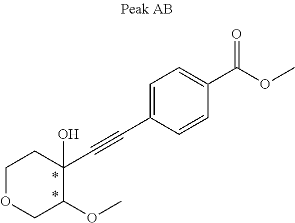 [SMILES: COC(=O)c1ccc(C#CC2(O)CCOCC2OC)cc1] | Methyl (rel)-4-((4- hydroxy-3- methoxy- tetrahydro- 2H- pyran-4- yl)ethynyl) benzoate | Chiralpak IG column, 5 μm particle size, 25 cm × 21.1 mm from Daicel Corporation (Mobile phase: 30% methanol (supplemented with 5 mM ammonia), 70% CO₂; Flow rate 80 mL/min); rt = 2.13 min. | | ¹H NMR (400 MHz, Chloroform-d) δ 8.05-7.93 (m, 2H), 7.58-7.44 (m, 2H), 3.92 (s, 3H), 3.86-3.75 (m, 2H), 3.75- 3.65 (m, 2H), 3.60 (s, 3H), 3.48-3.42 (m, 1H), 3.00 (s, 1H), 2.23-2.09 (m, 1H), 2.08- |
| | | | | | 1.90 (m, 1H) |
| | | | | | ppm. |

The following reagents were made using the methods described in Intermediate 1 except that, in Step 1, different ketones were used as starting materials in place of (rac)-2-methoxycyclohexan-1-one. Step 2 was omitted:

| | Structure | Name | LC/MS | NMR (shifts in ppm) |
|---|---|---|---|---|
| N22 | 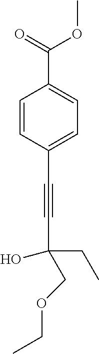 [SMILES: CCOCC(O)(C#Cc1ccc(C(=O)OC)cc1)CC] | Methyl (Rac)4- [3- (ethoxymethyl)- 3-hydroxy-pent- 1-ynyl]benzoate | Not reported due to lack of ionization | 1H NMR (400 MHz, chloroform-d) δ 7.98 (d, J = 8.6 Hz, 2H), 7.50 (d, J = 8.3 Hz, 2H), 3.92 (s, 3H), 3.74-3.67 (m, 1H), 3.66-3.61 (m, 2H), 3.51 (d, J = 9.3 Hz, 1H), 2.91 (s, 1H), 1.88-1.75 (m, 2H), 1.26 (t, J = 7.0 Hz, 3H), 1.13 (t, J = 7.5 Hz, 3H). |
| N81 | 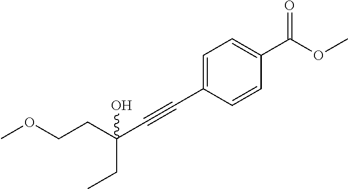 | Methyl (rac)-4- (3-ethyl-3- hydroxy-5- methoxypent-1- yn-1-yl)benzoate | ESI-MS m/z calc. 276.136, found 259.2 (M − 17)⁺. | ¹H NMR (400 MHz, Chloroform-d) δ 7.99 (d, J = 8.3 Hz, 2H), 7.50 (d, J = 8.3 Hz, 2H), 4.29 (s, 1H), 4.04 (td, J = 10.1, 3.3 Hz, 1H), 3.93 (s, 3H), 3.77-3.69 (m, 1H), 3.42 (s, 3H), 2.14-2.03 (m, 1H), 1.92-1.70 (m, 3H), 1.12 (t, J = 7.5 Hz, 3H) ppm. |

### Intermediate 2

### Methyl (rac)-4-(3-(cyclobutoxymethyl)-3-hydroxypent-1-yn-1-yl)benzoate (N35)

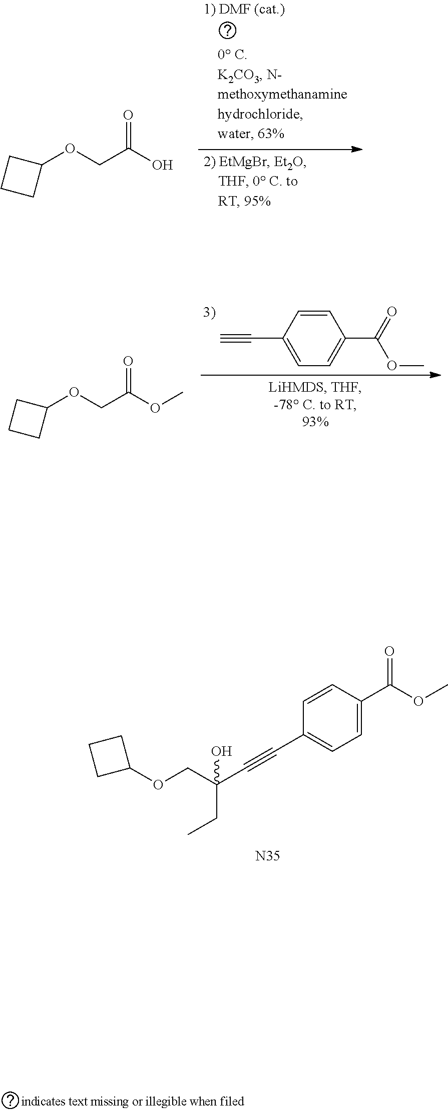

### Step 1

Oxalyl chloride (1.746 g, 1.2 mL, 13.756 mmol) was added dropwise over 5 min to a stirred solution of 2-cyclobutoxyacetic acid (1.5 g, 11.526 mmol) in DCM (35 mL) and DMF (84.016 mg, 0.089 mL, 1.149 mmol) at 0° C. and the reaction mixture was warmed to ambient temperature for 1 h. The resulting acid chloride solution was added to an ice cooled solution of N-methoxymethanamine hydrochloride (1.23 g, 12.610 mmol) and K₂CO₃ (4.77 g, 34.514 mmol) in H₂O (14 mL). The reaction mixture was warmed to ambient temperature and stirred for 18 h. The organic phase was separated. The residue was partitioned between water (30 mL) and EtOAc (2×100 mL) and the organic phase was separated. The combined organic extracts were washed with brine (50 mL), dried (Na₂SO₄), filtered and concentrated in vacuo. Purification by flash chromatography (24 g SiO₂, 0 to 25% EtOAc in heptane) gave 2-cyclobutoxy-N-methoxy-N-methylacetamide (1.5 g, 63%) as a light yellow oil. ESI-MS m/z calc. 173.105, found 174.2 (M+1)⁺.

### Step 2

EtMgBr solution (1.82 mL, 0.456 M solution in 2-MeTHF, 0.830 mmol) was added dropwise over 10 min to a solution of 2-cyclobutoxy-N-methoxy-N-methylacetamide (160 mg, 0.670 mmol) in Et₂O (3 mL) at 0° C. THF (1.5 mL) was added to dissolve a white solid formed during the addition. The mixture was stirred at 0° C. for 10 min. Then, the reaction was warm to ambient temperature and stirred for 4 h. The reaction was quenched by addition of a saturated aqueous NH₄Cl solution (10 mL) and water (5 mL) and extracted with DCM (2×30 mL). The combined organic extracts were washed with brine (20 mL), dried (Na₂SO₄), filtered and concentrated in vacuo to give 1-cyclobutoxybutan-2-one (100 mg, 95%) as a yellow oil. ¹H NMR (400 MHz, Chloroform-d) δ 3.99-392 (m, 3H), 2.50 (q, J=7.3 Hz, 2H), 2.26-2.16 (m, 2H), 2.03-1.93 (m, 2H), 1.76-1.67 (m, 1H), 1.56-1.43 (m, 2H), 1.08 (t, J=7.3 Hz, 3H) ppm.

### Step 3

LiHMDS (6.74 mL, 1 M solution in THF, 6.740 mmol) was added dropwise over 10 min to a stirred solution of methyl 4-ethynylbenzoate (900 mg, 5.6191 mmol) in THF (8 mL) at −78° C. and the mixture was stirred at −78° C. for 2 h. A solution of 1-cyclobutoxybutan-2-one (719 mg, 5.056 mmol) in THF (14 mL) was added over 10 min and the reaction was warmed to ambient temperature and stirred for 18 h. The mixture was quenched by addition of a saturated aqueous NH₄Cl solution (40 mL), water (5 mL) and brine (5 mL) and the mixture was extracted with DCM (2×100 mL). The combined organic extracts were dried (Na₂SO₄), filtered and concentrated in vacuo to give methyl (rac)-4-(3-(cyclobutoxymethyl)-3-hydroxypent-1-yn-1-yl)benzoate (N35, 2.5 g, 93%) as a brown oil. ESI-MS m/z calc. 302.152, found 285.2 (M−17)⁺.

The following reagent was made using the methods described in Intermediate 2 except that, in Step 1, 2-ethoxyacetic acid was used as Starting Material in place of 2-cyclobutoxyacetic acid. In Step 2, cyclopropylmagnesium bromide was used in place of ethylmagnesium bromide and the reaction was carried out in THF as the sole solvent:

| | Structure | Name | LC/MS | NMR (shifts in ppm) |
|---|---|---|---|---|
| N36 | 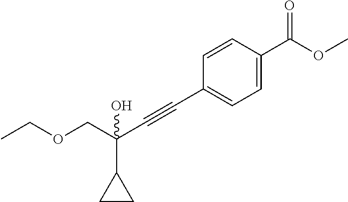 [SMILES: CCOCC(O)(C#Cc1ccc(C(=O)OC)cc1)C1CC1] | Methyl (rac)-4- (3-cyclopropyl-4- ethoxy-3- hydroxybut-1-yn- 1-yl)benzoate | ESI-MS m/z calc. 288.136, found 271.2 (M − 17)⁺. | |

### Intermediate 3

### Methyl (rac)-4-(3-hydroxy-3-(methoxymethyl)pent-1-yn-1-yl)-3-methoxybenzoate (N43), methyl (rel)-(R)-4-(3-hydroxy-3-(methoxymethyl)pent-1-yn-1-yl)-3-methoxybenzoate (Peak A, N44) and methyl (rel)-(S)-4-(3-hydroxy-3-(methoxymethyl)pent-1-yn-1-yl)-3-methoxybenzoate (Peak B, N45)

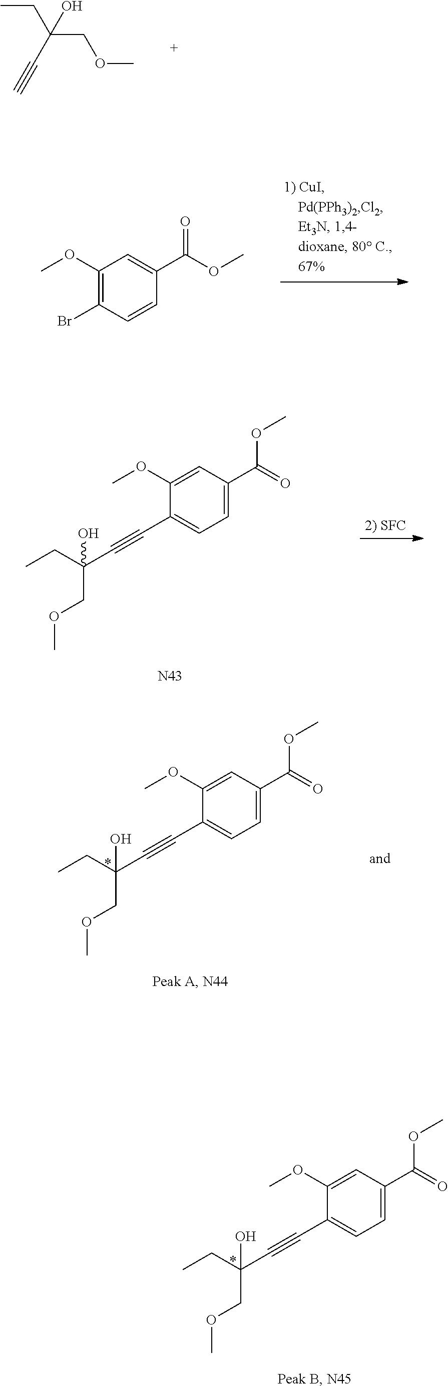

### Step 1

CuI (44 mg, 0.231 mmol) and Pd(PPh₃)₂Cl₂ (69 mg, 0.098 mmol) were added to a stirred and degassed solution of methyl 4-bromo-3-methoxybenzoate (513 mg, 2.093 mmol) and 3-(methoxymethyl)pent-1-yn-3-ol (402 mg, 3.136 mmol) in a mixture of 1,4-dioxane (5.5 mL) and Et₃N (5.5 mL) at ambient temperature and the reaction mixture was heated to 80° C. for 1 h. The mixture was cooled to ambient temperature and filtered through a pad of Celite. The mother liquors were concentrated in vacuo. Purification by flash chromatography (40 g SiO₂, 0 to 100% EtOAc in heptane) gave methyl (rac)-4-(3-hydroxy-3-(methoxymethyl)pent-1-yn-1-yl)-3-methoxybenzoate (N43, 409 mg, 67%) as a viscous pale yellow oil. ¹H NMR (400 MHz, DMSO-d₆) δ 7.61-7.41 (m, 3H), 5.45 (s, 1H), 3.89-3.84 (m, 6H), 3.46-3.37 (m, 2H), 3.36 (s, 3H), 1.79-1.49 (m, 2H), 1.00 (t, J=7.4 Hz, 3H) ppm. ESI-MS m/z calc. 292.131, found 293.1 (M+1)⁺.

### Step 2

The enantiomers of methyl (rac)-4-(3-hydroxy-3-(methoxymethyl)pent-1-yn-1-yl)-3-methoxybenzoate (N43, 340 mg, 1.159 mmol) were separated by chiral SFC using a Chiralpak IG column, 5 m particle size, 15 cm×30 mm from Daicel Corporation (Mobile phase: 20% methanol (supplemented with 5 mM ammonia), 80% CO₂; Flow rate 100 mL/min):

Peak A (rt=0.76 min): methyl (rel)-(R)-4-(3-hydroxy-3-(methoxymethyl)pent-1-yn-1-yl)-3-methoxybenzoate (N44, 145 mg, 43%) as a pale yellow viscous oil. ¹H NMR (400 MHz, DMSO-d₆) δ 7.56-7.43 (m, 3H), 5.45 (s, 1H), 3.87 (m, 6H), 3.45-3.37 (m, 2H), 3.36 (s, 3H), 1.79-1.55 (m, 2H), 1.00 (t, J=7.4 Hz, 3H) ppm. ESI-MS m/z calc. 292.131, found 293.0 (M+1)+.

Peak B (rt=0.85 min): methyl (rel)-(S)-4-(3-hydroxy-3-(methoxymethyl)pent-1-yn-1-yl)-3-methoxybenzoate (N45, 175 mg, 51%) as a pale yellow viscous oil. ¹H NMR (400 MHz, DMSO-d₆) δ 7.57-7.44 (m, 3H), 5.45 (s, 1H), 3.89-3.85 (m, 6H), 3.46-3.37 (m, 2H), 3.32-3.29 (m, 3H), 1.79-1.49 (m, 2H), 1.00 (t, J=7.4 Hz, 3H) ppm. ESI-MS m/z calc. 292.131, found 293.1 (M+1)+.

The following reagent was made using the methods described in Intermediate 3 except that, in Step 1, a different alkyne was used as the starting material in place of 3-(methoxymethyl)pent-1-yn-3-ol and the reaction was carried out in DMF as the solvent. Step 2 was omitted.

| | Structure | Name | LC/MS | NMR (shifts in ppm) |
|---|---|---|---|---|
| N55 | 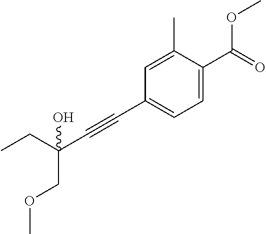 [SMILES: CCC(O)(C#Cc1ccc(C(=O)OC)c(C)c1)COC] | Methyl (rac)-4- (3-hydroxy-3- (methoxymethyl) pent-1-yn-1-yl)- 2- methylbenzoate | ESI-MS m/z calc. 276.166, found 277.1 (M + 1)⁺. | |

### Intermediate 4

### Methyl (rac)-4-(3-hydroxy-3-(methoxymethyl)pent-1-yn-1-yl-4,4,5,5,5-d₅)benzoate (N67)

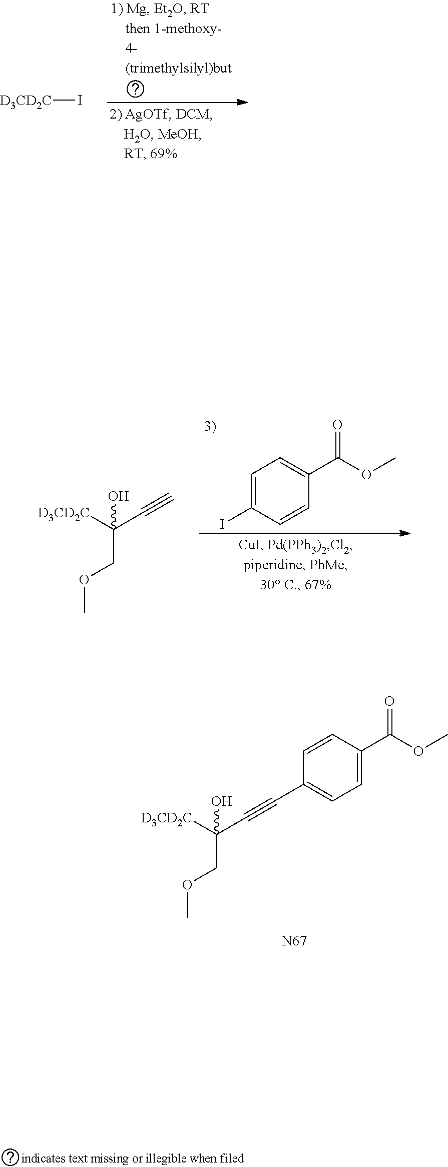

### Step 1

A solution of CD₃CD₂I (29.5 g, 183 mmol) in dry Et₂O (10 mL) was added dropwise to a stirred mixture of magnesium turnings (5.35 g, 220 mmol) and a small crystal of I₂ in dry Et₂O (380 mL) maintained at a steady reflux. The resulting grey mixture was stirred for 2 h at ambient temperature. The prepared Grignard solution was added dropwise over 5.5 h to a solution of methoxy-4-(trimethylsilyl)but-3-yn-2-one (25.0 g, 147 mmol) in dry Et₂O (700 mL) at −78° C. under a N₂ atmosphere and the resultant milky yellow mixture was stirred at 0° C. overnight. The mixture was quenched at −78° C. by addition of a saturated NH₄Cl solution (200 mL). The aqueous phase was separated and extracted with Et₂O (3×200 mL). The combined organic extracts were washed with brine (3×300 mL), dried (Na₂SO₄), filtered and concentrated in vacuo. Purification by flash chromatography (SiO₂, 25% Et₂O in hexanes) gave (rac)-3-(methoxymethyl)-1-(trimethylsilyl)pent-1-yn-4,4,5,5,5-d₅-3-ol (22.9 g, 76%) as a yellow oil. ¹H NMR (400 MHz, Chloroform-d) δ 3.48 (d, J=9.2 Hz, 1H), 3.46 (s, 3H), 3.38 (d, J=9.2 Hz, 1H), 2.61 (br d, 1H), 0.17 (s, 9H) ppm.

### Step 2

Silver triflate (4.30 g, 16.7 mmol) was added to a stirred solution of (rac)-3-(methoxymethyl)-1-(trimethylsilyl)pent-1-yn-4,4,5,5,5-d₅-3-ol (22.9 g, 112 mmol) in a mixture of DCM (595 mL), MeOH (340 mL) and deionized water (85 mL) and the reaction mixture was stirred under N₂ at ambient temperature for 15 h. The mixture was cooled to 0° C. before addition of a saturated NH₄Cl solution (290 mL). The organic layer was separated and concentrated in vacuo. The residue was dissolved in Et₂O (200 mL) and the aqueous phase was further extracted with Et₂O (3×250 mL). The combined organic extracts were washed with brine (2×300 mL), dried (Na₂SO₄), filtered and concentrated in vacuo. Purification by flash chromatography (SiO₂, 40% Et₂O in hexanes) gave (rac)-3-(methoxymethyl)pent-1-yn-4,4,5,5,5-d₅-3-ol (10.3 g, 69%) as a bright yellow oil. ¹H NMR (400 MHz, Chloroform-d) δ 3.50 (d, J=9.2 Hz, 1H), 3.47 (s, 3H), 3.36 (d, J=9.2 Hz, 1H), 2.72 (br d, 1H), 2.44 (s, 1H) ppm.

### Step 3

Piperidine (12.5 mL, 126.5 mmol) was added to a stirred mixture of methyl 4-iodobenzoate (26.1 g, 99.6 mmol), Pd(PPh₃)₂Cl₂ (2.19 g, 3.12 mmol), CuI (1.36 g, 7.13 mmol) in toluene (80 mL) and the mixture was flushed with N₂ for 15 min at ambient temperature. A nitrogen degassed solution of (rac)-3-(methoxymethyl)pent-1-yn-4,4,5,5,5-d₅-3-ol (13.0 g, 97.4 mmol) in toluene (40 mL) was added dropwise to the above mixture over 15 min. The flask was sealed with a Teflon screw cap and placed within an oil bath set to 30° C. The resultant yellow/brown mixture was stirred for 22 h. The dark brown suspension was filtered through a pad of Celite, rinsing with ethyl acetate. The filtrates were concentrated in vacuo to give a red residue (74.9 g). Purification by flash chromatography (SiO₂, 0 to 25% EtOAc in hexanes) gave methyl (rac)-4-(3-hydroxy-3-(methoxymethyl)pent-1-yn-1-yl-4,4,5,5,5-d₅)benzoate (N67, 17.5 g, 67%) as a pink solid. ¹H NMR (400 MHz, Chloroform-d) δ 7.97 (d, J=8.4 Hz, 2H), 7.49 (d, J=8.4 Hz, 2H), 3.92 (s, 3H), 3.58 (d, J=9.2 Hz, 1H), 3.50 (s, 3H), 3.46 (d, J=9.2 Hz, 1H), 2.82 (br s, 1H) ppm.

### Intermediate 5

### Methyl (rac)-4-((2-ethoxy-1-hydroxycyclopentyl)ethynyl)benzoate (Peak A, N82) and methyl (rac)-4-((2-ethoxy-1-hydroxycyclopentyl)ethynyl)benzoate (Peak B, N83)

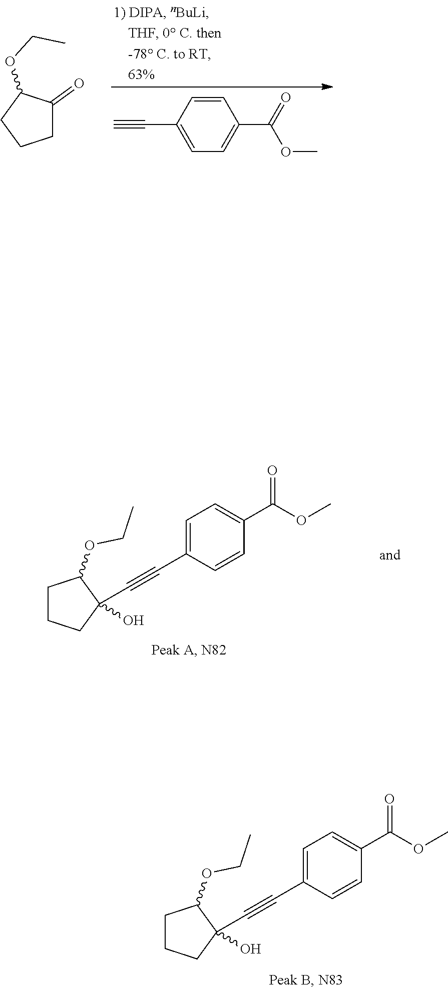

### Step 1

ⁿBuLi (4.2 mL, 1.6 M solution in hexanes, 6.720 mmol) was added dropwise to a stirred solution of DIPA (1.1 mL, 7.848 mmol) in THF (20 mL) at 0° C. After complete addition, the reaction was cooled in a dry ice/acetone bath. A solution of methyl 4-ethynylbenzoate (840 mg, 5.244 mmol) in THF (10 mL) was added dropwise and the reaction mixture was stirred at −78° C. for 30 min. A solution of (rac)-2-ethoxycyclopentan-1-one (860 mg, 6.710 mmol) in THF (10 mL) was added dropwise, the cooling bath was removed and the reaction was stirred for 4 h at ambient temperature. The reaction was quenched by addition of an aqueous saturated NH₄Cl solution and extracted with EtOAc (2×). The combined organic extracts were washed with brine, dried (Na₂SO₄), filtered and concentrated in vacuo. Purification by flash chromatography (40 g SiO₂, 0 to 50% EtOAc in heptane) gave:

Peak A: methyl (rac)-4-((2-ethoxy-1-hydroxycyclopentyl)ethynyl)benzoate (N82, 630 mg, 42%) as a yellow oil. ¹H NMR (400 MHz, Chloroform-d) δ 8.01-7.94 (m, 2H), 7.52-7.43 (m, 2H), 3.95 (t, J 7.3 Hz, 1H), 3.91 (s, 3H), 3.84 (dq, J 9.5, 7.0 Hz, 1H), 3.71 (dq, J 9.4, 7.0 Hz, 1H), 3.25 (s, 1H), 2.22-2.06 (m, 2H), 1.93-1.60 (m, 4H), 1.26 (t, J 7.0 Hz, 3H) ppm.

Peak B: methyl (rac)-4-((2-ethoxy-1-hydroxycyclopentyl)ethynyl)benzoate (N83, 310 mg, 21%) as a yellow oil. ¹H NMR (400 MHz, Chloroform-d) δ 8.05-7.95 (m, 2H), 7.55-7.49 (m, 2H), 3.94 (s, 3H), 3.92-3.81 (m, 1H), 3.80-3.74 (m, 1H), 3.68 (dq, J 9.4, 6.9 Hz, 1H), 2.29-2.12 (m, 2H), 2.04-1.97 (m, 1H), 1.89-1.75 (m, 3H), 1.25 (t, J=7.0 Hz, 3H) ppm; OH alcohol not observed.

The following reagents were made using the methods described in Intermediate 5 except that, in Step 1, different ketones were used as the starting material in place of (rac)-2-ethoxycyclopentan-1-one:

| | Structure | Name | LC/MS | NMR (shifts in ppm) |
|---|---|---|---|---|
| N84 | 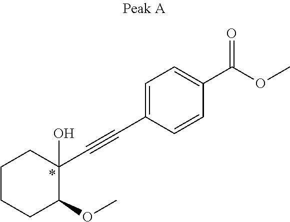 [SMILES: COC(=O)c1ccc(C#CC2(O)CCCC[C@@H]2OC)cc1] | Methyl (rel)-4- (((1S,2S*)-1- hydroxy-2- methoxy- cyclohexyl) ethynyl) benzoate | ESI-MS m/z calc. 288.136, found 271.9 (M − 17)⁺. | ¹H NMR (400 MHz, Chloroform-d) δ 7.98 (d, J = 8.6 Hz, 2H), 7.50 (d, J = 7.6 Hz, 2H), 3.92 (s, 3H), 3.52 (s, 3H), 3.47-3.34 (m, 1H), 2.94 (d, J = 5.9 Hz, 1H), 2.08-1.96 (m, 1H), 1.90-1.70 (m, 3H), 1.70-1.45 (m, 4H) ppm. |
| N85 | 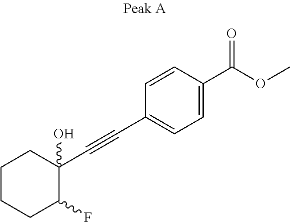 [SMILES: COC(=O)c1ccc(C#CC2(O)CCCCC2F)cc1] | Methyl (rac)-4- ((2-fluoro-1- hydroxy- cyclohexyl) ethynyl) benzoate | ESI-MS m/z calc. 276.116, found 277.3 (M − 17)⁺. | ¹H NMR (400 MHz, Chloroform-d) δ 7.98 (d, J = 8.6 Hz, 2H), 7.50 (d, J = 8.5 Hz, 2H), 4.68 (ddd, J = 48.7, 7.1, 4.0 Hz, 1H), 3.92 (s, 3H), 1.98-1.65 (m, 6H), 1.54-1.35 (m, 2H) ppm; exchangeable H not observed. |

### Intermediate 6

### Methyl (rel)-4-((1-hydroxy-2-(methoxy-d₃)cyclohexyl-2,3,3,4,4,5,5,6,6-d₉)ethynyl)benzoate (N86)

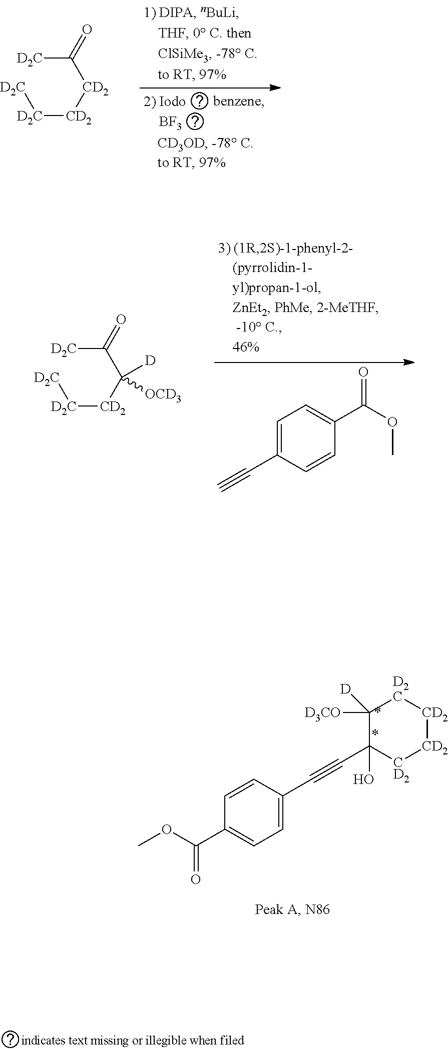

### Step 1

Under a nitrogen atmosphere, BuLi (41 mL, 2.5 M solution in hexanes, 102.5 mmol) was added dropwise to a stirred solution of DIPA (15.6 mL, 111.3 mmol) in THF (200 mL) at 0° C. and the reaction mixture was cooled to −78° C. Cyclohexan-1-one-dio (10 g, 92.42 mmol) was added dropwise and the mixture was stirred at −78° C. for 15 min. TMSCI (18 mL, 141.8 mmol) was added and the reaction was stirred at −78° C. for 15 min and at ambient temperature for 1 h. The reaction was quenched by addition of a saturated NaHCO₃ solution. The aqueous layer was separated and extracted with pentane (3×). The combined organic extracts were dried (Na₂SO₄), filtered and concentrated in vacuo to give ((cyclohex-1-en-1-yl-d₉)oxy)trimethylsilane (16.96 g, 97%) as a colourless oil, which was used in the next step without further purification. ¹H NMR (400 MHz, Chloroform-d) δ 0.17 (s, 9H) ppm.

### Step 2

BF₃·OEt₂ (7 mL, 56.72 mmol) was added to a stirred solution of iodosylbenzene (6.7 g, 30.45 mmol) in methanol-d₄ (22.5 mL) under nitrogen and the reaction mixture was cooled to −78° C. ((Cyclohex-1-en-1-yl-d₉)oxy)trimethylsilane (5 g, 27.87 mmol) was added and the mixture was slowly warmed to ambient temperature and stirred overnight. The reaction was quenched by addition of a saturated NaHCO₃ solution. The aqueous phase was separated and extracted with Et₂O (2×). The combined organic extracts were washed with water and brine, dried (Na₂SO₄), filtered and concentrated to give rac-2-(methoxy-d₃)cyclohexan-1-one-2,3,3,4,4,5,5,6,6-d₉ (16.11 g, 97%) as a yellow oil, which was used without further purification in the next step.

### Step 3

Under nitrogen, ZnEt₂ (13 mL, 1 M solution in hexanes, 13.0 mmol) and a solution of methyl 4-ethynylbenzoate (1 g, 6.243 mmol) in a mixture of toluene (5 mL) and 2-MeTHF (5 mL) were successively added dropwise to a stirred solution of (1R,2S)-1-phenyl-2-(pyrrolidin-1-yl)propan-1-ol (270 mg, 1.315 mmol) in 2-MeTHF (4 mL) at 0° C. and the reaction mixture was stirred at 0° C. for 1 h. The solution was cooled to −10° C. rac-2-(Methoxy-d₃)cyclohexan-1-one-2,3,3,4,4,5,5,6,6-d₉ (5.3 g, 7.596 mmol) was added dropwise and the mixture was stirred at −10° C. for 18 h. The reaction was quenched by addition of a saturated NH₄Cl solution. The aqueous layer was separated and extracted with EtOAc (2×). The combined organic extracts were washed with brine, dried (Na₂SO₄) and passed through a silica gel plug. The filtered cake was washed with EtOAc and the mother liquors were concentrated in vacuo. Purification by flash chromatography (330 g SiO₂, 0 to 60% EtOAc in heptane) gave methyl (rel)-4-((1-hydroxy-2-(methoxy-d₃)cyclohexyl-2,3,3,4,4,5,5,6,6-d₉)ethynyl)benzoate (Peak A, N86, 860 mg, 46%) as single diastereomer. ¹H NMR (400 MHz, Chloroform-d) δ 8.07-7.89 (m, 2H), 7.57-7.37 (m, 2H), 3.92 (s, 3H) ppm; OH alcohol not observed. The other diastereomer, Peak B, was observed but not collected.

The following reagent was made using the methods described in Intermediate 6 except that Steps 1 and 2 were omitted. In Steps 3, (rac)-2-methoxycyclohexan-1-one-2,3,3,4,4,5,5,6,6-d₉ was used as the starting material in place of 2-(methoxy-d₃)cyclohexan-1-one-2,3,3,4,4,5,5,6,6-d₉:

| | Structure | Name | LC/MS | NMR (shifts in ppm) |
|---|---|---|---|---|
| N87 | 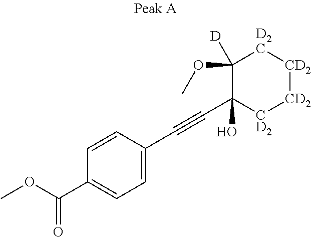 | Methyl 4- (((1S,2S)-1- hydroxy-2- methoxycyclohexyl- 2,3,3,4,4,5,5,6,6- d₉)ethynyl) benzoate | ESI-MS m/z calc. 276.166, found 277.1 (M + 1)⁺. | ¹H NMR (400 MHz, Chloroform-d) δ 8.07-7.90 (m, 2H), 7.54-7.43 (m, 2H), 3.92 (s, 3H), 3.51 (s, 3H), 3.12-2.76 (m, 1H) ppm. |

### Intermediate 7

### Methyl (rel)-(S)-4-(3-hydroxy-3-((methoxy-d₃)methyl)pent-1-yn-1-yl)benzoate (N88)

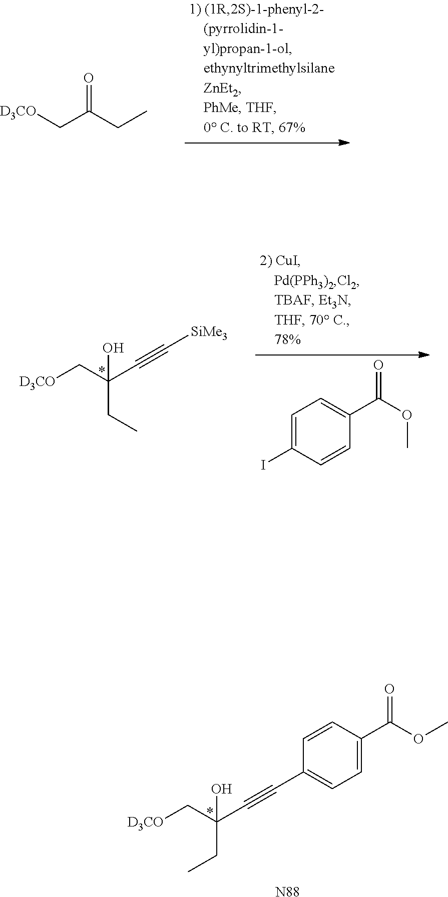

### Step 1

ZnEt₂ (15.5 mL, 1 M solution in hexanes, 15.5 mmol) was slowly added over 12 min to a stirred solution of (1R,2S)-1-phenyl-2-(1-pyrrolidinyl)propan-1-ol (315 mg, 1.504 mmol) in tetrahydrofuran (10 mL) at 0° C. and the resulting suspension was stirred at 0° C. for 22 min. A solution of ethynyltrimethylsilane (2.015 g, 2.9 mL, 20.105 mmol) in toluene (2.5 mL) was added via cannula and the reaction was stirred at 0° C. for 10 min. 1-(Methoxy-d₃)butan-2-one (108.8 g, 1.05% w/w solution in Et₂O, 10.864 mmol) was then added and the reaction mixture was slowly warmed up to ambient temperature over 20.5 h. The reaction was quenched by careful addition of a saturated aqueous NH₄Cl solution (100 mL). The aqueous layer was separated and extracted with MTBE (3×100 mL). The combined organic extracts were washed with brine (1×50 mL), dried (MgSO₄), filtered, and concentrated in vacuo. Purification by flash chromatography (50 g SiO₂, 0 to 15% EtOAc in heptane) gave rel-(S)-3-((methoxy-d₃)methyl)-1-(trimethylsilyl)pent-1-yn-3-ol (1.565 g, 67%) as a light yellow oil. ¹H NMR (400 MHz, Chloroform-d) δ 3.51-3.45 (m, 1H), 3.42-3.37 (m, 1H), 1.76-1.61 (m, 3H), 1.05 (t, J=7.5 Hz, 3H), 0.18 (s, 9H) ppm. ESI-MS m/z calc. 203.142, found 186.2 (M−17)*.

### Step 2

CuI (59.1 mg, 0.304 mmol) and Et₃N (3.612 g, 5 mL, 35.516 mmol) were successively added to a stirred mixture of methyl 4-iodobenzoate (1.654 g, 6.121 mmol) and rel-(S)-3-((methoxy-d₃)methyl)-1-(trimethylsilyl)pent-1-yn-3-ol (1.56 g, 7.287 mmol) in THF (12 mL) and the reaction mixture was flushed with nitrogen gas at ambient temperature for 10 min. PdCl₂(PPh₃)₂ (220.1 mg, 0.307 mmol) and TBAF (7 mL, 1 M solution in THF, 7.0 mmol) were added and the reaction was flushed with nitrogen gas for a further 10 min. The flask was sealed and the mixture was heated at 70° C. for 2 h. After cooling to ambient temperature, the mixture was stirred for 66.5 h. The mixture was partitioned between EtOAc (20 mL) and a half-saturated aqueous NH₄Cl solution (20 mL). The aqueous layer was separated and extracted with EtOAc (3×20 mL). The combined organic extracts were washed with brine (20 mL), dried (MgSO₄), filtered, and concentrated in vacuo. Purification by flash chromatography (50 g SiO₂, 0 to 200 EtOAc in heptane) gave methyl (re-(S)-4-(3-hydroxy-3-((methoxy-d₃)methyl)pent-1-yn-1-yl)benzoate (N88, 1.268 g, 78%) as an orange oil. ¹H NMR (400 z, DMSO-d₆) δ 7.96-7.91 (m, 2H), 7.55-7.50 (m, 2H), 5.52 (s, 1H), 3.86 (s, 3H), 3.45-3.36 (m, 2H), 1.78-1.57 (m, 2H), 0.99 (t, J 7.5 Hz, 3H) ppm. ESI-MS m/z calc. 265.139, found 248.1 (M−17)⁺.

The following reagents were made using the methods described in Intermediate 7 except that, different ketones starting materials were used in place of 1-(methoxy-d₃)butan-2-one in Step 1 and/or different halides were used in place of methyl 4-iodobenzoate in Step 2:

| | Structure | Name | LC/MS | NMR (shifts in ppm) |
|---|---|---|---|---|
| N89 | 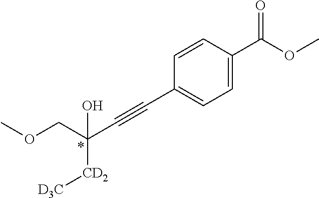 | Methyl (rel)-(S)- 4-(3-hydroxy-3- (methoxymethyl) pent-1-yn-1-yl- 4,4,5,5,5- d5)benzoate | ESI-MS m/z calc. 267.152, found 250.1 (M − 17)⁺. | ¹H NMR (400 MHz, DMSO-d₆) δ 7.98-7.92 (m, 2H), 7.55-7.50 (m, 2H), 5.51 (s, 1H), 3.85 (s, 3H), 3.45-3.36 (m, 2H), 3.35 (s, 3H) ppm. |
| N90 | 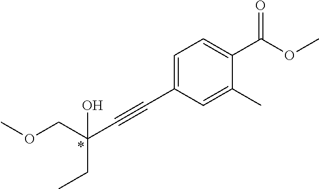 | Methyl (rel)- (S)-4-(3- hydroxy-3- (methoxymethyl) pent-1-yn-1-yl)- 2- methylbenzoate | ESI-MS m/z calc. 276.136, found 259.2 (M − 17)⁺. | ¹H NMR (400 MHz, DMSO-d₆) δ 7.80 (d, J = 8.0 Hz, 1H), 7.35 (s, 1H), 7.32 (d, J = 8.0 Hz, 1H), 5.50 (s, 1H), 3.81 (s, 3H), 3.41 (ABq, J = 9.6 Hz, 2H), 3.34 (s, 3H), 2.49 (s, 3H), 1.78-1.67 (m, 1H), 1.66-1.56 (m, 1H), 0.98 (t, J = 7.6 Hz, 3H) ppm. |
| N91 | 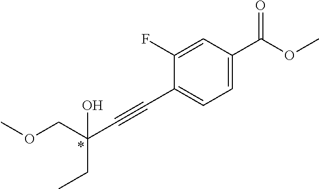 | Methyl (rel)- (S)-3-fluoro-4- (3-hydroxy-3- (methoxymethyl) pent-1-yn-1- yl)benzoate | ESI-MS m/z calc. 280.111, found 298.3 (M − 17)⁺. | ¹H NMR (400 MHz, DMSO-d₆) δ 7.82-7.31 (m, 2H), 7.63 (t, J = 7.6 Hz, 1H), 5.61 (s, 1H), 3.87 (s, 3H), 3.44 (ABq, J = 9.6 Hz, 2H), 3.36 (s, 3H), 1.82- 1.59 (m, 2H), 1.02 (t, J = 8 Hz, 3H) ppm. |
| N92 | 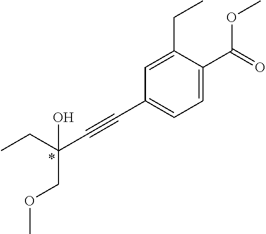 [SMILES: CCc1cc(C#CC(O)(CC)COC)ccc1C(=O)OC] | Methyl (rel)-(S)- 2-ethyl-4-(3- hydroxy-3- (methoxymethyl) pent-1-yn-1- yl)benzoate | ESI-MS m/z calc. 290.1518, found 273.3 (M − 17)⁺. | ¹H NMR (400 MHz, DMSO-d₆) δ 7.75 (d, J = 8 Hz, 1H), 7.35 (s, 1H), 7.31 (d, J = 8 Hz, 1H), 5.51 (s, 1H), 3.82 (s, 3H), 3.42 (ABq, J = 9.6 Hz, 2H), 3.34 (s, 3H), 2.87 (q, J = 7.2 Hz, 2H) 1.71-1.54 (m, 2H), 1.13 (t, J = 7.6 Hz, 3H), 0.98 (t, J = 7.2 Hz, 3H) ppm. |

### Intermediate 8

### Methyl 4-(((2S)-1-(rel)-hydroxy-2-methoxycyclohexyl)ethynyl)-2-methylbenzoate (N99)

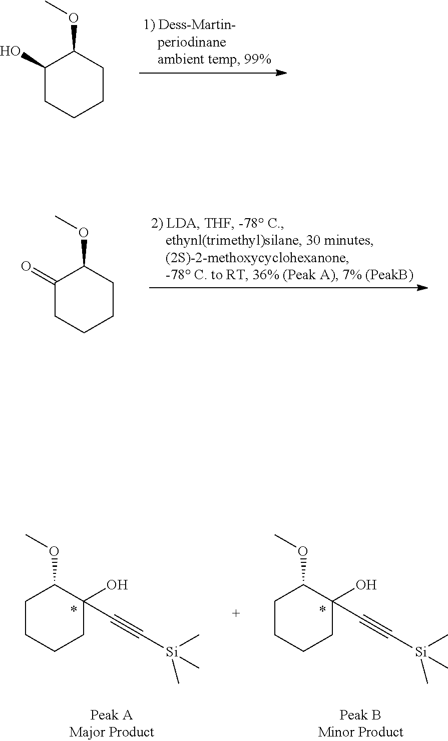

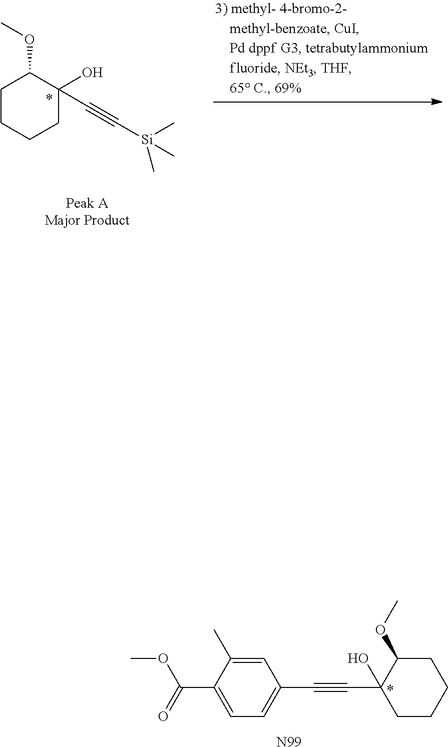

### Step 1

To a solution of (1R,2S)-2-methoxycyclohexanol (10.2 g, 70.515 mmol) in DCM (300 mL) at room temperature was added Dess-Martin periodinane (49.9 g, 105.88 mmol) in one portion. The resulting mixture was stirred at room temperature for 18 hrs. The reaction was quenched by adding saturated aqueous sodium thiosulfate (350 mL) and saturated aqueous sodium bicarbonate (350 mL) slowly. The resulting biphasic mixture was stirred vigorously for 20 minutes. The phases were separated, and the aqueous layer was extracted with dichloromethane (3×100 mL). The combined organic layers were washed with brine (100 mL), dried over anhydrous sodium sulfate, filtered, and concentrated in vacuo to afford (1-(rel), 2S)-2-methoxycyclohexanone (11.4 g, 99%) as a light-yellow oil. 1H NMR (400 MHz, CDCl3) δ 3.75-3.69 (m, 1H), 3.43 (s, 3H), 2.57-2.49 (m, 1H), 2.35-2.20 (m, 2H), 1.99-1.89 (m, 2H), 1.76-1.64 (m, 3H).

### Step 2

To a 100 mL flame-dried round bottom flask equipped with a magnetic stir bar and under inert atmosphere was added LDA (6.93 mL of 1 M, 6.9300 mmol). The solution was cooled to −78° C. and diluted with THF (25 mL). Ethynyl(trimethyl)silane (553.02 mg, 0.78 mL, 5.6305 mmol) was added dropwise while stirring under N2. Then, the reaction was allowed to stir at −78° C. for another 30 min. After that, (2S)-2-methoxycyclohexanone (1.11 g, 6.4953 mmol) was added dropwise via syringe. Upon completion, the cold bath was removed, and the solution was stirred for 2 hours before quenching with 20 mL 1:1 Sat. NH4Cl/H2O. The mixture was allowed to stir for 30 min before being extracted with EtOAc (3×20 mL). The combined organic layers were dried over sodium sulfate and concentrated under reduced pressure to furnish the crude product. The purification by normal phase silica gel chromatography (0-10% EtOAc-EtOAc/Heptanes) afforded (1-(rel), 2S)-2-methoxy-1-(2-trimethylsilylethynyl)cyclohexanol.

(Peak A) (486 mg, 36%) 1H NMR (400 MHz, CDCl3) δ 3.48 (s, 3H), 3.33 (dd, J=7.0, 4.0 Hz, 1H), 2.80 (s, 1H), 1.96-1.84 (m, 1H), 1.82-1.64 (m, 3H), 1.62-1.41 (m, 4H), 0.25-0.12 (m, 9H). and (1-(rel), 2S)-2-methoxy-1-(2-trimethylsilylethynyl)cyclohexanol (Peak B) (210 mg, 7%) 1H NMR (400 MHz, CDCl3) δ 3.44 (s, 3H), 3.10 (s, 1H), 3.00 (dd, J=11.2, 3.9 Hz, 1H), 2.08-1.97 (m, 3H), 1.82-1.70 (m, 1H), 1.68-1.36 (m, 4H), 0.19 (s, 9H).

Step 3:

A slurry of (2S)-2-methoxy-1-(2-trimethylsilylethynyl)cyclohexanol (1 g, 4.417 mmol), methyl 4-bromo-2-methyl-benzoate (1.1 g, 4.802 mmol), CuI (50 mg, 0.2625 mmol), Pd dppf G3 (400 mg, 0.4328 mmol), TBAF (4.5 mL of 1 M, 4.500 mmol) and triethylamine (3.8 mL, 27.26 mmol) in THF (20 mL) was heated at 65° C. for 15 hours under N2. Upon completion, the reaction was cooled to rt, passed through a silica gel plug with EtOAc and concentrated under reduced pressure. The crude product was purified by column chromatography (80 g silica, 0-50% EtOAc/heptane) to furnish methyl 4-[2-[(2S)-1-(rel)-hydroxy-2-methoxy-cyclohexyl]ethynyl]-2-methyl-benzoate (916 mg, 69%) as yellow oil. 1H NMR (400 MHz, CDCl3) δ 7.85 (d, J=8.0 Hz, 1H), 7.35-7.27 (m, 2H), 3.88 (s, 3H), 3.50 (s, 3H), 3.42 (dd, J=7.4, 3.7 Hz, 1H), 2.94 (s, 1H), 2.56 (s, 3H), 2.07-1.92 (m, 1H), 1.88-1.67 (m, 3H), 1.63-1.55 (m, 3H), 1.43-1.27 (m, 1H).

The following reagents were made using the methods described for Intermediate 8 except that, different halide starting material was used in place of methyl 4-bromo-2-methyl-benzoate in Step 3:

| | Structure | Name | LC/MS | NMR (shifts in ppm) |
|---|---|---|---|---|
| N101 | 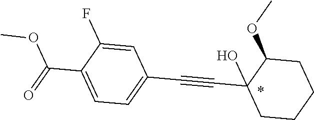 [SMILES: COC(=O)c1ccc(C#CC2(O)CCCC[C@@H]2OC)cc1F] | methyl 2-fluoro- 4-(((1-(rel), 2S)-1- hydroxy-2- methoxy- cyclohexyl) ethynyl) benzoate | | 1H NMR (400 MHz, CDCl3) δ 7.87 (t, J = 7.6 Hz, 1H), 7.25- 7.16 (m, 2H), 3.92 (s, 3H), 3.50 (s, 3H), 3.44-3.38 (m, 1H), 2.93 (s, 1H), 2.07- 1.97 (m, 1H), 1.89- |
| | | | | 1.71 (m, 3H), 1.69- |
| | | | | 1.56 (m, 2H), 1.55- |
| | | | | 1.47 (m, 1H), 1.39- |
| | | | | 1.27 (m, 1H). |

### Intermediate 9

### Methyl (S)-4-(3-hydroxy-3-((methoxy-d3)methyl)pent-1-yn-1-yl-4,4,5,5,5-d5)benzoate (N100)

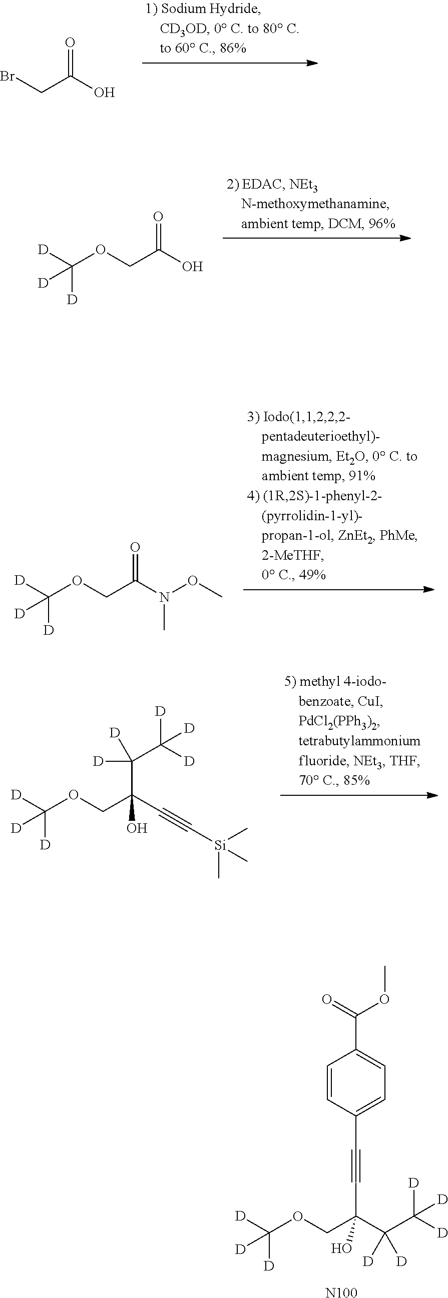 [SMILES: O=C(O)CBr ; [2H]C([2H])([2H])OCC(=O)O ; [2H]C([2H])([2H])OCC(=O)N(C)OC ; [2H]C([2H])([2H])OC[C@@](O)(C#C[Si](C)(C)C)C([2H])([2H])C([2H])([2H])[2H] ; [2H]C([2H])([2H])OC[C@@](O)(C#Cc1ccc(C(=O)OC)cc1)C([2H])([2H])C([2H])([2H])[2H] ; "N100"]

### Step 1

To a stirred solution of sodium hydride (6.91 g, 60% w/w, 172.77 mmol) in THF (55 mL) at 0° C. under N2 was added trideuterio(deuteriooxy)methane (26.973 g, 34.1 mL, 747.87 mmol). After the stirring for 0.5 h at 0° C. 2-bromoacetic acid (8 g, 57.575 mmol) was added and the resulting solution was stirred for 5 min at 0° C. followed by refluxing at 80° C. for 4 h. The reaction was stirred at 60° C. for 18 h. The reaction mixture was quenched with water (30 mL) and washed with hexane (30 mL). The aqueous layer was adjusted to pH 2-3 and extracted with DCM (3×50 mL). The combined organic layers were dried over anhydrous sodium sulfate, filtered, concentrated under vacuum up to 80% of total volume, rest solvent was reduced by flow of N2 and afforded 2-(trideuteriomethoxy)acetic acid (5.43 g, 86%) as a light yellow oil. 1H NMR (400 MHz, DMSO-d6) δ 12.58 (s, 1H), 3.92 (s, 2H).

### Step 2: N-methoxy-N-methyl-2-(trideuteriomethoxy)acetamide

2-(Trideuteriomethoxy)acetic acid (65.18 g, 32.906 mmol) was dissolved in DCM (70 mL) and cooled down to 0° C. followed by addition of triethylamine (4.9368 g, 6.8 mL, 48.787 mmol), 1-(3-dimethylaminopropyl)-3-ethylcarbodiimide (hydrochloric acid salt, 9.5 g, 49.556 mmol), N-methoxymethanamine (hydrochloric acid salt, 3.21 g, 32.908 mmol) and DMAP (41 mg, 0.3356 mmol). The reaction mixture was stirred at room temperature for 18 h. The reaction mixture was washed with 1M aqueous HCl (25 mL), 1M aqueous NaOH (25 mL), dried over anhydrous Na2SO4 and concentrated in vacuo to afford N-methoxy-N-methyl-2-(trideuteriomethoxy)acetamide (4.63 g, 96%) as a pale yellow color oil. 1H NMR (400 MHz, CDCl3) δ 4.22 (s, 2H), 3.69 (s, 3H), 3.20 (s, 3H).

### Step 3: 3,3,4,4,4-pentadeuterio-1-(trideuteriomethoxy)butan-2-one

To a solution of N-methoxy-N-methyl-2-(trideuteriomethoxy)acetamide (7 g, 44.211 mmol) in diethyl ether (200 mL) at 0° C. was added iodo(1,1,2,2,2-pentadeuterioethyl)magnesium (50 mL of 1.6149 M, 80.745 mmol) dropwise over 25 min. The reaction mixture was slowly warmed to room temperature during 18 hours. The reaction mixture was cooled 0° C. and quenched with 3M aq. HCl (22 mL):brine (15 mL). The layers were separated and the organic layer was washed with a mixture of 3M aq. HCl (15 mL) and brine (15 mL) and finally with brine (20 mL). All aqueous layers were combined and extracted with diethyl ether (3×20 mL). The organic layers were combined and dried over anhydrous Na2SO4 to afforded 3,3,4,4,4-pentadeuterio-1-(trideuteriomethoxy)butan-2-one (338 g, 92%) as a solution in diethyl ether. 1H NMR (400 MHz, CDCl3) δ 4.01 (s, 2H). The product was stored over molecular sieves and used as it in the next step.

### Step 4: (3S)-4,4,5,5,5-pentadeuterio-3-(trideuteriomethoxymethyl)-1-trimethylsilyl-pent-1-yn-3-ol

In a round-bottomed flask, (1R,2S)-1-Phenyl-2-(1-pyrrolidinyl)propan-1-ol (1.08 g, 5.2607 mmol) was dissolved in THF (30 mL) and the solution was cooled down to 0° C. A diethylzinc solution in hexane (52.3 mL of 1 M, 52.300 mmol) was slowly added over 10 minutes, and the resulting suspension was stirred at 0° C. for 5 minutes. A solution of ethynyl(trimethyl)silane (6.4094 g, 9.04 mL, 65.257 mmol) in toluene (10 mL) was added, and the reaction was stirred at 0° C. for 5 minutes. A solution of 3,3,4,4,4-pentadeuterio-1-(trideuteriomethoxy)butan-2-one (218 g, 26.117 mmol) was finally added. The reaction was stirred and allowed to warm to room temperature over the period of 18 hrs. The reaction mixture was quenched with 1:1 v/v mixture of sat. aqueous NH4Cl solution and water (total of 80 mL) and the aqueous layer was extracted with ethyl acetate (3×40 mL). The organic layers were combined and washed with brine (50 mL), dried over anhydrous sodium sulfate, filtered and evaporated under reduced pressure. Purification by silica gel chromatography (gradient: 0-30% EtOAc in heptanes) afforded (3S)-4,4,5,5,5-pentadeuterio-3-(trideuteriomethoxymethyl)-1-trimethylsilyl-pent-1-yn-3-ol (3.102 g, 49%) as a clear oil. 1H NMR (400 MHz, CDCl3) δ 3.51-3.45 (m, 1H), 3.42-3.36 (m, 1H), 0.18 (s, 9H), exchangeable H not observed.

### Step 5: methyl 4-[(3S)-4,4,5,5,5-pentadeuterio-3-hydroxy-3-(trideuteriomethoxymethyl)pent-1-ynyl]benzoate (N100)

To a sealed tube, methyl 4-iodobenzoate (3.7 g, 14.120 mmol), (3S)-4,4,5,5,5-pentadeuterio-3-(trideuteriomethoxymethyl)-1-trimethylsilyl-pent-1-yn-3-ol (3.102 g, 12.801 mmol), CuI (176.8 mg, 0.9283 mmol), triethylamine (7.7682 g, 10.7 mL, 76.768 mmol) and THF (40 mL) were added. The mixture was purged with nitrogen for 20 min. PdCl2(PPh3)2 (539.2 mg, 0.7682 mmol) and TBAF solution in THF (14 mL of 1 M, 14.000 mmol) were added to the mixture and it was purged with nitrogen for another 5 min. The mixture was heated at 70° C. for 20 h. After cooling to room temperature, the mixture was partitioned between EtOAc (100 mL) and half-saturated aqueous ammonium chloride (100 mL). The phases were separated and the aqueous layer was extracted with EtOAc (2×50 mL). The combined organic layers were washed with brine (50 mL), dried over anhydrous sodium sulfate, filtered, and concentrated in vacuo. Purification by silica gel chromatography (0-30% EtOAc/Heptanes) afforded methyl 4-[(3S)-4,4,5,5,5-pentadeuterio-3-hydroxy-3-(trideuteriomethoxymethyl)pent-1-ynyl]benzoate (3 g, 85%) as a yellow oil. 1H NMR (400 MHz, DMSO-d6) δ 7.97-7.92 (m, 2H), 7.56-7.51 (m, 2H), 5.51 (s, 1H), 3.86 (s, 3H), 3.45-3.37 (m, 2H). ESI-MS m/z calc. 270.1707, found 253.2 (M−17)+; Retention time: 2.55 minutes.

### Example 1

### (S)-4-(8-Fluoro-5-(4-fluoro-3-methylphenyl)-6-(2-hydroxy-1-methoxybutan-2-yl)-1,5-dihydropyrrolo[2,3-J]indazol-7-yl)benzoic acid (Compound 2)

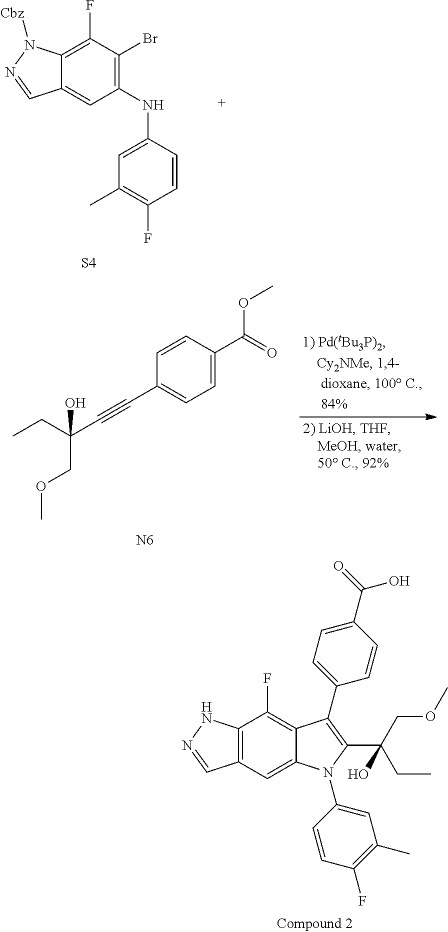

### Step 1

In a 20 L jacketed reactor, N,N-dicyclohexylmethylamine (416.09 g, 2.130 mol) was added to a solution of benzyl 6-bromo-7-fluoro-5-((4-fluoro-3-methylphenyl)amino)-1H-indazole-1-carboxylate (S4, 330.02 g, 691.8 mmol) and methyl (S)-4-(3-hydroxy-3-(methoxymethyl)pent-1-yn-1-yl)benzoate (Peak B, N6, 190.4 g, 725.9 mmol) in 1,4-dioxane (3.5 L) and the reaction mixture was flushed with nitrogen for 30 min. Pd(^{t}Bu₃P)₂ (17.9 g, 35.03 mmol) was added and the mixture was flushed with nitrogen for a further 5 min. The reaction was heated to 100° C. for 90 min, then cooled to ambient temperature. The reaction was quenched by addition of 2 M HCl (2.5 L) and extracted with EtOAc (2.5 L). The organic phase was washed with 2 M HCl (1.5 L) and a saturated brine solution (1.5 L). The aqueous phase was back-extracted with EtOAc (2.5 L). The combined organic extracts were concentrated in vacuo. Purification by flash chromatography (3×3 kg SiO₂ then 1×750 g SiO₂, 0 to 40% EtOAc in heptane) gave benzyl (S)-8-fluoro-5-(4-fluoro-3-methylphenyl)-6-(2-hydroxy-1-methoxybutan-2-yl)-7-(4-(methoxycarbonyl)phenyl)pyrrolo[2,3-J]indazole-1(5H)-carboxylate (381 g, 84%). ¹H NMR (400 MHz, DMSO-d₆) δ 8.40 (d, J=1.6 Hz, 1H), 8.16-7.92 (m, 2H), 7.58 (ddd, J=10.0, 7.3, 1.9 Hz, 2H), 7.51-7.23 (m, 8H), 6.90 (d, J=1.0 Hz, 1H), 5.37 (s, 2H), 4.60 (d, J=7.7 Hz, 1H), 3.92 (s, 3H), 3.27-3.15 (m, 2H), 3.12 (s, 3H), 2.33 (m, 3H), 1.59-1.36 (m, 2H), 0.73 (t, J=7.3 Hz, 3H) ppm. ESI-MS m/z calc. 653.234, found 654.0 (M+1)*; 652.0 (M−1)⁻.

### Step 2

A solution of LiOH monohydrate (193 g, 4.599 mol) in water (1.6 L) was added to a solution of benzyl (S)-8-fluoro-5-(4-fluoro-3-methylphenyl)-6-(2-hydroxy-1-methoxybutan-2-yl)-7-(4-(methoxycarbonyl)phenyl)pyrrolo[2,3-f]indazole-1(5H)-carboxylate (380 g, 581.3 mmol) in THF (3.5 L) and MeOH (1.6 L) and the mixture was heated to 50° C. for 30 min. The mixture was cooled to ambient temperature and the volatiles were removed in vacuo. The aqueous suspension was diluted with EtOAc (1.2 L) and acidified to pH 4 by addition of citric acid (600 mL, 2 M aqueous solution, 1.200 mol). The mixture was vigorously stirred at 20° C. A white solid crashed out of solution. The solid was filtered off and dried on the filter for a few hours. The solid was suspended in DCM (4 L), heated under reflux for 4 h, cooled to ambient temperature, filtered and dried in vacuo overnight to give (S)-4-(8-fluoro-5-(4-fluoro-3-methylphenyl)-6-(2-hydroxy-1-methoxybutan-2-yl)-1,5-dihydropyrrolo[2,3-f]indazol-7-yl)benzoic acid (Compound 2, 271.38 g, 92%). ¹H NMR (400 MHz, DMSO-d₆) δ 12.95 (s, 1H), 8.04 (d, J=3.3 Hz, 1H), 7.97 (dt, J=8.9, 2.4 Hz, 2H), 7.57 (t, J=7.3 Hz, 2H), 7.43-7.26 (m, 3H), 6.69 (s, 1H), 4.47 (d, J=7.8 Hz, 1H), 3.31-3.14 (m, 2H), 3.13 (s, 3H), 2.33 (m, 3H), 1.48 (m, 2H), 0.73 (t, J=7.3 Hz, 3H) ppm; exchangeable H not observed. ESI-MS m/z calc. 505.181, found 506.2 (M+1)⁺.

The following compounds were made using the methods described in Example 1 except that, different starting material and reagents were used in Step 1 in place of benzyl 6-bromo-7-fluoro-5-((4-fluoro-3-methylphenyl)amino)-1H-indazole-1-carboxylate and methyl (S)-4-(3-hydroxy-3-(methoxymethyl)pent-1-yn-1-yl)benzoate, respectively, and the reaction was carried out at 110° C.:

| | | | | LC/ | |
|---|---|---|---|---|---|
| | | | Start- | MS | |
| | | | ing | m/z | |
| Cmpd | | | mate- | [M + | NMR (shifts in |
| No. | Compound | Reagent | rial | H]⁺ | ppm) |
| 7 | 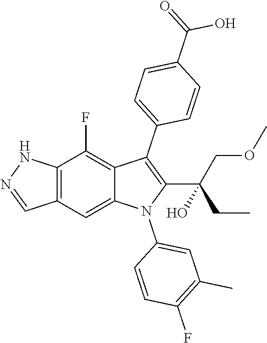 [SMILES: CC[C@](O)(COC)c1c(-c2ccc(C(=O)O)cc2)c2c(F)c3[nH]ncc3cc2n1-c1ccc(F)c(C)c1] | 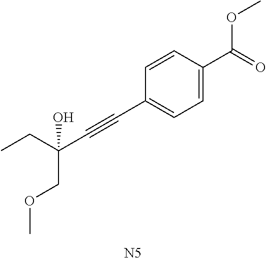 | S26 | 506.4 | ¹H NMR (400 MHz, DMSO-d₆) δ 12.94 (s, 1H), 12.90 (s, 1H), 8.04 (d, J = 3.3 Hz, 1H), 7.97 (d, J = 7.5 Hz, 2H), 7.58 (t, J = 7.9 Hz, 2H), 7.46- 7.23 (m, 3H), 6.68 (s, 1H), 4.48 (d, J = 7.5 Hz, 1H), 3.29- 3.15 (m, 2H), 3.12 (s, 3H), 2.32 (m, 3H), 1.55-1.37 (m, 2H), 0.72 (t, J = 7.3 Hz, 3H) ppm. |
| 53 | 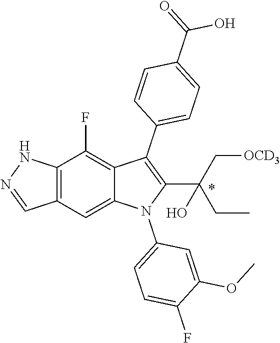 | 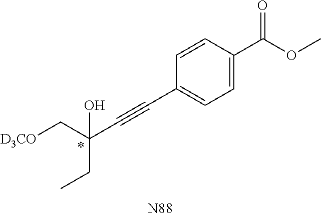 | S37 | 525.3 | ¹H NMR (400 MHz, Methanol-d₄) δ 8.07 (td, J = 6.2, 3.2 Hz, 2H), 7.99 (d, J = 3.2 Hz, 1H), 7.62 (d, J = 8.1 Hz, 2H), 7.33- 7.14 (m, 2H), 7.04 (dddd, J = 28.6, 8.5, 3.9, 2.4 Hz, 1H), 6.79 (d, J = 5.8 Hz, 1H), 3.88 (d, J = 2.8 Hz, 3H), 3.40 (dd, J = 14.2, 9.6 Hz, 1H), 3.18 (dd, J = 9.6, 6.1 Hz, 1H), 1.73-1.55 (m, 2H), 0.88-0.80 (m, 3H) ppm; exchangeable H not observed. |

The following compound was made using the methods described in Example 1 except that a different starting material and reagent were used in Step 1 in place of benzyl 6-bromo-7-fluoro-5-((4-fluoro-3-methylphenyl)amino)-1H-indazole-1-carboxylate and methyl (S′)-4-(3-hydroxy-3-(methoxymethyl)pent-1-yn-1-yl)benzoate and the reaction was carried out at 110° C. Step 2 was carried out at 65° C.:

| | | | | LC/MS | |
|---|---|---|---|---|---|
| Cmpd | | | Starting | m/z | NMR (shifts in |
| No. | Compound | Reagent | material | [M + H]⁺ | ppm) |
| 27 | 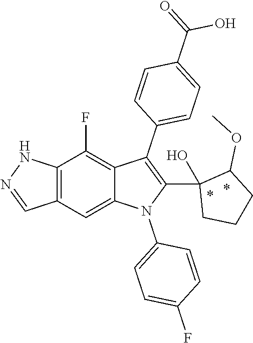 [SMILES: COC1CCCC1(O)c1c(-c2ccc(C(=O)O)cc2)c2c(F)c3[nH]ncc3cc2n1-c1ccc(F)cc1] | 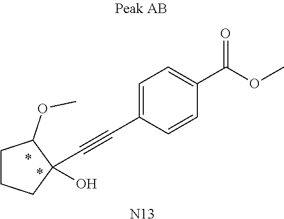 | S6 | 504.5 | ¹H NMR (400 MHz, DMSO-d₆) δ 12.98 (s, 2H), 8.14-7.90 (m, 3H), 7.75-7.67 (m, 1H), 7.63 (d, J = 7.9 Hz, 2H), 7.46- 7.29 (m, 3H), 6.73 (s, 1H), 4.48 (s, 1H), 3.63 (dd, J = 8.8, 6.9 Hz, 1H), 2.65 (s, 3H), 2.04-1.90 (m, 1H), 1.81-1.68 (m, 1H), 1.68-1.58 (m, 1H), 1.58-1.45 (m, 1H), 1.45-1.21 (m, 2H) ppm. |

The following compound was made using the methods described in Example 1 except that, different starting materials and/or reagents were used in Step 1 in place of benzyl 6-bromo-7-fluoro-5-((4-fluoro-3-methylphenyl)amino)-1H-indazole-1-carboxylate and methyl (S′)-4-(3-hydroxy-3-(methoxymethyl)pent-1-yn-1-yl)benzoate. Step 2 was carried out at ambient temperature:

| | | | | LC/MS | |
|---|---|---|---|---|---|
| Cmpd | | | Starting | m/z | NMR (shifts in |
| No. | Compound | Reagent | material | [M + H]⁺ | ppm) |
| 4 | 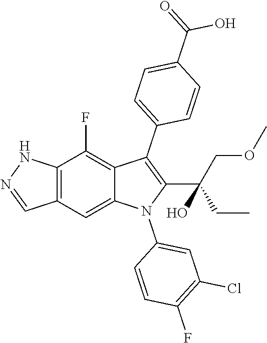 [SMILES: CC[C@@](O)(COC)c1c(-c2ccc(C(=O)O)cc2)c2c(F)c3[nH]ncc3cc2n1-c1ccc(F)c(Cl)c1] | 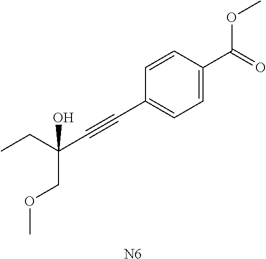 | S5 | 526.1 | ¹H NMR (400 MHz, DMSO-d₆) δ 12.99 (s, 2H), 8.09-7.94 (m, 3H), 7.71 (ddd, J = 38.4, 6.7, 2.5 Hz, 1H), 7.64-7.55 (m, 3H), 7.55-7.39 (m, 1H), 6.78 (d, J = 2.2 Hz, 1H), 4.73 (d, J = 24.3 Hz, 1H), 3.32- 3.16 (m, 2H), 3.13 (d, J = 8.7 Hz, 3H), 1.53-1.37 (m, 2H), 0.80-0.64 (m, 3H) ppm. |
| 21 | 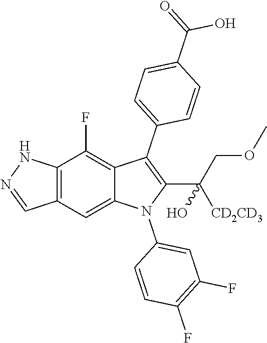 | 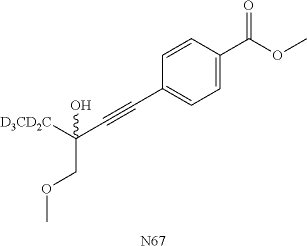 | S3 | 515.1 | 1H NMR (400 MHz, Methanol-d₄) δ 8.09 (ddd, J = 8.5, 5.4, 1.8 Hz, 2H), 7.99 (d, J = 3.1 Hz, 1H), 7.61 (d, J = 8.4 Hz, 2H), 7.52-7.23 (m, 3H), 6.79 (d, J = 1.6 Hz, 1H), 3.38 (m, 1H), 3.23-3.15 (m, 4H) ppm; exchangeable H not observed. |
| 20 | 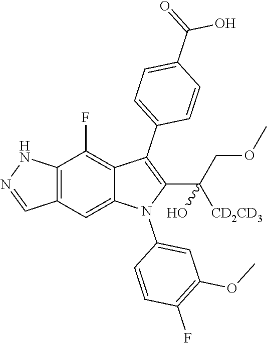 | 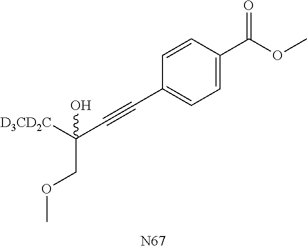 | S2 | 527.0 | 1H NMR (400 MHz, Methanol-d₄) δ 8.10-8.03 (m, 2H), 7.98 (d, J = 3.2 Hz, 1H), 7.61 (d, J = 8.0 Hz, 2H), 7.31- 7.14 (m, 2H), 7.10- 6.98 (m, 1H), 6.79 (d, J = 5.7 Hz, 1H), 3.89 (m, 3H), 3.41 (m, 1H), 3.25-3.15 (m, 4H) ppm; exchangeable H not observed. |
| 12 | 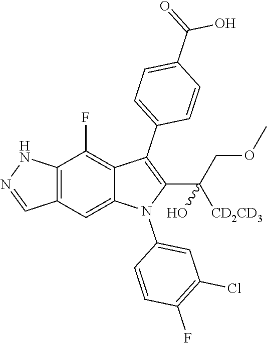 | 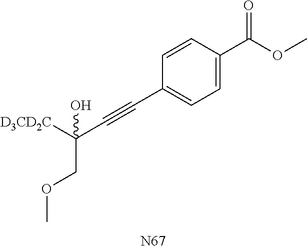 | S5 | 531.0 | 1H NMR (400 MHz, Methanol-d₄) δ 8.13-8.04 (m, 2H), 8.00 (dd, J = 3.2, 0.9 Hz, 1H), 7.70-7.53 (m, 3H), 7.51-7.37 (m, 2H), 6.78 (d, J = 0.9 Hz, 1H), 3.37 (m, 1H), 3.23-3.15 (m, 4H) ppm; exchangeable H not observed. |
| 18 | 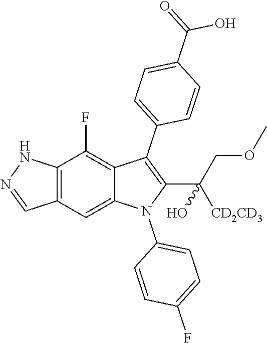 | 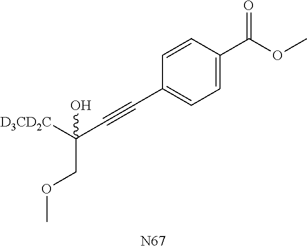 | S6 | 497.0 | 1H NMR (400 MHz, Methanol-d₄) δ 8.11-8.03 (m, 2H), 7.97 (d, J = 3.2 Hz, 1H), 7.65-7.59 (m, 2H), 7.55-7.42 (m, 2H), 7.31 (tt, J = 8.8, 1.4 Hz, 2H), 6.73 (s, 1H), 3.40- 3.34 (m, 1H), 3.20- 3.09 (m, 4H) ppm; exchangeable H not observed. |
| 19 | 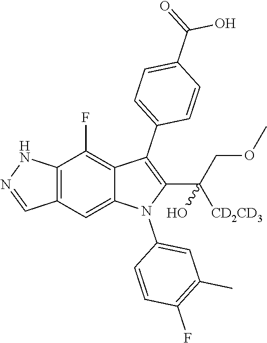 | 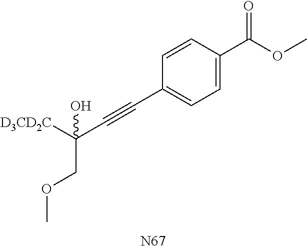 | S4 | 511.0 | 1H NMR (400 MHz, Methanol-d₄) δ 8.11-8.01 (m, 2H), 7.97 (d, J = 3.2 Hz, 1H), 7.61 (d, J = 8.0 Hz, 2H), 7.41- 7.20 (m, 3H), 6.73 (s, 1H), 3.41-3.33 (m, 1H), 3.22-3.10 (m, 4H), 2.37 (m, 3H) ppm; exchangeable H not observed. |

The following compounds were made using the methods described in Example 1 except that, different starting materials and/or reagents were used in Step 1 in place of benzyl 6-bromo-7-fluoro-5-((4-fluoro-3-methylphenyl)amino)-1H-indazole-1-carboxylate and methyl (S′)-4-(3-hydroxy-3-(methoxymethyl)pent-1-yn-1-yl)benzoate, respectively, and the reaction was carried out at 110° C. Step 2 was carried out at 60° C.:

| | | | | LC/ | |
|---|---|---|---|---|---|
| | | | Start- | MS | |
| | | | ing | m/z | |
| Cmpd | | | mate- | [M + | NMR (shifts in |
| No. | Compound | Reagent | rial | H]⁺ | ppm) |
| 15 | 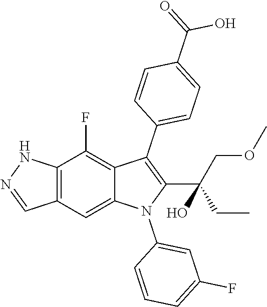 [SMILES: CC[C@@](O)(COC)c1c(-c2ccc(C(=O)O)cc2)c2c(F)c3[nH]ncc3cc2n1-c1cccc(F)c1] | 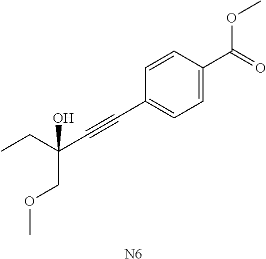 | S13 | 492.4 | ¹H NMR (400 MHz, DMSO-d₆) δ 13.06-12.80 (m, 2H), 8.11-7.89 (m, 3H), 7.69- 7.50 (m, 3H), 7.50- 7.20 (m, 3H), 6.72 (d, J = 2.2 Hz, 1H), 4.65 (d, J = 23.6 Hz, 1H), 3.33- 3.16 (m, 2H), 3.12 (m, 3H), 1.53- 1.32 (m, 2H), 0.72 (t, J = 7.3 Hz, 3H) ppm. |
| 16 | 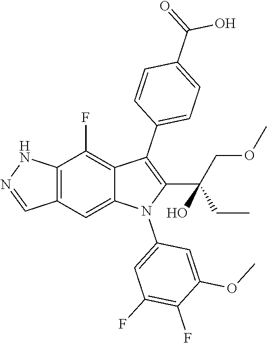 [SMILES: CC[C@@](O)(COC)c1c(-c2ccc(C(=O)O)cc2)c2c(F)c3[nH]ncc3cc2n1-c1cc(F)c(F)c(OC)c1] | 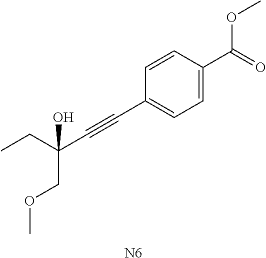 | S15 | 540.4 | ¹H NMR (400 MHz, DMSO- d₆) δ 13.05 (s, 2H), 8.11- 7.94 (m, 2H), 7.84-7.47 (m, 4H), 7.40-7.27 (m, 1H), 6.79 (d, J = 1.3 Hz, 1H), 4.89-4.76 (m, 1H), 3.32-3.07 (m, 8H), 1.51-1.36 (m, 2H), 0.72 (t, J = 7.3 Hz, 3H) ppm. |
| 17 | 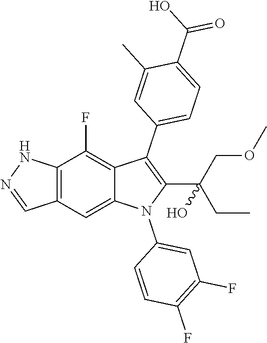 [SMILES: CCC(O)(COC)c1c(-c2ccc(C(=O)O)c(C)c2)c2c(F)c3[nH]ncc3cc2n1-c1ccc(F)c(F)c1] | 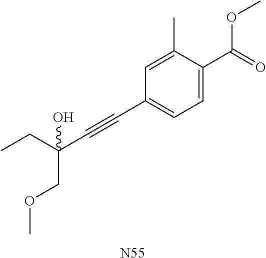 [SMILES: CCC(O)(C#Cc1ccc(C(=O)OC)c(C)c1)COC ; "N55"] | S25 | 524.4 | ¹H NMR (400 MHz, DMSO-d₆) δ 12.99 (s, 1H), 12.83 (s, 1H), 8.04 (d, J = 3.3 Hz, 1H), 7.96-7.82 (m, 1H), 7.72-7.48 (m, 2H), 7.43- 7.21 (m, 3H), 6.77 (d, J = 1.5 Hz, 1H), 4.78-4.63 (m, 1H), 3.31-3.06 (m, 5H), 2.64- 2.56 (m, 3H), 1.56- 1.35 (m, 2H), 0.72 (t, J = 7.4 Hz, 3H) ppm. |
| 25 | 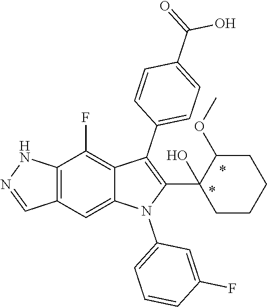 [SMILES: COC1CCCCC1(O)c1c(-c2ccc(C(=O)O)cc2)c2c(F)c3[nH]ncc3cc2n1-c1cccc(F)c1] | 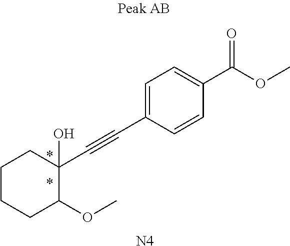 | S13 | 518.4 | ¹H NMR (400 MHz, Methanol- d₄) δ 8.16-8.05 (m, 2H), 7.98 (d, J = 3.2 Hz, 1H), 7.69-7.52 (m, 3H), 7.41-7.25 (m, 2H), 7.25-7.13 (m, 1H), 6.75 (d, J = 1.8 Hz, 1H), 3.11 (m, 3H), 2.16- 2.04 (m, 1H), 1.91- 1.70 (m, 2H), 1.62-1.20 (m, 5H), 0.97-0.76 (m, 1H). ppm; indazole NH, and acid OH, and alcohol OH not observed. |
| 26 | 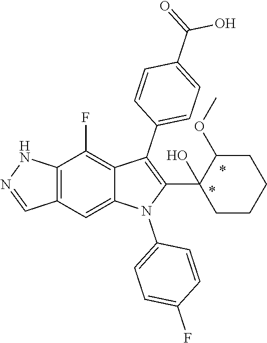 [SMILES: COC1CCCCC1(O)c1c(-c2ccc(C(=O)O)cc2)c2c(F)c3[nH]ncc3cc2n1-c1ccc(F)cc1] | 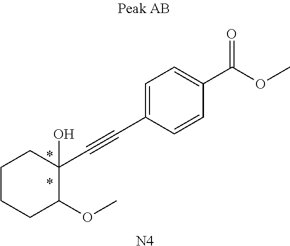 | S6 | 518.5 | ¹H NMR (400 MHz, DMSO-d₆) δ 12.96 (s, 2H), 8.13- 7.91 (m, 3H), 7.75-7.53 (m, 3H), 7.47-7.28 (m, 3H), 6.66 (s, 1H), 3.95 (s, 1H), 3.15 (m, 1H), 2.93 (s, 3H), 2.00 (d, J = 13.5 Hz, 1H), 1.76-1.60 (m, 2H), 1.50 (d, J = 13.1 Hz, 1H), 1.42-1.12 (m, 3H), 0.91-0.66 (m, 1H) ppm. |
| 28 | 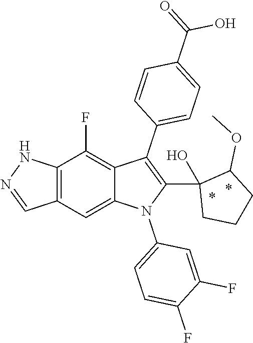 [SMILES: COC1CCCC1(O)c1c(-c2ccc(C(=O)O)cc2)c2c(F)c3[nH]ncc3cc2n1-c1ccc(F)c(F)c1] | 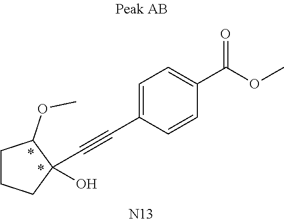 | S14 | 522.6 | ¹H NMR (400 MHz, DMSO-d₆) δ 13.00 (s, 2H), 8.21- 7.91 (m, 3H), 7.90-7.12 (m, 5H), 6.83 (d, J = 2.3 Hz, 1H), 4.73- 4.49 (m, 1H), 3.60 (dd, J = 8.9, 6.9 Hz, 1H), 2.67 (d, J = 7.7 Hz, 3H), 2.06-1.93 (m, 1H), 1.81-1.69 (m, 1H), 1.69- 1.60 (m, 1H), 1.60- 1.47 (m, 1H), 1.46-1.27 (m, 2H) ppm. |
| 42 | 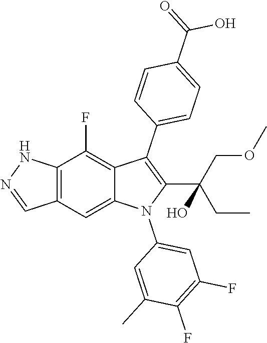 [SMILES: CC[C@@](O)(COC)c1c(-c2ccc(C(=O)O)cc2)c2c(F)c3[nH]ncc3cc2n1-c1cc(C)c(F)c(F)c1] | 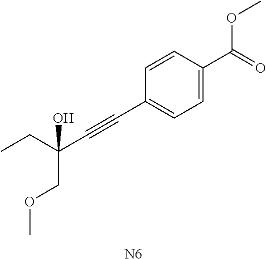 | S16 | 524.5 | ¹H NMR (400 MHz, Methanol- d₄) δ 8.15-8.03 (m, 2H), 7.99 (d, J = 3.2 Hz, 1H), 7.61 (d, J = 8.1 Hz, 2H), 7.35- 7.08 (m, 3H), 6.80 (d, J = 2.1 Hz, 1H), 3.41-3.34 (m, 1H), 3.23- 3.14 (m, 4H), 2.41 (d, J = 2.0 Hz, 3H), 1.71-1.53 (m, 2H), 0.89- 0.80 (m, 3H) ppm; exchangeable H not observed. |
| 64 | 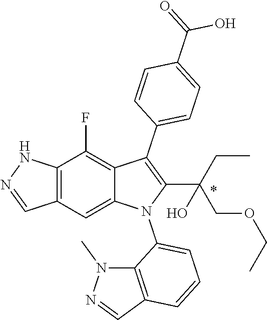 [SMILES: CCOCC(O)(CC)c1c(-c2ccc(C(=O)O)cc2)c2c(F)c3[nH]ncc3cc2n1-c1cccc2cnn(C)c12] | 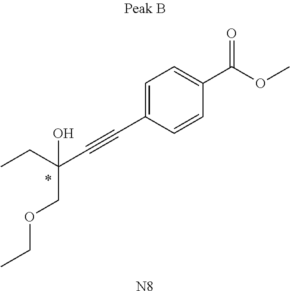 | S24 | 542.4 | ¹H NMR (400 MHz, DMSO- d₆) δ 13.04 (s, 2H), 8.19- 8.11 (m, 1H), 8.08-7.97 (m, 3H), 7.95-7.86 (m, 1H), 7.78- 7.62 (m, 2H), 7.53-7.47 (m, 1H), 7.33-7.24 (m, 1H), 6.52-6.42 (m, 1H), 3.50- 2.99 (m, 7H), 1.74-1.30 (m, 2H), 1.15-0.97 (m, 3H), 0.85-0.64 (m, 3H) ppm. |
| 43 | 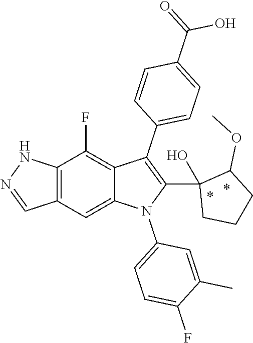 [SMILES: COC1CCCC1(O)c1c(-c2ccc(C(=O)O)cc2)c2c(F)c3[nH]ncc3cc2n1-c1ccc(F)c(C)c1] | 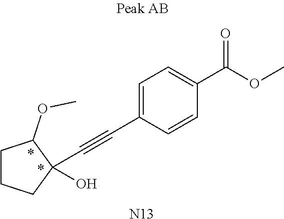 | S21 | 518.5 | ¹H NMR (400 MHz, DMSO- d₆) δ 12.97 (s, 2H), 8.10- 7.93 (m, 3H), 7.70-7.44 (m, 3H), 7.38-7.18 (m, 2H), 6.75 (d, J = 1.1 Hz, 1H), 4.49-4.32 (m, 1H), 3.72-3.56 (m, 1H), 2.68 (d, J = 7.7 Hz, 3H), 2.34- 2.28 (m, 3H), 2.07- 1.92 (m, 1H), 1.85-1.71 (m, 1H), 1.71-1.59 (m, 1H), 1.59- 1.45 (m, 1H), 1.45- 1.18 (m, 2H) ppm. |
| 56 | 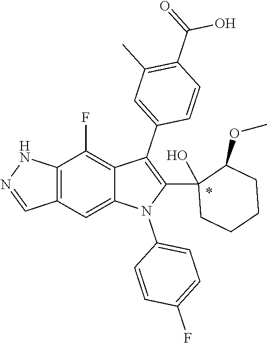 [SMILES: CO[C@H]1CCCCC1(O)c1c(-c2ccc(C(=O)O)c(C)c2)c2c(F)c3[nH]ncc3cc2n1-c1ccc(F)cc1] | 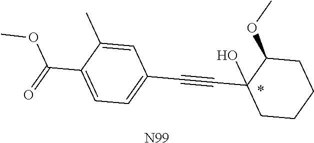 [SMILES: COC(=O)c1ccc(C#CC2(O)CCCC[C@@H]2OC)cc1C ; "N99"] | S6 | 532.17 | ¹H NMR (400 MHz, DMSO-d6) δ 13.17-12.52 (m, 2H), 8.03 (d, J = 3.2 Hz, 1H), 7.96- 7.61 (m, 2H), 7.54- 7.29 (m, 5H), 6.65 (s, 1H), 4.02 (s, 1H), 3.26-3.10 (m, 1H), 3.00- 2.84 (m, 3H), 2.65- 2.58 (m, 3H), 2.00 (s, 1H), 1.77- 1.60 (m, 2H), 1.51 (d, J = 13.4 Hz, 1H), 1.38-1.18 (m, 3H), 0.79 (s, 1H). |
| 55 | 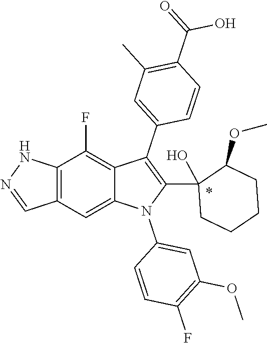 [SMILES: COc1cc(-n2c(C3(O)CCCC[C@@H]3OC)c(-c3ccc(C(=O)O)c(C)c3)c3c(F)c4[nH]ncc4cc32)ccc1F] | 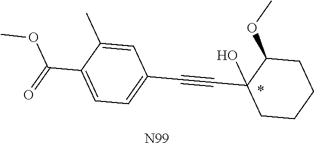 [SMILES: COC(=O)c1ccc(C#CC2(O)CCCC[C@@H]2OC)cc1C ; "N99"] | S99 | 562.19 | ¹H NMR (400 MHz, DMSO-d6) δ 13.41-12.73 (m, 2H), 8.08-8.00 (m, 1H), 8.01-7.85 (m, 1H), 7.56-7.28 (m, 4H), 7.16-6.89 (m, 1H), 6.77-6.68 (m, 1H), 4.52-3.74 (m, 4H), 3.25-3.10 (m, 1H), 3.02-2.79 (m, 3H), 2.65-2.58 (m, 3H), 2.16-1.96 (m, 1H), 1.84-1.63 (m, 2H), 1.59-1.46 (m, 2H), 1.32-1.22 (m, 2H), 1.02-0.45 (m, 1H). |
| 54 | 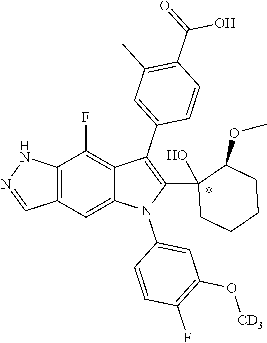 [SMILES: [2H]C([2H])([2H])Oc1cc(-n2c(C3(O)CCCC[C@@H]3OC)c(-c3ccc(C(=O)O)c(C)c3)c3c(F)c4[nH]ncc4cc32)ccc1F] | 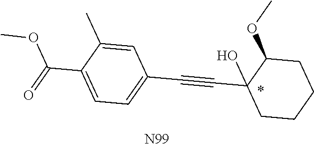 [SMILES: COC(=O)c1ccc(C#CC2(O)CCCC[C@@H]2OC)cc1C ; "N99"] | S8 | 565.18 | ¹H NMR (400 MHz, DMSO-d6) δ 13.18-12.51 (m, 2H), 8.10-7.99 (m, 1H), 7.99-7.84 (m, 1H), 7.52-6.86 (m, 5H), 6.75-6.66 (m, 1H), 4.61-3.80 (m, 1H), 3.22-3.10 (m, 1H), 3.04-2.80 (m, 3H), 2.65-2.56 (m, 3H), 2.14-1.97 (m, 1H), 1.81-1.62 (m, 2H), 1.51 (s, 1H), 1.28 (d, J = 17.1 Hz, 3H), 0.93- 0.63 (m, 1H). |
| 67 | 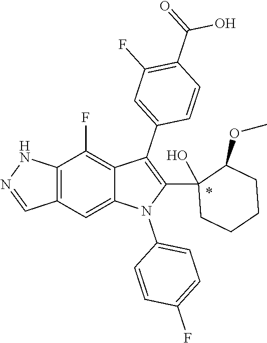 [SMILES: CO[C@H]1CCCCC1(O)c1c(-c2ccc(C(=O)O)c(F)c2)c2c(F)c3[nH]ncc3cc2n1-c1ccc(F)cc1] | 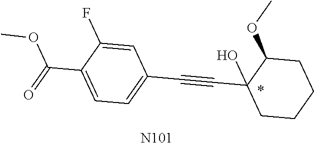 [SMILES: COC(=O)c1ccc(C#CC2(O)CCCC[C@@H]2OC)cc1F ; "N101"] | S8 | 536.45 | ¹H NMR (400 MHz, DMSO-d6) δ 13.70-12.83 (m, 2H), 8.13-8.03 (m, 1H), 7.97-7.60 (m, 2H), 7.58-7.25 (m, 5H), 6.66 (s, 1H), 4.18 (s, 1H), 3.20- 2.86 (m, 4H), 2.03- 1.88 (m, 1H), 1.81- 1.60 (m, 2H), 1.60- 1.43 (m, 1H), 1.43- 1.15 (m, 3H), 1.00- 0.64 (m, 1H). |
| 57 | 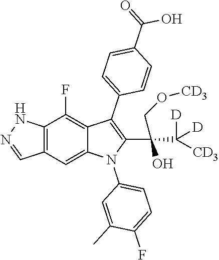 [SMILES: [2H]C([2H])([2H])OC[C@@](O)(c1c(-c2ccc(C(=O)O)cc2)c2c(F)c3[nH]ncc3cc2n1-c1ccc(F)c(C)c1)C([2H])([2H])C([2H])([2H])[2H]] | 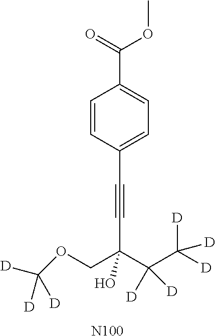 [SMILES: [2H]C([2H])([2H])OC[C@@](O)(C#Cc1ccc(C(=O)OC)cc1)C([2H])([2H])C([2H])([2H])[2H] ; "N100"] | S21 | 514.50 | ¹H NMR (400 MHz, DMSO-d6) δ 12.97 (s, 1H), 8.08-7.90 (m, 3H), 7.64-7.51 (m, 2H), 7.48- 7.19 (m, 3H), 6.68 (s, 1H), 4.51 (d, J = 7.0 Hz, 1H), 3.24-3.15 (m, 2H), 2.32 (s, 3H). |

The following compounds were made using the methods described in Example 1 except that, different starting materials and reagents were used in Step 1 in place of benzyl 6-bromo-7-fluoro-5-((4-fluoro-3-methylphenyl)amino)-1H-indazole-1-carboxylate and methyl (S′)-4-(3-hydroxy-3-(methoxymethyl)pent-1-yn-1-yl)benzoate respectively. Step 2 was carried out at 60° C. In the case of Compound 66, a final SFC Step was introduced to separate the enantiomers (Chiralpak IG column, 5 μm particle size, 25 cm×10 mm from Daicel Corporation (Mobile phase: 3000 methanol (supplemented with 0.100 ammonia), 7000 CO₂; Flow rate 15 mL/min); rt=2.05 min):

| | | | | LC/MS | |
|---|---|---|---|---|---|
| Cmpd | | | Starting | m/z | NMR |
| No. | Compound | Reagent | material | [M + H]⁺ | (shifts in ppm) |
| 24 | 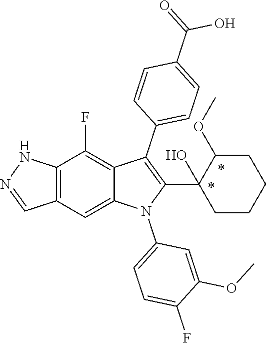 [SMILES: COc1cc(-n2c(C3(O)CCCCC3OC)c(-c3ccc(C(=O)O)cc3)c3c(F)c4[nH]ncc4cc32)ccc1F] | 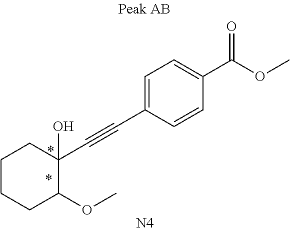 | S1 | 548.4 | ¹H NMR (400 MHz, DMSO-d₆) δ 12.97 (m, 2H), 8.12-7.88 (m, 3H), 7.75-7.54 (m, 2H), 7.50-7.37 (m, 1H), 7.37-6.84 (m, 2H), 6.80-6.68 (m, 1H), 4.18 (s, 1H), 3.92-3.82 (m, 3H), 3.25-3.11 (m, 1H), 2.94 (m, 3H), 2.07 (m, 1H), 1.82- 1.62 (m, 2H), 1.53 (d, J = 12.9 Hz, 1H), 1.44-1.12 (m, 3H), 0.99-0.68 (m, 1H) ppm. |
| 58 | 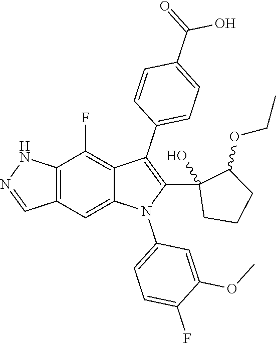 [SMILES: CCOC1CCCC1(O)c1c(-c2ccc(C(=O)O)cc2)c2c(F)c3[nH]ncc3cc2n1-c1ccc(F)c(OC)c1] | 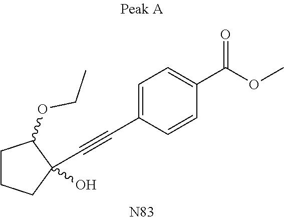 [SMILES: CCOC1CCCC1(O)C#Cc1ccc(C(=O)OC)cc1 ; "N83"] | S1 | 548.6 | ¹H NMR (300 MHz, DMSO-d₆) δ 12.98 (s, 2H), 8.10-7.98 (m, 3H), 7.71-6.89 (m, 5H), 6.87-6.77 (m, 1H), 4.44-4.12 (m, 1H), 3.92-3.78 (m, 3H), 3.72 (q, J = 8.2 Hz, 1H), 3.17- 3.05 (m, 1H), 2.45- 2.27 (m, 1H), 2.06- 1.25 (m, 6H), 0.97 (t, J = 6.9 Hz, 3H) ppm. |
| 66 | 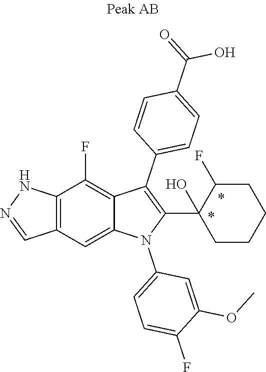 [SMILES: COc1cc(-n2c(C3(O)CCCCC3F)c(-c3ccc(C(=O)O)cc3)c3c(F)c4[nH]ncc4cc32)ccc1F] | 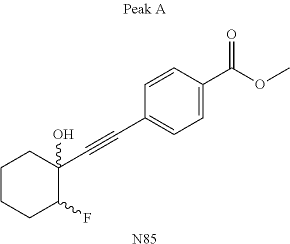 [SMILES: COC(=O)c1ccc(C#CC2(O)CCCCC2F)cc1 ; "N85"] | S37 | 536.3 | |

The following compound was made using the methods described in Example 1 except that, different starting materials and reagents were respectively used in Step 1 in place of benzyl 6-bromo-7-fluoro-5-((4-fluoro-3-methyl phenyl)amino)-1H-indazole-1-carboxylate and methyl (S)-4-(3-hydroxy-3-(methoxymethyl)pent-1-yn-1-yl)benzoate and the reaction was carried out at 110° C. Step 2 was carried out at ambient temperature:

| | | | | LC/MS | |
|---|---|---|---|---|---|
| Cmpd | | | Starting | m/z | NMR |
| No. | Compound | Reagent | material | [M + H]⁺ | (shifts in ppm) |
| 14 | 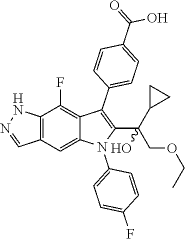 [SMILES: CCOCC(O)(c1c(-c2ccc(C(=O)O)cc2)c2c(F)c3[nH]ncc3cc2n1-c1ccc(F)cc1)C1CC1] | 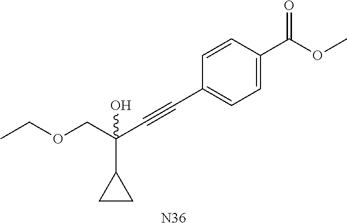 | S6 | 518.2 | ¹H NMR (400 MHz, DMSO-d₆) δ 13.00-12.86 (m, 2H), 8.05-8.02 (m, 1H), 7.95 (d, J = 7.8 Hz, 2H), 7.66- 7.60 (m, 2H), 7.56-7.48 (m, 2H), 7.43-7.35 (m, 2H), 6.68 (s, 1H), 4.44 (s, 1H), 3.38-3.21 (m, 4H, overlapped with water), 1.09 (t, J = 7.0 Hz, 3H), 0.95 |
| | | | | | (m, 1H), 0.29 (m, |
| | | | | | 2H), 0.06 (m, |
| | | | | | 1H), −0.17-−0.24 |
| | | | | | (m, 1H) ppm. |
| 41 | 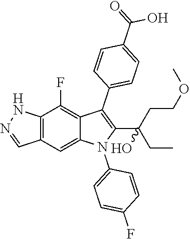 [SMILES: CCC(O)(CCOC)c1c(-c2ccc(C(=O)O)cc2)c2c(F)c3[nH]ncc3cc2n1-c1ccc(F)cc1] | 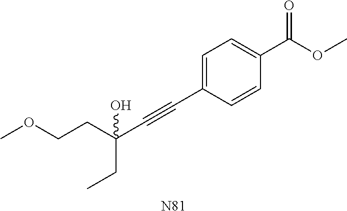 | S6 | 506.2 | ¹H NMR (400 MHz, DMSO-d₆) δ 12.96 (br s, 2H), 8.03 (d, J = 2.9 Hz, 1H), 8.01-7.93 (m, 2H), 7.58-7.51 (m, 2H), 7.50-7.38 (m, 4H), 6.65 (s, 1H), 4.30 (s, 1H), 3.51- 3.41 (m, 1H), 3.30 (m, 1H, overlapped with water), 3.13 (s, 3H), 1.84-1.73 (m, 1H), 1.72-1.62 (m, 1H), 1.60-1.48 (m, |
| | | | | | 1H), 1.48-1.35 (m, |
| | | | | | 1H), 0.78 (t, J = 7.2 |
| | | | | | Hz, 3H) ppm. |
| 52 | 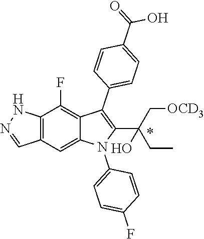 | 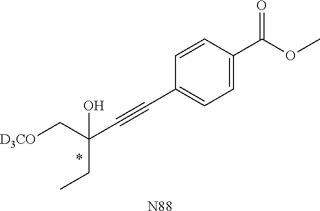 | S28 | 495.2 | ¹H NMR (400 MHz, Methanol-d₄) δ 8.10- 8.04 (m, 2H), 7.97 (d, J = 3.2 Hz, 1H), 7.62 (d, J = 8.1 Hz, 2H), 7.56-7.49 (m, 1H), 7.48-7.42 (m, 1H), 7.31 (tt, J = 8.7, 1.4 Hz, 2H), 6.73 (s, 1H), 3.36 (d, J = 9.7 Hz, 1H), 3.16 (d, J = 9.7 Hz, 1H), 1.69-1.52 (m, 2H), 0.84 (t, J = 7.4 Hz, 3H) ppm; exchangeable H not |
| | | | | | observed. |

The following compounds were made using the methods described in Example 1 except that, different starting materials and reagents were respectively used in Step 1 in place of benzyl 6-bromo-7-fluoro-5-((4-fluoro-3-methyl phenyl)amino)-1H-indazole-1-carboxylate and methyl (S)-4-(3-hydroxy-3-(methoxymethyl)pent-1-yn-1-yl)benzoate. Step 2 was carried out at 65° C.:

| | | | | LC/MS | |
|---|---|---|---|---|---|
| Cmpd | | | Starting | m/z | NMR (shifts in |
| No. | Compound | Reagent | material | [M + H]⁺ | ppm) |
| 22 | 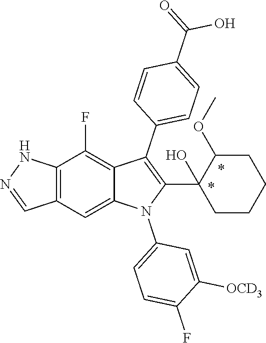 | 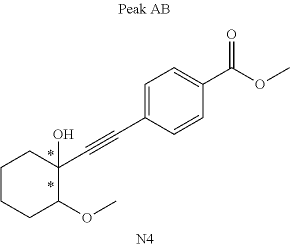 | S8 | 551.3 | ¹H NMR (400 MHz, DMSO-d₆) δ 12.98 (s, 2H), 8.11- 7.94 (m, 3H), 7.76-7.56 (m, 2H), 7.50-6.88 (m, 3H), 6.75 (d, J = 6.0 Hz, 1H), 4.56- 3.74 (m, 1H), 3.23-3.07 (m, 1H), 3.03-2.81 (m, 3H), 2.17- 1.97 (m, 1H), 1.84- 1.61 (m, 2H), 1.60-1.43 (m, 1H), 1.43-1.17 (m, 3H), 0.99- 0.67 (m, 1H) ppm. |
| 23 | 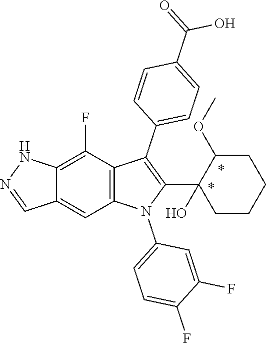 [SMILES: COC1CCCCC1(O)c1c(-c2ccc(C(=O)O)cc2)c2c(F)c3[nH]ncc3cc2n1-c1ccc(F)c(F)c1] | 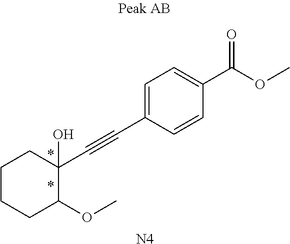 | S14 | 536.3 | ¹H NMR (400 MHz, DMSO-d₆) δ 13.01 (s, 2H), 8.11- 7.97 (m, 3H), 7.80-7.54 (m, 4H), 7.47-7.20 (m, 1H), 6.76 (d, J = 1.3 Hz, 1H), 3.20- 3.07 (m, 1H), 2.92 (m, 3H), 2.01 (d, J = 13.8 Hz, 1H), 1.68 (s, 2H), 1.52 (d, J = 13.1 Hz, 1H), 1.40- 1.17 (m, 3H), 0.98- 0.67 (m, 1H) ppm; exchangeable H not observed. |

The following Compounds were made using the methods described in Example 1 except that, different Starting Materials and Reagents were respectively used in Step 1 in place of benzyl 6-bromo-7-fluoro-5-((4-fluoro-3-methylphenyl)amino)-1H-indazole-1-carboxylate and methyl (S′)-4-(3-hydroxy-3-(methoxymethyl)pent-1-yn-1-yl)benzoate and the reaction was carried out at 110° C. Step 2 was carried out using 1,4-dioxane in place of THF:

| | | | | LC/MS | |
|---|---|---|---|---|---|
| Cmpd | | | Starting | m/z | NMR (shifts in |
| No. | Compound | Reagent | material | [M + H]⁺ | ppm) |
| 34 | 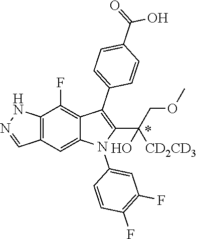 | 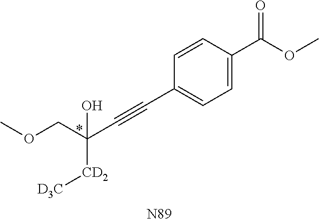 | S14 | 515.3 | ¹H NMR (400 MHz, Methanol- d₄) δ 8.13-8.05 (m, 2H), 8.00 (d, J = 3.1 Hz, 1H), 7.64-7.58 (m, 2H), 7.52-7.37 (m, 2H), 7.29 (m, , 1H), 6.79 (d, J = 1.7 Hz, 1H), 3.38 (m, 1H), 3.19 (m, 4H) ppm; exchangeable H not observed. |
| 45 | 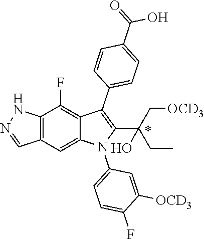 | 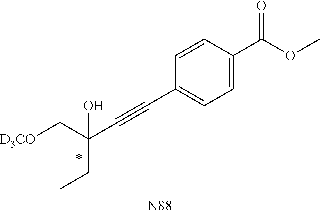 | S8 | 528.3 | ¹H NMR (400 MHz, Methanol- d₄) δ 8.11-8.04 (m, 2H), 7.99 (d, J = 3.2 Hz, 1H), 7.62 (d, J = 8.1 Hz, 2H), 7.33- 7.13 (m, 2H), 7.04 (m, 1H), 6.80 (d, J = 5.9 Hz, 1H), 3.40 (m, 1H), 3.18 (m, 1H), 1.72- 1.56 (m, 2H), 0.86 (m, 3H) ppm; exchangeable H not observed. |
| 46 | 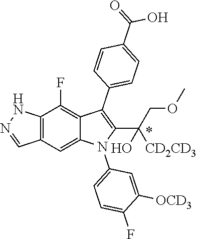 | 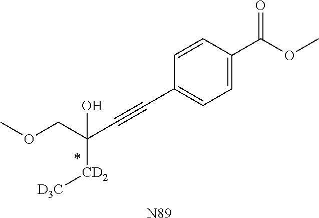 | S8 | | ¹H NMR (400 MHz, Methanol- d₄) δ 8.11-8.05 (m, 2H), 7.99 (d, J = 3.2 Hz, 1H), 7.62 (d, J = 8.1 Hz, 2H), 7.34- 7.14 (m, 2H), 7.04 (m, 1H), 6.80 (d, J = 5.8 Hz, 1H), 3.40 (m, 1H), 3.23- 3.15 (m, 4H) ppm; exchangeable H not observed. |
| 50 | 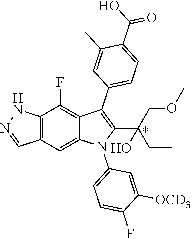 | 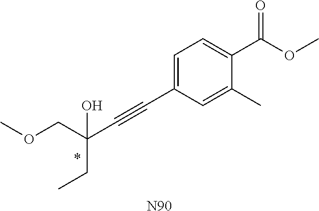 | S8 | 539.4 | ¹H NMR (400 MHz, DMSO-d₆) δ 13.03-12.72 (m, 2H), 8.10-7.99 (m, 1H), 7.94- 7.81 (m, 1H), 7.46- 7.15 (m, 4H), 7.12-6.92 (m, 1H), 6.78-6.68 (m, 1H), 4.69- 4.49 (m, 1H), 3.31- 3.17 (m, 2H), 3.16-3.05 (m, 3H), 2.62-2.57 (m, 3H), 1.58- |
| | | | | | 1.37 (m, 2H), 0.80- |
| | | | | | 0.65 (m, 3H) ppm. |
| 65 | 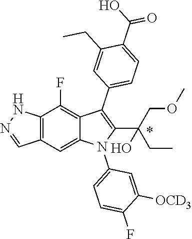 | 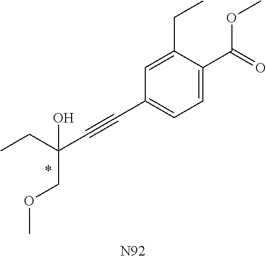 [SMILES: CCc1cc(C#CC(O)(CC)COC)ccc1C(=O)OC ; "N92"] | S8 | 553.5 | ¹H NMR (400 MHz, DMSO-d₆) δ 13.02-12.75 (m, 2H), 8.09-8.00 (m, 1H), 7.91- 7.78 (m, 1H), 7.47- 7.35 (m, 3H), 7.35-7.13 (m, 1H), 7.12-6.92 (m, 1H), 6.79- 6.71 (m, 1H), 4.68- 4.47 (m, 1H), 3.32-3.16 (m, 2H), 3.16-3.08 (m, 3H), 3.07-2.90 (m, 2H), 1.58- |
| | | | | | 1.39 (m, 2H), 1.28- |
| | | | | | 1.14 (m, 3H), |
| | | | | | 0.81-0.63 (m, 3H) |
| | | | | | ppm. |
| 51 | 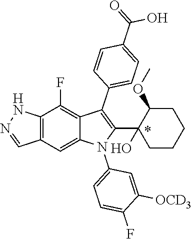 | 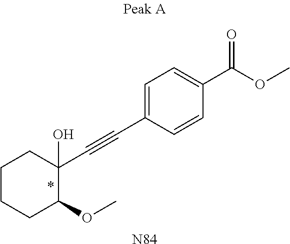 [SMILES: COC(=O)c1ccc(C#CC2(O)CCCC[C@@H]2OC)cc1 ; "N84"] | S8 | 551.4 | ¹H NMR (400 MHz, DMSO-d₆) δ 13.16-12.81 (m, 2H), 8.09-7.93 (m, 3H), 7.70- 7.54 (m, 2H), 7.49- 7.28 (m, 2H), 7.19-6.86 (m, 1H), 6.81-6.63 (m, 1H), 4.49-3.73 (m, 1H), 3.21- 3.10 (m, 1H), 2.97 (s, 1H), 2.87 (s, 2H), 2.14-1.95 (m, 1H), 1.81- |
| | | | | | 1.59 (m, 2H), 1.59- |
| | | | | | 1.42 (m, 1H), |
| | | | | | 1.43-1.03 (m, |
| | | | | | 3H), 0.94-0.68 |
| | | | | | (m, 1H) ppm. |

The following compounds were made using the methods described in Example 1 except that, different starting materials and reagents were respectively used in Step 1 in place of benzyl 6-bromo-7-fluoro-5-((4-fluoro-3-methyl phenyl)amino)-1H-indazole-1-carboxylate and methyl (S)-4-(3-hydroxy-3-(methoxymethyl)pent-1-yn-1-yl)benzoate and the reaction was carried out at 110° C. Step 2 was carried out in absence of THY:

| | | | | LC/MS | |
|---|---|---|---|---|---|
| Cmpd | | | Starting | m/z | NMR (shifts in |
| No. | Compound | Reagent | material | [M + H]⁺ | ppm) |
| 35 | 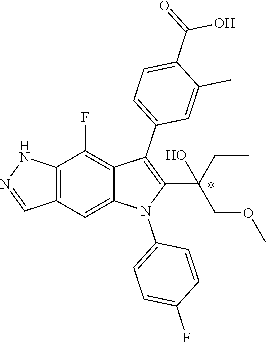 [SMILES: CCC(O)(COC)c1c(-c2ccc(C(=O)O)c(C)c2)c2c(F)c3[nH]ncc3cc2n1-c1ccc(F)cc1] | 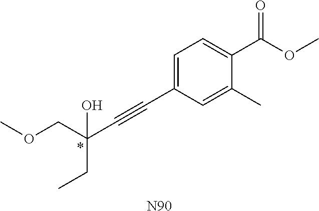 | S6 | 506.3 | ¹H NMR (400 MHz, DMSO-d₆) δ 12.97 (s, 1H), 12.81 (s, 1H), 8.04 (d, J = 3.2 Hz, 1H), 7.91-7.85 (m, 1H), 7.56-7.49 (m, 1H), 7.46-7.35 (m, 5H), 6.66 (s, 1H), 4.57 (m, 1H), 3.25 (m, 1H), 3.18 (m, 1H), 3.11 (m, 3H), 2.59 (m, 3H), 1.46 (m, 2H), 0.72 (m, 3H) ppm. |
| 36 | 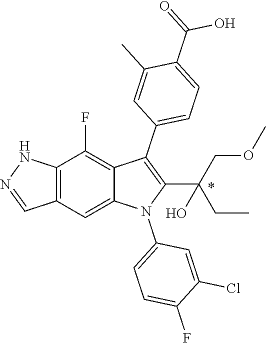 [SMILES: CCC(O)(COC)c1c(-c2ccc(C(=O)O)c(C)c2)c2c(F)c3[nH]ncc3cc2n1-c1ccc(F)c(Cl)c1] | 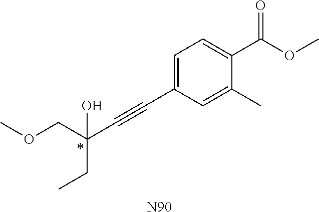 | S7 | 540.3 | ¹H NMR (300 MHz, Methanol-d₄) δ 8.01-7.95 (m, 2H), 7.67-7.53 (m, 1H), 7.48-7.37 (m, 4H), 6.78 (s, 1H), 3.39 (m, 1H), 3.23- 3.15 (m, 4H), 2.68- 2.64 (m, 3H), 1.70- 1.53 (m, 2H), 0.84 (m, 3H) ppm; exchangeable H not observed. |
| 62 | 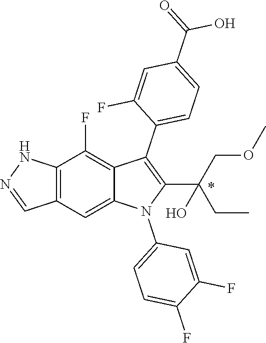 [SMILES: CCC(O)(COC)c1c(-c2ccc(C(=O)O)cc2F)c2c(F)c3[nH]ncc3cc2n1-c1ccc(F)c(F)c1] | 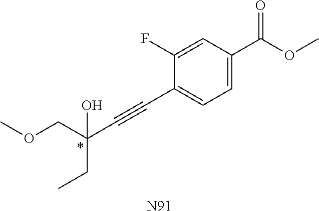 | S14 | 528.3 | ¹H NMR (400 MHz, Methanol-d₄) δ 8.01 (dd, J = 3.2, 1.2 Hz, 1H), 7.92- 7.86 (m, 1H), 7.79 (m, 1H), 7.63 (m, 1H), 7.60-7.35 (m, 2H), 7.28 (m, 1H), 6.85-6.76 (m, 1H), 3.47-3.34 (m, 1H), 3.26-3.10 (m, 4H), 1.73-1.56 (m, 2H), 0.91-0.77 (m, 3H) ppm; exchangeable H not observed. |
| 63 | 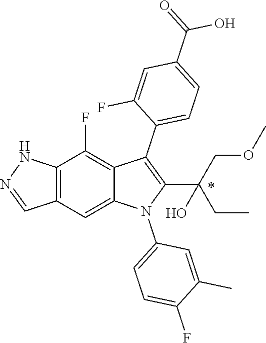 [SMILES: CCC(O)(COC)c1c(-c2ccc(C(=O)O)cc2F)c2c(F)c3[nH]ncc3cc2n1-c1ccc(F)c(C)c1] | 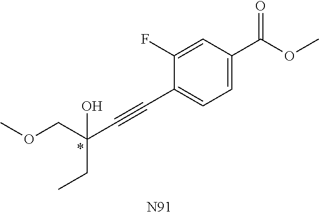 | S21 | 524.3 | ¹H NMR (400 MHz, Methanol-d₄) δ 7.98 (m, 1H), 7.85 (m, 1H), 7.76 (m, 1H), 7.62 (m, 1H), 7.47-7.22 (m, 3H), 6.76-6.72 (m, 1H), 3.41-3.34 (m, 1H), 3.26-3.13 (m, 4H), 2.37 (m, 3H), 1.69-1.56 (m, 2H), 0.85 (m, 3H) ppm; exchangeable H not observed. |
| 37 | 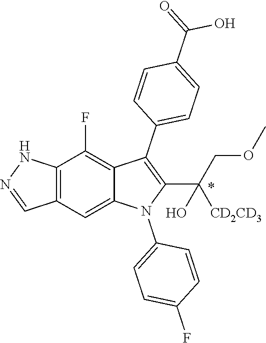 | 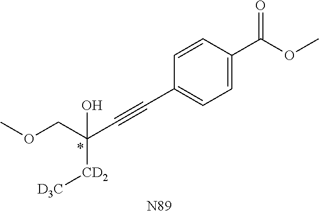 | S6 | 497.2 | ¹H NMR (400 MHz, Methanol-d₄) δ 8.10-8.04 (m, 2H), 7.97 (d, J = 3.2 Hz, 1H), 7.62 (d, J = 8.1 Hz, 2H), 7.55-7.49 (m, 1H), 7.44 (m, 1H), 7.31 (m, 2H), 6.73 (s, 1H), 3.35 (d, J = 9.7 Hz, 1H), 3.18- 3.13 (m, 4H) ppm; exchangeable H not observed. |
| 38 | 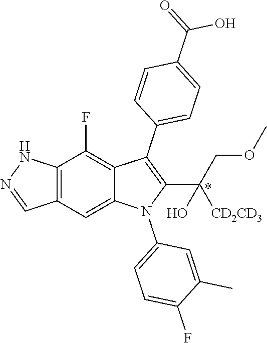 | 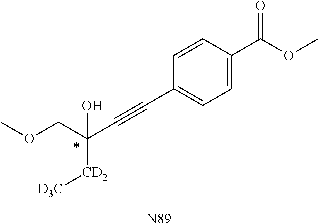 | S21 | 511.3 | ¹H NMR (400 MHz, Methanol-d₄) δ 8.10-8.03 (m, 2H), 7.97 (d, J = 3.2 Hz, 1H), 7.62 (d, J = 8.0 Hz, 2H), 7.41-7.21 (m, 3H), 6.73 (s, 1H), 3.35 (m, 1H), 3.17 (m, 3H), 3.16-3.13 (m, 1H), 2.37 (m, 3H) ppm; exchangeable H not observed. |
| 39 | 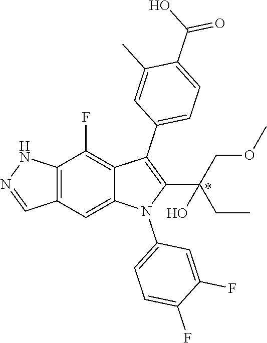 [SMILES: CCC(O)(COC)c1c(-c2ccc(C(=O)O)c(C)c2)c2c(F)c3[nH]ncc3cc2n1-c1ccc(F)c(F)c1] | 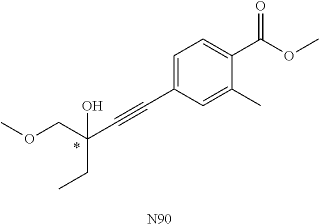 | S14 | 524.3 | ¹H NMR (400 MHz, Methanol-d₄) δ 7.99 (m, 2H), 7.51-7.35 (m, 4H), 7.35-7.22 (m, 1H), 6.79 (d, J = 1.7 Hz, 1H), 3.43-3.35 (m, 1H), 3.24-3.14 (m, 4H), 2.70-2.63 (m, 3H), 1.69-1.55 (m, 2H), 0.84 (m, 3H) ppm; exchangeable H not observed. |
| 40 | 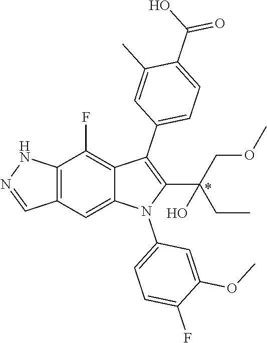 [SMILES: CCC(O)(COC)c1c(-c2ccc(C(=O)O)c(C)c2)c2c(F)c3[nH]ncc3cc2n1-c1ccc(F)c(OC)c1] | 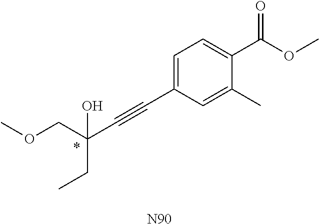 | S1 | 536.3 | ¹H NMR (400 MHz, DMSO-d₆) δ 12.96 (s, 1H), 12.81 (s, 1H), 8.04 (d, J = 3.2 Hz, 1H), 7.91-7.85 (m, 1H), 7.44-7.17 (m, 4H), 7.09-6.96 (m, 1H), 6.78-6.73 (m, 1H), 4.66-4.50 (m, 1H), 3.84 (m, 3H), 3.30- 3.16 (m, 2H), 3.16- 3.09 (m, 3H), 2.60 (m, 3H), 1.56-1.41 (m, 2H), 0.78-0.68 (m, 3H) ppm. |
| 44 | 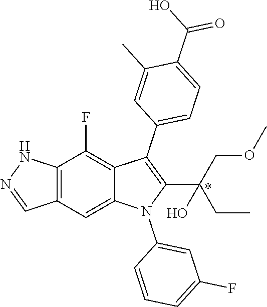 [SMILES: CCC(O)(COC)c1c(-c2ccc(C(=O)O)c(C)c2)c2c(F)c3[nH]ncc3cc2n1-c1cccc(F)c1] | 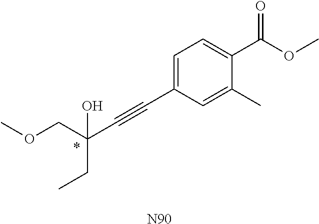 | S13 | 506.3 | ¹H NMR (300 MHz, Methanol-d₄) δ 7.98 (s, 1H), 7.97 (d, J = 3.7 Hz, 1H), 7.62-7.53 (m, 1H), 7.42 (d, J = 9.7 Hz, 2H), 7.36-7.26 (m, 2H), 7.26-7.18 (m, 1H), 6.75 (s, 1H), 3.44-3.35 (m, 1H), 3.19 (m, 4H), 2.70- 2.62 (m, 3H), 1.71- 1.54 (m, 2H), 0.90- 0.80 (m, 3H) ppm; exchangeable H not observed. |
| 47 | 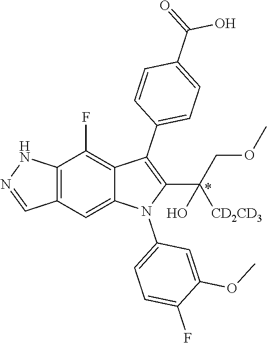 | 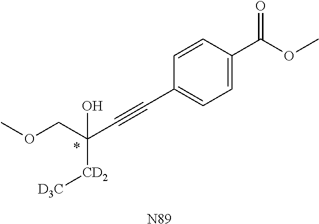 | S1 | 527.4 | ¹H NMR (400 MHz, Methanol-d₄) δ 8.11-8.03 (m, 2H), 7.99 (d, J = 3.3 Hz, 1H), 7.61 (d, J = 8.2 Hz, 2H), 7.34-7.13 (m, 2H), 7.04 (dddd, J = 21.1, 8.5, 4.0, 2.4 Hz, 1H), 6.79 (d, J = 4.2 Hz, 1H), 3.89 (m, 3H), 3.41 (t, J = 9.9 Hz, 1H), 3.23- 3.15 (m, 4H) ppm; exchangeable H not observed. |
| 48 | 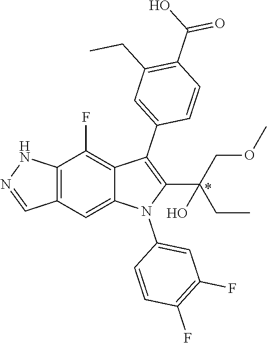 [SMILES: CCc1cc(-c2c(C(O)(CC)COC)n(-c3ccc(F)c(F)c3)c3cc4cn[nH]c4c(F)c23)ccc1C(=O)O] | 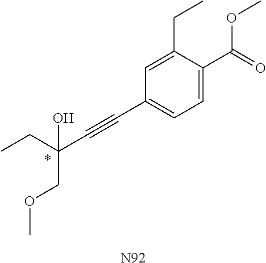 [SMILES: CCc1cc(C#CC(O)(CC)COC)ccc1C(=O)OC ; "N92"] | S14 | 538.4 | ¹H NMR (400 MHz, DMSO-d₆) δ 13.00 (s, 1H), 12.89 (s, 1H), 8.05 (d, J = 3.2 Hz, 1H), 7.85 (dd, J = 7.9, 5.9 Hz, 1H), 7.68- 7.51 (m, 2H), 7.43- 7.36 (m, 2H), 7.35- 7.12 (m, 1H), 6.78- 4.63 (m, 1H), 4.72 (m, 1H), 3.33-3.27 (m, 1H), 3.22 (m, 1H), 3.12 (m, 3H), 3.07-2.91 (m, 2H), 1.54-1.38 (m, 2H), 1.25-1.14 (m, 3H), 0.76-0.68 (m, 3H) ppm. |
| 49 | 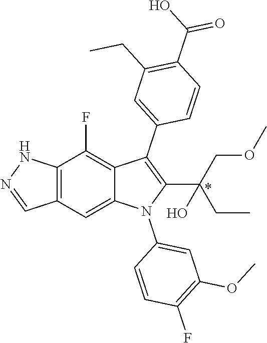 [SMILES: CCc1cc(-c2c(C(O)(CC)COC)n(-c3ccc(F)c(OC)c3)c3cc4cn[nH]c4c(F)c23)ccc1C(=O)O] | 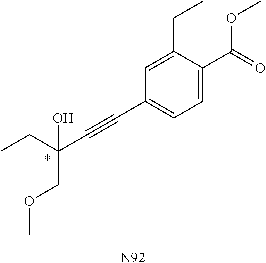 [SMILES: CCc1cc(C#CC(O)(CC)COC)ccc1C(=O)OC ; "N92"] | S1 | 550.4 | ¹H NMR (400 MHz, DMSO-d₆) δ 12.97 (s, 1H), 12.85 (s, 1H), 8.05 (d, J = 3.3 Hz, 1H), 7.87-7.80 (m, 1H), 7.45-7.35 (m, 3H), 7.35-7.15 (m, 1H), 7.11-6.94 (m, 1H), 6.79-6.72 (m, 1H), 4.58 (m, 1H), 3.84 (m, 3H), 3.30 (d, J = 9.8 Hz, 1H), 3.21 (t, J = 10.0 Hz, 1H), 3.17-3.06 (m, 3H), 3.00 (m, 2H), 1.57-1.39 (m, 2H), 1.25-1.16 (m, 3H), 0.78-0.66 (m, 3H) ppm. |

### Example 2

### (S)-4-(8-Fluoro-5-(4-fluorophenyl)-6-(2-hydroxy-1-methoxybutan-2-yl)-1,5-dihydropyrrolo[2,3-f]indazol-7-yl)benzoic acid (Compound 1)

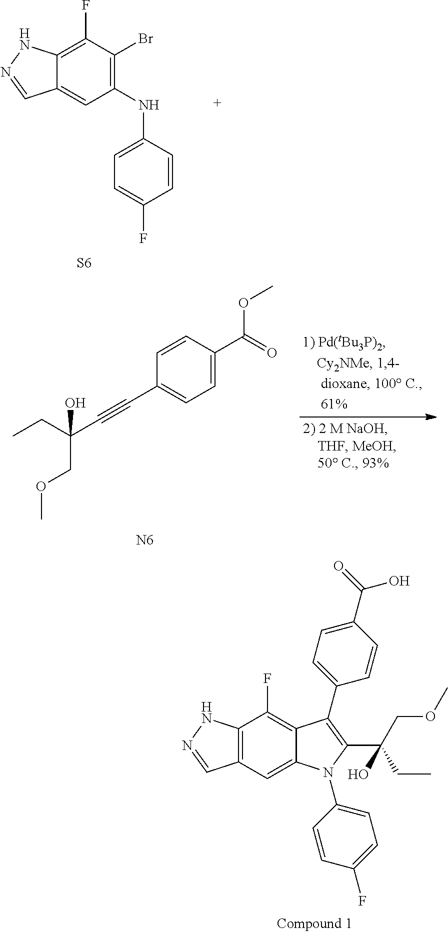

### Step 1

N,N-Dicyclohexylmethylamine (800 μL, 3.735 mmol) was added to a stirred mixture of 6-bromo-7-fluoro-N-(4-fluorophenyl)-1H-indazol-5-amine (S6, 505 mg, 1.480 mmol) and methyl (S)-4-(3-hydroxy-3-(methoxymethyl)pent-1-yn-1-yl)benzoate (Peak B, N6, 463 mg, 1.765 mmol) in 1,4-dioxane (9.5 mL) and the reaction mixture was flushed with nitrogen for 15 min. Pd(^{t}Bu₃P)₂ (78 mg, 0.1526 mmol) was added in one portion and the reaction was heated to 110° C. for 80 min. The reaction was cooled to ambient temperature and diluted with EtOAc (10 mL). The solution was washed with a saturated aqueous NH₄Cl solution (10 mL), water (10 mL) and brine (10 mL), and the combined organic extracts were dried through phase separation cartridge. The filtrates were concentrated in vacuo. Purification by flash chromatography (40 g SiO₂, 0 to 40% EtOAc in heptane) gave methyl (S)-4-(8-fluoro-5-(4-fluorophenyl)-6-(2-hydroxy-1-methoxybutan-2-yl)-1,5-dihydropyrrolo[2,3-f]indazol-7-yl)benzoate (453 mg, 61%) as a pale yellow solid. ¹H NMR (400 MHz, DMSO-d₆) δ 12.99-12.94 (m, 1H), 8.07-7.97 (m, 3H), 7.63 (t, J=7.5 Hz, 2H), 7.54 (m, 1H), 7.49-7.37 (m, 3H), 6.68 (s, 1H), 4.56 (s, 1H), 3.90 (s, 3H), 3.29-3.14 (m, 2H), 3.11 (s, 3H), 1.54-1.36 (m, 2H), 0.72 (t, J=7.3 Hz, 3H) ppm. ¹⁹F NMR (376 MHz, DMSO-d₆) δ −113.76, −143.65 ppm. ESI-MS m/z calc. 505.181, found 506.11 (M+1)⁺.

### Step 2

2 M Aqueous NaOH (154 mL, 308.0 mmol) was added to a stirred solution of methyl (S)-4-(8-fluoro-5-(4-fluorophenyl)-6-(2-hydroxy-1-methoxybutan-2-yl)-1,5-dihydropyrrolo[2,3-f]indazol-7-yl)benzoate (15.71 g, 31.08 mmol) in a mixture of THF (312 mL) and MeOH (156 mL) and the reaction was heated to 50° C. for 1 h. The reaction was cooled to ambient temperature and the volatiles were removed in vacuo. The resulting aqueous solution was diluted with water (312 mL) and extracted with DCM (2×300 mL). The aqueous layer was collected and acidified to pH 3.05 (pH meter) by addition of 2 M HCl. A fluffy white solid was filtered, washed with water (3×300 mL) and dried in the vacuum oven at 50-60° C. overnight to give (S)-4-(8-fluoro-5-(4-fluorophenyl)-6-(2-hydroxy-1-methoxybutan-2-yl)-1,5-dihydropyrrolo[2,3-f]indazol-7-yl)benzoic acid (Compound 1, 14.165 g, 93%) as a white solid. ¹H NMR (400 MHz, DMSO-d₆) δ 12.94 (m, 2H), 8.04 (d, J=3.3 Hz, 1H), 8.00-7.95 (m, 2H), 7.63-7.49 (m, 3H), 7.49-7.35 (m, 3H), 6.67 (s, 1H), 4.55 (s, 1H), 3.28-3.15 (m, 2H), 3.11 (s, 3H), 1.55-1.36 (m, J=7.2 Hz, 2H), 0.72 (t, J=7.3 Hz, 3H) ppm. 19F NMR (376 MHz, DMSO-d₆) δ −113.86, −143.68 ppm. ESI-MS m/z calc. 491.166, found 492.1 (M+1)⁺.

The following compounds were made using the methods described in Example 2 except that, different starting materials and reagents were respectively used in Step 1 in place of 6-bromo-7-fluoro-N-(4-fluorophenyl)-1H-indazol-5-amine and methyl (S)-4-(3-hydroxy-3-(methoxymethyl)pent-1-yn-1-yl)benzoate:

| | | | | LC/MS | |
|---|---|---|---|---|---|
| Cmpd | | | Starting | m/z | NMR (shifts in |
| No. | Compound | Reagent | material | [M + H]⁺ | ppm) |
| 6 | 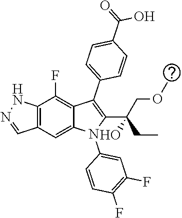 [SMILES: CC[C@](O)(COC)c1c(-c2ccc(C(=O)O)cc2)c2c(F)c3[nH]ncc3cc2n1-c1ccc(F)c(F)c1] | 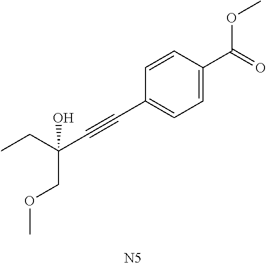 [SMILES: CC[C@@](O)(C#Cc1ccc(C(=O)OC)cc1)COC ; "N5"] | S25 | 510.0 | ¹H NMR (400 MHz, Methanol- d₄) δ 8.11-8.05 (m, 2H), 7.99 (d, J = 3.2 Hz, 1H), 7.60 (d, J = 8.2 Hz, 2H), 7.49-7.43 (m, 1H), 7.41-7.30 (m, 1H), 7.26 (s, 1H), 6.79 (d, J = 1.6 Hz, 1H), 3.43- 3.35 (m, 2H), 3.18 (d, J = 11.0 Hz, 3H), 1.68-1.55 (m, 2H), 0.84 (m, 3H) ppm; NH |
| | | | | | indazole and OH |
| | | | | | acid and alcohol |
| | | | | | not observed. |
| 8 | 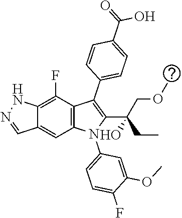 [SMILES: CC[C@](O)(COC)c1c(-c2ccc(C(=O)O)cc2)c2c(F)c3[nH]ncc3cc2n1-c1ccc(F)c(OC)c1] | 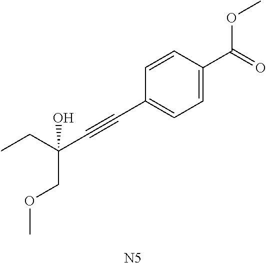 [SMILES: CC[C@@](O)(C#Cc1ccc(C(=O)OC)cc1)COC ; "N5"] | S1 | 522.4 | ¹H NMR (400 MHz, DMSO-d₆) δ 12.94 (s, 2H), 8.02 (d, J = 3.2 Hz, 1H), 7.94-7.81 (m, 2H), 7.42-7.28 (m, 3H), 7.24- 6.87 (m, 2H), 6.74 (d, J = 6.2 Hz, 1H), 4.44 (d, J = 54.5 Hz, 1H), 3.84 (d, J = 4.6 Hz, 3H), 3.38- 3.35 (m, 1H), 3.23-3.15 (m, 1H), 3.11 (d, J = 9.4 Hz, 3H), 1.57- 1.39 (m, 2H), 0.81- |
| | | | | | 0.62 (m, 3H) |
| | | | | | ppm. |
| 10 | 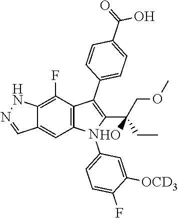 | 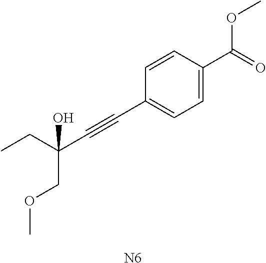 | S8 | 525.1 | 1H NMR (400 MHz, DMSO) δ 12.95 (brs, 1H), 12.92 (brs, 1H), 8.04 (m, 1H), 8.02- 7.94 (m, 2H), 7.58 (m, 2H), 7.39 (m, 1H), 7.26 (m, 1H), 7.03 (m, 1H), 6.76 (m 1H), 4.54 (m, 1H), 3.29 (m, 1H), 3.19 (m, 1H), 3.12 (m, 3H), 1.48 (m, 2H), 0.73 (m, 3H). |

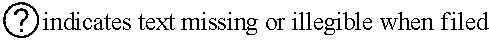

The following compounds were made using the methods described in Example 2 except that, different starting materials and reagents were respectively used in Step 1 in place of 6-bromo-7-fluoro-N-(4-fluorophenyl)-1H-indazol-5-amine and methyl (S′)-4-(3-hydroxy-3-(methoxymethyl)pent-1-yn-1-yl)benzoate. In Step 2, the reaction was carried out in the presence of 1M NaOH:

| | | | | LC/MS | |
|---|---|---|---|---|---|
| | | | | m/z | |
| Cmpd | | | Starting | [M + | NMR (shifts in |
| No. | Compound | Reagent | material | H]⁺ | ppm) |
| 9 | 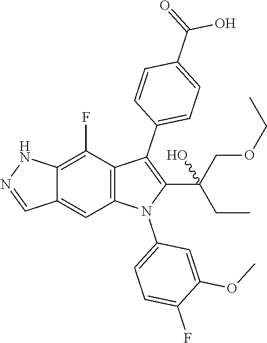 [SMILES: CCOCC(O)(CC)c1c(-c2ccc(C(=O)O)cc2)c2c(F)c3[nH]ncc3cc2n1-c1ccc(F)c(OC)c1] | 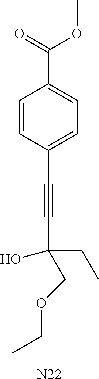 [SMILES: CCOCC(O)(C#Cc1ccc(C(=O)OC)cc1)CC ; "N22"] | S1 | 536.3 | 1H NMR (400 MHz, Methanol- d₄) δ 8.11-8.03 (m, 2H), 7.99 (d, J = 3.2 Hz, 1H), 7.65- 7.60 (m, 2H), 7.32-7.26 (m, 1H), 7.18-6.99 (m, 2H), 6.80 (d, J = 3.5 Hz, 1H), 3.89 (d, J = 3.8 Hz, 3H), 3.53-3.42 (m, 2H), 3.28- 3.21 (m, 2H), 1.69- 1.57 (m, 2H), 1.14 (q, J = 7.2 Hz, 3H), 0.85 (m, 3H). ppm; indazole NH, and acid OH, and alcohol OH not observed. |
| 13 | 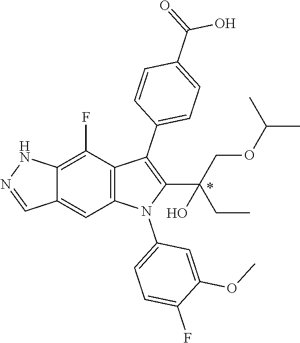 [SMILES: CCC(O)(COC(C)C)c1c(-c2ccc(C(=O)O)cc2)c2c(F)c3[nH]ncc3cc2n1-c1ccc(F)c(OC)c1] | 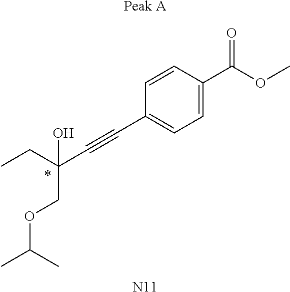 | S1 | 550.1 | ¹H NMR (400 MHz, Methanol- d₄) δ 8.12-8.07 (m, 2H), 8.01 (d, J = 3.2 Hz, 1H), 7.72- 7.61 (m, 2H), 7.35-7.18 (m, 2H), 7.13-7.02 (m, 1H), 6.82 (d, J = 2.5 Hz, 1H), 3.91 (d, J = 0.9 Hz, 3H), 3.60-3.42 (m, 2H), 3.29 (t, J = 9.2 Hz, 1H), 1.75- 1.63 (m, 1H), 1.63- 1.53 (m, 1H), 1.14-1.04 (m, 6H), 0.87 (m, 3H) ppm; indazole NH, and acid OH, and alcohol OH not observed. |
| 11 | 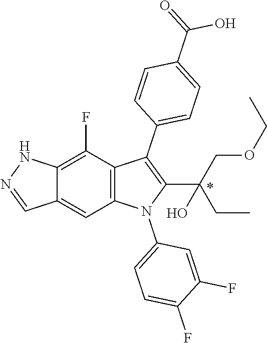 [SMILES: CCOCC(O)(CC)c1c(-c2ccc(C(=O)O)cc2)c2c(F)c3[nH]ncc3cc2n1-c1ccc(F)c(F)c1] | 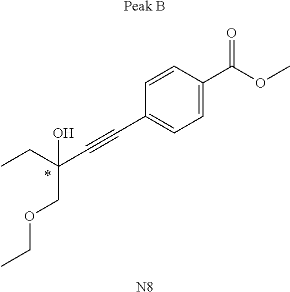 | S14 | 524.0 | ¹H NMR (400 MHz, Methanol-d₄) δ 8.08 (dd, J = 8.3, 2.7 Hz, 2H), 8.00 (d, J = 3.− Hz, 1H), 7.65-7.59 (m, 2H), 7.54-7.38 (m, 2H), 7.36-7.24 (m, 1H), 6.80 (d, J = 3.6 Hz, 1H), 3.50-3.36 (m, 2H), 3.28-3.22 (m, 2H), 1.69-1.54 (m, 2H), 1.14 (t, J = 7.0 Hz, 3H), 0.83 (m, 3H) ppm; indazole NH, and acid OH, and alcohol OH not observed. |

The following compound was made using the methods described in Example 2 except that a different starting material and reagent were used in Step 1 in place of 6-bromo-7-fluoro-N-(4-fluorophenyl)-1H-indazol-5-amine and methyl (S′)-4-(3-hydroxy-3-(methoxymethyl)pent-1-yn-1-yl)benzoate, respectively, and the reaction was carried out in DMA in place of 1,4-dioxane. Step 2 was carried out at 60° C.:

| | | | | LC/MS | |
|---|---|---|---|---|---|
| Cmpd | | | Starting | m/z | NMR (shifts in |
| No. | Compound | Reagent | material | [M + H]⁺ | ppm) |
| 59 | 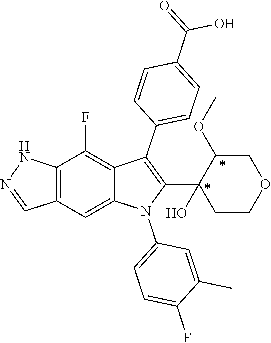 [SMILES: COC1COCCC1(O)c1c(-c2ccc(C(=O)O)cc2)c2c(F)c3[nH]ncc3cc2n1-c1ccc(F)c(C)c1] | 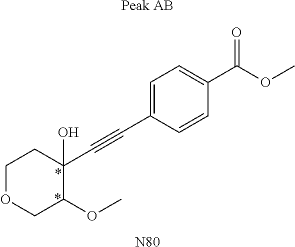 [SMILES: "Peak AB" ; COC(=O)c1ccc(C#CC2(O)CCOCC2OC)cc1 ; "N80"] | S21 | 534.5 | ¹H NMR (400 MHz, DMSO-d₆) δ 12.99 (s, 2H), 8.13- 7.89 (m, 3H), 7.77-7.43 (m, 3H), 7.41-7.12 (m, 2H), 6.70 (d, J = 1.6 Hz, 1H), 4.47 (s, 1H), 3.97- 3.66 (m, 2H), 3.59- 3.10 (m, 6H), 2.40-2.23 (m, 3H), 2.16-1.67 (m, 2H) ppm. |

The following compounds were made using the methods described in Example 2 except that, different starting materials and reagents were used in Step 1 in place of 6-bromo-7-fluoro-N-(4-fluorophenyl)-1H-indazol-5-amine and methyl (S)-4-(3-hydroxy-3-(methoxymethyl)pent-1-yn-1-yl)benzoate, respectively, and the reaction was carried out in DMA in place of 1,4-dioxane. Step 2 was carried out at ambient temperature:

| | | | | LC/MS | |
|---|---|---|---|---|---|
| Cmpd | | | Starting | m/z | NMR (shifts in |
| No. | Compound | Reagent | material | [M + H]⁺ | ppm) |
| 29 | 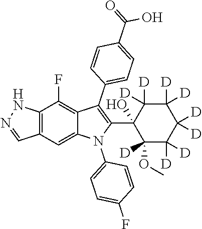 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)cc2)[C@@]([2H])(OC)C1([2H])[2H]] | 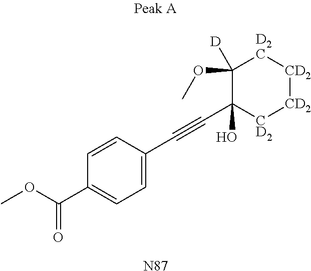 | S6 | 527.4 | ¹H NMR (400 MHz, DMSO-d₆) δ 12.97 (s, 2H), 8.08- 7.95 (m, 3H), 7.75-7.53 (m, 3H), 7.39 (s, 3H), 6.66 (s, 1H), 3.98 (s, 1H), 2.92 (s, 3H) ppm. |
| 30 | 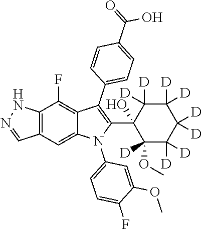 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)c(OC)c2)[C@@]([2H])(OC)C1([2H])[2H]] | 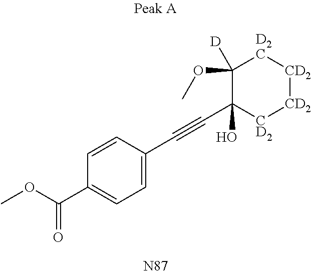 | S1 | 557.4 | ¹H NMR (400 MHz, DMSO-d₆) δ 12.95 (s, 2H), 8.08- 7.97 (m, 3H), 7.71-7.58 (m, 2H), 7.49-7.37 (m, 1H), 7.36- 6.87 (m, 2H), 6.76- 6.71 (m, 1H), 4.33-4.16 (m, 1H), 3.91-3.82 (m, 3H), 3.00- 2.85 (m, 3H) ppm. |
| 31 | 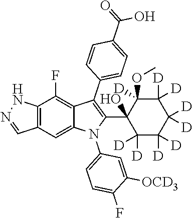 | 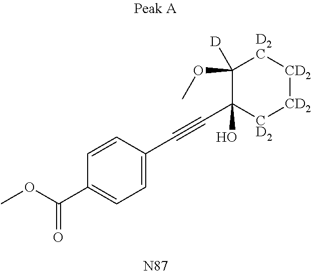 | S8 | 560.4 | ¹H NMR (400 MHz, DMSO-d₆) δ 12.96 (s, 2H), 8.06- 7.98 (m, 3H), 7.69-7.57 (m, 2H), 7.48-7.36 (m, 1H), 7.36- 6.85 (m, 2H), 6.74 (d, J = 5.7 Hz, 1H), 4.38-3.73 (m, 1H), 3.03-2.83 (m, 3H) ppm. |

The following compounds were made using the methods described in Example 2 except that, different starting materials and reagents were used in Step 1 in place of 6-bromo-7-fluoro-N-(4-fluorophenyl)-1H-indazol-5-amine and methyl (S′)-4-(3-hydroxy-3-(methoxymethyl)pent-1-yn-1-yl)benzoate, respectively. In Step 2, the reaction was carried at ambient temperature:

| | | | | LC/MS | |
|---|---|---|---|---|---|
| | | | | m/z | |
| Cmpd | | | Starting | [M + | NMR (shifts in |
| No. | Compound | Reagent | material | H]⁺ | ppm) |
| 32 | 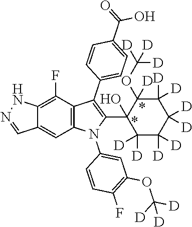 [SMILES: [2H]C([2H])([2H])Oc1cc(-n2c(C3(O)C([2H])([2H])C([2H])([2H])C([2H])([2H])C([2H])([2H])C3([2H])OC([2H])([2H])[2H])c(-c3ccc(C(=O)O)cc3)c3c(F)c4[nH]ncc4cc32)ccc1F] | 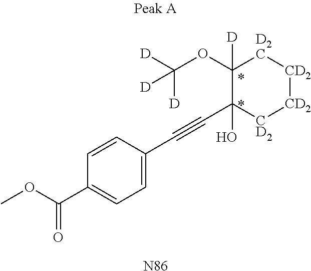 | S8 | 563.2 | ¹H NMR (400 MHz, DMSO-d₆) δ 12.97 (s, 2H), 8.07- 7.87 (m, 3H), 7.51- 7.27 (m, 4H), 7.17- 6.84 (m, 1H), 6.72 (d, J = 5.6 Hz, 1H), 4.30-3.72 (m, 1H) ppm. |
| 60 | 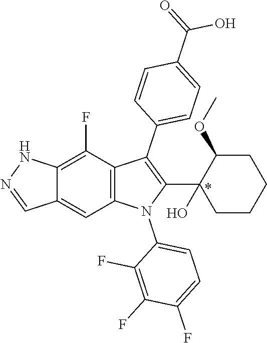 [SMILES: CO[C@H]1CCCCC1(O)c1c(-c2ccc(C(=O)O)cc2)c2c(F)c3[nH]ncc3cc2n1-c1ccc(F)c(F)c1F] | 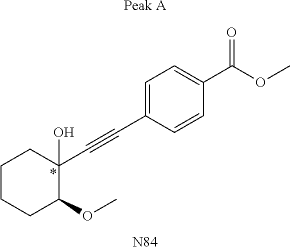 [SMILES: "Peak A" ; COC(=O)c1ccc(C#CC2(O)CCCC[C@@H]2OC)cc1 ; "N84"] | S35 | 554.3 | ¹H NMR (400 MHz, DMSO-d₆) δ 13.06 (s, 2H), 8.14- 7.96 (m, 3H), 7.69- 7.42 (m, 4H), 6.92- 6.80 (m, 1H), 4.37 (d, J = 68.1 Hz, 1H), 3.22-3.07 (m, 1H), 2.93 (d, J = 33.3 Hz, 3H), 2.11- 1.81 (m, 1H), 1.67 (s, 2H), 1.57-1.44 (m, 1H), 1.37-1.15 (m, 3H), 0.96-0.66 (m, 1H) ppm. |
| 61 | 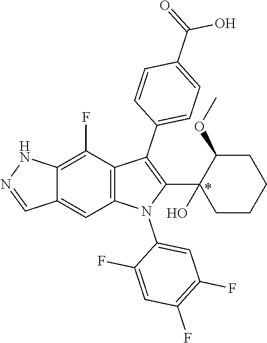 [SMILES: CO[C@H]1CCCCC1(O)c1c(-c2ccc(C(=O)O)cc2)c2c(F)c3[nH]ncc3cc2n1-c1cc(F)c(F)cc1F] | 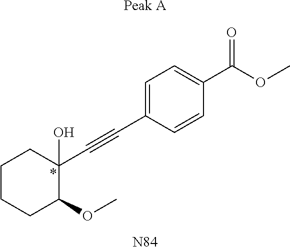 [SMILES: "Peak A" ; COC(=O)c1ccc(C#CC2(O)CCCC[C@@H]2OC)cc1 ; "N84"] | S36 | 554.3 | ¹H NMR (400 MHz, DMSO-d₆) δ 13.06 (s, 2H), 8.10- 7.97 (m, 3H), 7.95- 7.73 (m, 2H), 7.70- 7.53 (m, 2H), 6.84 (d, J = 5.3 Hz, 1H), 4.70-4.07 (m, 1H), 3.24-3.07 (m, 1H), 2.91 (s, 3H), 2.15- 1.81 (m, 1H), 1.79- 1.58 (m, 2H), 1.56- 1.46 (m, 1H), 1.46- 1.20 (m, 3H), 0.96- 0.72 (m, 1H) ppm. |

The following compound was made using the methods described in Example 2 except that, different starting material and reagent were used in Step 1 in place of 6-bromo-7-fluoro-N-(4-fluorophenyl)-1H-indazol-5-amine and methyl (S′)-4-(3-hydroxy-3-(methoxymethyl)pent-1-yn-1-yl)benzoate, respectively, and the reaction was carried out using ^{t}BuNMe₂ as the base in place of N,N-dicyclohexylmethylamine. Step 2 was carried out at ambient temperature:

| | | | | LC/MS | |
|---|---|---|---|---|---|
| Cmpd | | | Starting | m/z | NMR (shifts in |
| No. | Compound | Reagent | material | [M + H]⁺ | ppm) |
| 33 | 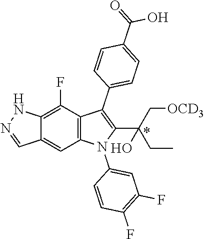 | 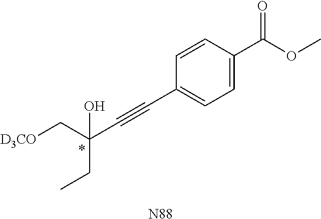 | S25 | 513.3 | ¹H NMR (400 MHz, DMSO-d₆) δ 13.02 (s, 2H), 8.15- 7.94 (m, 3H), 7.75-7.49 (m, 4H), 7.48-7.16 (m, 1H), 6.78 (d, J = 1.5 Hz, 1H), 4.89-4.60 (m, 1H), 3.30-3.16 (m, 2H), 1.58- 1.33 (m, 2H), 0.72 (t, J = 7.3 Hz, 3H) ppm. |

### Example 3

### (S)-4-(5-(3,4-Difluorophenyl)-8-fluoro-6-(2-hydroxy-1-methoxybutan-2-yl)-1,5-dihydropyrrolo[2,3-f]indazol-7-yl)benzoic acid (Compound 5)

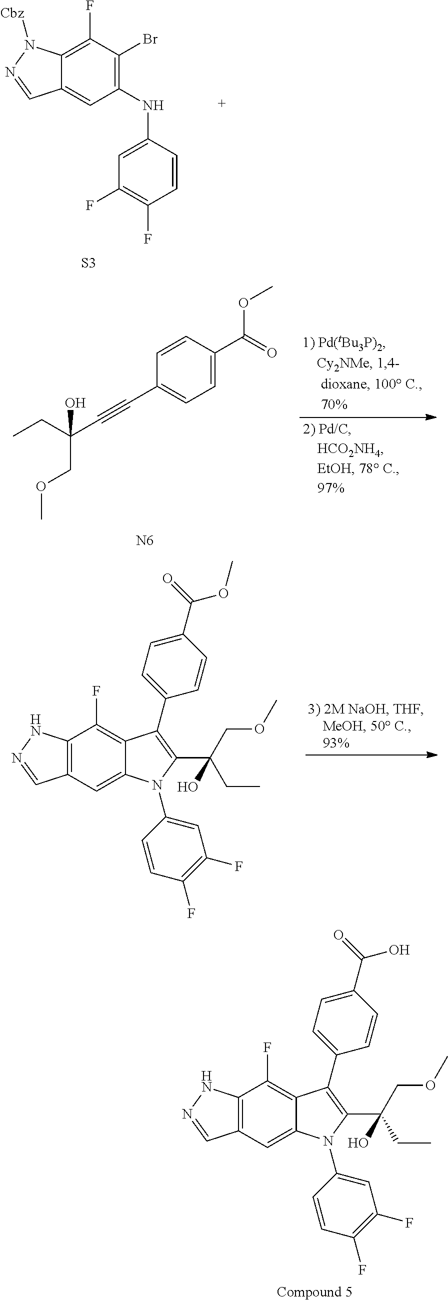

### Step 1

Pd(^{t}Bu₃P)₂ (10 g, 19.57 mmol) was added to a nitrogen flushed solution of benzyl 6-bromo-5-((3,4-difluorophenyl)amino)-7-fluoro-1H-indazole-1-carboxylate (S3, 137 g, 287.7 mmol), methyl (S)-4-(3-hydroxy-3-(methoxymethyl)pent-1-yn-1-yl)benzoate (Peak B, N6, 77.3 g, 294.7 mmol) and N,N-dicyclohexylmethylamine (160 mL, 747.0 mmol) in 1,4-dioxane (1.4 L) and the reaction mixture was heated to 100° C. for 2.5 h. The mixture was cooled to ambient temperature overnight and partitioned between EtOAc (1.4 L) and a 1 M aqueous HCl solution (1 L). The organic layer was separated, washed with a 1 M aqueous HCl solution (1 L) and with a mixture of water and a saturated brine solution (2:1, 1.5 L), dried (MgSO₄) filtered and concentrated in vacuo. Purification by flash chromatography (3 Kg SiO₂, 0 to 60% EtOAc in heptane) gave benzyl (S)-5-(3,4-difluorophenyl)-8-fluoro-6-(2-hydroxy-1-methoxybutan-2-yl)-7-(4-(methoxycarbonyl)phenyl)pyrrolo[2,3-J]indazole-1(5H)-carboxylate (132 g, 70%) as a yellow glassy oil. ESI-MS m/z calc. 657.209, found 658.3 (M+1)+.

### Step 2

Pd/C (27 g, 5% w/w, 12.69 mmol) and ammonium formate (135 g, 2.141 mol) were successively added to a stirred solution of benzyl (S)-5-(3,4-difluorophenyl)-8-fluoro-6-(2-hydroxy-1-methoxybutan-2-yl)-7-(4-(methoxycarbonyl)phenyl)pyrrolo[2,3-J]indazole-1(5H)-carboxylate (140 g, 212.9 mmol) in EtOH (1.6 L) and the reaction mixture was heated at reflux for 90 min. The mixture was cooled slowly while standing at ambient temperature overnight. The mixture was diluted with EtOAc (1 L) and heated to ˜ 85° C. The mixture was filtered hot through a pad of Celite, washing with hot EtOAc (3×500 mL). The reaction was concentrated in vacuo to a volume of approximately 300 mL. The mixture was cooled and filtered to give a first crop of methyl (S)-4-(5-(3,4-difluorophenyl)-8-fluoro-6-(2-hydroxy-1-methoxybutan-2-yl)-1,5-dihydropyrrolo[2,3-J]indazol-7-yl)benzoate. The mother liquors were concentrated in vacuo to give a gold solid (75 g). The solid was recrystallised from EtOAc (˜150 mL, 2 vol) and heptane (3 vol) to give a second crop of methyl (S)-4-(5-(3,4-difluorophenyl)-8-fluoro-6-(2-hydroxy-1-methoxybutan-2-yl)-1,5-dihydropyrrolo[2,3-fj]indazol-7-yl)benzoate. The crops were combined to give methyl (S)-4-(5-(3,4-difluorophenyl)-8-fluoro-6-(2-hydroxy-1-methoxybutan-2-yl)-1,5-dihydropyrrolo[2,3-fj]indazol-7-yl)benzoate (114 g, 97%) as a beige solid. ESI-MS m/z calc. 523.172, found 524.2 (M+1)⁺.

### Step 3

2 M NaOH (70 mL of, 140.0 mmol) was added to a stirred solution of methyl (S)-4-(5-(3,4-difluorophenyl)-8-fluoro-6-(2-hydroxy-1-methoxybutan-2-yl)-1,5-dihydropyrrolo[2,3-J]indazol-7-yl)benzoate (7.4 g, 14.14 mmol) in a mixture of MeOH (70 mL) and THF (140 mL) and the reaction mixture was heated to 50° C. for 30 min. Alternatively, LiOh may be used in the final hydrolysis step. The mixture was concentrated in vacuo. Water was added and the pH of the solution was adjusted to 3 by addition of 1 M HCl. The formed solid was filtered, washed with water and dried overnight to give (S)-4-(5-(3,4-difluorophenyl)-8-fluoro-6-(2-hydroxy-1-methoxybutan-2-yl)-1,5-dihydropyrrolo[2,3-f]indazol-7-yl)benzoic acid (Compound 5, 6.705 g, 93%) as an off-white solid. ¹H NMR (400 MHz, DMSO-d₆) δ 12.97 (m, 2H), 8.11-7.93 (m, 3H), 7.73-7.43 (m, 4H), 7.48-7.13 (m, 1H), 6.78 (d, J=1.6 Hz, 1H), 4.69 (d, J=23.5 Hz, 1H), 3.27-3.16 (m, 2H), 3.12 (d, J=9.4 Hz, 3H), 1.52-1.37 (m, 2H), 0.72 (t, J=7.3 Hz, 3H) ppm. ESI-MS m/z calc. 509.156, found 510.1 (M+1)⁺.

The following compound was made using the methods described above in Example 3 except that, benzyl 6-bromo-7-fluoro-5-((4-fluoro-3-methoxyphenyl)amino)-1H-indazole-1-carboxylate was used as the starting material in Step 1 in place of benzyl 6-bromo-5-((3,4-difluorophenyl) amino)-7-fluoro-1H-indazole-1-carboxylate. Step 2 was carried out at 50° C. in MeOH as the solvent in place of EtOH. The conditions used for the saponification Step 3 were those described in Example 1 Step 2 and the reaction was carried out at ambient temperature:

| | | | | LC/MS | |
|---|---|---|---|---|---|
| Cmpd | | | Starting | m/z | NMR (shifts in |
| No. | Compound | Reagent | material | [M + H]⁺ | ppm) |
| 3 | 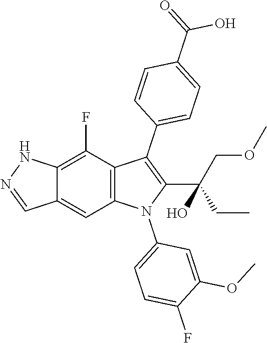 [SMILES: CC[C@@](O)(COC)c1c(-c2ccc(C(=O)O)cc2)c2c(F)c3[nH]ncc3cc2n1-c1ccc(F)c(OC)c1] | 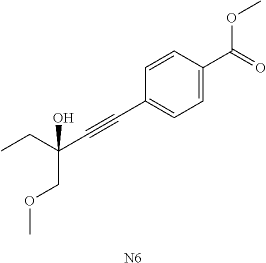 | S2 | 522.1 | ¹H NMR (300 MHz, DMSO-d₆) δ 12.93 (m, 2H), 8.04 (d, J = 3.4 Hz, 1H), 8.03- 7.92 (m, 2H), 7.63- 7.50 (m, 2H), 7.46- 7.35 (m, 1H), 7.35- 7.18 (m, 1H), 7.11- 6.94 (m, 1H), 6.76 (d, J = 5.4 Hz, 1H), 4.55 (d, J = 33.2 Hz, 1H), 3.85 (d, J = 1.9 Hz, 3H), 3.30-3.16 (m, 2H), 3.12 (m, 3H), 1.56-1.41 (m, 2H), 0.79-0.68 (m, 3H).ppm. |

The compounds were analyzed by LC/MS according to one of the following methods, as shown in Table 1.

| LC/MS | |
|---|---|
| Method | Method Description |
| A | Waters ™ CSH C18 1.7 mm 2.1 × 50 mm, 5 to |
| | 95% MeCN in H₂O (0.1% TFA), |
| | 45° C., Flow rate 1.0 mL/min over 4 min |
| B | Waters ™ BEH C8 1.7 mm 2.1 × 50 mm, |
| | 60 to 95% MeCN in H₂O (0.1% TFA), |
| | 45° C., Flow rate 1.0 mL/min over 4.0 min |
| C | Kinetex® EVO C18 2.6 mm 2.1 × 50 mm, 2 |
| | to 98% MeCN in H₂O (10 mM |
| | Ammonium Formate, pH 9), 45° C., Flow |
| | rate 1.0 mL/min over 1.5 min |
| D | Waters ™ XBridge C18 5.0 mm 4.6 × 50 mm, |
| | 10 to 90% MeCN in H₂O (10 mM |
| | Ammonium Acetate), Flow rate |
| | 1.2 mL/min over 5 min |
| E | XBridge C18 5 mm 4.6 × 75 mm, Initial |
| | Gradient 5 to 95% MeCN (NH₄HCO₃), |
| | 6 min run with 1 min equilibration |
| | Gradient 0 to 3 min at 95% MeCN and hold |
| | for 3 min, Flow rate 1.5 mL/min |
| F | Kinetex ® Polar C18 2.6 mm 3.0 × 50 mm, |
| | 5 to 95% MeCN in H₂O (0.1% |
| | Formic Acid), Flow rate 1.2 mL/min over 6 min |
| G | Kinetex ® Polar C18 2.6 mm 3.0 × 50 mm, |
| | 5 to 95% MeCN in H₂O (0.1% |
| | Formic Acid), Flow rate 1.2 mL/min over 3 min |
| H | Kinetex ® Polar C18 2.6 mm 3.0 × 50 mm, |
| | 5 to 95% MeCN in H₂O (0.1% |
| | Formic Acid), Flow rate 1.2 mL/min over 10 min |
| I | Waters ™ CSH C18 1.7 mm 2.1 × 50 mm, |
| | 2 to 98% MeCN in H₂O (0.1% TFA, |
| | pH 2), 45° C., Flow rate 0.6 mL/min over 5.0 min |
| J | Waters ™ BEH C8 1.7 mm 2.1 × 50 mm, |
| | 2 to 98% MeCN in H₂O (10 mM |
| | Ammonium Formate, pH 9), 45° C., |
| | Flow rate 0.6 mL/min over 5.0 min |
| K | Waters ™ CSH C18 1.7 mm 2.1 × 50 mm, |
| | 5 to 95% MeCN in H₂O (0.1% TFA), |
| | 60° C., Flow rate 1.25 mL/min over 2.25 min |
| L | Acquity C18 1.7 mm 50 × 2.1 mm, 5 to |
| | 95% MeCN:H₂O (0.1% Formic Acid), |
| | Flow rate 0.5 mL/min over 12 min |
| M | Poroshell 120 EC C18 2.7 mm 50 × 3.0 mm, |
| | 15 to 100% MeCN:H₂O (0.1% |
| | Formic Acid), Flow rate 0.5 mL/min over 12 min |
| N | Waters ™ BEH C18 1.7 um 2.1 × 50 mm, |
| | 5 to 95% MeCN in H₂O (5 mM Ammonium |
| | Acetate), Flow rate 0.6 mL/min over 4 min |
| O | Acquity BEH C8 1.7 um 2.1 × 50 mm, |
| | 60 to 95% MeCN in H₂O (0.1% TFA), |
| | Flow rate 0.6 mL/min over 0.6 min |

### Example 4

### Alternate Synthesis of Compound 3 and Compound 5

### A. Synthesis of (S)-4-(5-(3,4-Difluorophenyl)-8-fluoro-6-(2-hydroxy-1-meth-oxybutan-2-yl)-1,5-dihydropyrrolo[2,3-f]indazol-7-yl)benzoic acid (Compound 5)

### Step 1—Reductive Coupling

Procedure A

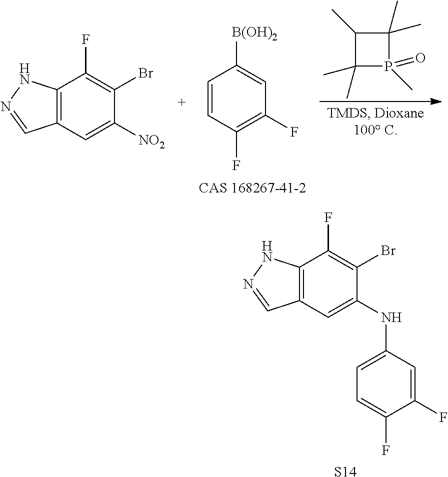 [SMILES: O=[N+]([O-])c1cc2cn[nH]c2c(F)c1Br ; OB(O)c1ccc(F)c(F)c1 ; CC1C(C)(C)P(C)(=O)C1(C)C ; Fc1ccc(Nc2cc3cn[nH]c3c(F)c2Br)cc1F ; "S14"]

To a heated solution of 3,4-difluorophenylboronic acid (2 equiv), 1,2,2,3,4,4-hexamethylphosphetane-1-oxide (0.3 equiv) and 6-bromo-7-fluoro-5-nitro-1(H)-indazole (0.1 equiv) in dioxane were added concomitantly, a solution of nitroindazole 6-bromo-7-fluoro-5-nitro-1(H)-indazole (0.9 equiv) in dioxane as well as neat TMDS (6 equiv). The mixture was stirred at 100° C. until the reaction was deemed complete. The reaction was quenched with aqueous NaOH. 2-MeTHF was added and the phases were separated. The organic phase was washed with aqueous HCl. The organic phase was concentrated, and the residue taken up in toluene. The product S14 was isolated by crystallization, filtration and drying.

Procedure B

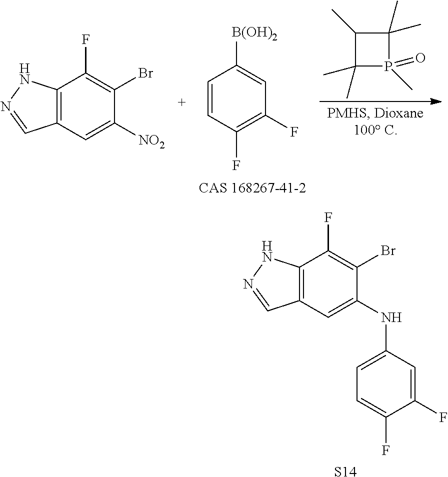 [SMILES: O=[N+]([O-])c1cc2cn[nH]c2c(F)c1Br ; OB(O)c1ccc(F)c(F)c1 ; CC1C(C)(C)P(C)(=O)C1(C)C ; Fc1ccc(Nc2cc3cn[nH]c3c(F)c2Br)cc1F ; "S14"]

To a heated solution of 6-bromo-7-fluoro-5-nitro-1(H)-indazole (1 equiv), 3,4-difluorophenylboronic acid (2 equiv), 1,2,2,3,4,4-hexamethylphosphetane-1-oxide (0.3 equiv) was added neat PMHS (6 equiv). The mixture was stirred at 100° C. until the reaction was deemed complete. The reaction mixture was quenched with aqueous NaOH and the phases were separated. The dioxane is distilled off and 2-MeTHF is added. The mixture was stirred with aqueous sorbitol and then the aqueous phase was separated. The solvent was distilled off, and the product S14 was isolated by crystallization from toluene and acetonitrile, filtration and drying.

### Step 2—Larock Annulation

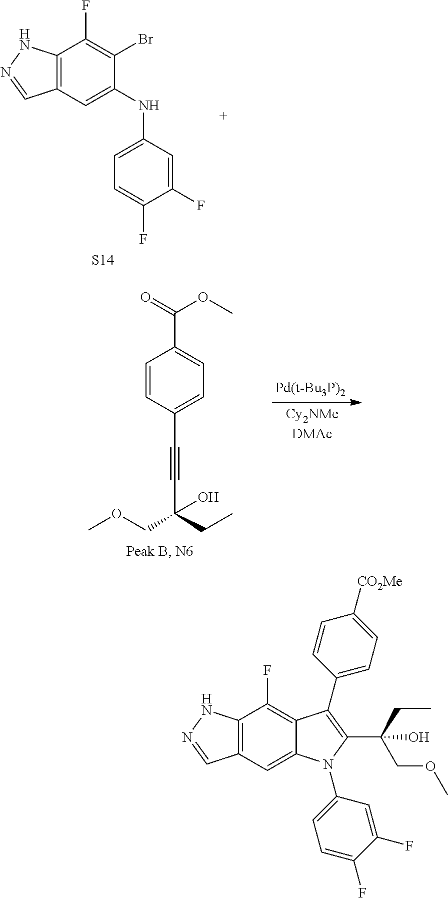 [SMILES: Fc1ccc(Nc2cc3cn[nH]c3c(F)c2Br)cc1F ; "S14" ; CC[C@](O)(C#Cc1ccc(C(=O)OC)cc1)COC ; CC[C@@](O)(COC)c1c(-c2ccc(C(=O)OC)cc2)c2c(F)c3[nH]ncc3cc2n1-c1ccc(F)c(F)c1]

Procedure A: N,N-Dicyclohexylmethylamine (2.5 equiv) was added to a heated mixture of S14 (1 equiv), Peak B N6 (1.25 equiv) and Pd(^{t}Bu₃P)₂ (0.03 equiv) in DMAc. The reaction mixture was stirred at 120° C. until complete. The mixture was cooled and diluted with 2-MeTHF, and then washed with aqueous HCl. The mixture was concentrated and the residue was taken up in methanol. Water was added and the product methyl (S)-4-(5-(3,4-difluorophenyl)-8-fluoro-6-(2-hydroxy-1-methoxybutan-2-yl)-1,5-dihydropyrrolo[2,3-f]indazol-7-yl)benzoate was isolated by crystallization, filtration and drying.

Procedure B: This procedure can be carried out with a catalyst mixture consisting of AmPhos and (MeCN)₂PdCl₂ instead of Pd(^{t}Bu₃P)₂.

### Step 3—Ester Hydrolysis

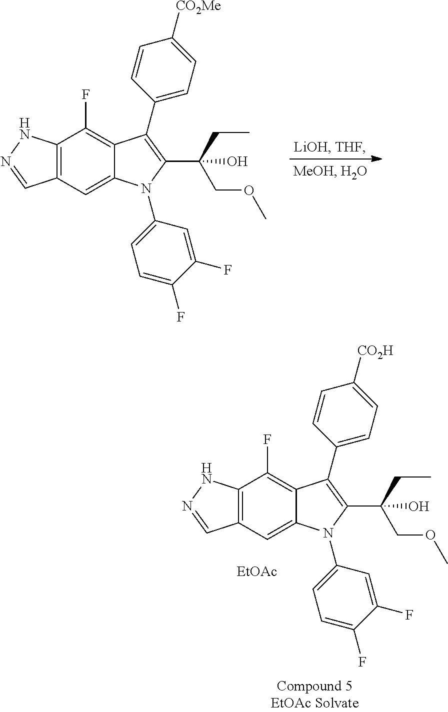 [SMILES: "EtOAc" ; CC[C@@](O)(COC)c1c(-c2ccc(C(=O)OC)cc2)c2c(F)c3[nH]ncc3cc2n1-c1ccc(F)c(F)c1 ; CC[C@@](O)(COC)c1c(-c2ccc(C(=O)O)cc2)c2c(F)c3[nH]ncc3cc2n1-c1ccc(F)c(F)c1]

To a solution of methyl (S)-4-(5-(3,4-difluorophenyl)-8-fluoro-6-(2-hydroxy-1-methoxybutan-2-yl)-1,5-dihydropyrrolo[2,3-f]indazol-7-yl)benzoate (1 equiv) in THF and MeOH was added aqueous LiOH (4 equiv). The mixture was stirred at 20° C. until the reaction was complete. The mixture concentrated and the product was extracted into MTBE. After layer separation, the organic phase was washed with aqueous citric acid followed by aqueous NaCl. The organic phase was concentrated and then redissolved in EtOAc. After heating and adding heptane, the product Compound 5 was isolated as the free form EtOAc Heptane Solvate Form A by crystallization, filtration and drying.

### Step 4—Form Conversion to Monohydrate

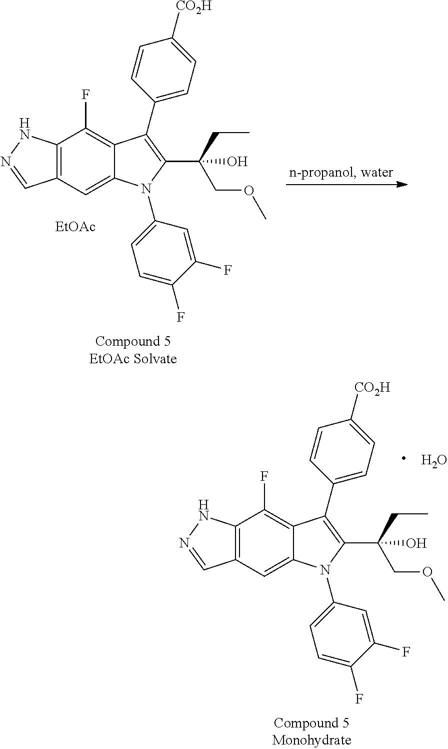

A solution of Compound 5 free form EtOAc Heptane Solvate Form Ain n-propanol was heated to 55° C. and then diluted with water. The mixture was seeded at 47° C. with Compound 5 Monohydrate Form A and then diluted with more water. The mixture was cooled to 18° C. and then the product Compound 5 Monohydrate Form A was isolated by filtration and drying.

### B. Synthesis of (S)-4-(8-fluoro-5-(4-fluoro-3-methoxyphenyl)-6-(2-hydroxy-1-methoxybutan-2-yl)-1,5-dihydropyrrolo[2,3-f]indazol-7-yl)benzoic acid (Compound 3)

### Step 1—Buchwald Coupling

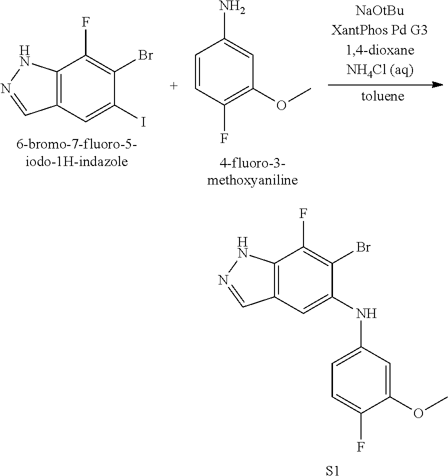

6-bromo-7-fluoro-5-iodo-1H-indazole and 4-fluoro-3-methoxyaniline were suspended in degassed 1,4-dioxane. XantPhos-Pd-G3 and sodium tert-butoxide were added and the mixture was heated until deemed complete. Once complete, the reaction was cooled and quenched with aqueous ammonium chloride. The mixture was extracted with ethyl acetate and concentrated. The product S1 was isolated by trituration with toluene, filtration and drying.

### Step 2—Larock Annulation

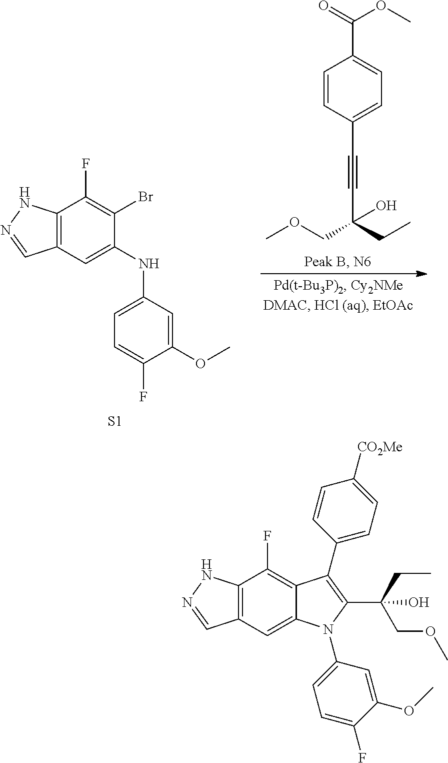

N,N-Dicyclohexylmethylamine (2.5 equiv) was added to a heated mixture of S1, Peak B, N6 and Pd(t-Bu₃P)₂ in DMAc. The reaction mixture was stirred at 120° C. until complete. The mixture was cooled and diluted with ethyl acetate, and then washed with aqueous HCl. The organic layer can be treated with a palladium scavenging resin such as Silia-MetS-thiol or Silia-MetS-DMT to remove residual palladium catalyst. The mixture was filtered, concentrated and the residue was taken up in methanol. Water was added and the product (methyl (S)-4-(8-fluoro-5-(4-fluoro-3-methoxyphenyl)-6-(2-hydroxy-1-methoxybutan-2-yl)-1,5-dihydropyrrolo[2,3-f]indazol-7-yl)benzoate was isolated by filtration and drying.

### Step 3—Ester Hydrolysis

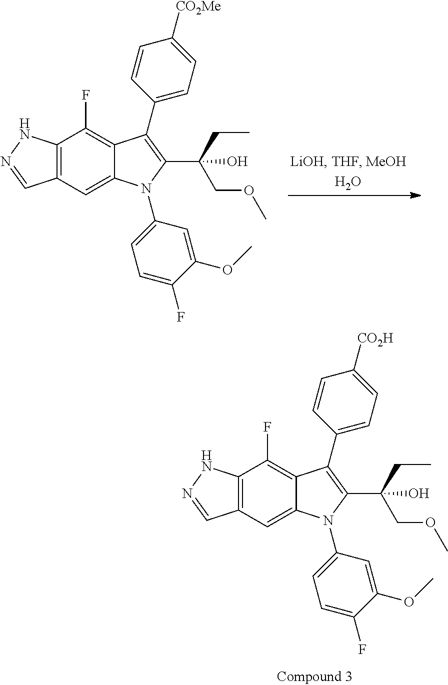 [SMILES: CC[C@@](O)(COC)c1c(-c2ccc(C(=O)OC)cc2)c2c(F)c3[nH]ncc3cc2n1-c1ccc(F)c(OC)c1 ; CC[C@@](O)(COC)c1c(-c2ccc(C(=O)O)cc2)c2c(F)c3[nH]ncc3cc2n1-c1ccc(F)c(OC)c1]

To a solution of (methyl (S)-4-(8-fluoro-5-(4-fluoro-3-methoxyphenyl)-6-(2-hydroxy-1-methoxybutan-2-yl)-1,5-dihydropyrrolo[2,3-f]indazol-7-yl)benzoate (1 equiv) in THF and MeOH was added aqueous LiOH (4 equiv). The mixture was stirred at 20° C. until the reaction was complete. The mixture concentrated and the product was extracted into 2-methyl tetrahydrofuran. After layer separation, the organic phase was washed with aqueous citric acid followed by water. The organic phase was treated with SiliaMetS-thiol and SiliaMetS-DMT resins and filtered. The filtrate was concentrated to a foam and crystallized from acetonitrile and water. The solids were isolated by filtration and drying to provide Compound 3.

### Step 4—Reslurry

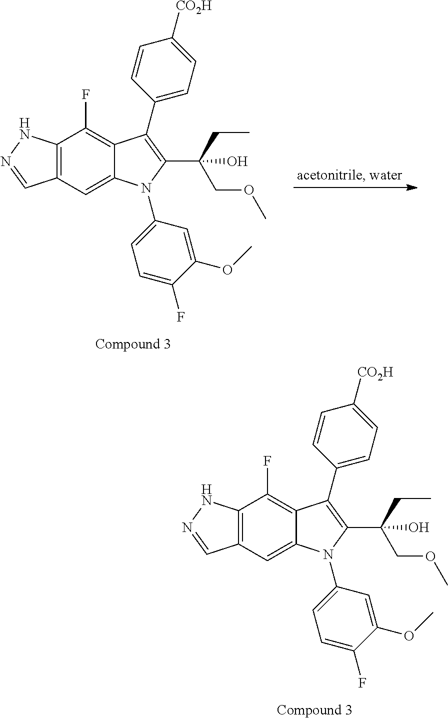 [SMILES: CC[C@@](O)(COC)c1c(-c2ccc(C(=O)O)cc2)c2c(F)c3[nH]ncc3cc2n1-c1ccc(F)c(OC)c1 ; CC[C@@](O)(COC)c1c(-c2ccc(C(=O)O)cc2)c2c(F)c3[nH]ncc3cc2n1-c1ccc(F)c(OC)c1]

Compound 3 was dissolved in acetonitrile at reflux. Water was charged and the mixture was brought back up to reflux. The mixture was slowly cooled over 12 hours. Compound 3 was isolated by filtration, a wash with 1:1 acetonitrile:water, and drying.

### Example 5

### Assays for Detecting and Measuring AAT Modulator Properties of Compounds

### A. AAT Function Assay (MSD Assay NL20-SI Cell Line)

Alpha-1 antitrypsin (AAT) is a SERPIN (serine protease inhibitor) that inactivates enzymes by binding to them covalently. This assay measured the amount of functionally active AAT in a sample in the presence of the disclosed compounds 1-210 by determining the ability of AAT to form an irreversible complex with human neutrophil Elastase (hNE). In practice, the sample (cell supernatant, blood sample, or other) was incubated with excess hNE to allow AAT-Elastase complex to be formed with all functional AAT in the sample. This complex was then captured to a microplate coated with an anti-AAT antibody. The complex captured to the plate was detected with a labeled anti-Elastase antibody and quantitated using a set of AAT standards spanning the concentration range present in the sample. Meso Scale Discovery (MSD) plate reader, Sulfo-tag labeling, and microplates were used to provide high sensitivity and wide dynamic range.

MATERIALS:

| Reagents/Plates | Concentration |
|---|---|
| Goat anti-human Alpha-1-Antitrypsin | 1 mL @ 1 mg/mL |
| Polyclonal Antibody | |
| Use at 5 μg/mL in phosphate | |
| buffered saline (PBS) | |
| Human Neutrophil Elastase | 100 μg lyophilized |
| Stock at 3.4 μM (0.1 mg + 1 mL PBS) | |
| Working at 1 μg/mL (34 nm) in MSD Assay | |
| buffer (1% bovine serum albumin (BSA)) | |
| Mouse anti-human Neutrophil Elastase | 900 μg/mL |
| Monoclonal Antibody | |
| Sulfo-tagged @ 12:1 using | |
| MSD Gold Sulfo-tag N- | |
| hydroxysuccinimide (NHS) ester; use at | |
| 0.45 μg/mL in MSD Assay buffer (1% BSA) | |
| M-AAT (Alpha-1-Antitrypsin) | 5 mg lyophilized |
| MSD Blocker A (BSA) | 250 mL |
| 5% solution in PBS for blocking | |
| 1% solution in PBS for assay buffer | |
| MSD Read Buffer T (4X) with Surfactant | 1 L or 250 mL |
| MSD 384 high bind plates | |
| Polypropylene for dilution 384 well plate | |
| Tissue culture treated black well 384 well plate | |
| INSTRUMENT(S): | |
| Meso Sector S600 | |
| Bravo | |
| Washer dispenser | |
| Multidrop Combi | |

Assay Protocol

Day 1 Cell Culture
1. Harvest NL20 human bronchial epithelial cells expressing human Z-AAT in OptiMEM™ containing Pen/Strep (P/S).
2. Seed at 16,000 cells/well in 30 μL (384 well plate).
3. Centrifuge plates briefly up to speed (1200 rpm) and place into 37° C. incubator overnight.

Day 2: Compound Addition and Coating Plates with Capture Antibody Compound Addition:
1. Dispense 40 μL of OptiMEM™ (P/S) with doxycycline (1:1000 stock=0.1 μM final) to each well of the compound plate using a multidrop Combi in hood.
2. Remove cell plate from incubator, flip/blot and take immediately to Bravo to transfer compounds.
3. Return plates to incubator overnight.

Coat MSD Plates
1. Dilute capture antibody (Polyclonal Goat anti-AAT) to 5 μg/mL (1:200) in PBS (no BSA).
2. Dispense 25 μL of diluted capture antibody into all wells of MSD 384-well High Bind plate using the Multidrop equipped with a standard cassette.
3. Incubate overnight at 4° C.

Prepare Blocker A (BSA) Solutions
1. Prepare solution of 5% MSD Blocker A (BSA) following the manufacturer's instructions.
2. Further dilute the 5% MSD Blocker A in PBS to 1% (Blocker A) as needed.

Day 3: Run MSD Assay

Block Plates
1. Wash plate 1× with 50 μL Wash buffer (PBS+0.5% Tween 20), and add 35 μL 5% Block A buffer to block non-specific binding on washer dispenser.
2. Rotate plates on shaker for 1 hour at 600 rpm.

Prepare M-AAT Standards
1. Dilute M-AAT stock to 1.6 μg/mL in 1% BSA Blocker A (Stock in −70° C.); then prepare 12×1:2 serial dilutions in 1% Blocker A.
2. The top starting final concentration on MSD plate is 320 ng/mL. These dilutions correspond to a final concentration of 320, 160, 80, 40, 20, 10, 5, 2.5, 1.25, 0.625, 0.312, 0.156 ng/mL.

Dilution Plate
1. Add 80 μL of 1% Assay buffer to all wells except columns 1/24 (standards) with Multidrop Combi.
2. Add diluted standards to columns 1 and 24.
3. Centrifuge dilution plates 1200 rpm briefly.

Cell Plate
1. Aspirate columns which will have the standards from the cell plates in the hood using 16-pin aspirator.

Prepare Human Neutrophil Elastase (hNE)
1. Prepare 1 μg/mL Human Neutrophil Elastase by diluting in 1% Blocker A.
   a. Small 100 μg vial—add 1 mL PBS (100 μg/mL).
      i. This can then be diluted 1:100 in 1% Assay Buffer for a final 1 μg/mL concentration.

MSD—add hNE (20 μL/well)
1. After the MSD plate has blocked for at least 1 hour, wash plate 1× with 50 μL. Wash buffer (PBS+0.5% Tween 20) and then add 20 μL hNE to each well.

Bravo-Cell Plate-Dilution Plate-MSD Plate

Using the Bravo aspirate 10 μL from the cell plate, transfer to the dilution plate (9-fold dilution)
1. Mix 25 μL 3×, then aspirate 5 μL, transfer to MSD plate (5-fold dilution).
2. Mix 10 μL 3×. Total dilution is 45 fold.
3. Shake plates at 600 rpm for 1.5 hours

Add Functional Detection hNE Antibody
1. Wash plate 1× with wash buffer.
2. Add 25 μL Sulfo-tagged anti-Elastase Monoclonal Mouse anti-Elastase) diluted to 0.45 μg/mL (1:2000) in 1% Blocker A into all wells of the functional activity MSD plates using the washer/dispenser.
   Note: The dilution required for sufficient signal must be determined for each new lot of labeled antibody.
3. Incubate at room temperature shaking at 600 rpm for 1 hour.

Final Wash and MSD Imager Read
1. Wash the plate 1×, and add 25 μL of Wash Buffer to the plate.
2. Make 2× Read buffer.
3. Remove wash buffer from MSD plate.
4. Transfer 35 μL 2× Read Buffer to MSD plate using Bravo and take to MSD to read immediately.
Data analysis in MSD Discovery Workbench 4.0 software and EC₅₀ values were determined using Genedata.

### B. Human Hepatocyte Clearance Assay

### 1. Determination of Human Hepatocyte CLint

Test compound was prepared to 10 mM in 100% DMSO and further diluted to 100 M in 50% acetonitrile:50% water (v/v). The cryopreserved human hepatocytes were prepared in CHRM (cryopreserved hepatocyte recovery medium). After cell viability was determined using a Nexcelom cell counter, hepatocytes were suspended and incubated in Williams' E media (pH 7.4) containing 0.5 million hepatocytes/mL and a final compound concentration of 1 μM. The compound/cell suspension (500 μl) was incubated for 2 hours at 37° C. in a humidified incubator with 5% CO₂ and 85% humidity and shaken at 900 rpm on an Eppendorf Thermomixer Comfort plate shaker. Samples (50 μl) were taken at 5, 30, 60, and 120 minutes and quenched with 100 μl of 100% acetonitrile with internal standard. Samples were vortexed for 5 minutes and centrifuged at 3700 rpm for 20 minutes to pellet precipitated protein. The supernatant fraction was diluted 1:1 with deionized water before LCMS analysis.

### 2. Determination of Protein Binding to Hepatocytes (Fu Hep)

Rat hepatocyte binding assay was completed using a 96-well rapid equilibrium dialysis (RED) plate. Test compound was diluted to 100 μM in 48% acetonitrile:48% water:4% DMSO (v/v/v) from 2.5 mM working stock solution in 100% DMSO, and further diluted in 300 μL of 0.6λ106 heat inactivated cell suspension in 150 mM phosphate buffer pH 7.4 to achieve a final compound concentration of 1 μM in the incubation. Immediately, 50 μl of the spiked cell suspension was aliquoted as a control T=0 sample. The T=0 sample was matrix matched with 50 μl of blank phosphate buffered solution (pH 7.4) and quenched with 300 μl of 100% acetonitrile with internal standard. Phosphate buffered solution (pH 7.4; 500 l) was added to the receiver chamber of the dialysis block and spiked rat hepatocyte suspension (300 l) was added to the donor chamber. The plate was covered with a gas-permeable lid and incubated for 18 hours at 37° C. in a humidified incubator (no CO2) on an Ohaus shaker at 300 rpm. At the end of incubation, 50 μl of post-dialysis sample from the donor and receiver wells were matrix-matched with 50 μl of phosphate buffered solution (pH 7.4) or blank rat hepatocyte suspension, respectively. The samples were subsequently quenched separately in 300 μl of 100% acetonitrile with internal standard. Quenched samples were shaken using a plate mixer for 10 minutes and centrifuged for 10 minutes at 4000 rpm to pellet precipitated protein. The supernatant fraction was further diluted 1:1 with deionized water for analysis by LCMS. Compound recovery and stability in the matrix were determined using the T=0 and T=18 samples.

### 3. LC-MS/MS Analysis

Compounds were analyzed by LC/hybrid Quadrupole-Orbitrap™ mass spectrometry using electrospray ionization on a QExactive (ThermoFisher Scientific, Waltham, MA). Aliquots of extracts (5 mL) were injected on an Acquity UPLC BEH C18 1.7 μm column, (2.1×50 mm) (Waters Corporation, Milford, MA) using a Vanquish auto-sampler and Vanquish binary pumps (ThermoFisher Scientific, Waltham, MA). A gradient of 15% to 99% B over 0.85 minutes was used with a flow rate of 0.8 mL/min. Mobile phases are A: 0.1% formic acid in water and B: 0.1% formic acid in acetonitrile. Compounds was monitored using their exact masses. The peak area ratio in each sample was determined by comparison of its peak area to the peak area of internal standard in the sample.

### 4. Data Analysis

The t_{1/2} and, subsequently, the CLint of the compounds incubated in human hepatocytes were calculated according to equations 1 and 2:

$$t 1 / 2 = \frac{Ln ( 2 )}{- slope} \qquad ( 1 )$$

$$CLint ( mL / min / million cells ) = \frac{Ln ( 2 ) \times V}{t 1 / 2} \qquad ( 2 )$$

In which V (μl/×10⁶ cells) is the incubation volume (μl) divided by the number of cells (×10⁶) in the incubation.

The unbound fraction (fu) of the compounds in rat hepatocyte was calculated according to equation 3:

$$fu , hep = \frac{peak area in buffer chamber}{peak area in cell suspenstion chamber} \qquad ( 3 )$$

Compound recovery in the relevant matrix were determined according to equation 4:

$$\% recovery = \frac{( average area buffer chamber * ( \frac{500}{300} ) + average area cell chamber ) \times 100}{standard area at T 0} \qquad ( 4 )$$

### C. Dose Projection

The potency measure in NL20 cells (EC₅₀) is used to derive target efficacious exposure for AAT compounds. The target plasma efficacious exposure at steady state in human for each compound is set to target free average concentration (C_{avg}) equivalent to the AAT NL20 EC₅₀. The daily target free efficacious exposure (AUC₀₋₂₄ₕ) is estimated as target C_{avg}*24 and the target total AUC₀₋₂₄ₕ is estimated as target free AUC_{0-24h,ss}/fup (fraction unbound in human plasma). The anticipated therapeutic dose in human (Dose) is estimated based on Equation 1:

Dose=CL*AUC_{0-24h,ss}/F Equation 1

and that,

AUC_{0-24h,ss}=Cavg*24/fup=EC₅₀*24/fup Equation 2
where CL is total clearance, AUC_{0-24h,ss} is daily exposure at steady state, and F is bioavailability.

Thus, to calculate the dose required to achieve daily Cavg,

Dose=CL*EC₅₀*24/fup/F Equation 3

Adam J. Lucas, Joanne L. Sproston, Patrick Barton & Robert J. Riley (2019):Estimating human ADME properties, pharmacokinetic parameters and likely clinical dose in drug discovery, Expert Opinion on Drug Discovery, DOI: 10.1080/17460441.2019.1660642.

A key consideration for the selection of compounds suitable for clinical development is the projection of human dose as described in Equations 1-3. Two key parameters that comprise the dose equation are the unbound clearance of a compound and the plasma efficacious exposure (also referred to herein as compound potency). Together these parameters are known to one skilled in the art as a measurement of Compound Quality and suitability for advancement into clinical development. Thus, when evaluating AAT modulator compounds, one needs to consider both the potency and unbound clearance parameters (i.e., Compound Quality). The relationship between these two parameters can be challenging to predict.

The compounds disclosed within exhibit an unanticipated improvement in the compound potency and unbound clearance relative to compounds previously disclosed which resulted in lower human projected doses. For example, comparing compound 33 from WO 2020/247160 (which has a hydrogen substituent at C8) with compound 45 from WO 2020/247160 (identical to compound 33 except that it has a fluorine substituent at C8) as shown in the table in Example 6 below, established that the H to F substitution at C8 was not anticipated to improve subsequent molecules from the series with respect to projected human dose.

Evaluating Compound Quality, this disclosure identifies select compounds that exhibit superiority over compounds disclosed in WO 2020/247160. For example, the compounds claimed herein have a significantly superior Compound Quality (i.e., lower Compound Quality score) relative to compounds of the prior art.

### Example 6

### A. Identification of Compounds with Superior Compound Quality Score

The closest prior art compounds can be found in WO 2020/247160, which discloses compounds of the general formula:

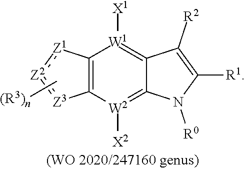

As noted above, WO 2020/247160, compound 33:

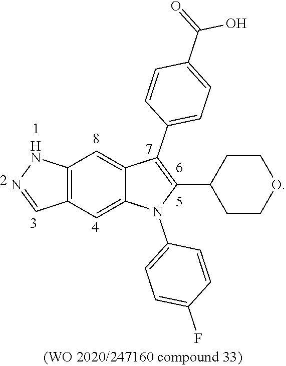 [SMILES: O=C(O)c1ccc(-c2c(C3CCOCC3)n(-c3ccc(F)cc3)c3cc4cn[nH]c4cc23)cc1]

has a Compound Quality score of 1.73. After synthesis of many compounds structurally similar to WO 2020/247160 compound 33, it became clear that the Compound Quality scores of compounds having the same structural core as the compounds of WO 2020/247160, is not predictable.

For example, preparation of many structurally similar compounds and analogs of WO 2020/247160 compound 33 (some of which were specifically exemplified in WO 2020/247160), demonstrated the tremendous variability in Compound Quality score as shown in the table below:

| WO | | | | | | |
|---|---|---|---|---|---|---|
| 2020/247160 | Pyrazole | Substituent | Substituent | Substituent | Substituent | Compound |
| Cmpd No.* | Ring | at N5 | at C6 | at C7 | at C8 | Quality |
| 33 | 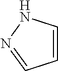 | 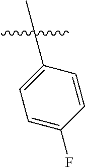 [SMILES: *c1ccc(F)cc1] | 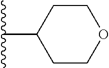 [SMILES: *C1CCOCC1] | 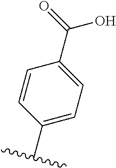 [SMILES: *c1ccc(C(=O)O)cc1] | H | 1.73 |
| 45 | 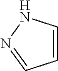 | 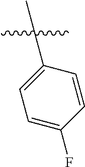 [SMILES: *c1ccc(F)cc1] | 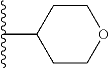 [SMILES: *C1CCOCC1] | 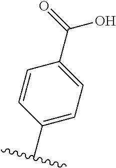 [SMILES: *c1ccc(C(=O)O)cc1] | F | 2.85 |
| | 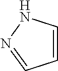 | 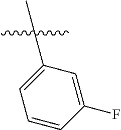 [SMILES: *c1cccc(F)c1] | 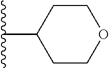 [SMILES: *C1CCOCC1] | 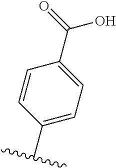 [SMILES: *c1ccc(C(=O)O)cc1] | H | 6.18 |
| 1 | 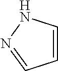 | 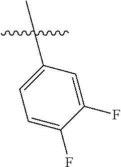 [SMILES: *c1ccc(F)c(F)c1] | 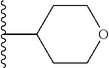 [SMILES: *C1CCOCC1] | 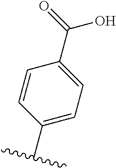 [SMILES: *c1ccc(C(=O)O)cc1] | H | 2.32 |
| 185 | 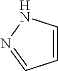 | 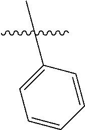 [SMILES: *c1ccccc1] | 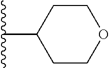 [SMILES: *C1CCOCC1] | 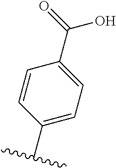 [SMILES: *c1ccc(C(=O)O)cc1] | H | 3.15 |
| 35 | 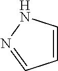 | 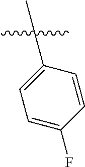 [SMILES: *c1ccc(F)cc1] | 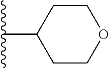 [SMILES: *C1CCOCC1] | 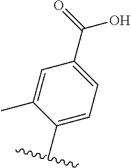 [SMILES: *c1ccc(C(=O)O)cc1C] | H | 2.68 |
| 37 | 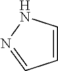 | 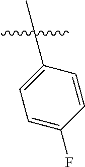 [SMILES: *c1ccc(F)cc1] | 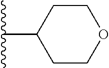 [SMILES: *C1CCOCC1] | 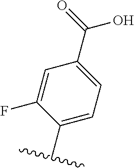 [SMILES: *c1ccc(C(=O)O)cc1F] | H | 2.91 |
| 284 | 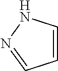 | 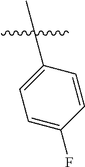 [SMILES: *c1ccc(F)cc1] | 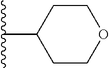 [SMILES: *C1CCOCC1] | 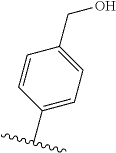 [SMILES: *c1ccc(CO)cc1] | H | 18.98 |
| 150 | 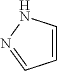 | 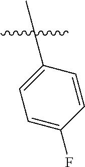 [SMILES: *c1ccc(F)cc1] | 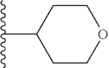 [SMILES: *C1CCOCC1] | 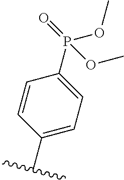 [SMILES: *c1ccc(P(=O)(OC)OC)cc1] | H | 7.78 |
| 108 | 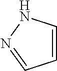 | 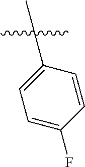 [SMILES: *c1ccc(F)cc1] | 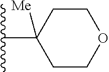 [SMILES: *C1(C)CCOCC1] | 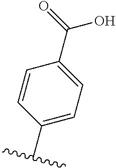 [SMILES: *c1ccc(C(=O)O)cc1] | H | 3.34 |
| 109 | 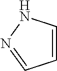 | 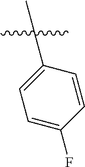 [SMILES: *c1ccc(F)cc1] | 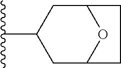 [SMILES: *C1CC2CCC(C1)O2] | 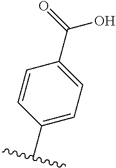 [SMILES: *c1ccc(C(=O)O)cc1] | H | 8.67 |
| 46 | 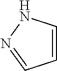 | 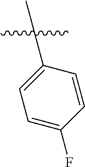 [SMILES: *c1ccc(F)cc1] | 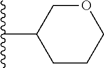 [SMILES: *C1CCCOC1] | 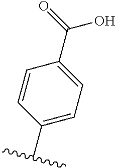 [SMILES: *c1ccc(C(=O)O)cc1] | H | 3.88 |
| 47 | 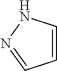 | 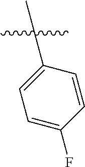 [SMILES: *c1ccc(F)cc1] | 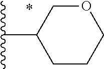 [SMILES: *C1CCCOC1] | 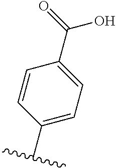 [SMILES: *c1ccc(C(=O)O)cc1] | H | 32.60 |
| 112 | 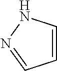 | 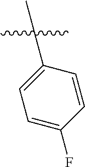 [SMILES: *c1ccc(F)cc1] | 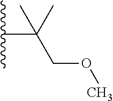 [SMILES: *C(C)(C)COC] | 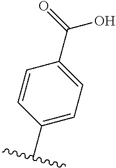 [SMILES: *c1ccc(C(=O)O)cc1] | H | 0.67 |
| 124 | 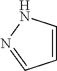 | 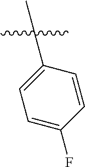 [SMILES: *c1ccc(F)cc1] | 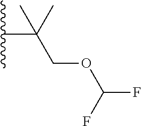 [SMILES: *C(C)(C)COC(F)F] | 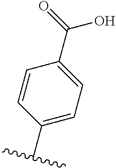 [SMILES: *c1ccc(C(=O)O)cc1] | H | 3.18 |
| 125 | 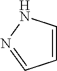 | 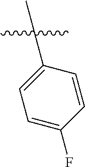 [SMILES: *c1ccc(F)cc1] | 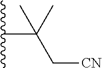 [SMILES: *C(C)(C)CC#N] | 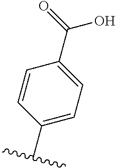 [SMILES: *c1ccc(C(=O)O)cc1] | H | 0.76 |
| | 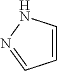 | 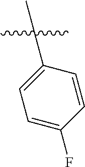 [SMILES: *c1ccc(F)cc1] | 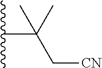 [SMILES: *C(C)(C)CC#N] | 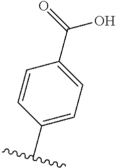 [SMILES: *c1ccc(C(=O)O)cc1] | F | 3.23 |
| | 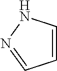 | 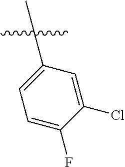 [SMILES: *c1ccc(F)c(Cl)c1] | 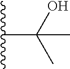 [SMILES: *C(C)(C)O] | 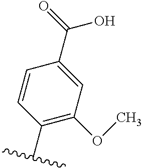 [SMILES: *c1ccc(C(=O)O)cc1OC] | F | 11.55 |
| | 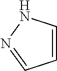 | 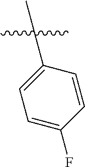 [SMILES: *c1ccc(F)cc1] | 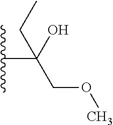 [SMILES: *C(O)(CC)COC] | 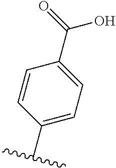 [SMILES: *c1ccc(C(=O)O)cc1] | F | 1.63 |
| | 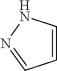 | 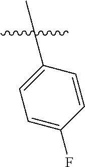 [SMILES: *c1ccc(F)cc1] | 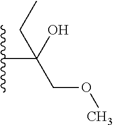 [SMILES: *C(O)(CC)COC] | 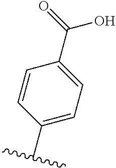 [SMILES: *c1ccc(C(=O)O)cc1] | F | 1.10 |
| | 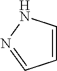 | 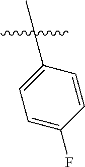 [SMILES: *c1ccc(F)cc1] | 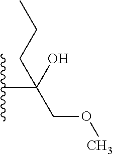 [SMILES: *C(O)(CCC)COC] | 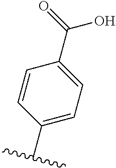 [SMILES: *c1ccc(C(=O)O)cc1] | F | 2.70 |
| | 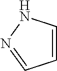 | 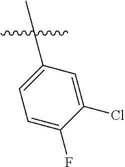 [SMILES: *c1ccc(F)c(Cl)c1] | 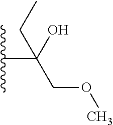 [SMILES: *C(O)(CC)COC] | 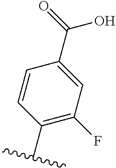 [SMILES: *c1ccc(C(=O)O)cc1F] | F | 8.43 |
| | 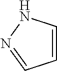 | 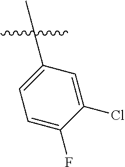 [SMILES: *c1ccc(F)c(Cl)c1] | 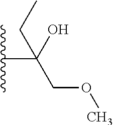 [SMILES: *C(O)(CC)COC] | 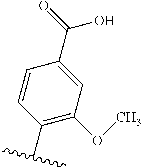 [SMILES: *c1ccc(C(=O)O)cc1OC] | F | 3.78 |
| | 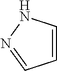 | 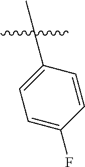 [SMILES: *c1ccc(F)cc1] | 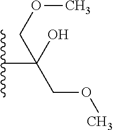 [SMILES: *C(O)(COC)COC] | 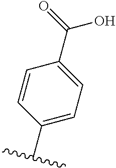 [SMILES: *c1ccc(C(=O)O)cc1] | F | 2.97 |
| 117 | 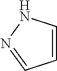 | 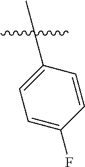 [SMILES: *c1ccc(F)cc1] | 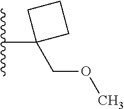 [SMILES: *C1(COC)CCC1] | 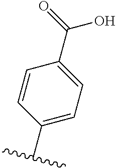 [SMILES: *c1ccc(C(=O)O)cc1] | H | 6.90 |
| 124 | 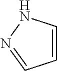 | 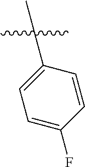 [SMILES: *c1ccc(F)cc1] | 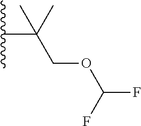 [SMILES: *C(C)(C)COC(F)F] | 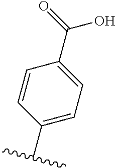 [SMILES: *c1ccc(C(=O)O)cc1] | H | 3.18 |
| | 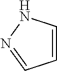 | 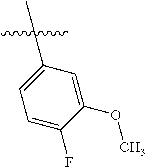 [SMILES: *c1ccc(F)c(OC)c1] | 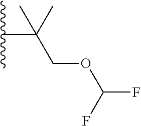 [SMILES: *C(C)(C)COC(F)F] | 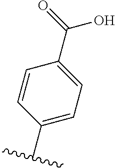 [SMILES: *c1ccc(C(=O)O)cc1] | F | 15.93 |
| 54 | 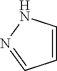 | 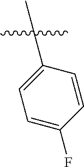 [SMILES: *c1ccc(F)cc1] | 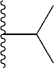 [SMILES: *C(C)C] | 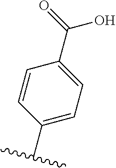 [SMILES: *c1ccc(C(=O)O)cc1] | H | 10.36 |
| 70 | 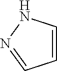 | 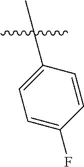 [SMILES: *c1ccc(F)cc1] | 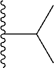 [SMILES: *C(C)C] | 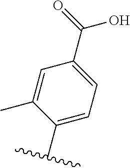 [SMILES: *c1ccc(C(=O)O)cc1C] | H | 10.54 |
| 49 | 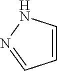 | 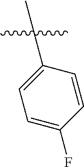 [SMILES: *c1ccc(F)cc1] | 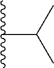 [SMILES: *C(C)C] | 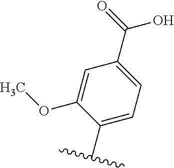 [SMILES: *c1ccc(C(=O)O)cc1OC] | H | 4.82 |
| 67 | 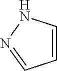 | 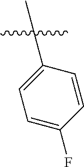 [SMILES: *c1ccc(F)cc1] | 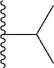 [SMILES: *C(C)C] | 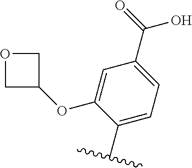 [SMILES: *c1ccc(C(=O)O)cc1OC1COC1] | H | 5.92 |
| 97 | 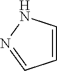 | 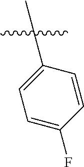 [SMILES: *c1ccc(F)cc1] | 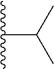 [SMILES: *C(C)C] | 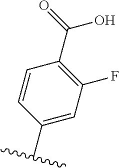 [SMILES: *c1ccc(C(=O)O)c(F)c1] | H | 23.55 |
| 53 | 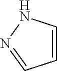 | 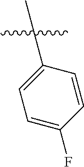 [SMILES: *c1ccc(F)cc1] | 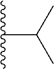 [SMILES: *C(C)C] | 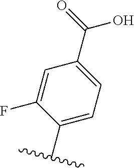 [SMILES: *c1ccc(C(=O)O)cc1F] | H | 33.53 |
| 51 | 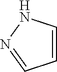 | 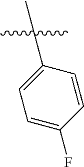 [SMILES: *c1ccc(F)cc1] | 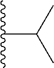 [SMILES: *C(C)C] | 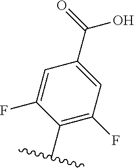 [SMILES: *c1c(F)cc(C(=O)O)cc1F] | H | 71.48 |
| 55 | 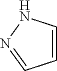 | 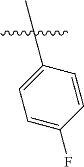 [SMILES: *c1ccc(F)cc1] | 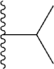 [SMILES: *C(C)C] | 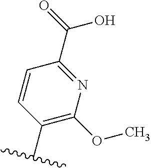 [SMILES: *c1ccc(C(=O)O)nc1OC] | H | 4.07 |
| 102 | 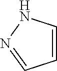 | 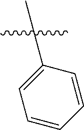 [SMILES: *c1ccccc1] | 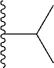 [SMILES: *C(C)C] | 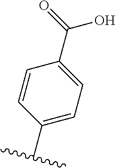 [SMILES: *c1ccc(C(=O)O)cc1] | H | 14.46 |
| 113 | 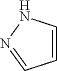 | 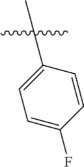 [SMILES: *c1ccc(F)cc1] | 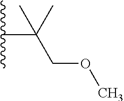 [SMILES: *C(C)(C)COC] | 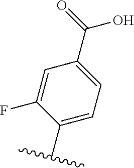 [SMILES: *c1ccc(C(=O)O)cc1F] | H | 2.32 |
| 114 | 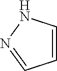 | 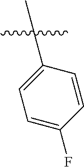 [SMILES: *c1ccc(F)cc1] | 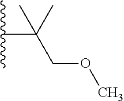 [SMILES: *C(C)(C)COC] | 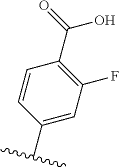 [SMILES: *c1ccc(C(=O)O)c(F)c1] | H | 5.57 |
| | 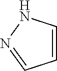 | 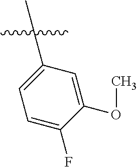 [SMILES: *c1ccc(F)c(OC)c1] | 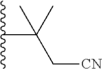 [SMILES: *C(C)(C)CC#N] | 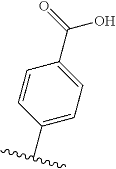 [SMILES: *c1ccc(C(=O)O)cc1] | F | 3.96 |
| | 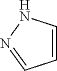 | 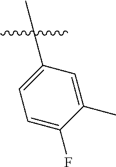 [SMILES: *c1ccc(F)c(C)c1] | 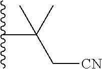 [SMILES: *C(C)(C)CC#N] | 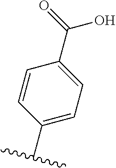 [SMILES: *c1ccc(C(=O)O)cc1] | F | 5.76 |
| 182 | 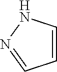 | 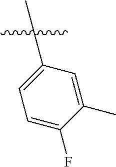 [SMILES: *c1ccc(F)c(C)c1] | 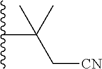 [SMILES: *C(C)(C)CC#N] | 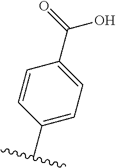 [SMILES: *c1ccc(C(=O)O)cc1] | H | 15.95 |
| 183 | 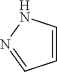 | 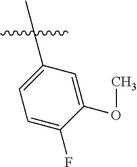 [SMILES: *c1ccc(F)c(OC)c1] | 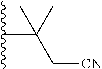 [SMILES: *C(C)(C)CC#N] | 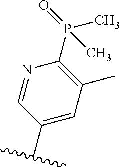 [SMILES: *c1cnc(P(C)(C)=O)c(C)c1] | H | 0.64 |
| 184 | 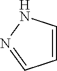 | 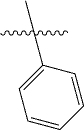 [SMILES: *c1ccccc1] | 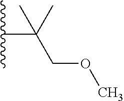 [SMILES: *C(C)(C)COC] | 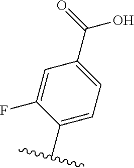 [SMILES: *c1ccc(C(=O)O)cc1F] | H | 1.26 |
| 189 | 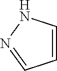 | 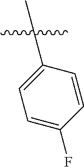 [SMILES: *c1ccc(F)cc1] | 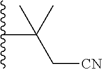 [SMILES: *C(C)(C)CC#N] | 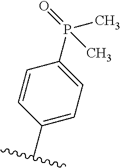 [SMILES: *c1ccc(P(C)(C)=O)cc1] | H | 1.24 |
| 197 | 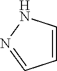 | 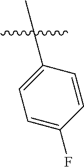 [SMILES: *c1ccc(F)cc1] | 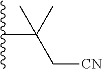 [SMILES: *C(C)(C)CC#N] | 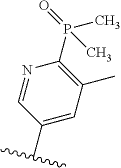 [SMILES: *c1cnc(P(C)(C)=O)c(C)c1] | H | 0.82 |
| 253 | 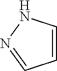 | 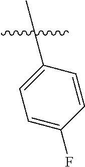 [SMILES: *c1ccc(F)cc1] | 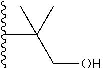 [SMILES: *C(C)(C)CO] | 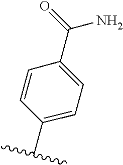 [SMILES: *c1ccc(C(N)=O)cc1] | H | 17.55 |
| 270 | 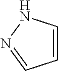 | 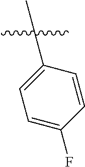 [SMILES: *c1ccc(F)cc1] | 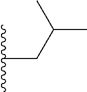 [SMILES: *CC(C)C] | 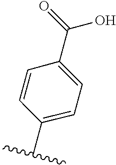 [SMILES: *c1ccc(C(=O)O)cc1] | H | 131.55 |
| 271 | 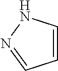 | 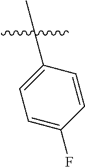 [SMILES: *c1ccc(F)cc1] | 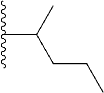 [SMILES: *C(C)CCC] | 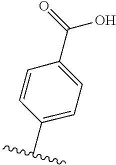 [SMILES: *c1ccc(C(=O)O)cc1] | H | 1125.88 |
| 320 | 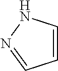 | 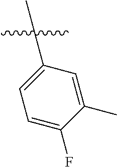 [SMILES: *c1ccc(F)c(C)c1] | 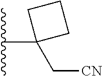 [SMILES: *C1(CC#N)CCC1] | 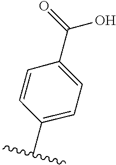 [SMILES: *c1ccc(C(=O)O)cc1] | H | 7.30 |
| 341 | 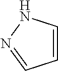 | 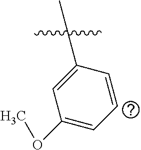 [SMILES: *c1cccc(OC)c1] | 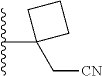 [SMILES: *C1(CC#N)CCC1] | 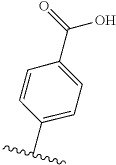 [SMILES: *c1ccc(C(=O)O)cc1] | F | 8.09 |
| | 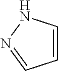 | 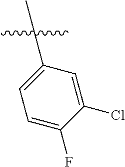 [SMILES: *c1ccc(F)c(Cl)c1] | 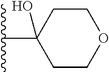 [SMILES: *C1(O)CCOCC1] | 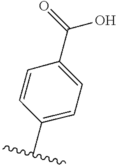 [SMILES: *c1ccc(C(=O)O)cc1] | F | 32.65 |
| | 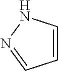 | 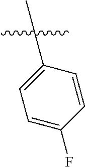 [SMILES: *c1ccc(F)cc1] | 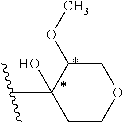 [SMILES: *C1(O)CCOCC1OC] | 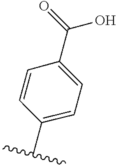 [SMILES: *c1ccc(C(=O)O)cc1] | F | 4.34 |
| | 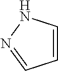 | 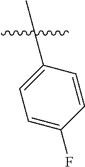 [SMILES: *c1ccc(F)cc1] | 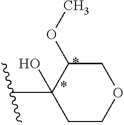 [SMILES: *C1(O)CCOCC1OC] | 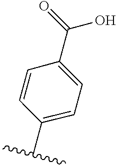 [SMILES: *c1ccc(C(=O)O)cc1] | F | 37.99 |
| | 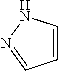 | 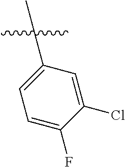 [SMILES: *c1ccc(F)c(Cl)c1] | 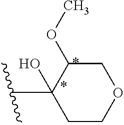 [SMILES: *C1(O)CCOCC1OC] | 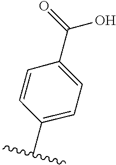 [SMILES: *c1ccc(C(=O)O)cc1] | F | 51.42 |
| | 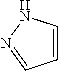 | 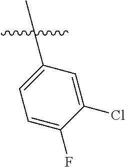 [SMILES: *c1ccc(F)c(Cl)c1] | 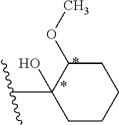 [SMILES: *C1(O)CCCCC1OC] | 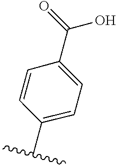 [SMILES: *c1ccc(C(=O)O)cc1] | F | 207.84 |
| | 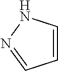 | 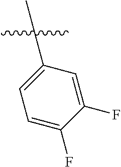 [SMILES: *c1ccc(F)c(F)c1] | 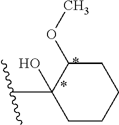 [SMILES: *C1(O)CCCCC1OC] | 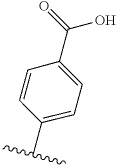 [SMILES: *c1ccc(C(=O)O)cc1] | F | 6.06 |
| | 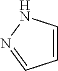 | 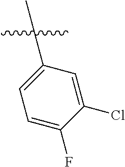 [SMILES: *c1ccc(F)c(Cl)c1] | 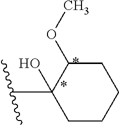 [SMILES: *C1(O)CCCCC1OC] | 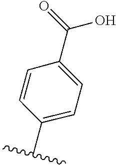 [SMILES: *c1ccc(C(=O)O)cc1] | F | 2.46 |
| | 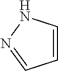 | 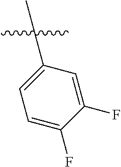 [SMILES: *c1ccc(F)c(F)c1] | 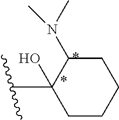 [SMILES: *C1(O)CCCCC1N(C)C] | 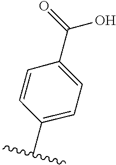 [SMILES: *c1ccc(C(=O)O)cc1] | F | 4.05 |
| | 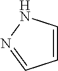 | 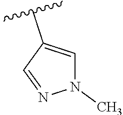 [SMILES: *c1cnn(C)c1] | 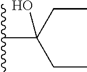 [SMILES: *C(O)(CC)CC] | 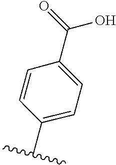 [SMILES: *c1ccc(C(=O)O)cc1] | F | 13.92 |
| 291 | 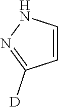 | 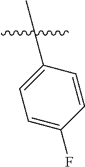 [SMILES: *c1ccc(F)cc1] | 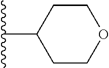 [SMILES: *C1CCOCC1] | 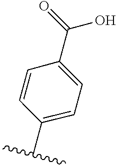 [SMILES: *c1ccc(C(=O)O)cc1] | H | 2.01 |
| 136 | 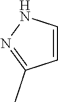 | 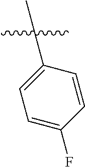 [SMILES: *c1ccc(F)cc1] | 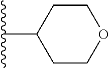 [SMILES: *C1CCOCC1] | 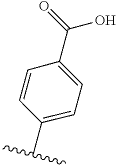 [SMILES: *c1ccc(C(=O)O)cc1] | H | 38.16 |
| 129 | 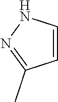 | 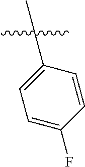 [SMILES: *c1ccc(F)cc1] | 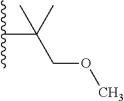 [SMILES: *C(C)(C)COC] | 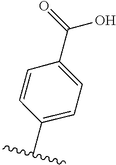 [SMILES: *c1ccc(C(=O)O)cc1] | H | 1.03 |
| 342 | 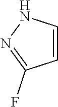 | 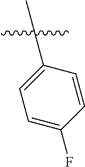 [SMILES: *c1ccc(F)cc1] | 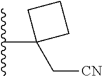 [SMILES: *C1(CC#N)CCC1] | 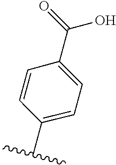 [SMILES: *c1ccc(C(=O)O)cc1] | H | 0.82 |
| | 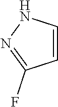 | 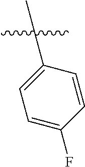 [SMILES: *c1ccc(F)cc1] | 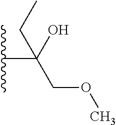 [SMILES: *C(O)(CC)COC] | 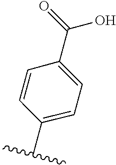 [SMILES: *c1ccc(C(=O)O)cc1] | H | 1.73 |

*Compounds without a number in first column were not specifically exemplified in WO 2020/247160

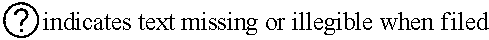

These results demonstrate the extreme unpredictability involved in identifying compounds with Compound Quality score below 0.40, rendering the search for compounds with low projected human dose very challenging.

### B. Compound Quality Data for Compounds of the Invention

The compounds of Formulae I and II are useful as modulators of AAT activity and are unexpectedly superior to prior art compounds. The values provided in the table below are calculated by multiplying the EC₅₀ of each of the compounds by the result from the clearance assay procedures described in Section C above and rounded to two significant figures. In the table below, the following meanings apply for Compound Quality (i.e., compound potency x unbound clearance) values: “++++” means ≤0.2; “+++” means >0.20 and ≤0.30; “++” means >0.30 and ≤0.40; “+” means >0.40 and ≤0.50.

| Compound No. | Compound Quality |
|---|---|
| 1 | ++++ |
| 2 | +++ |
| 3 | +++ |
| 4 | +++ |
| 5 | ++++ |
| 6 | ++ |
| 7 | +++ |
| 8 | + |
| 9 | +++ |
| 10 | ++ |
| 11 | +++ |
| 12 | +++ |
| 13 | +++ |
| 14 | ++ |
| 15 | ++ |
| 16 | ++ |
| 17 | +++ |
| 18 | +++ |
| 19 | +++ |
| 20 | ++ |
| 21 | ++ |
| 22 | ++++ |
| 23 | +++ |
| 24 | +++ |
| 25 | +++ |
| 26 | ++++ |
| 27 | ++ |
| 28 | +++ |
| 29 | ++++ |
| 30 | ++++ |
| 31 | ++ |
| 32 | +++ |
| 33 | +++ |
| 34 | ++++ |
| 35 | ++++ |
| 36 | +++ |
| 37 | +++ |
| 38 | +++ |
| 39 | +++ |
| 40 | +++ |
| 41 | + |
| 42 | ++ |
| 43 | + |
| 44 | + |
| 45 | +++ |
| 46 | +++ |
| 47 | +++ |
| 48 | + |
| 49 | ++ |
| 50 | ++ |
| 51 | ++ |
| 52 | ++ |
| 53 | ++ |
| 54 | ++++ |
| 55 | ++ |
| 56 | +++ |
| 57 | + |
| 58 | + |
| 59 | ++ |
| 60 | +++ |
| 61 | +++ |
| 62 | + |
| 63 | + |
| 64 | ++ |
| 65 | + |
| 66 | ++ |
| 67 | ++ |

### Example 7

Enhancement of Exposure Multiple

Irrespective of a superior Compound Quality score, it has been unexpectedly discovered that substitution of hydrogen with fluorine at C8 of the core rings of the disclosed Formulae results in enhancement of drug exposure multiple. The calculation of an exposure multiple is accomplished by comparing the plasma exposure (AUC) achieved in a toxicology species relative to the target efficacious exposure at steady state (AUCss). The larger this exposure multiple, the superior the molecule because it provides the potential opportunity to explore higher exposures in clinical development if a larger window is established in toxicology studies. In addition, this exposure multiple can allow for further exploration of the desired pharmacology due to the opportunity for a larger window established in toxicology studies. However, the exposure that results in a toxicological outcome in preclinical toxicology studies is unpredictable.

As previously noted, SAR based on compounds disclosed in WO 2020/247160 did not support exploration of 8F analogs. WO 2020/247160 suggested no superiority in 8F compounds over 8 H compounds, and in fact, disclosed only two such compounds. When further evaluated, the two 8F compounds disclosed in WO 2020/247160 provided the following data:

WO 2020/247160 compound 45
45

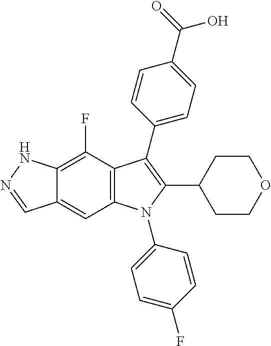 [SMILES: O=C(O)c1ccc(-c2c(C3CCOCC3)n(-c3ccc(F)cc3)c3cc4cn[nH]c4c(F)c23)cc1]

Compound Quality = 2.85
WO 2020/247160 compound 341
341

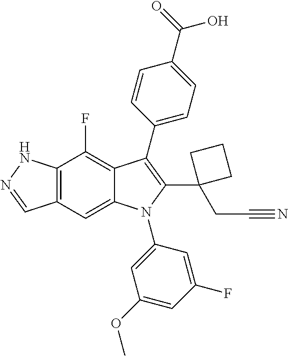 [SMILES: COc1cc(F)cc(-n2c(C3(CC#N)CCC3)c(-c3ccc(C(=O)O)cc3)c3c(F)c4[nH]ncc4cc32)c1]

Compound Quality = 8.09

Moreover, as previously noted, a side-by-side comparison of an 8F and an 8H compound from WO 2020/247160 also showed no reason to further explore 8F compounds:

WO 2020/247160 compound 45
45

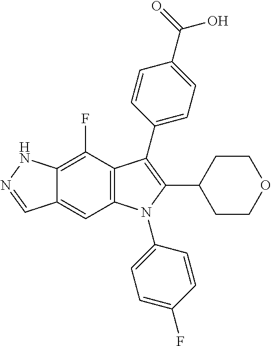 [SMILES: O=C(O)c1ccc(-c2c(C3CCOCC3)n(-c3ccc(F)cc3)c3cc4cn[nH]c4c(F)c23)cc1]

Compound Quality = 2.85
WO 2020/247160 compound 33
33

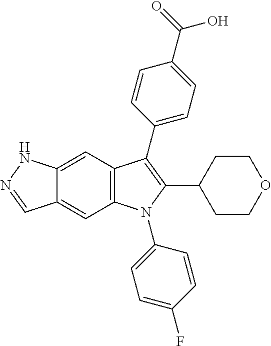 [SMILES: O=C(O)c1ccc(-c2c(C3CCOCC3)n(-c3ccc(F)cc3)c3cc4cn[nH]c4cc23)cc1]

Compound Quality = 1.73

The chart below shows the overall enhancement of in vivo exposure multiple resulting from the substitution of fluorine at the C8 position on the core ring structure. as compared to the same compound having a hydrogen at that position. Human exposure multiple (EM) values shown relate to the fold increase in exposure achieved in rat 5-day toxicology studies for a given dose relative to the predicted efficacious exposure anticipated in human (based on cellular NL20 EC₅₀ in vitro efficacy). The exposure multiple score is based on the maximum exposure multiple achieved in the given study. This allows relative comparison across compounds when also considering the administered dose. The 8F compounds surprisingly demonstrated enhanced exposure multiples when compared to their 811 counterparts. In the tables below, the following meanings apply for exposure multiple scores: “−” means <1; “+” means 1-10; “++” means 10-20; “+++” means 20-30; “++++” means 30-50; and “+++++” means >50.

| Cmpd | | Rat Bid (Daily) | Exposure | |
|---|---|---|---|---|
| No. | Structure | Doses | Multiple | EM Score |
| | 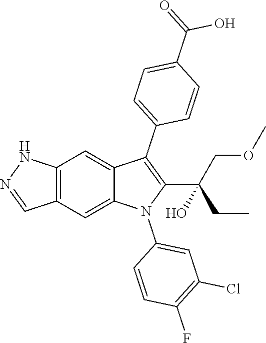 [SMILES: CC[C@](O)(COC)c1c(-c2ccc(C(=O)O)cc2)c2cc3[nH]ncc3cc2n1-c1ccc(F)c(Cl)c1] | 100, 250, 500 (200, 500, 1000) | 2, 1, 2 | + |
| 4 | 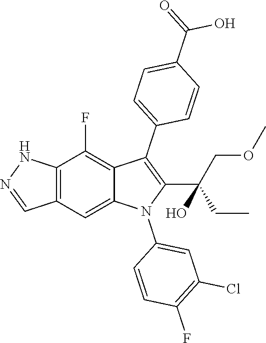 [SMILES: CC[C@@](O)(COC)c1c(-c2ccc(C(=O)O)cc2)c2c(F)c3[nH]ncc3cc2n1-c1ccc(F)c(Cl)c1] | 50, 200, 400 (100, 400, 800) | 8, 21, 32 | ++++ |
| | 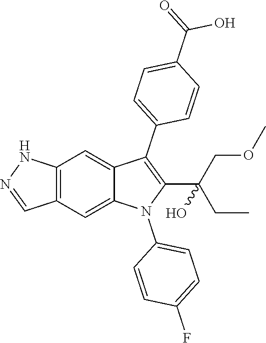 [SMILES: CCC(O)(COC)c1c(-c2ccc(C(=O)O)cc2)c2cc3[nH]ncc3cc2n1-c1ccc(F)cc1] | 250 (500) | 14 | ++ |
| 1 | 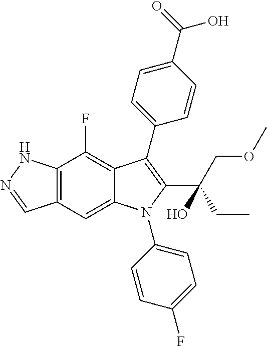 [SMILES: CC[C@@](O)(COC)c1c(-c2ccc(C(=O)O)cc2)c2c(F)c3[nH]ncc3cc2n1-c1ccc(F)cc1] | 50, 200, 500 (100, 400, 1000) | 10, 82, 126 | +++++ |

Once this side-by-side result was observed, the trend continued to show that the 8F compounds demonstrated enhanced exposure multiples compared to similar 811 compounds, and thus, superior profiles relative to the prior art.

| Cmpd | | Rat Bid (Daily) | Exposure | |
|---|---|---|---|---|
| No. | Structure | Doses | Multiple | EM Score |
| | 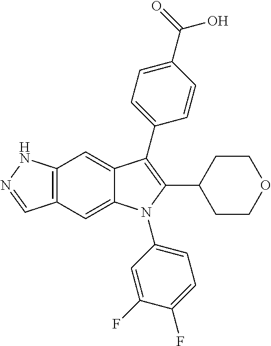 [SMILES: O=C(O)c1ccc(-c2c(C3CCOCC3)n(-c3ccc(F)c(F)c3)c3cc4cn[nH]c4cc23)cc1] | 300 (600) | 0.4 | − |
| | 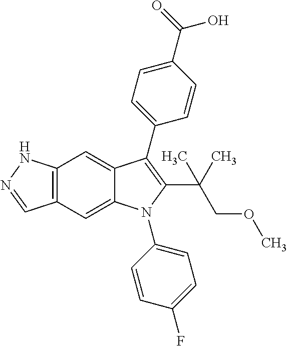 [SMILES: COCC(C)(C)c1c(-c2ccc(C(=O)O)cc2)c2cc3[nH]ncc3cc2n1-c1ccc(F)cc1] | 25, 50, 100 (50, 100, 200) | 5, 13, 18 | ++ |
| | 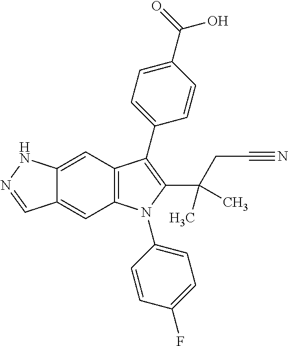 [SMILES: CC(C)(CC#N)c1c(-c2ccc(C(=O)O)cc2)c2cc3[nH]ncc3cc2n1-c1ccc(F)cc1] | 287.5 (575) | 1.8 | + |
| 2 | 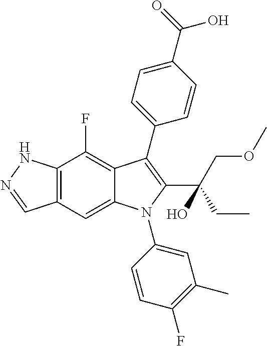 [SMILES: CC[C@@](O)(COC)c1c(-c2ccc(C(=O)O)cc2)c2c(F)c3[nH]ncc3cc2n1-c1ccc(F)c(C)c1] | 50, 200, 500 (100, 400, 1000) | 2, 10, 40 | ++++ |
| 3 | 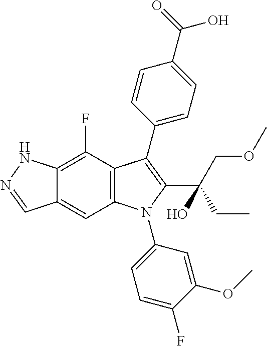 [SMILES: CC[C@@](O)(COC)c1c(-c2ccc(C(=O)O)cc2)c2c(F)c3[nH]ncc3cc2n1-c1ccc(F)c(OC)c1] | 50, 200, 500 | 2, 17, 60 | +++++ |
| 5 | 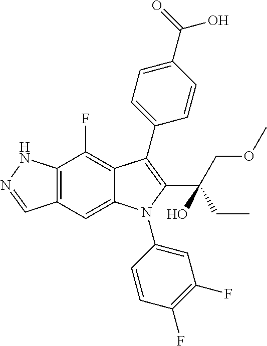 [SMILES: CC[C@@](O)(COC)c1c(-c2ccc(C(=O)O)cc2)c2c(F)c3[nH]ncc3cc2n1-c1ccc(F)c(F)c1] | 50, 150, 300 (100, 300, 600) | 39, 114, 173 | +++++ |

### Example 8—Procedures for Analysis of Solid Forms

Unless otherwise stated, the following procedures were employed for the analysis of all solid forms.

### X-Ray Powder Diffraction

X-ray powder diffraction (XRPD) spectra were recorded at room temperature (25±2° C.) in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 30 to about 40020 with a step size of 0.0131303° and 49 s per step.

### Solid State NMR

Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiments. The fluorine relaxation time was measured using ¹⁹F MAS T₁ saturation recovery relaxation experiment in order to set up proper recycle delay of the ¹⁹F MAS experiment. The CP contact time of carbon CPMAS experiments was set to 2 ms. A CP proton pulse with linear ramp (from 50% to 100%) was employed. The carbon Hartmann-Hahn match was optimized on external reference sample (glycine). All carbon and fluorine spectra were recorded with proton decoupling using TPPM15 or SPINAL 64 decoupling sequence with the field strength of approximately 100 kHz.

### Example 9—Solid Forms of Compound 5

### A. Compound 5 Monohydrate Form A

### 1. Synthetic Procedures

5 g of Compound 5 free form Form A and 25 mL of 1-propanol were added to a reactor. The slurry was stirred and heated to 50° C. to obtain a clear solution. To the clear solution, 25 mL of water was added over 2 hours and during this addition, a slurry was formed. The slurry was then cooled to 20° C. over 5 hours, agitated for another 8 hours, and filtered. Filtered solids were washed with a 15 ml mixture of 1:1 (v/v) 1-propanol:water and then dried at 50° C. in a vacuum oven with a nitrogen bleed. Final solids were confirmed to be Compound 5 free form Monohydrate Form A via XRPD and ssNMR.

Alternatively, 5 g of Compound 5 EtOAc-heptane solvate and 20 mL of 1-propanol were added to a reactor. The slurry was stirred and heated to 50° C. to obtain a clear solution. To the clear solution, 5 mL of water was added over 1 hour. The clear solution was then seeded with 1w % free form Monohydrate Form A seeds and agitated for 1 hour. To the slurry, 25 mL of water was added over 5 hours, then cooled to 20° C. over 5 hours, agitated for another 8 hours, and filtered. Filtered solids were washed with a 15 ml mixture of 2:3 (v/v) 1-propanol:water and then dried at 50° C. in a vacuum oven with a nitrogen bleed to provide Compound 5 free form Monohydrate Form A.

### 2. X-Ray Powder Diffraction

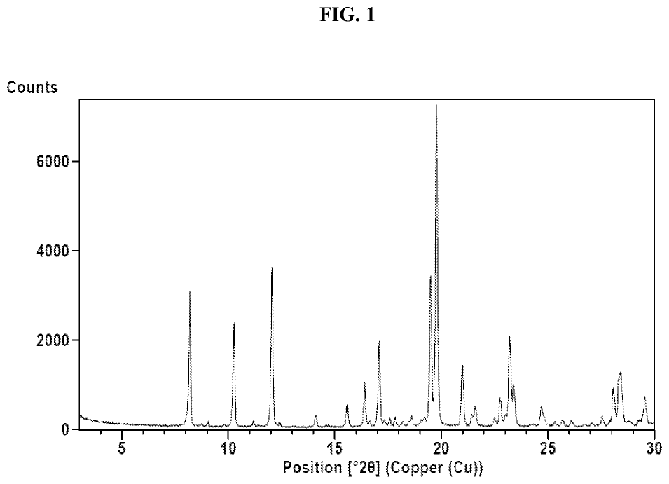

The XRPD results are shown in FIG. 1 and the table below:

XRPD Signals for Compound 5 free form Monohydrate Form A

| No. | Pos. [±0.2, °2θ] | Rel. Int. [%] |
|---|---|---|
| 1 | 19.8 | 100.0 |
| 2 | 12.1 | 49.4 |
| 3 | 19.5 | 46.4 |
| 4 | 8.2 | 41.6 |
| 5 | 10.3 | 32.0 |
| 6 | 23.2 | 27.7 |
| 7 | 17.1 | 27.1 |
| 8 | 21.0 | 19.0 |
| 9 | 28.4 | 17.0 |
| 10 | 16.4 | 13.9 |
| 11 | 28.3 | 13.9 |
| 12 | 23.4 | 13.2 |
| 13 | 28.1 | 11.8 |

### 3. Thermogravimetric Analysis

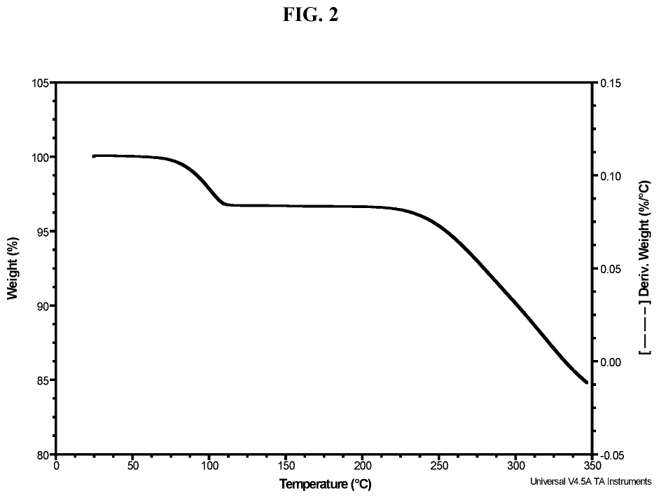

Thermogravimetric analysis of Compound 5 free form Monohydrate Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 2) showed 3.3% weight loss from ambient temperature up to 160° C.

### 4. Differential Scanning Calorimetry Analysis

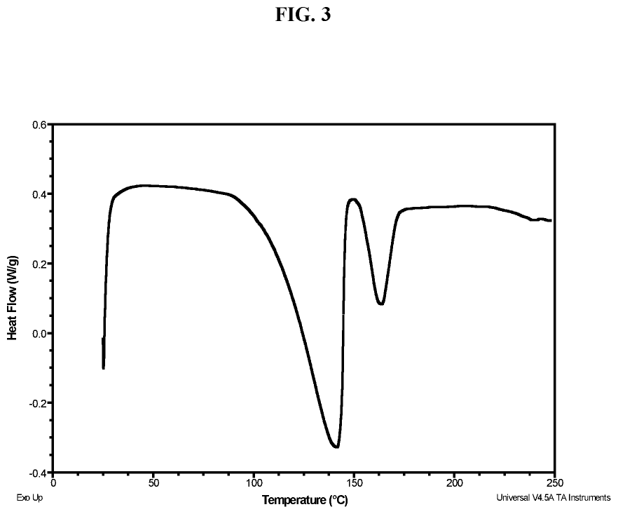

DSC of Compound 5 monohydrate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 250° C. When the run was completed, the data were analyzed by Trios and/or Universal Analysis software (TA Instruments, New Castle, DE). The thermogram (FIG. 3) showed endothermic peaks around 141° C. and 163° C.

### 5. Solid State NMR

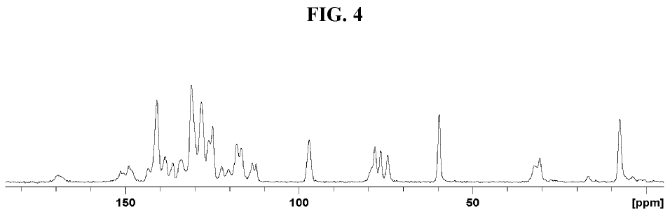

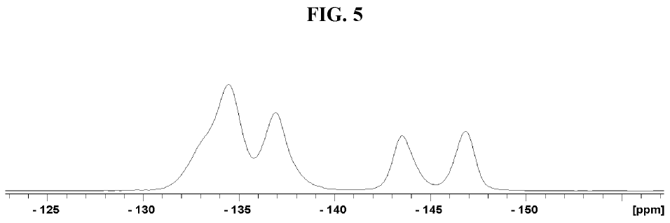

All carbon and fluorine spectra were recorded with proton decoupling using TPPM15 decoupling sequence. The ¹³C CPMAS and ¹⁹F MAS on Compound 5 free form Monohydrate Form A results are shown in FIG. 4 and FIG. 5, respectively and in the tables below:

¹³C CPMAS on Compound 5 free form Monohydrate Form A

| Peak # | Chem Shift [±0.2 ppm] | Intensity [rel] |
|---|---|---|
| 1 | 169.5 | 0.7 |
| 2 | 151.4 | 1.1 |
| 3 | 149.0 | 1.7 |
| 4 | 143.4 | 1.4 |
| 5 | 140.9 | 8.5 |
| 6 | 138.5 | 2.7 |
| 7 | 136.4 | 2.0 |
| 8 | 133.9 | 2.3 |
| 9 | 131.0 | 10.0 |
| 10 | 128.1 | 8.3 |
| 11 | 125.9 | 4.3 |
| 12 | 124.8 | 5.8 |
| 13 | 122.3 | 1.6 |
| 14 | 120.2 | 1.3 |
| 15 | 117.9 | 3.9 |
| 16 | 116.6 | 3.5 |
| 17 | 113.5 | 2.0 |
| 18 | 112.4 | 1.9 |
| 19 | 97.1 | 4.4 |
| 20 | 79.2 | 1.4 |
| 21 | 78.1 | 3.7 |
| 22 | 76.5 | 3.3 |
| 23 | 74.4 | 2.8 |
| 24 | 59.6 | 6.9 |
| 25 | 32.2 | 1.7 |
| 26 | 30.6 | 2.5 |
| 27 | 7.6 | 6.5 |

¹⁹F MAS on Compound 5 free form Monohydrate Form A

| Peak # | Chem Shift [±0.2 ppm] | Intensity [rel] |
|---|---|---|
| 1 | −134.4 | 10.0 |
| 2 | −136.9 | 7.4 |
| 3 | −143.5 | 5.2 |
| 4 | −146.8 | 5.6 |

### 6. Single Crystal Elucidation

Single crystals having the Compound 5 free form Monohydrate Form A structure were grown from a mixture of 1-propanol and water. X-ray diffraction data were acquired at 100K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122) and results are summarized in the table below.

Single Crystal Elucidation of Compound
5 free form Monohydrate Form A

| | |
|---|---|
| Crystal System | Monoclinic |
| Space Group | P2₁ |
| a (Å) | 9.6704(2) |
| b (Å) | 12.6954(3) |
| c (Å) | 20.0838(4) |
| α (°) | 90 |
| β (°) | 92.4079(12) |
| γ (°) | 90 |
| V (Å³) | 2463.50(9) |
| Z/Z′ | 2/2 |
| Temperature | 100 K |

Single crystals having the Compound 5 free form Monohydrate Form A structure were grown from a mixture of 1-propanol and water. X-ray diffraction data were acquired at 293K on a Bruker diffractometer equipped with Cu K, radiation (λ=1.54178 Å) and a CMOS detector. The structure was solved and refined using SHELX programs (Sheldrick, G. M., Acta Cryst., (2008) A64, 112-122) and results are summarized in the table below.

Single Crystal Elucidation of Compound
5 free form Monohydrate Form A

| | |
|---|---|
| Crystal System | Monoclinic |
| Space Group | P2₁ |
| a (Å) | 9.7812(3) |
| b (Å) | 12.7384(4) |
| c (Å) | 20.2509(6) |
| α (°) | 90 |
| β (°) | 93.011(2) |
| γ (°) | 90 |
| V (Å³) | 2519.71(13) |
| Z/Z′ | 2/2 |
| Temperature | 293 K |

### B. Compound 5 Free Form Form A

### 1. Synthetic Procedure

Compound 5 free form EtOH solvate Form A was dissolved in 4:1 DCM:MeOH (500 mL), evaporated to dryness. Residue was dissolved in 4:1 DCM:MeOH (500 mL), evaporated to dryness. Residue was dissolved in DCM (500 mL), evaporated to dryness. Residue was dissolved/suspended in DCM (250 mL), refluxed for 2 h to give a uniform suspension. Slowly allowed to cool to room temp, stood at room temp overnight. After 16 h, suspension was filtered, washing with DCM (100 mL). Collected solid was dried under suction for 1 h, then on rotovap (2 mbar), followed by vacuum oven dried at 75-90° C. for days until DCM level was below International Conference on Harmonization (ICH) limit.

### 2. X-Ray Powder Diffraction

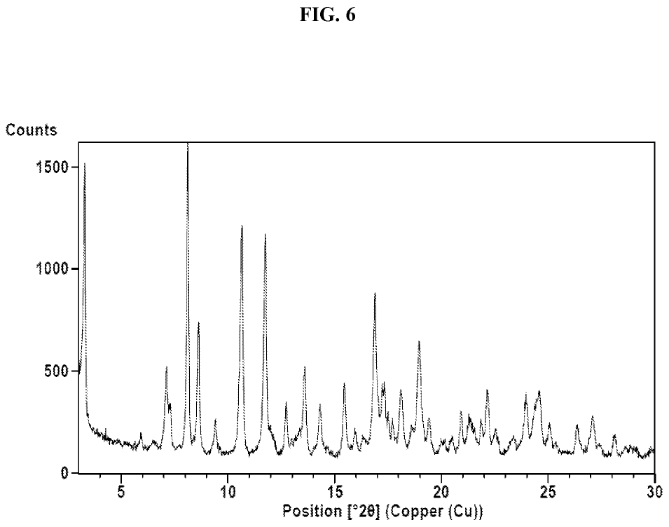

The XRPD results are shown in FIG. 6 and the table below.

XRPD Signals for Compound 5 free form Form A

| No. | Pos. [±0.2, °2θ] | Rel. Int. [%] |
|---|---|---|
| 1 | 8.1 | 100.0 |
| 2 | 3.3 | 99.0 |
| 3 | 10.7 | 74.1 |
| 4 | 11.8 | 70.4 |
| 5 | 16.9 | 50.5 |
| 6 | 8.6 | 43.5 |
| 7 | 19.0 | 35.7 |
| 8 | 3.0 | 34.1 |
| 9 | 7.1 | 26.5 |
| 10 | 13.6 | 26.4 |
| 11 | 15.5 | 22.7 |
| 12 | 22.1 | 20.2 |
| 13 | 17.3 | 19.8 |
| 14 | 24.6 | 18.7 |
| 15 | 18.1 | 18.2 |
| 16 | 24.0 | 16.3 |
| 17 | 12.7 | 15.8 |
| 18 | 7.3 | 14.3 |
| 19 | 14.3 | 13.9 |
| 20 | 20.9 | 13.2 |
| 21 | 27.1 | 12.4 |
| 22 | 9.4 | 11.2 |
| 23 | 19.4 | 10.9 |
| 24 | 21.8 | 10.1 |

### 3. Thermogravimetric Analysis

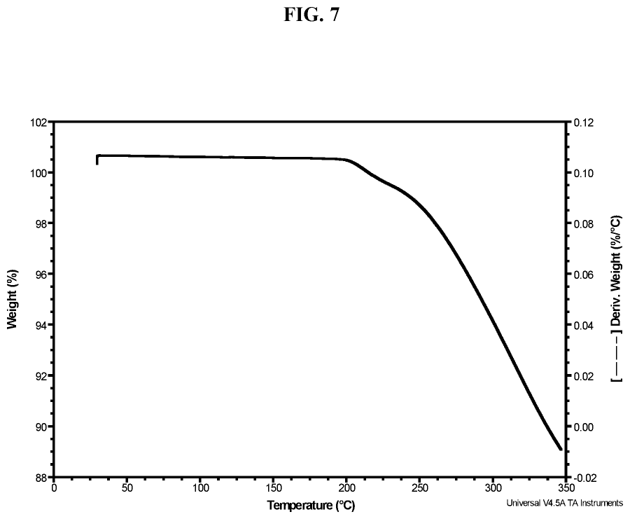

Thermogravimetric analysis of Compound 5 free form Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 7) showed 0.2% weight loss from ambient temperature up to 180° C.

### 4. Differential Scanning Calorimetry Analysis

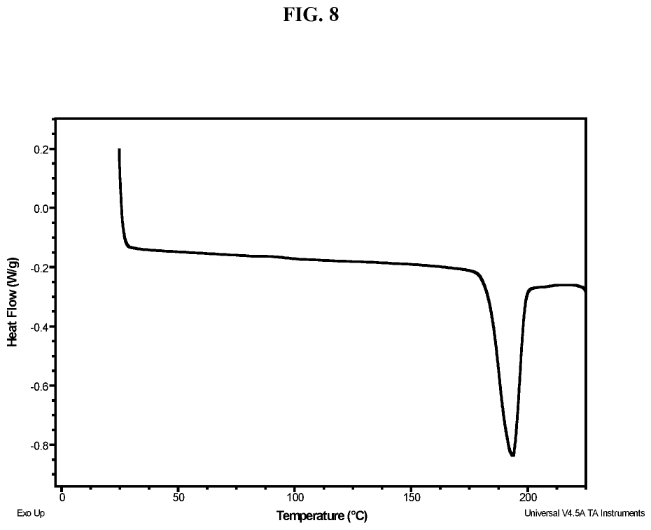

DSC of Compound 5 free form Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 220° C. When the run was completed, the data were analyzed by Trios and/or Universal Analysis software (TA Instruments, New Castle, DE). The thermogram (FIG. 8) showed endothermic peaks around 193° C.

### 5. Solid State NMR

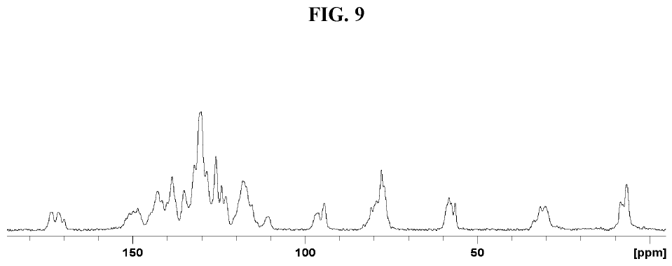

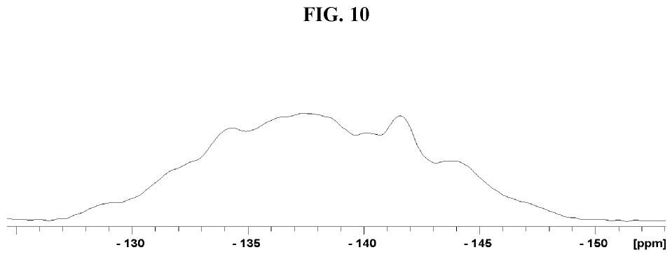

All carbon, phosphorus and fluorine spectra were recorded with proton decoupling using TPPM15 decoupling sequence. The ¹³C CPMAS and ¹⁹F MAS on Compound 5 free form Form A results are shown in FIG. 9 and FIG. 10, respectively and in the tables below:

¹³C CPMAS on Compound 5 free form Form A

| Peak # | Chem Shift [ppm] | Intensity [rel] |
|---|---|---|
| 1 | 173.6 | 1.6 |
| 2 | 171.5 | 1.6 |
| 3 | 169.8 | 1.0 |
| 4 | 150.8 | 1.5 |
| 5 | 149.7 | 1.7 |
| 6 | 148.4 | 1.9 |
| 7 | 142.7 | 3.3 |
| 8 | 141.4 | 2.6 |
| 9 | 139.9 | 2.4 |
| 10 | 138.4 | 4.6 |
| 11 | 135.0 | 3.4 |
| 12 | 132.0 | 5.5 |
| 13 | 130.1 | 10.0 |
| 14 | 128.5 | 5.0 |
| 15 | 125.8 | 6.2 |
| 16 | 124.1 | 3.8 |
| 17 | 122.9 | 2.9 |
| 18 | 117.9 | 4.2 |
| 19 | 115.2 | 2.2 |
| 20 | 110.6 | 1.2 |
| 21 | 96.0 | 1.5 |
| 22 | 94.3 | 2.3 |
| 23 | 80.7 | 2.0 |
| 24 | 79.1 | 2.5 |
| 25 | 77.7 | 5.1 |
| 26 | 77.0 | 3.8 |
| 27 | 58.1 | 2.8 |
| 28 | 57.4 | 2.4 |
| 29 | 56.3 | 2.3 |
| 30 | 33.5 | 0.9 |
| 31 | 31.6 | 2.0 |
| 32 | 30.1 | 2.1 |
| 33 | 8.3 | 2.4 |
| 34 | 7.6 | 2.2 |
| 35 | 6.6 | 3.9 |

¹⁹F MAS on Compound 5 free form Form A

| Peak # | Chem Shift [ppm] | Intensity [rel] |
|---|---|---|
| 1 | −128.9 | 1.9 |
| 2 | −131.7 | 4.9 |
| 3 | −132.6 | 5.7 |
| 4 | −134.3 | 8.7 |
| 5 | −136.4 | 9.7 |
| 6 | −137.4 | 10.0 |
| 7 | −138.6 | 9.5 |
| 8 | −140.1 | 8.2 |
| 9 | −141.6 | 9.8 |
| 10 | −143.9 | 5.7 |
| 11 | −146.9 | 2.0 |

### 6. Single Crystal Elucidation

Single crystals having the Compound 5 free form Form A structure were grown from a mixture of acetonitrile and water. X-ray diffraction data were acquired at 100K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122) and results are summarized in the table below.

Single Crystal Elucidation of Compound 5 free form Form A

| Crystal System | Triclinic |
|---|---|
| Space Group | P1 |
| a (Å) | 12.9439(3) |
| b (Å) | 15.6031(4) |
| c (Å) | 26.6410(7) |
| α (°) | 93.8255(16) |
| β (°) | 99.6537(16) |
| γ (°) | 108.7650(18) |
| V (Å³) | 4980.3(2) |
| Z/Z′ | 1/8 |
| Temperature | 100(2) |

### C. Compound 5 Free Form Form B

### 1. Synthetic Procedure

150 mg of Compound 5 free form Form A was placed in a glass vial along with a stir bar, 0.9 mL of octanol was added to this vial and let the sample stir at 80° C. for 3 days while protected from exposure to light. After 3 days stirring, solids were collected by centrifuge filtration (0.22 um, 14 k rpm) providing Compound 5 free form Form B for XRPD analysis.

### 2. X-Ray Powder Diffraction

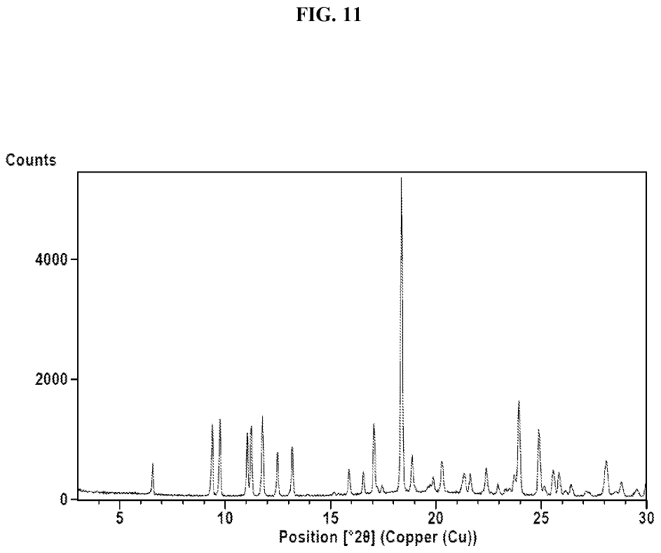

The XRPD results are shown in FIG. 11 and the table below:

XRPD Signals for Compound 5 free form Form B

| No. | Pos. [±0.2, °2θ] | Rel. Int. [%] |
|---|---|---|
| 1 | 18.4 | 100.0 |
| 2 | 23.9 | 29.2 |
| 3 | 11.8 | 24.9 |
| 4 | 9.8 | 24.0 |
| 5 | 17.1 | 22.5 |
| 6 | 11.2 | 21.9 |
| 7 | 9.4 | 21.5 |
| 8 | 24.9 | 19.9 |
| 9 | 11.0 | 19.7 |
| 10 | 13.2 | 14.9 |
| 11 | 12.5 | 13.5 |
| 12 | 18.9 | 12.9 |
| 13 | 28.1 | 10.6 |
| 14 | 20.3 | 10.6 |

### 3. Thermogravimetric Analysis

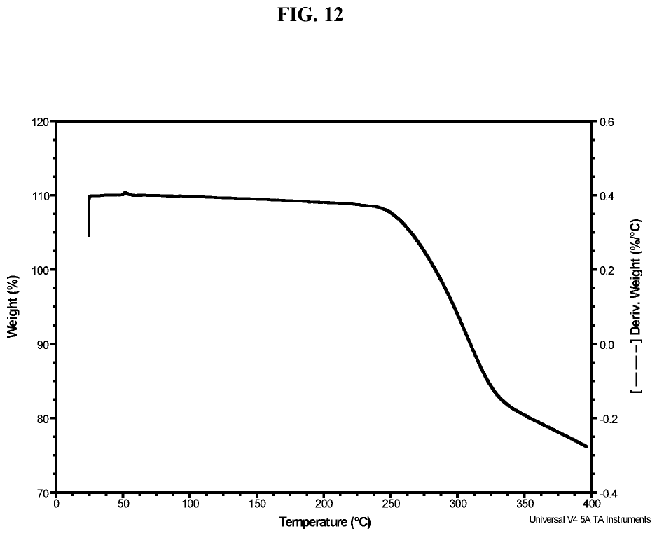

Thermogravimetric analysis of Compound 5 free form Form B was measured using TA Discovery 550 TGA from TA Instrument. A sample with weight of approximately 1-10 mg was scanned from 25° C. to 400° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 12) showed 1.0% weight loss from ambient temperature up to 200° C.

### 4. Differential Scanning Calorimetry Analysis

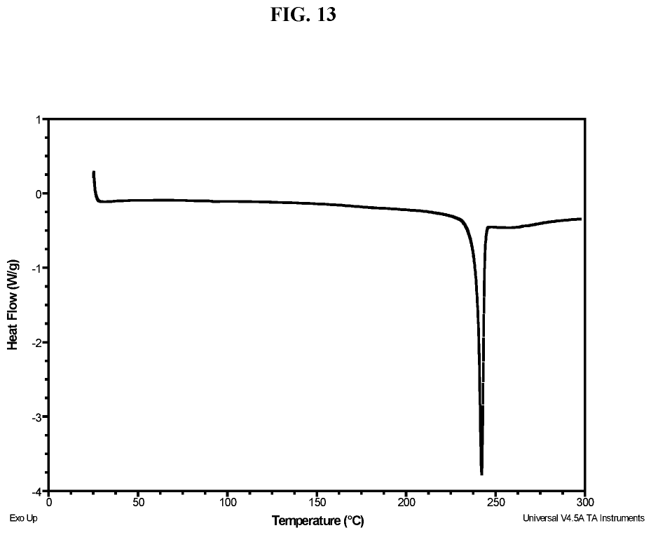

DSC of Compound 5 free form Form B was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 13) showed an endothermic peak around 240° C.

### D. Compound 5 Free Form NPA Solvate Form A

### 1. Synthetic Procedure

Placed 66 mg of Compound 5 free form Form A in a glass vial along with a stir bar, 0.15 mL of 1-propanol was added to this vial and let the sample stir at 20° C. shaker for 7 days while protected from exposure to light. After 7 days stirring, solids were collected by centrifuge filtration (0.22 um, 14 k rpm) for XRPD analysis.

### 2. X-Ray Powder Diffraction

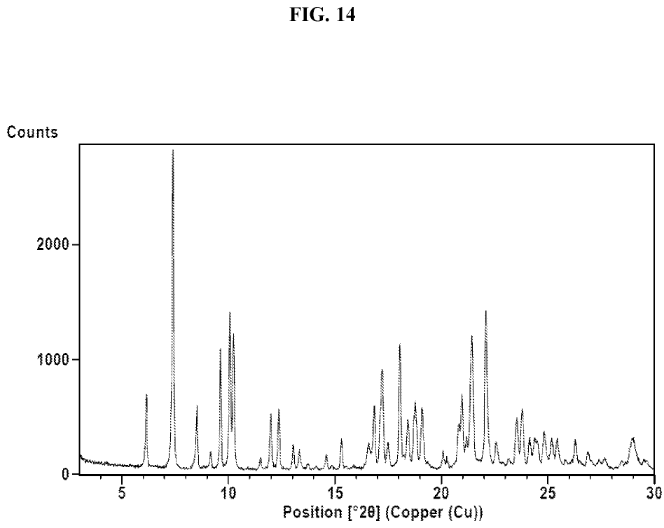

X-ray powder diffraction (XRPD) spectra results are shown in FIG. 14 and the table below:

XRPD Signals for Compound I free form NPA Solvate Form A

| No. | Pos. [±0.2, °2θ] | Rel. Int. [%] |
|---|---|---|
| 1 | 7.4 | 100.0 |
| 2 | 10.1 | 50.0 |
| 3 | 22.1 | 48.0 |
| 4 | 10.2 | 42.5 |
| 5 | 21.4 | 42.2 |
| 6 | 9.6 | 38.4 |
| 7 | 18.0 | 36.3 |
| 8 | 17.2 | 31.5 |
| 9 | 21.0 | 23.3 |
| 10 | 6.2 | 22.5 |
| 11 | 18.8 | 21.2 |
| 12 | 16.8 | 20.2 |
| 13 | 8.5 | 20.1 |
| 14 | 19.1 | 19.3 |
| 15 | 12.4 | 19.0 |
| 16 | 23.8 | 17.4 |
| 17 | 12.0 | 17.0 |
| 18 | 17.1 | 17.0 |
| 19 | 23.5 | 15.4 |
| 20 | 18.4 | 14.4 |
| 21 | 20.8 | 13.9 |
| 22 | 23.5 | 13.0 |
| 23 | 24.8 | 11.5 |

### 3. Thermogravimetric Analysis

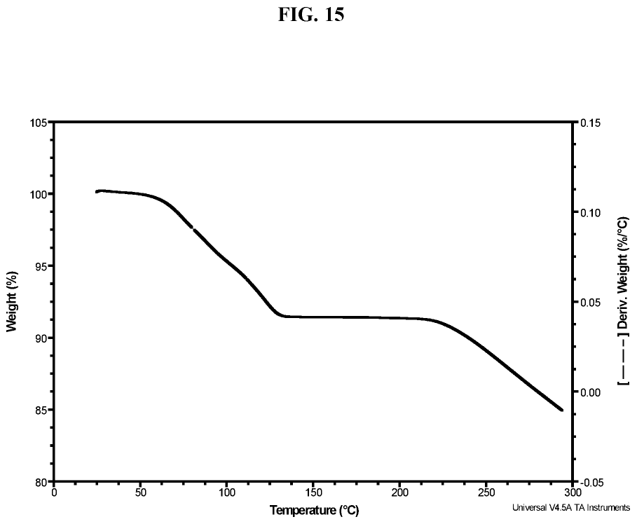

Thermogravimetric analysis of Compound I free form NPA solvate Form A was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 15) showed 8.7% weight loss from ambient temperature up to 160° C.

### 4. Differential Scanning Calorimetry Analysis

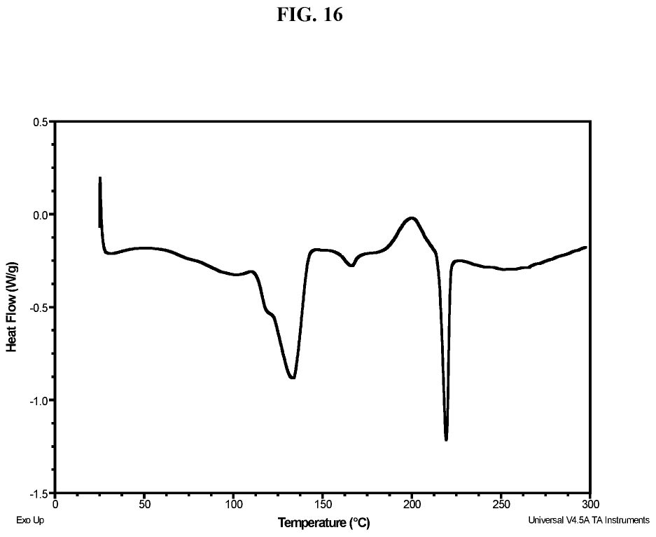

DSC of Compound 5 free form NPA solvate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 16) showed endothermic peaks around 133° C., 166° C., and 219° C.

### E. Compound 5 Free Form EtOH Solvate Form A

### 1. Synthetic Procedure

Raw Compound 5 (80 g, 157.0 mmol) was diluted in EtOAc (500 mL) and treated with activated charcoal (10 g, 832.6 mmol). Refluxed for 30 min, then cooled to room temp, then filtered through Celite (65 mm dia×30 mm h), washing with EtOAc (250 mL). Combined filtrate was concentrated to give a yellow foam.

In a 3 L RBF, foam was treated with EtOH (310 mL, 70% histology grade); heated to reflux in a metal heating bath. Refluxed for 45 min to give a uniform suspension. Power to heating bath was switched off, slowly allowed to cool to rt, stirred overnight. After 16 h, suspension was filtered, collected solid was washed with EtOH (50 mL, 70% histology grade), dried under suction for 30 min then on rotovap (2 mbar, 75° C.) for 1 h. 63.5 g pale yellow crystals, Compound 5 free form EtOH Solvate Form A, was obtained.

### 2. X-Ray Powder Diffraction

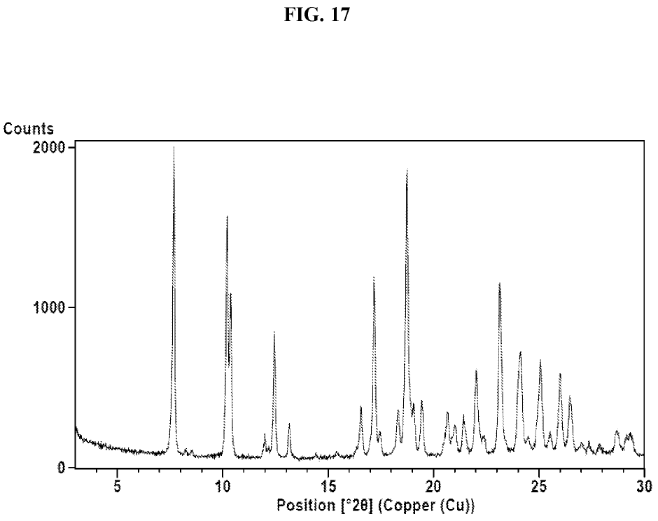

X-ray powder diffraction (XRPD) spectra results are shown in FIG. 17 and the table below.

XRPD Signals for Compound 5 free form EtOH Solvate Form A

| No. | Pos. [±0.2, °2θ] | Rel. Int. [%] |
|---|---|---|
| 1 | 7.7 | 100.0 |
| 2 | 18.7 | 92.4 |
| 3 | 10.2 | 78.9 |
| 4 | 17.2 | 59.7 |
| 5 | 23.1 | 57.5 |
| 6 | 10.4 | 53.5 |
| 7 | 12.4 | 41.5 |
| 8 | 25.1 | 32.6 |
| 9 | 24.1 | 30.7 |
| 10 | 22.0 | 29.3 |
| 11 | 26.0 | 27.2 |
| 12 | 19.4 | 19.3 |
| 13 | 26.4 | 19.3 |
| 14 | 16.6 | 17.2 |
| 15 | 19.1 | 17.0 |
| 16 | 18.3 | 15.9 |
| 17 | 20.7 | 14.8 |
| 18 | 21.4 | 12.9 |
| 19 | 13.2 | 11.0 |
| 20 | 21.0 | 10.1 |

### 3. Thermogravimetric Analysis

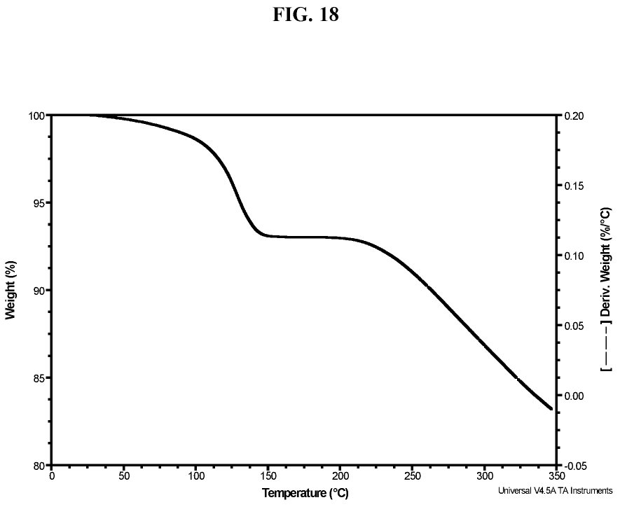

Thermogravimetric analysis of Compound 5 free form EtOH Solvate Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 18) showed 7.0% weight loss from ambient temperature up to 180° C.

### 4. Differential Scanning Calorimetry Analysis

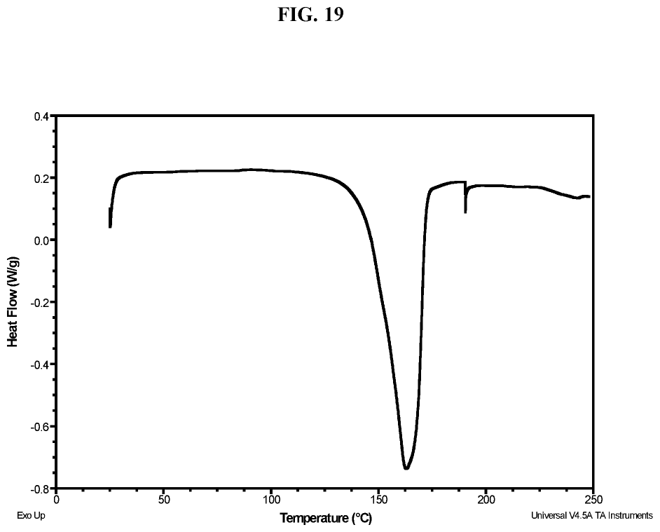

DSC of Compound 5 free form EtOH Solvate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 250° C. When the run was completed, the data were analyzed by Trios and/or Universal Analysis software (TA Instruments, New Castle, DE). The thermogram (FIG. 19) showed an endothermic peak around 163° C.

### 5. Solid State NMR

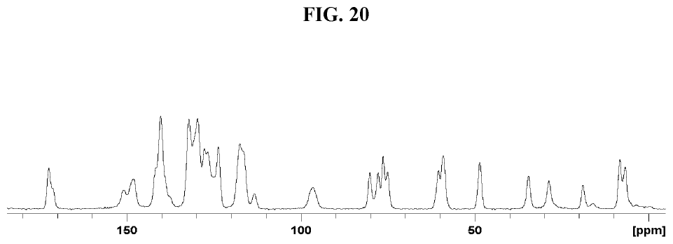

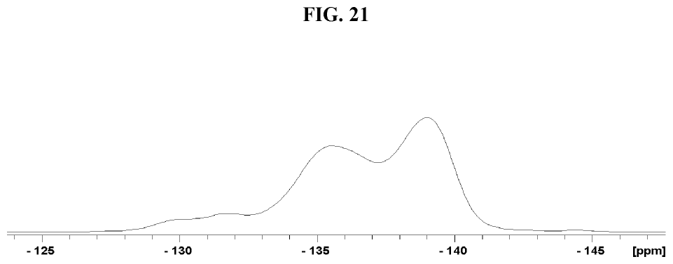

All carbon, phosphorus and fluorine spectra were recorded with proton decoupling using TPPM15 decoupling sequence. The ¹³C CPMAS and ¹⁹F MAS on Compound 5 free form EtOH Solvate Form A results are shown in FIG. 20 and FIG. 21, respectively and in the tables below:

¹³C CPMAS on Compound 5 free form EtOH Solvate Form A

| Peak # | Chem Shift [ppm] | Intensity [rel] |
|---|---|---|
| 1 | 172.3 | 4.4 |
| 2 | 171.3 | 2.2 |
| 3 | 151.0 | 2.0 |
| 4 | 147.9 | 3.2 |
| 5 | 141.8 | 4.1 |
| 6 | 140.2 | 10.0 |
| 7 | 137.6 | 1.3 |
| 8 | 132.1 | 9.7 |
| 9 | 129.6 | 9.7 |
| 10 | 127.6 | 6.5 |
| 11 | 126.7 | 6.1 |
| 12 | 123.6 | 6.7 |
| 13 | 117.4 | 7.0 |
| 14 | 113.2 | 1.6 |
| 15 | 96.4 | 2.3 |
| 16 | 80.1 | 3.9 |
| 17 | 77.6 | 3.9 |
| 18 | 76.2 | 5.7 |
| 19 | 75.0 | 4.0 |
| 20 | 60.3 | 4.1 |
| 21 | 59.0 | 5.7 |
| 22 | 48.4 | 5.0 |
| 23 | 34.4 | 3.6 |
| 24 | 28.6 | 3.0 |
| 25 | 18.8 | 2.6 |
| 26 | 8.2 | 5.3 |
| 27 | 6.6 | 4.5 |

¹⁹F MAS on Compound 5 free form EtOH Solvate Form A

| Peak # | Chem Shift [ppm] | Intensity [rel] |
|---|---|---|
| 1 | −130.0 | 1.1 |
| 2 | −131.7 | 1.6 |
| 3 | −135.6 | 7.5 |
| 4 | −139.0 | 10.0 |

### 6. Single Crystal Elucidation

Single crystals having the Compound 5 free form EtOH Solvate Form A structure were grown from a mixture of ethanol and water. X-ray diffraction data were acquired at 100K on a Bruker diffractometer equipped with Cu K, radiation (λ=1.54178 Å) and a CMOS detector. The structure was solved and refined using SHELX programs (Sheldrick, G. M., Acta Cryst., (2008) A64, 112-122) and results are summarized in the table below.

Single Crystal Elucidation of Compound
5 free form EtOH Solvate Form A

| Crystal System | Triclinic |
|---|---|
| Space Group | P1 |
| a (Å) | 10.4929(3) |
| b (Å) | 11.0808(3) |
| c (Å) | 11.4412(3) |
| α (°) | 98.3009(11) |
| β (°) | 92.4416(11) |
| γ (°) | 104.2288(10) |
| V (Å³) | 1271.71(6) |
| Z/Z′ | 1/2 |
| Temperature | 100(2) |

### F. Compound 5 Free Form MeOH Solvate Hydrate Form A

### 1. Synthetic Procedure

Method 1: 2 ml of EtOH was added to 736.8 mg of amorphous Compound 5 followed by 3 minutes water-bath sonication. The mixture was allowed to stir at room temperature. Then, isolated 55 mg of the above solids, followed by adding 0.2 ml of 9:1 (v/v) MeOH:water solvent with 10 minutes sonication. The sample was allowed to stir at ambient temperature for 5 days followed by isolating the solids for analysis.

Method 2: 46 mg of Compound 5 amorphous dried solids were added with 0.2 mL of 9:1 (v/v) MeOH:water with 10 minutes sonication. The sample was allowed to stir at ambient temperature for 5 days, followed by isolating the solids for analysis.

### 2. X-Ray Powder Diffraction

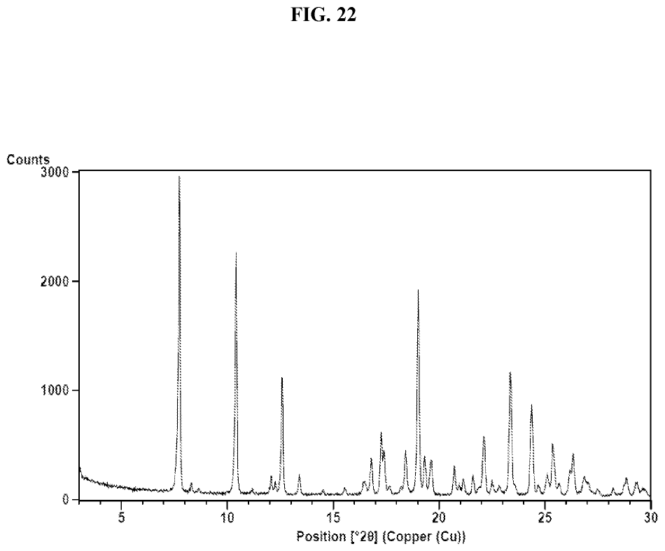

X-ray powder diffraction spectra results are shown in FIG. 22 and the table below.

XRPD Signals for Compound 5 free
form MeOH Solvate Hydrate Form A

| No. | Pos. [±0.2, °2θ] | Rel. Int. [%] |
|---|---|---|
| 1 | 7.7 | 100.0 |
| 2 | 10.4 | 77.7 |
| 3 | 19.0 | 66.5 |
| 4 | 23.4 | 39.4 |
| 5 | 12.6 | 37.9 |
| 6 | 24.4 | 28.8 |
| 7 | 17.3 | 20.1 |
| 8 | 22.1 | 18.9 |
| 9 | 25.4 | 15.8 |
| 10 | 18.4 | 14.2 |
| 11 | 17.4 | 14.1 |
| 12 | 26.3 | 13.3 |
| 13 | 19.3 | 11.8 |
| 14 | 16.8 | 11.8 |
| 15 | 19.6 | 11.0 |

### 3. Thermogravimetric Analysis

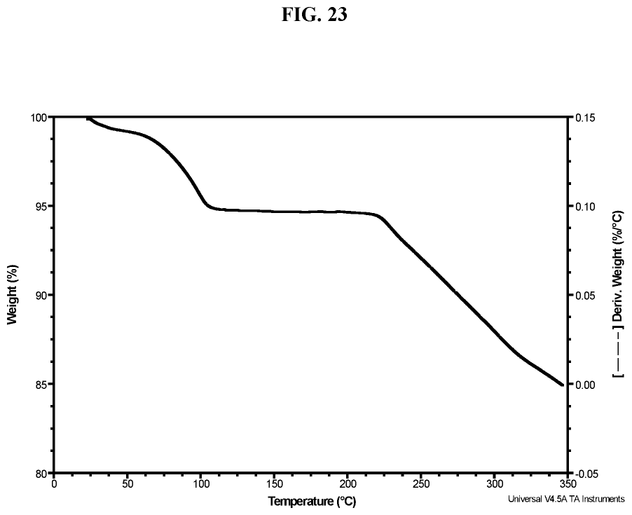

Thermogravimetric analysis of Compound 5 free form MeOH Solvate Hydrate Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 23) showed 5.0% weight loss from ambient temperature up to 150° C.

### 4. Differential Scanning Calorimetry Analysis

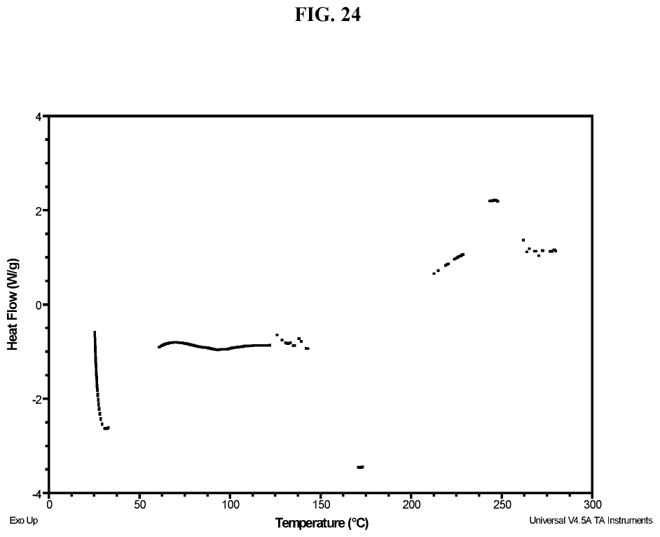

DSC of Compound 5 free form MeOH Solvate Hydrate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 290° C. When the run was completed, the data were analyzed by Trios and/or Universal Analysis software (TA Instruments, New Castle, DE). The thermogram (FIG. 24) showed endothermic peaks around 31° C., 174° C., and an exothermic peak around 246° C.

### 5. Solid State NMR

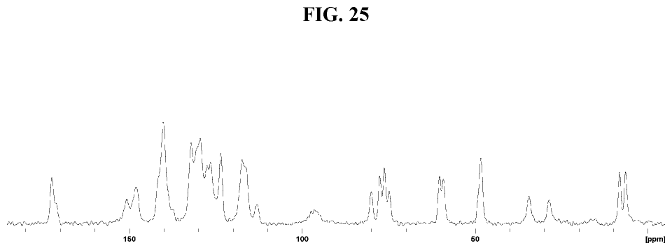

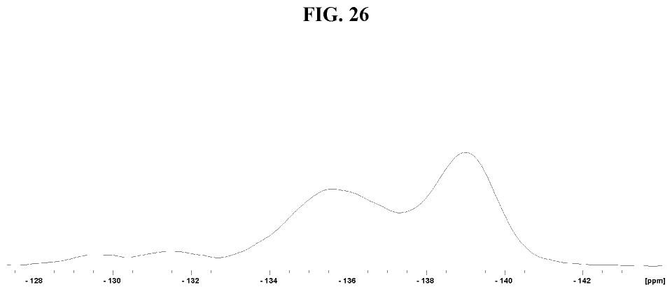

All carbon, phosphorus and fluorine spectra were recorded with proton decoupling using TPPM15 decoupling sequence. The ¹³C CPMAS and ¹⁹F MAS on Compound 5 free form MeOH Solvate Hydrate Form A results are shown in FIG. 25 and FIG. 26, respectively and in the tables below.

¹³C CPMAS on Compound 5 free form
MeOH Solvate Hydrate Form A

| Peak # | Chem Shift [ppm] | Intensity [rel] |
|---|---|---|
| 1 | 172.4 | 4.6 |
| 2 | 171.3 | 2.1 |
| 3 | 150.7 | 2.5 |
| 4 | 148.0 | 3.6 |
| 5 | 141.9 | 3.8 |
| 6 | 140.1 | 10.0 |
| 7 | 137.5 | 1.5 |
| 8 | 132.1 | 8.0 |
| 9 | 129.5 | 8.4 |
| 10 | 127.5 | 5.8 |
| 11 | 126.5 | 6.0 |
| 12 | 123.5 | 6.9 |
| 13 | 117.4 | 6.3 |
| 14 | 116.5 | 5.6 |
| 15 | 113.0 | 1.9 |
| 16 | 96.4 | 1.5 |
| 17 | 80.0 | 3.2 |
| 18 | 77.5 | 4.8 |
| 19 | 76.2 | 5.5 |
| 20 | 74.8 | 3.3 |
| 21 | 60.3 | 4.7 |
| 22 | 59.2 | 4.5 |
| 23 | 48.3 | 6.5 |
| 24 | 34.3 | 2.8 |
| 25 | 28.6 | 2.4 |
| 26 | 8.1 | 5.1 |
| 27 | 6.4 | 5.2 |

¹⁹F MAS on Compound 5 free form
MeOH Solvate Hydrate Form A

| Peak # | Chem Shift [ppm] | Intensity [rel] |
|---|---|---|
| 1 | −129.4 | 1.1 |
| 2 | −129.9 | 1.1 |
| 3 | −131.5 | 1.4 |
| 4 | −135.6 | 6.8 |

### G. Compound 5 Free Form DCM Solvate Form A

### 1. Synthetic Procedure

The reaction of Compound 5 synthesis was split into two parts for workup. Each part was poured into 3 L of water, acidified to pH 3 with 2N HCl and extracted with 4 L of EtOAc. The extracts were combined, dried (MgSO₄), filtered, and evaporated in vacuo to afford an orange-red oil. The oil was dissolved in 4 L of DCM to form a clear red solution. After swirling the solution for a few minutes, the product began to crystallize. Some extra DCM was added to make the solids filterable. The suspension was filtered in small portions and the resulting cake was washed with DCM. This process was repeated, and the cakes combined to form the final batch.

### 2. X-Ray Powder Diffraction

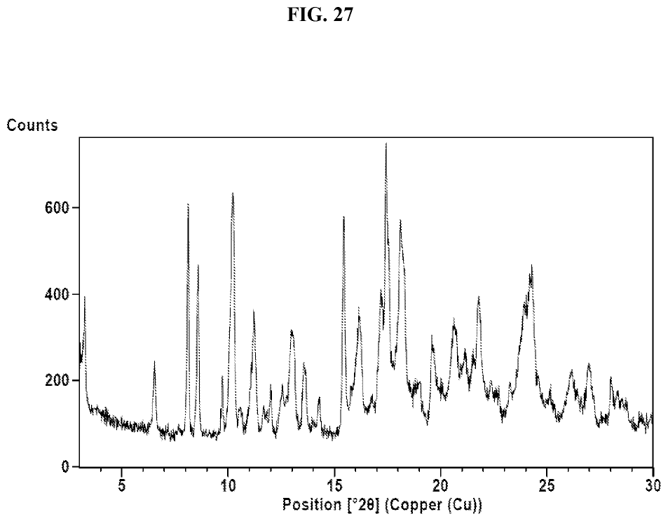

X-ray powder diffraction (XRPD) spectra results are shown in FIG. 27 and the table below.

XRPD Signals for Compound 5 free form DCM Solvate Form A

| No. | Pos. [±0.2, °2θ] | Rel. Int. [%] |
|---|---|---|
| 1 | 17.4 | 100.0 |
| 2 | 8.1 | 78.4 |
| 3 | 10.3 | 76.6 |
| 4 | 15.5 | 75.7 |
| 5 | 18.1 | 70.1 |
| 6 | 8.6 | 59.5 |
| 7 | 18.3 | 56.5 |
| 8 | 24.3 | 53.2 |
| 9 | 17.2 | 48.7 |
| 10 | 21.8 | 44.5 |
| 11 | 3.3 | 42.5 |
| 12 | 11.2 | 42.2 |
| 13 | 16.2 | 40.2 |
| 14 | 20.6 | 37.4 |
| 15 | 19.6 | 31.6 |
| 16 | 13.1 | 27.7 |
| 17 | 21.2 | 26.7 |
| 18 | 13.5 | 24.4 |
| 19 | 6.6 | 22.6 |
| 20 | 9.7 | 20.6 |
| 21 | 26.9 | 19.5 |
| 22 | 26.1 | 19.2 |
| 23 | 19.0 | 16.4 |
| 24 | 28.0 | 16.1 |
| 25 | 23.2 | 14.9 |
| 26 | 12.0 | 14.8 |
| 27 | 12.5 | 13.9 |
| 28 | 14.3 | 11.7 |

### 3. Thermogravimetric Analysis

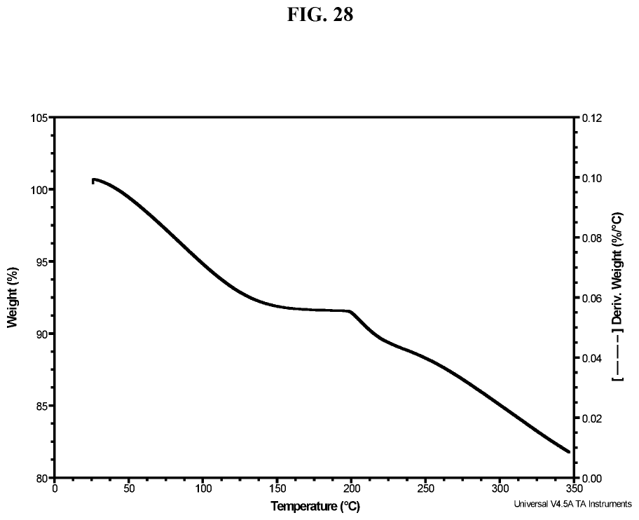

Thermogravimetric analysis of Compound 5 free form DCM Solvate Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 28) showed 8.7% weight loss from ambient temperature up to 180° C.

### 4. Solid State NMR

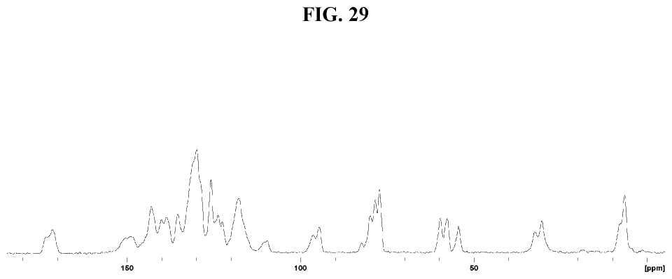

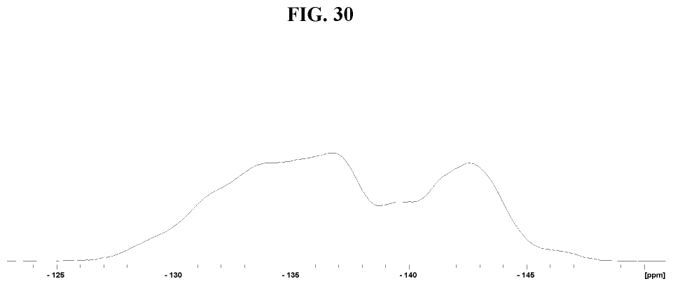

All carbon, phosphorus and fluorine spectra were recorded with proton decoupling using TPPM15 decoupling sequence. The ¹³C CPMAS and ¹⁹F MAS on Compound 5 DCM solvate Form A results are shown in FIG. 29 and FIG. 30, respectively and in the tables below.

¹³C CPMAS on Compound 5 free form DCM Solvate Form A

| Peak # | Chem Shift [ppm] | Intensity [rel] |
|---|---|---|
| 1 | 173.3 | 1.6 |
| 2 | 171.5 | 2.3 |
| 3 | 150.4 | 1.5 |
| 4 | 149.0 | 1.6 |
| 5 | 143.0 | 4.5 |
| 6 | 140.0 | 3.3 |
| 7 | 138.6 | 3.5 |
| 8 | 135.3 | 3.8 |
| 9 | 132.6 | 4.8 |
| 10 | 131.2 | 8.4 |
| 11 | 129.9 | 10.0 |
| 12 | 128.8 | 6.9 |
| 13 | 125.8 | 7.1 |
| 14 | 123.7 | 3.7 |
| 15 | 122.4 | 3.0 |
| 16 | 117.7 | 5.3 |
| 17 | 116.5 | 3.1 |
| 18 | 109.6 | 1.2 |
| 19 | 96.3 | 1.7 |
| 20 | 94.5 | 2.5 |
| 21 | 82.3 | 1.0 |
| 22 | 79.8 | 3.6 |
| 23 | 78.4 | 5.1 |
| 24 | 77.1 | 6.1 |
| 25 | 59.6 | 3.4 |
| 26 | 57.6 | 3.4 |
| 27 | 54.3 | 2.6 |
| 28 | 32.3 | 2.1 |
| 29 | 30.4 | 3.1 |
| 30 | 7.9 | 2.9 |
| 31 | 6.5 | 5.6 |

¹⁹F MAS on Compound 5 free form DCM Solvate Form A

| Peak # | Chem Shift [ppm] | Intensity [rel] |
|---|---|---|
| 1 | −129.4 | 2.5 |
| 2 | −131.8 | 6.6 |
| 3 | −133.8 | 9.0 |
| 4 | −136.8 | 10.0 |
| 5 | −139.7 | 5.5 |
| 6 | −141.5 | 7.9 |
| 7 | −142.6 | 9.1 |
| 8 | −146.5 | 1.0 |

### H. Compound 5 Free Form EtOAc Heptane Solvate Form A

### 1. Synthetic Procedure

First, raw Compound 5 was distilled via the following procedure: 1) About 15 g of Compound 1 was dissolved in 6.0 volumes of EtOAc; 2) concentrated to 3 volumes under vacuum (100-150 mmHg) maintaining internal temperature to NMT 40° C., 3) added 7 volumes of dry EtOAc (KF NMT 500 ppm); 4) concentrated to 3 volumes under vacuum maintaining internal temperature to NMT 40° C., S) added 7 volumes of dry EtOAc; 6) concentrated to 3 volumes under vacuum maintaining internal temperature to NMT 30° C.; 7) added 3 volumes of EtOAc.

Second, the reactor content was heated to 55° C., followed by transferring 18 volumes of heptane over 12 h at 55° C. The reaction temperature was decreased to 20° C. over 2 h and aged for 4 h at 20° C. The solids were isolated and washed with 4 volumes of EtOAc:n-Heptane (1:3) at 20° C., followed by drying under vacuum at NMT 50° C.

### 2. X-Ray Powder Diffraction

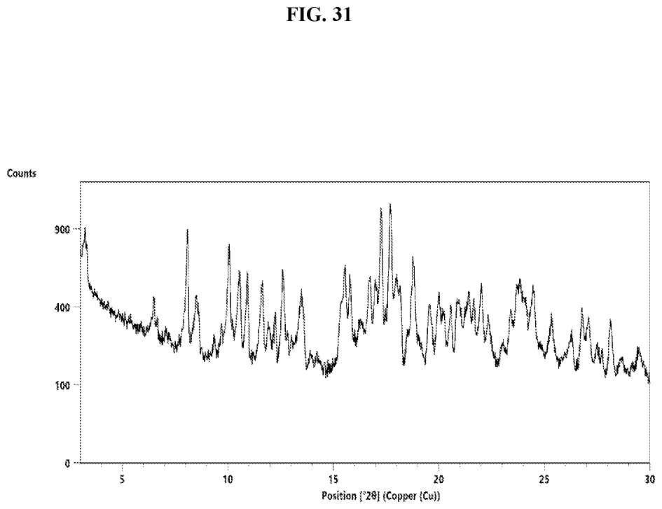

X-ray powder diffraction (XRPD) spectra results are shown in FIG. 31 and in the table below.

XRPD Signals for Compound 5 free
form EtOAc Heptane Solvate Form A

| No. | Pos. [±0.2, °2θ] | Rel. Int. [%] |
|---|---|---|
| 1 | 17.7 | 100.0 |
| 2 | 17.2 | 95.8 |
| 3 | 8.1 | 63.5 |
| 4 | 15.5 | 55.0 |
| 5 | 10.0 | 53.7 |
| 6 | 18.8 | 53.1 |
| 7 | 12.6 | 44.8 |
| 8 | 23.8 | 42.0 |
| 9 | 16.7 | 41.6 |
| 10 | 10.9 | 40.8 |
| 11 | 10.5 | 38.0 |
| 12 | 11.6 | 37.7 |
| 13 | 18.0 | 34.8 |
| 14 | 17.0 | 34.6 |
| 15 | 22.0 | 34.0 |
| 16 | 15.8 | 33.7 |
| 17 | 13.4 | 31.3 |
| 18 | 20.0 | 30.4 |
| 19 | 20.9 | 29.0 |
| 20 | 3.2 | 28.6 |
| 21 | 24.4 | 28.4 |
| 22 | 18.2 | 28.0 |
| 23 | 21.3 | 27.3 |
| 24 | 19.5 | 25.3 |
| 25 | 28.1 | 22.6 |
| 26 | 21.6 | 22.4 |
| 27 | 26.8 | 22.2 |
| 28 | 8.5 | 22.0 |
| 29 | 20.5 | 20.8 |
| 30 | 20.2 | 19.9 |
| 31 | 25.3 | 19.3 |
| 32 | 27.1 | 18.9 |
| 33 | 6.5 | 18.8 |
| 34 | 22.3 | 17.7 |
| 35 | 12.2 | 16.2 |
| 36 | 23.4 | 15.5 |
| 37 | 16.3 | 15.0 |
| 38 | 11.9 | 12.5 |
| 39 | 26.2 | 10.7 |

### 3. Thermogravimetric Analysis

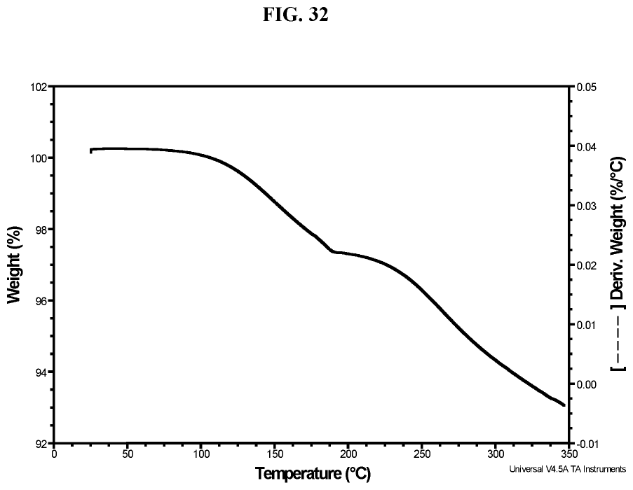

Thermal gravimetric analysis of Compound 5 EtOAc-heptane solvate Form A was measured using TA Discovery 550 TGA from TA Instrument. A sample with weight of approximately 1-20 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 32) showed 2.9% weight loss from ambient temperature up to 200° C.

### 4. Differential Scanning Calorimetry Analysis

DSC of Compound 5 EtOAc-heptane solvate Form A was measured using the TA Instruments Q2000 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram showed endothermic peaks around 195° C.

### 5. Solid State NMR

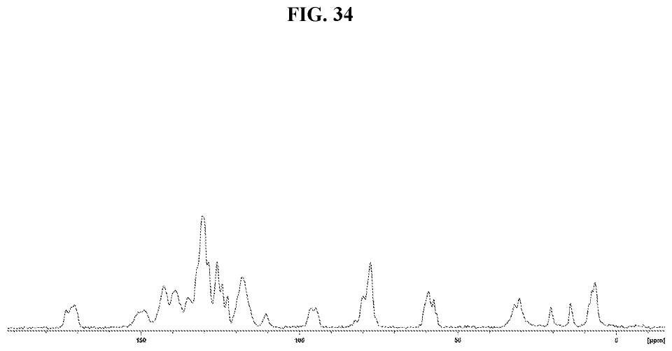

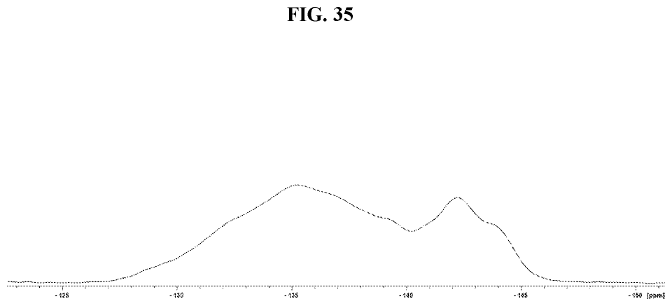

All carbon and fluorine spectra were recorded with proton decoupling using TPPM15 decoupling sequence. The ¹³C CPMAS and ¹⁹F MAS on Compound 5 free form EtOAc Heptane Solvate Form A results are shown in FIG. 34 and FIG. 35, respectively and in the tables below.

¹³C CPMAS on Compound 5 free form
EtOAc Heptane Solvate Form A

| Peak # | Chem Shift [ppm] | Intensity [rel] |
|---|---|---|
| 1 | 173.5 | 1.6 |
| 2 | 171.8 | 1.9 |
| 3 | 170.9 | 2.1 |
| 4 | 170.2 | 1.5 |
| 5 | 150.8 | 1.4 |
| 6 | 149.0 | 1.6 |
| 7 | 142.7 | 3.8 |
| 8 | 139.0 | 3.4 |
| 9 | 135.1 | 2.7 |
| 10 | 132.3 | 5.3 |
| 11 | 130.4 | 10.0 |
| 12 | 128.6 | 5.8 |
| 13 | 125.9 | 6.0 |
| 14 | 124.3 | 3.9 |
| 15 | 122.6 | 2.8 |
| 16 | 119.7 | 2.4 |
| 17 | 118.0 | 4.5 |
| 18 | 110.5 | 1.26 |
| 19 | 96.2 | 1.76 |
| 20 | 94.7 | 1.75 |
| 21 | 82.3 | 0.66 |
| 22 | 79.8 | 2.79 |
| 23 | 77.4 | 5.84 |
| 24 | 59.2 | 3.23 |
| 25 | 57.5 | 2.55 |
| 26 | 56.8 | 1.26 |
| 27 | 31.9 | 2.07 |
| 28 | 30.5 | 2.62 |
| 29 | 20.6 | 1.82 |
| 30 | 20.2 | 1.24 |
| 31 | 14.3 | 2.21 |
| 32 | 8.8 | 1.77 |
| 33 | 7.5 | 3.35 |
| 34 | 6.6 | 4.04 |

¹⁹F MAS on Compound 5 free form
EtOAc Heptane Solvate Form A

| Peak # | Chem Shift [ppm] | Intensity [rel] |
|---|---|---|
| 1 | −129.6 | 2.2 |
| 2 | −132.2 | 6.2 |
| 3 | −135.3 | 10.0 |
| 4 | −136.9 | 8.9 |
| 5 | −139.2 | 6.6 |
| 6 | −141.0 | 6.2 |
| 7 | −142.2 | 8.7 |
| 8 | −143.7 | 6.0 |

### Example 10—Solid Forms of Compound 3

### A. Compound 3 Free Form Form A

### 1. Synthetic Procedure

Reaction mixture was concentrated in vacuo to remove THF and methanol. Raw Compound 3 was treated with water (800 mL) acidified with aqueous 2 N HCl to pH=3, and cooled to 0° C. with ice/water bath. Reaction mixture was further treated with EtOAc (1 L), stirred for 10 minutes, and extracted with EtOAc (500 mL). Combined organic phase was washed with water (400 mL), brine (600 mL), dried over MgSO₄, filtered, and concentrated in vacuo to afford Compound 3 (80 g) as a peach-colored crispy foam. Foam was further treated with acetonitrile (800 mL), refluxed for 30 minutes, treated with water (1 L), refluxed for additional 1 h, and allowed to cool down to room temperature over 12 h. Suspension was filtered. Collected solid was washed with acetonitrile:water (1:1, 1 L), dried under air suction for 2 h, and further dried in vacuum oven at 90° C. for 2 days. The resulting solid was collected to provide Compound 3 free form Form A.

### 2. X-Ray Powder Diffraction

X-ray powder diffraction (XRPD) spectra results are shown in FIG. 34 and the table below.

XRPD Signals for Compound 3 free form Form A

| No. | Pos. [±0.2, °2θ] | Rel. Int. [%] |
|---|---|---|
| 1 | 18.2 | 100.0 |
| 2 | 12.3 | 92.1 |
| 3 | 22.4 | 71.4 |
| 4 | 11.0 | 68.8 |
| 5 | 15.9 | 60.7 |
| 6 | 11.3 | 48.1 |
| 7 | 15.7 | 44.8 |
| 8 | 5.7 | 44.1 |
| 9 | 23.2 | 39.2 |
| 10 | 23.2 | 37.1 |
| 11 | 10.1 | 33.9 |
| 12 | 20.2 | 32.1 |
| 13 | 15.0 | 29.3 |
| 14 | 11.5 | 25.5 |
| 15 | 19.5 | 22.6 |
| 16 | 15.3 | 18.3 |
| 17 | 22.8 | 16.8 |
| 18 | 25.0 | 15.5 |
| 19 | 17.9 | 15.4 |
| 20 | 25.2 | 15.0 |
| 21 | 25.6 | 14.6 |
| 22 | 21.0 | 12.7 |
| 23 | 23.6 | 11.1 |

### 3. Thermogravimetric Analysis

Thermogravimetric analysis of Compound 3 free form Form A was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 35) showed 0.4% weight loss from ambient temperature up to about 230° C.

### 4. Differential Scanning Calorimetry Analysis

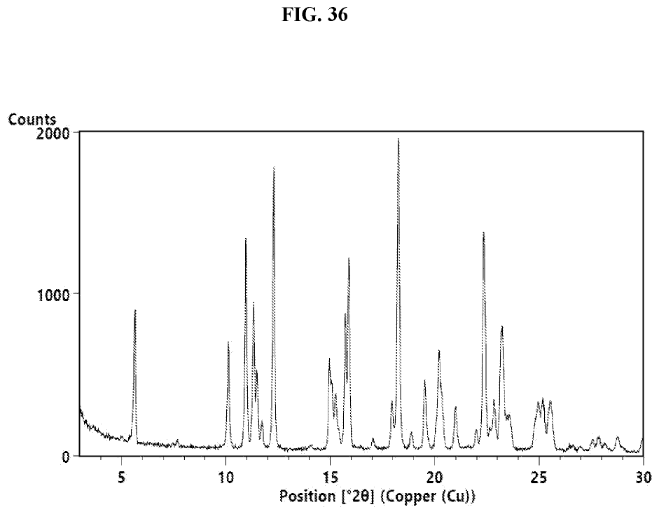

DSC of Compound 3 Free Form Form A was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 36) showed an endothermic peak at about 244° C.

### 5. Solid State NMR

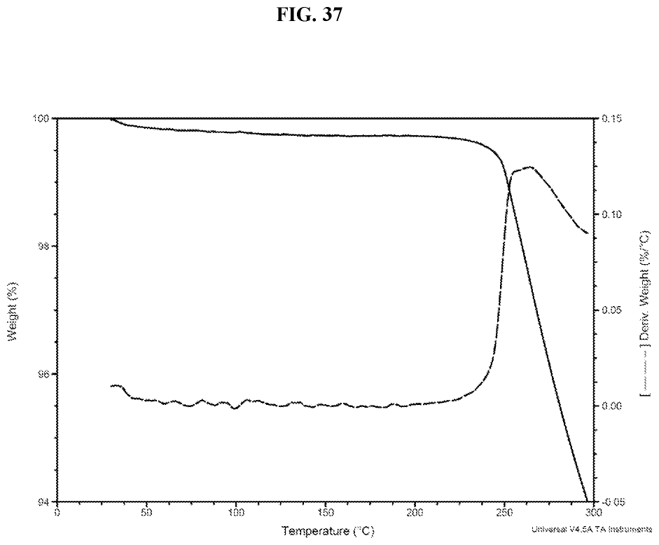

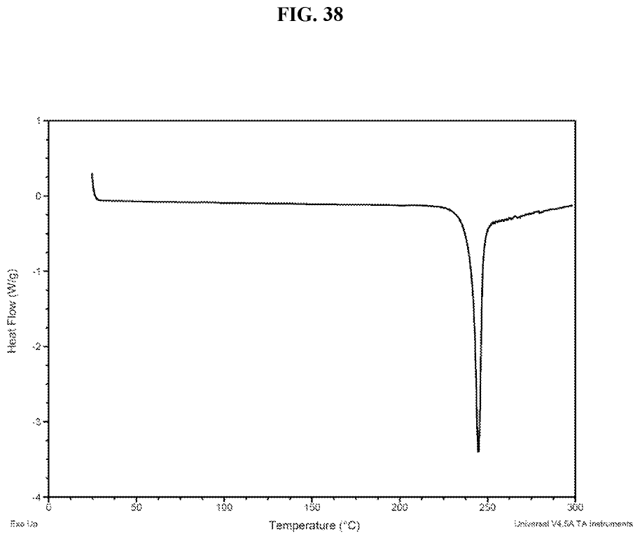

All carbon and fluorine spectra were recorded with proton decoupling using SPINAL64 decoupling sequence. The ¹³C CPMAS and ¹⁹F MAS on Compound 3 free form Form A results are shown in FIG. 37 and FIG. 38, respectively, and in the tables below:

¹³C CPMAS on Compound 3 free form Form A

| Peak # | Chem Shift [ppm] | Intensity [rel] |
|---|---|---|
| 1 | 170.6 | 7.7 |
| 2 | 153.2 | 1.1 |
| 3 | 150.6 | 1.5 |
| 4 | 147.6 | 5.2 |
| 5 | 141.1 | 5.5 |
| 6 | 139.4 | 3.4 |
| 7 | 138.4 | 4.6 |
| 8 | 136.3 | 3.3 |
| 9 | 133.5 | 6.1 |
| 10 | 132.6 | 6.1 |
| 11 | 130.7 | 5.0 |
| 12 | 128.1 | 10.0 |
| 13 | 126.7 | 6.0 |
| 14 | 124.9 | 6.7 |
| 15 | 123.7 | 4.9 |
| 16 | 116.8 | 7.9 |
| 17 | 114.7 | 5.5 |
| 18 | 93.9 | 4.5 |
| 19 | 82.4 | 3.2 |
| 20 | 80.0 | 6.1 |
| 21 | 79.3 | 5.1 |
| 22 | 78.0 | 3.3 |
| 23 | 60.7 | 4.6 |
| 24 | 59.7 | 4.1 |
| 25 | 54.6 | 5.9 |
| 26 | 32.9 | 2.7 |
| 27 | 28.4 | 3.3 |
| 28 | 9.3 | 4.9 |
| 29 | 7.9 | 4.7 |

¹⁹F MAS on Compound 3 free form Form A

| Peak # | Chem Shift [ppm] | Intensity [rel] |
|---|---|---|
| 1 | −132.8 | 10.0 |
| 2 | −134.8 | 9.1 |
| 3 | −138.3 | 6.3 |

### B. Compound 3 Free Form Form B

### 1. Synthetic Procedure

50 mg of amorphous Compound 3 was slurried in 0.6 ml of chlorobenzene or toluene solution for 2 weeks at room temperature in a shaker box equipped with a stir bar. The resulting solid was isolated to provide Compound 3 free form Form B.

### 2. X-Ray Powder Diffraction

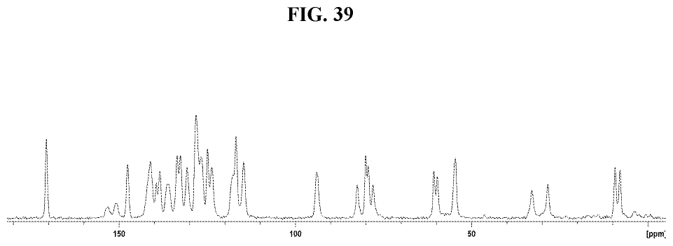

X-ray powder diffraction (XRPD) spectra results are shown in FIG. 39 and the table below.

XRPD Signals for Compound 3 free form Form B

| No. | Pos. [±0.2, °2θ] | Rel. Int. [%] |
|---|---|---|
| 1 | 10.5 | 100.0 |
| 2 | 11.6 | 98.9 |
| 3 | 16.6 | 59.2 |
| 4 | 8.2 | 58.0 |
| 5 | 18.6 | 55.3 |
| 6 | 13.5 | 48.7 |
| 7 | 21.0 | 42.7 |
| 8 | 22.1 | 41.2 |
| 9 | 24.7 | 40.3 |
| 10 | 8.5 | 30.6 |
| 11 | 17.1 | 27.0 |
| 12 | 21.8 | 26.9 |
| 13 | 25.1 | 26.2 |
| 14 | 27.3 | 24.4 |
| 15 | 23.6 | 24.4 |
| 16 | 6.6 | 19.2 |
| 17 | 24.3 | 17.6 |
| 18 | 23.3 | 17.3 |
| 19 | 17.8 | 16.5 |
| 20 | 12.8 | 14.2 |
| 21 | 17.6 | 13.9 |
| 22 | 15.2 | 13.3 |
| 23 | 14.1 | 13.1 |
| 24 | 28.0 | 12.6 |
| 25 | 25.8 | 12.4 |
| 26 | 26.4 | 12.3 |
| 27 | 19.1 | 12.1 |

### 3. Thermogravimetric Analysis

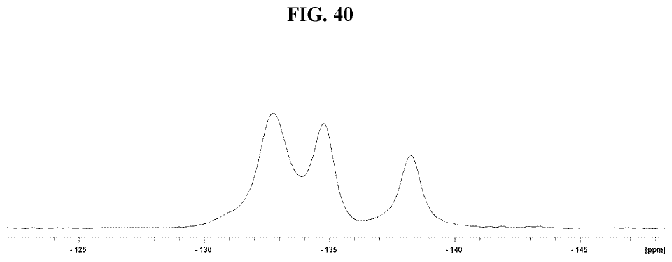

Thermogravimetric analysis of Compound 3 Free Form Form B was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 40) showed 0.6% weight loss from ambient temperature up to about 260° C.

### 4. Differential Scanning Calorimetry Analysis

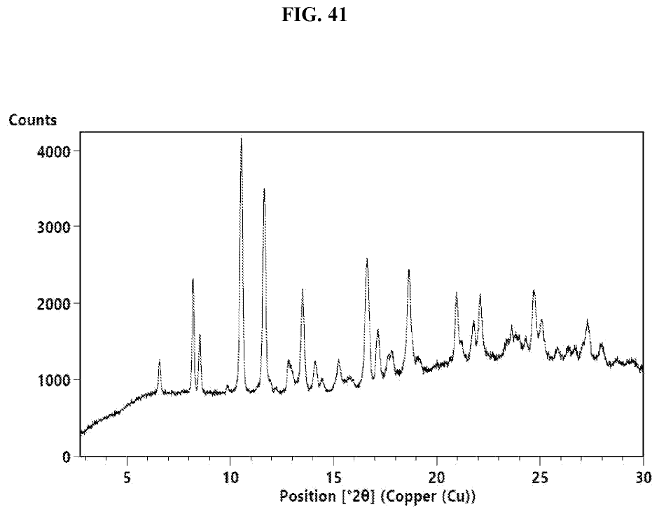

DSC of Compound 3 Free Form Form B was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 41) showed endothermic peaks at about 231° C. and 255° C.

### 5. Solid State NMR

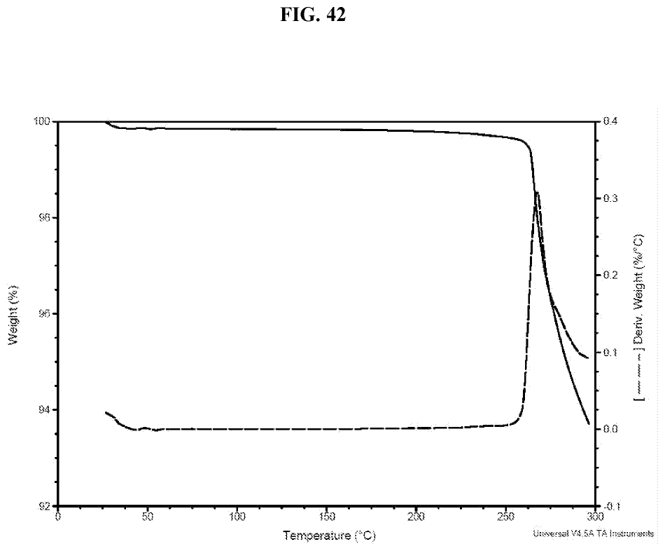

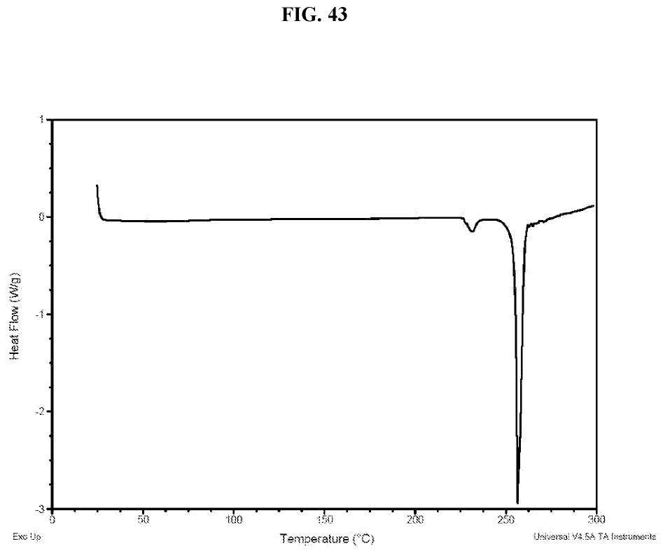

All carbon and fluorine spectra were recorded with proton decoupling using SPINAL64 decoupling sequence. The ¹³C CPMAS and ¹⁹F MAS on Compound 3 free form Form B results are shown in FIG. 42 and FIG. 43, respectively, and in the tables below:

¹³C CPMAS on Compound 3 free form Form B

| Peak # | Chem Shift [ppm] | Intensity [rel] |
|---|---|---|
| 1 | 173.0 | 1.9 |
| 2 | 171.9 | 2.7 |
| 3 | 170.9 | 2.3 |
| 4 | 153.2 | 1.0 |
| 5 | 152.0 | 1.3 |
| 6 | 150.8 | 1.5 |
| 7 | 149.7 | 1.3 |
| 8 | 147.6 | 3.7 |
| 9 | 145.2 | 0.9 |
| 10 | 143.4 | 4.3 |
| 11 | 142.5 | 5.7 |
| 12 | 140.5 | 1.0 |
| 13 | 138.3 | 5.2 |
| 14 | 134.2 | 3.8 |
| 15 | 132.9 | 6.7 |
| 16 | 130.4 | 10.0 |
| 17 | 128.4 | 5.3 |
| 18 | 125.8 | 4.1 |
| 19 | 123.7 | 4.7 |
| 20 | 121.6 | 2.9 |
| 21 | 119.3 | 2.6 |
| 22 | 118.4 | 3.1 |
| 23 | 117.4 | 3.4 |
| 24 | 115.2 | 4.6 |
| 25 | 113.7 | 2.5 |
| 26 | 110.1 | 0.8 |
| 27 | 108.6 | 1.5 |
| 28 | 97.9 | 2.1 |
| 29 | 94.5 | 3.2 |
| 30 | 80.4 | 5.3 |
| 31 | 78.0 | 6.2 |
| 32 | 76.9 | 4.3 |
| 33 | 60.9 | 0.9 |
| 34 | 58.0 | 4.9 |
| 35 | 57.0 | 3.3 |
| 36 | 55.6 | 3.9 |
| 37 | 54.6 | 2.2 |
| 38 | 32.3 | 2.1 |
| 39 | 30.7 | 2.0 |
| 40 | 29.2 | 3.0 |
| 41 | 7.2 | 6.8 |
| 42 | 6.7 | 4.4 |

¹⁹F MAS on Compound 3 free form Form B

| Peak # | Chem Shift [ppm] | Intensity [rel] |
|---|---|---|
| 1 | −129.4 | 10.0 |
| 2 | −132.3 | 7.7 |
| 3 | −137.6 | 7.1 |
| 4 | −142.0 | 8.0 |
| 5 | −145.1 | 6.6 |
| 6 | −146.9 | 5.0 |

### C. Compound 3 Free Form Hydrate Form A

### 1. Synthetic Procedure

30 mg of Compound 3 free form Form A was dissolved in 1.2 mL of 1,2-dimethoxyethane upon stirring and slowly added to a vial containing 2.4 mL of water as anti-solvent. Resulting solution was stirred until the precipitate appeared. The precipitate was isolated and dried to provide Compound 3 free form Hydrate Form A.

### 2. X-Ray Powder Diffraction

XRPD was performed with a Panalytical X′Pert3 Powder XRPD on a Si zero-background holder. The 20 position was calibrated against a Panalytical Si reference standard disc. The parameters used are tabulated below.

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

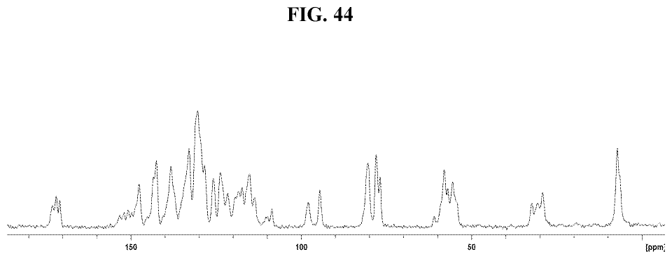

The results are shown in FIG. 44 and the table below.

XRPD Signals for Compound 3 free form Hydrate Form A

| No. | Pos. [±0.2, °2θ] | Rel. Int. [%] |
|---|---|---|
| 1 | 15.3 | 100.0 |
| 2 | 12.3 | 99.8 |
| 3 | 17.9 | 84.9 |
| 4 | 22.2 | 72.0 |
| 5 | 11.6 | 65.9 |
| 6 | 18.2 | 60.1 |
| 7 | 12.1 | 39.2 |
| 8 | 23.3 | 37.0 |
| 9 | 10.9 | 35.7 |
| 10 | 15.9 | 35.1 |
| 11 | 4.7 | 29.6 |
| 12 | 9.5 | 23.7 |
| 13 | 20.3 | 23.3 |
| 14 | 24.4 | 22.0 |
| 15 | 10.0 | 21.4 |
| 16 | 16.7 | 14.7 |
| 17 | 25.3 | 12.8 |

### 3. Thermogravimetric Analysis

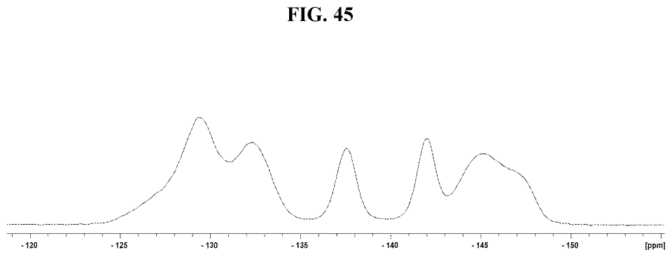

Thermogravimetric analysis of Compound 3 Free Form Hydrate Form A was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 45) showed 1.6% weight loss from ambient temperature up to about 170° C.

### 4. Differential Scanning Calorimetry Analysis

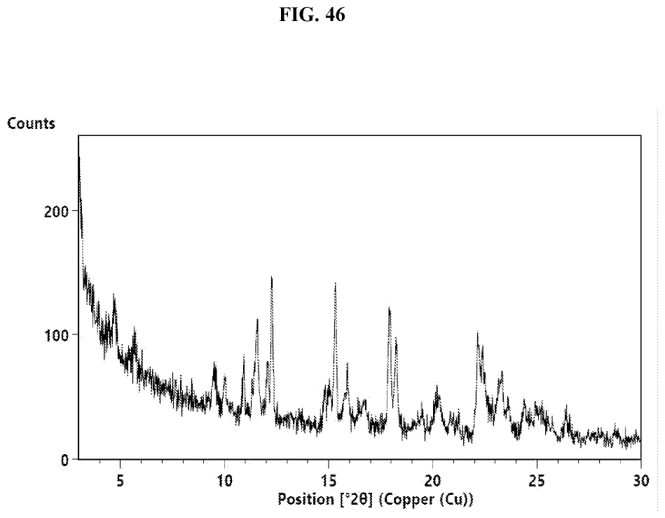

DSC of Compound 3 Free Form Hydrate Form A was measured using the TA Instruments Q2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 46) showed endothermic peaks at about 155, 242° C. and an exothermic peak at about 172° C.

### D. Compound 3 Free Form Hydrate Form B

### 1. Synthetic Procedure

30 mg of Compound 3 free form Form A was dissolved in 1.2 mL of acetone upon stirring and slowly added to a vial containing 2.4 mL of water as anti-solvent. Resulting solution was stirred until the precipitate appeared. The precipitate was isolated and dried to provide Compound 3 free form Hydrate Form B.

### 2. X-Ray Powder Diffraction

XRPD was performed with a Panalytical X′Pert3 Powder XRPD on a Si zero-background holder. The 20 position was calibrated against a Panalytical Si reference standard disc. The parameters used are tabulated below.

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

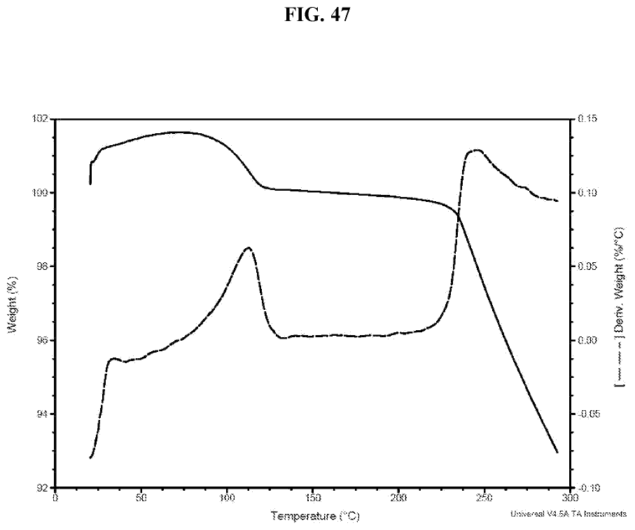

The results are shown in FIG. 47 and the table below.

XRPD Signals for Compound 3 free form Form B

| No. | Pos. [±0.2, °2θ] | Rel. Int. [%] |
|---|---|---|
| 1 | 15.3 | 100.0 |
| 2 | 11.5 | 85.9 |
| 3 | 15.8 | 69.9 |
| 4 | 17.9 | 69.8 |
| 5 | 16.6 | 57.7 |
| 6 | 22.2 | 57.2 |
| 7 | 23.3 | 51.7 |
| 8 | 12.3 | 48.0 |
| 9 | 17.6 | 35.9 |
| 10 | 23.7 | 31.1 |
| 11 | 9.5 | 29.1 |
| 12 | 14.8 | 28.9 |
| 13 | 25.4 | 28.5 |
| 14 | 10.7 | 25.2 |
| 15 | 22.9 | 23.3 |
| 16 | 24.4 | 23.0 |
| 17 | 4.5 | 21.7 |
| 18 | 9.0 | 21.4 |
| 19 | 21.1 | 16.7 |
| 20 | 20.4 | 15.2 |
| 21 | 26.4 | 15.1 |
| 22 | 27.4 | 11.9 |
| 23 | 19.0 | 11.6 |

### 3. Thermogravimetric Analysis

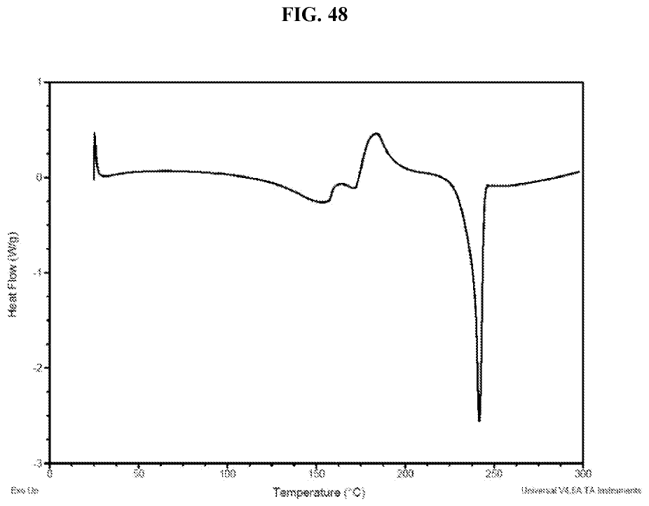

Thermogravimetric analysis of Compound 3 Free Form Hydrate Form B was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 48) showed 4.8% weight loss from ambient temperature up to 200° C.

### 4. Differential Scanning Calorimetry Analysis

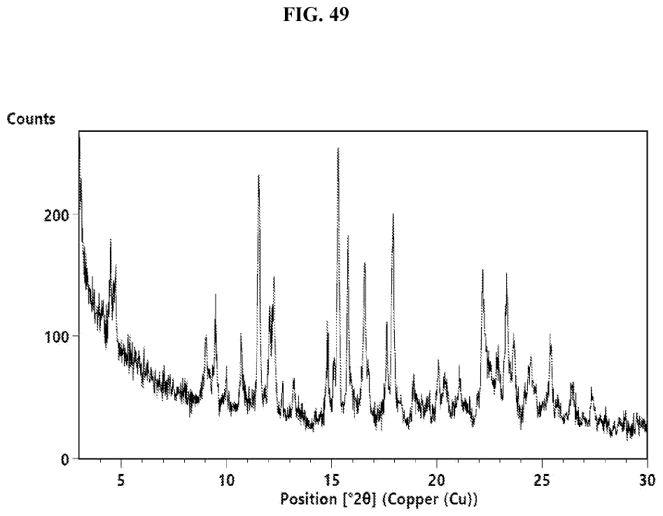

DSC of Compound 3 Free Form Hydrate Form B was measured using the TA Instruments Q2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 49) showed endothermic peaks at about 79 and 244° C., and an exothermic peak at about 184° C.

### E. Compound 3 Free Form Hydrate Form C

### 1. Synthetic Procedure

30 mg of Compound 3 free form Form A was dissolved in 2 mL of THF. 4 mL of MTBE as anti-solvent was slowly added upon stirring until precipitate appeared. The precipitate was isolated and dried to provide Compound 3 free form Hydrate Form C.

### 2. X-Ray Powder Diffraction

XRPD was performed with a Panalytical X′Pert3 Powder XRPD on a Si zero-background holder. The 20 position was calibrated against a Panalytical Si reference standard disc. The parameters used are tabulated below.

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

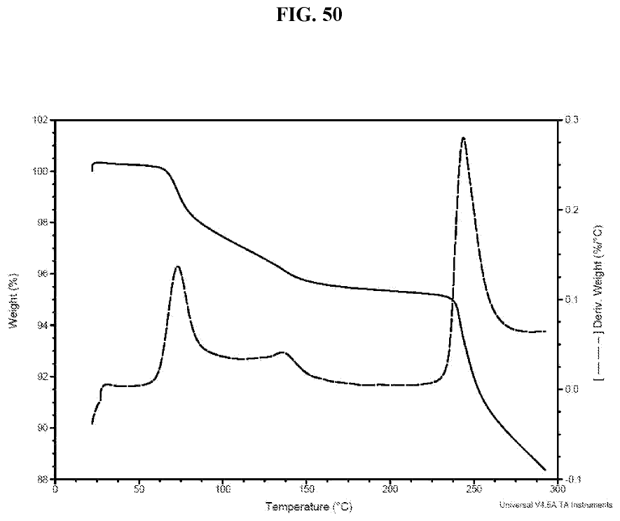

The results are shown in FIG. 50 and the table below.

XRPD Signals for Compound 3 free form Hydrate Form C

| No. | Pos. [±0.2, °2θ] | Rel. Int. [%] |
|---|---|---|
| 1 | 15.2 | 100.0 |
| 2 | 22.0 | 80.7 |
| 3 | 11.4 | 67.2 |
| 4 | 22.9 | 59.5 |
| 5 | 18.5 | 55.0 |
| 6 | 19.7 | 46.7 |
| 7 | 16.3 | 45.2 |
| 8 | 20.2 | 37.6 |
| 9 | 10.9 | 34.1 |
| 10 | 25.2 | 32.9 |
| 11 | 4.6 | 32.2 |
| 12 | 12.5 | 28.8 |
| 13 | 9.2 | 19.8 |
| 14 | 5.7 | 12.7 |

### 3. Thermogravimetric Analysis

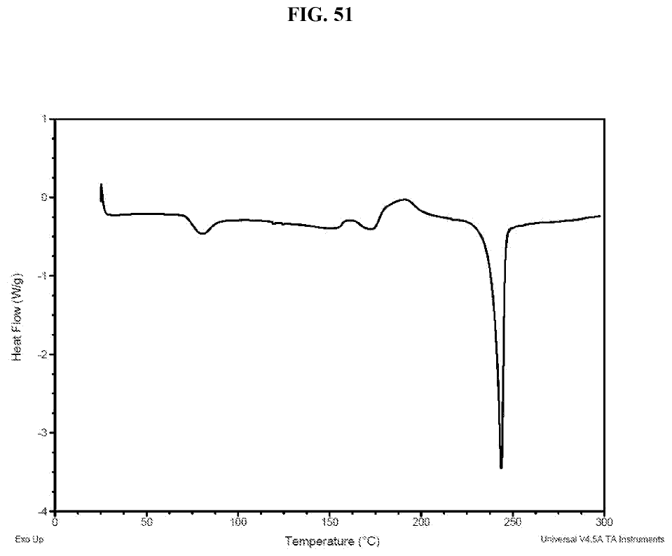

Thermogravimetric analysis of Compound 3 free form Hydrate Form C was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series' software and analyzed by Trios and/or Universal Analysis software (TA Instruments, New Castle, DE). The thermogram (FIG. 51) showed 4.2% weight loss from ambient temperature up to about 180° C.

### 4. Differential Scanning Calorimetry Analysis

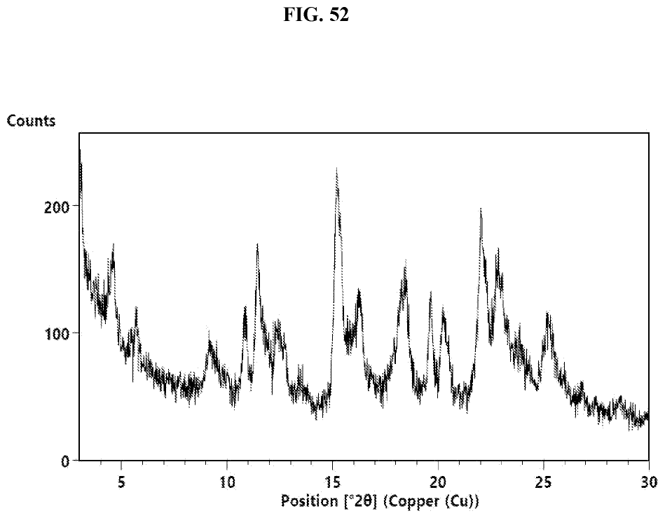

DSC of Compound 3 Free Form Hydrate Form C was measured using the TA Instruments Q2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 52) showed an endothermic peak at about 241° C.

### F. Compound 3 Free Form MTBE Solvate Form A

### 1. Synthetic Procedure

Compound 3 Free Form MTBE Solvate Form A was precipitated from triturating raw Compound 3 with MTBE, followed by dissolving isolated solids in THF at 50° C. for resin treatment, concentrating THF filtrate, and re-crystallizing from MTBE. The crystalline material was collected and determined to be Compound 3 free form MTBE Solvate Form A.

### 2. X-Ray Powder Diffraction

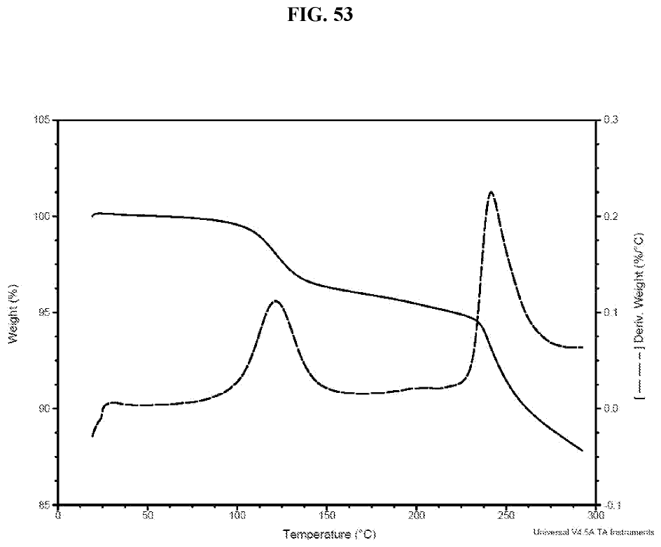

X-ray powder diffraction (XRPD) spectra results are shown in FIG. 53 and the table below.

| No. | Pos. [±0.2, °2θ] | Rel. Int. [%] |
|---|---|---|
| 1 | 15.6 | 100.0 |
| 2 | 22.1 | 37.5 |
| 3 | 17.9 | 33.2 |
| 4 | 19.7 | 31.1 |
| 5 | 9.1 | 26.9 |
| 6 | 13.6 | 26.2 |
| 7 | 10.2 | 22.1 |
| 8 | 17.0 | 19.5 |
| 9 | 19.4 | 19.1 |
| 10 | 14.2 | 18.2 |
| 11 | 18.5 | 18.0 |
| 12 | 10.7 | 17.8 |
| 13 | 17.0 | 16.5 |
| 14 | 9.6 | 15.4 |
| 15 | 19.1 | 13.9 |
| 16 | 25.2 | 12.8 |
| 17 | 22.7 | 11.3 |

### 3. Thermogravimetric Analysis

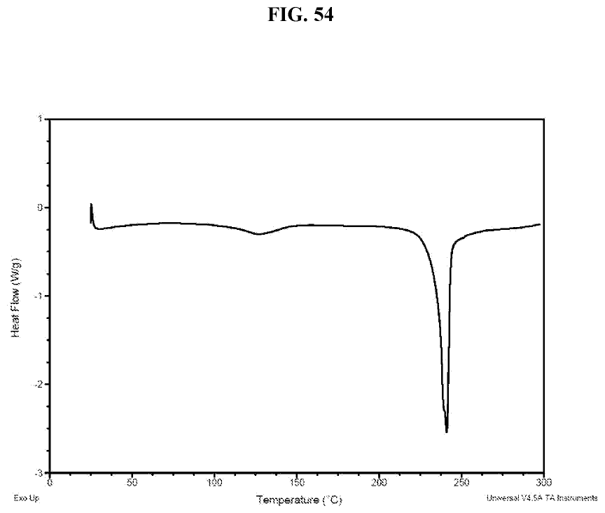

Thermogravimetric analysis of Compound 3 Free Form MTBE Solvate Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series' software and analyzed by Trios and/or Universal Analysis software (TA Instruments, New Castle, DE). The thermogram (FIG. 54) showed 14.7% weight loss from ambient temperature up to about 175° C.

### 4. Differential Scanning Calorimetry Analysis

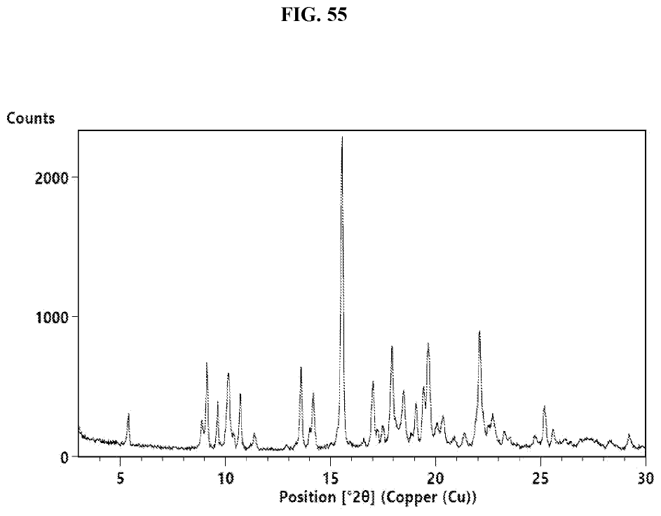

DSC of Compound 3 Free Form MTBE Solvate Form A was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 55) showed endothermic peaks at about 159° C. and 251° C., and an exothermic peak at about 201° C.

### 5. Solid State NMR

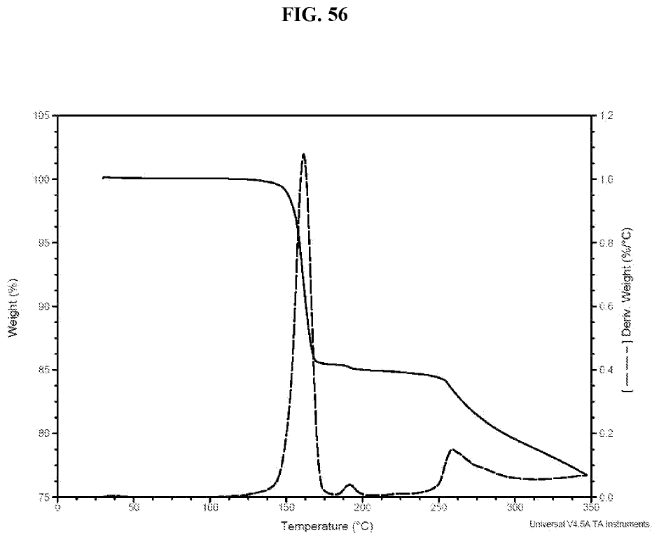

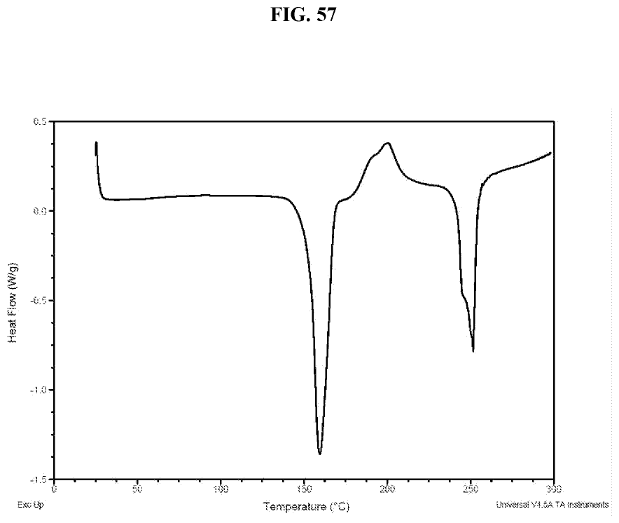

All carbon and fluorine spectra were recorded with proton decoupling using SPINAL64 decoupling sequence. The ¹³C CPMAS and ¹⁹F MAS on Compound 3 Free Form MTBE Solvate Form A results are shown in FIG. 56 and FIG. 57, respectively, and in the tables below:

¹³C CPMAS on Compound 3 free form MTBE Solvate Form A

| Peak # | Chem Shift [ppm] | Intensity [rel] |
|---|---|---|
| 1 | 169.6 | 1.6 |
| 2 | 167.4 | 2.6 |
| 3 | 153.0 | 1.1 |
| 4 | 150.5 | 1.6 |
| 5 | 147.2 | 2.9 |
| 6 | 143.6 | 2.3 |
| 7 | 142.2 | 3.2 |
| 8 | 140.0 | 3.2 |
| 9 | 139.1 | 2.9 |
| 10 | 135.6 | 3.8 |
| 11 | 131.0 | 10.0 |
| 12 | 129.1 | 5.1 |
| 13 | 125.2 | 4.3 |
| 14 | 124.5 | 4.4 |
| 15 | 121.5 | 2.5 |
| 16 | 117.2 | 3.6 |
| 17 | 115.5 | 3.5 |
| 18 | 114.0 | 3.3 |
| 19 | 110.7 | 0.8 |
| 20 | 109.5 | 1.0 |
| 21 | 96.7 | 2.7 |
| 22 | 81.6 | 1.2 |
| 23 | 78.2 | 3.4 |
| 24 | 76.6 | 4.7 |
| 25 | 75.2 | 3.1 |
| 26 | 60.1 | 2.7 |
| 27 | 58.5 | 1.8 |
| 28 | 57.0 | 1.0 |
| 29 | 55.1 | 4.6 |
| 30 | 48.1 | 5.7 |
| 31 | 35.1 | 1.3 |
| 32 | 34.4 | 1.4 |
| 33 | 30.6 | 1.3 |
| 34 | 29.4 | 2.0 |
| 35 | 25.9 | 7.8 |
| 36 | 8.3 | 3.6 |
| 37 | 7.2 | 4.2 |

¹⁹F MAS on Compound 3 free form MTBE Solvate Form A

| Peak # | Chem Shift [ppm | Intensity [rel] |
|---|---|---|
| 1 | −131.4 | 3.6 |
| 2 | −135.0 | 9.1 |
| 3 | −146.5 | 10.0 |

### Example 11—Solid Forms of Compound 4

### A. Compound 4 Free Form Form A

### 1. Synthetic Procedure

First, 207 mg of amorphous Compound 4 was placed in a 20 mL vial along with a stir bar, followed by adding 4 mL of 1:2 EtOH:water (v/v). The reaction was stirred at room temperature for 2 hours, followed by centrifuging to collect the solids as the seeds.

Second, 3.314 g of amorphous Compound 4 was placed in a 120 mL container along with a stir bar, followed by adding 60 mL of 1:2 EtOH:water (v/v). Seeds from the small-scale conversion was added into this reaction, which was later stirred at room temperature for 2 days. Solids were isolated via centrifugation and Compound 4 free form Form A was confirmed by XRPD.

### 2. X-Ray Powder Diffraction

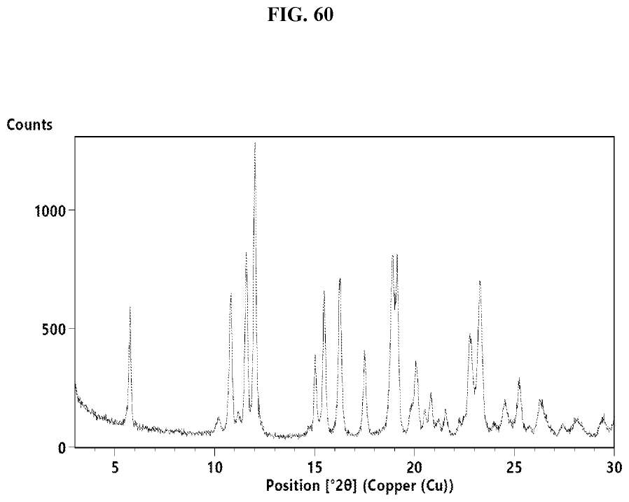

X-ray powder diffraction (XRPD) spectra results are shown in FIG. 60 and the table below.

XRPD Signals for Compound 4 Free Form Form A

| No | Pos. [±0.2, °2θ] | Rel. Int. [%] |
|---|---|---|
| 1 | 12.0 | 100.0 |
| 2 | 18.9 | 76.1 |
| 3 | 11.6 | 59.7 |
| 4 | 16.2 | 52.2 |
| 5 | 15.5 | 49.2 |
| 6 | 23.3 | 48.1 |
| 7 | 10.8 | 46.3 |
| 8 | 19.1 | 43.9 |
| 9 | 5.7 | 36.2 |
| 10 | 22.8 | 33.1 |
| 11 | 15.0 | 27.6 |
| 12 | 17.5 | 26.8 |
| 13 | 20.0 | 22.2 |
| 14 | 25.2 | 17.3 |
| 15 | 20.8 | 12.2 |
| 16 | 26.3 | 10.7 |
| 17 | 24.5 | 10.0 |

### 3. Thermogravimetric Analysis

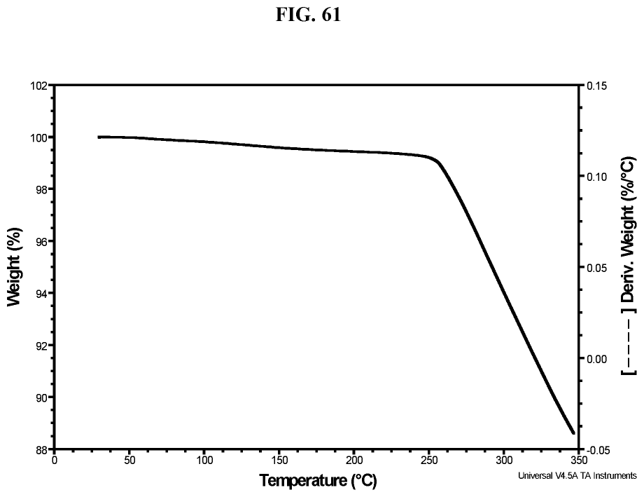

Thermal gravimetric analysis of Compound 4 free form Form A was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 61) showed 0.6% weight loss from ambient temperature up to 200° C.

### 4. Differential Scanning Calorimetry Analysis

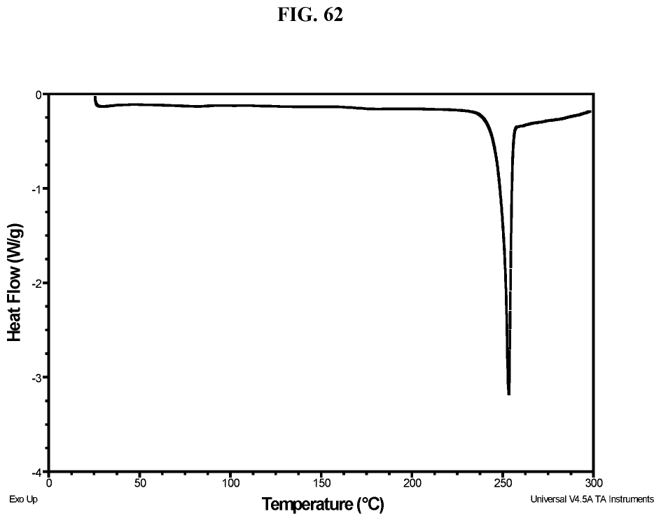

DSC of Compound 4 free form Form A was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 62) showed an endothermic peak around 253° C.

### 5. Solid State NMR

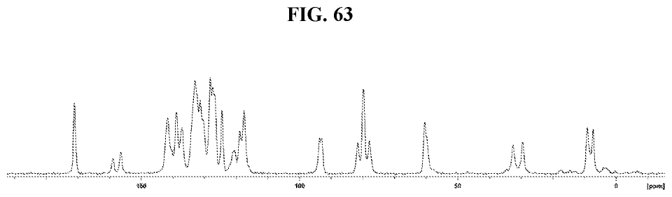

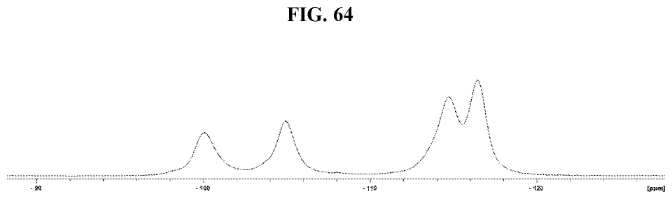

The ¹³C CPMAS and ¹⁹F MAS on Compound 4 free form Form A results are shown in FIG. 63 and FIG. 64, respectively, and in the tables below:

¹³C CPMAS on Compound 4 free form Form A

| Peak # | Chem Shift [ppm] | Intensity [rel] |
|---|---|---|
| 1 | 171.0 | 7.4 |
| 2 | 158.8 | 1.5 |
| 3 | 156.3 | 2.2 |
| 4 | 141.5 | 5.8 |
| 5 | 138.7 | 6.4 |
| 6 | 137.0 | 4.7 |
| 7 | 132.9 | 9.7 |
| 8 | 131.3 | 7.7 |
| 9 | 130.5 | 5.7 |
| 10 | 128.1 | 10.0 |
| 11 | 127.3 | 9.0 |
| 12 | 126.8 | 8.3 |
| 13 | 124.4 | 6.5 |
| 14 | 121.2 | 2.1 |
| 15 | 120.4 | 2.4 |
| 16 | 118.7 | 4.5 |
| 17 | 117.4 | 6.6 |
| 18 | 93.6 | 3.7 |
| 19 | 92.9 | 3.7 |
| 20 | 81.5 | 3.2 |
| 21 | 79.7 | 8.8 |
| 22 | 77.8 | 3.4 |
| 23 | 60.3 | 5.4 |
| 24 | 59.9 | 4.0 |
| 25 | 32.5 | 3.0 |
| 26 | 29.4 | 3.3 |
| 27 | 9.0 | 4.8 |
| 28 | 7.1 | 4.6 |

¹⁹F MAS on Compound 4 free form Form A

| Peak # | Chem Shift [ppm] | Intensity [rel] |
|---|---|---|
| 1 | −100.1 | 4.5 |
| 2 | −105.0 | 5.7 |
| 3 | −114.7 | 8.2 |
| 4 | −116.5 | 10.0 |

### 6. Single Crystal Elucidation

A single crystal having the Compound 4 free form Form A structure was obtained from an acetone solution of Compound 4. X-ray diffraction data were acquired at 100K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122) and results are summarized in the table below.

Single Crystal Elucidation of Compound 4 free form Form A

| Crystal System | Monoclinic |
|---|---|
| Space Group | P2₁ |
| a (Å) | 15.3269(5) |
| b (Å) | 9.1535(3) |
| c (Å) | 17.1006(5) |
| α (°) | 90 |
| β (°) | 97.1785(17) |
| γ (°) | 90 |
| V (Å³) | 2380.32(13) |
| Z/Z′ | 2/2 |
| Temperature (K) | 100(2) |

### B. Compound 4 Free Form Form B

### 1. Synthetic Procedure

Crude Compound 4 solid was treated with 500 mL water and acidified with aqueous 3 N HCl until pH at 4, followed by adding with 1 L ethyl acetate and stirring for 20 minutes. The organic phase was separated, and the aqueous phase was extracted with 1 L ethyl acetate. The combined organic phase was sequentially washed with water (800 mL), brine (˜800 mL), dried over MgSO4, filtered and concentrated under reduced pressure to afford desired product as an oil. The crude residue was treated with methylene chloride (1 L) and concentrated under reduced pressure to afford desired product as a tan colored crispy foam. Foam was treated with methylene chloride (2 L), heated to reflux for 4 h, and then stirred at room temperature for 1 hour. Suspension was filtered, and the collected solid was washed with DCM (500 mL) and dried under air suction for 30 min. The collected solid was further dried in vacuum oven at 80° C. for 3 days followed by 100° C. for 12 hours to remove DCM.

### 2. X-Ray Powder Diffraction

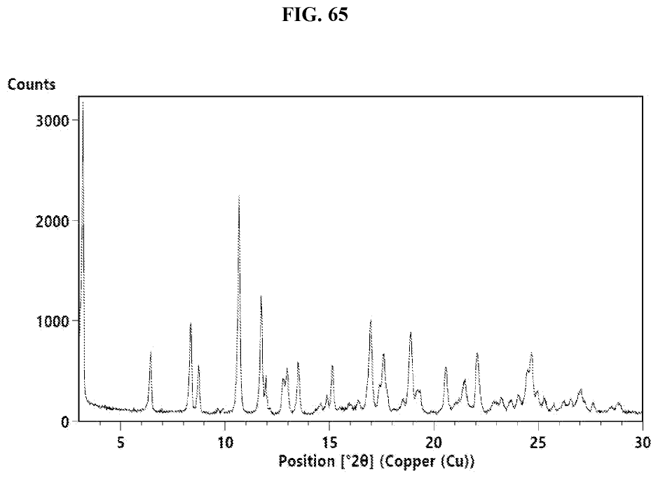

XRPD spectra results for Compound 4 free form Form B are shown in FIG. 65 and the table below.

XRPD Signals for Compound 4 Free Form Form B

| No. | Pos. [±0.2, °2θ] | Rel. Int. [%] |
|---|---|---|
| 1 | 3.2 | 100.0 |
| 2 | 10.7 | 86.2 |
| 3 | 11.7 | 49.2 |
| 4 | 8.3 | 39.2 |
| 5 | 16.9 | 38.7 |
| 6 | 18.9 | 34.3 |
| 7 | 24.7 | 24.8 |
| 8 | 6.4 | 24.8 |
| 9 | 22.1 | 23.7 |
| 10 | 13.5 | 21.5 |
| 11 | 17.6 | 21.4 |
| 12 | 15.1 | 21.1 |
| 13 | 20.6 | 20.1 |
| 14 | 13.0 | 19.1 |
| 15 | 8.7 | 18.5 |
| 16 | 24.5 | 16.5 |
| 17 | 11.9 | 14.4 |
| 18 | 12.8 | 13.8 |
| 19 | 21.4 | 12.9 |

### 3. Thermogravimetric Analysis

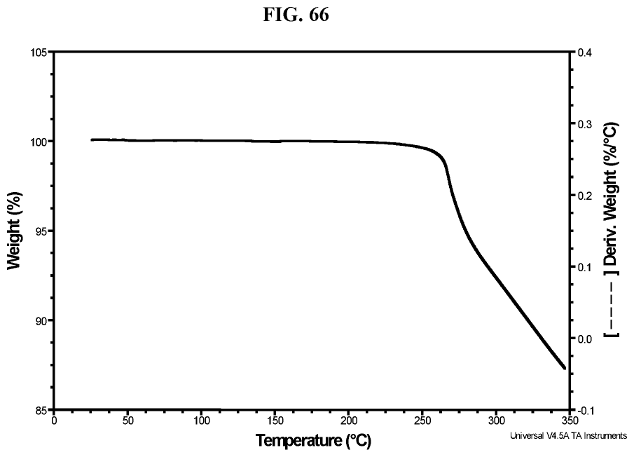

Thermal gravimetric analysis of Compound 4 Free Form Form B was measured using TA Discovery 550 TGA from TA Instrument. A sample with weight of approximately 1-10 mg was scanned from 25° C. to 350° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 66) showed minimal weight loss from ambient temperature up to 200° C.

### 4. Differential Scanning Calorimetry Analysis

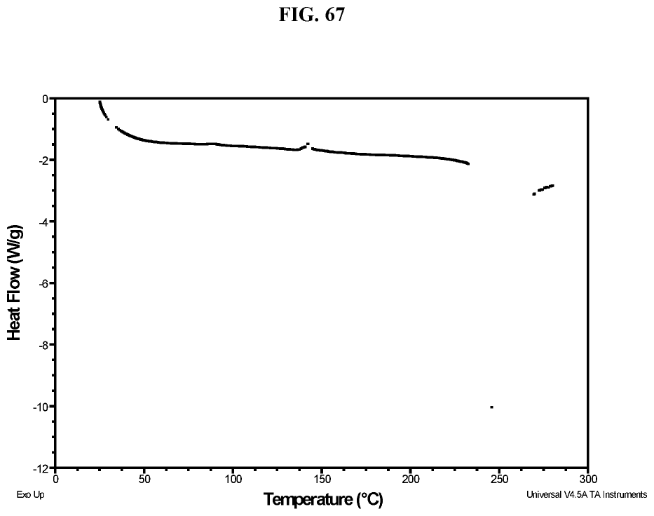

DSC of Compound 4 Free Form Form B was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 67) showed an endothermic peak around 246° C.

### 5. Solid State NMR

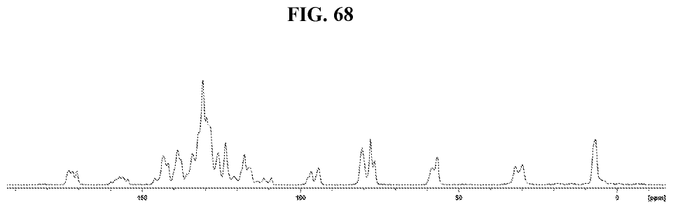

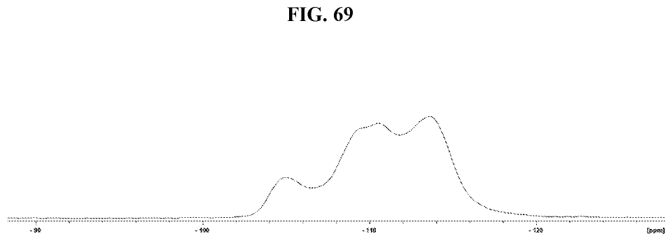

The ¹³C CPMAS and ¹⁹F MAS on Compound 4 free form Form B results are shown in FIG. 68 and FIG. 69, respectively, and in the tables below:

¹³C CPMAS on Compound 4 free form Form B

| Peak # | Chem Shift [ppm] | Intensity [rel] |
|---|---|---|
| 1 | 173.0 | 1.4 |
| 2 | 171.9 | 1.3 |
| 3 | 170.5 | 1.3 |
| 4 | 159.8 | 0.3 |
| 5 | 158.3 | 0.6 |
| 6 | 157.0 | 0.8 |
| 7 | 156.0 | 0.8 |
| 8 | 154.5 | 0.6 |
| 9 | 145.9 | 0.6 |
| 10 | 143.3 | 2.8 |
| 11 | 141.7 | 2.1 |
| 12 | 140.1 | 1.1 |
| 13 | 138.7 | 3.4 |
| 14 | 137.7 | 2.4 |
| 15 | 135.7 | 1.0 |
| 16 | 134.0 | 3.0 |
| 17 | 130.7 | 10.0 |
| 18 | 129.5 | 6.4 |
| 19 | 128.9 | 5.8 |
| 20 | 125.9 | 3.1 |
| 21 | 123.6 | 4.0 |
| 22 | 120.8 | 0.8 |
| 23 | 117.6 | 2.9 |
| 24 | 116.1 | 1.7 |
| 25 | 113.8 | 0.4 |
| 26 | 111.5 | 0.6 |
| 27 | 109.1 | 0.7 |
| 28 | 97.4 | 0.8 |
| 29 | 96.5 | 1.4 |
| 30 | 94.2 | 1.6 |
| 31 | 80.4 | 3.6 |
| 32 | 77.8 | 4.4 |
| 33 | 76.5 | 2.3 |
| 34 | 58.5 | 1.7 |
| 35 | 56.7 | 2.7 |
| 36 | 32.0 | 1.8 |
| 37 | 29.8 | 1.9 |
| 38 | 6.7 | 4.4 |

¹⁹F MAS on Compound 4 free form Form B

| Peak # | Chem Shift [ppm] | Intensity [rel] |
|---|---|---|
| 1 | −105.0 | 4.0 |
| 2 | −109.4 | 8.8 |
| 3 | −110.5 | 9.3 |
| 4 | −113.6 | 10.0 |

### C. Compound 4 Free Form Form C

### 1. Synthetic Procedure

About 30 mg of Compound 4 free form Form B was suspended in 0.2 mL of MTBE in a 2 mL glass vial. After the suspension was stirred for 3 days at 50° C., the solids were isolated and heated at 200° C. until all MTBE was dried out to provide Compound 4 free form Form C.

### 2. X-Ray Powder Diffraction

XRPD was performed with a Panalytical X′Pert³ Powder XRPD on a Si zero-background holder. The 20 position was calibrated against a Panalytical Si reference standard disc. The parameters used are listed in the table below.

Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

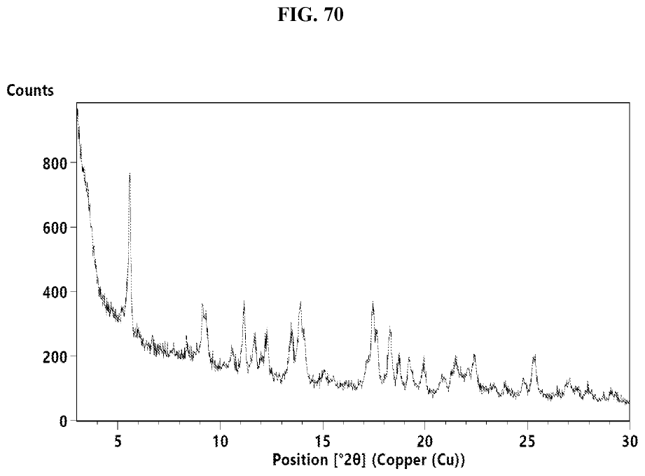

XRPD spectra results for Compound 4 free form Form C are shown in FIG. 70 and the table below.

XRPD Signals for Compound 4 Free Form Form C

| No. | Pos. [±0.2, °2θ] | Rel. Int. [%] |
|---|---|---|
| 1 | 5.6 | 100.0 |
| 2 | 17.4 | 56.8 |
| 3 | 13.9 | 50.3 |
| 4 | 11.1 | 45.2 |
| 5 | 9.2 | 36.4 |
| 6 | 18.3 | 34.5 |
| 7 | 25.3 | 28.0 |
| 8 | 12.2 | 26.9 |
| 9 | 11.7 | 24.9 |
| 10 | 13.4 | 24.2 |
| 11 | 22.2 | 17.0 |
| 12 | 17.6 | 16.3 |
| 13 | 19.2 | 16.0 |
| 14 | 18.7 | 14.4 |
| 15 | 21.5 | 14.4 |
| 16 | 10.6 | 14.2 |
| 17 | 19.9 | 11.8 |
| 18 | 24.8 | 11.2 |

### 3. Thermogravimetric Analysis

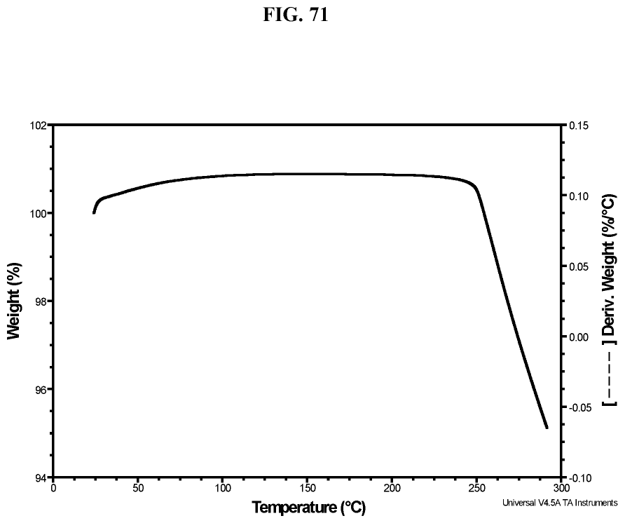

Thermal gravimetric analysis of Compound 4 free form Form C was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 71) showed negligible weight loss from ambient temperature up to 200° C.

### 4. Differential Scanning Calorimetry Analysis

DSC of Compound 4 Free Form Form C was measured using a TA D2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram showed an endothermic peak around 252° C.

### D. Compound 4 Free Form Form D

### 1. Synthetic Procedure

About 30 mg of amorphous Compound 4 was suspended in 0.2 mL of n-propyl acetate in a 2-mL glass vial. After stirring the suspension at 5° C. for 4 days, the solids were isolated, followed by drying at 150° C. until all n-propyl acetate was dried out to provide Compound 4 free form Form D.

### 2. X-Ray Powder Diffraction

XRPD was performed with a Panalytical X′Pert³ Powder XRPD on a Si zero-background holder. The 20 position was calibrated against a Panalytical Si reference standard disc. The parameters used are listed in the following table.

Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

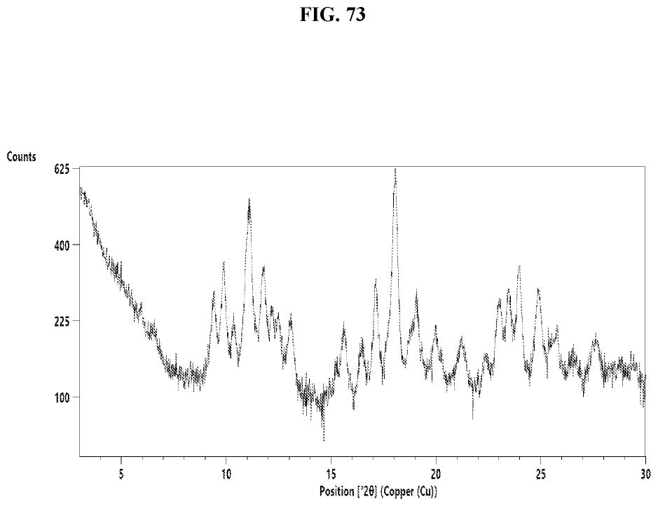

XRPD spectra results for Compound 4 free form Form D are shown in FIG. 73 and the table below.

XRPD Signals for Compound 4 Free Form Form D

| No. | Pos. [±0.2, °2θ] | Rel. Int. [%] |
|---|---|---|
| 1 | 18.0 | 100.0 |
| 2 | 11.1 | 74.2 |
| 3 | 11.8 | 39.5 |
| 4 | 23.9 | 37.1 |
| 5 | 17.1 | 35.7 |
| 6 | 9.9 | 35.7 |
| 7 | 19.0 | 29.1 |
| 8 | 24.9 | 26.9 |
| 9 | 23.5 | 26.6 |
| 10 | 9.4 | 25.6 |
| 11 | 23.0 | 22.2 |
| 12 | 13.1 | 19.8 |
| 13 | 12.4 | 18.5 |
| 14 | 12.2 | 16.5 |
| 15 | 20.0 | 15.9 |
| 16 | 15.6 | 14.7 |
| 17 | 10.3 | 14.0 |
| 18 | 25.6 | 12.0 |
| 19 | 21.2 | 11.8 |
| 20 | 27.6 | 10.4 |

### 3. Thermogravimetric Analysis

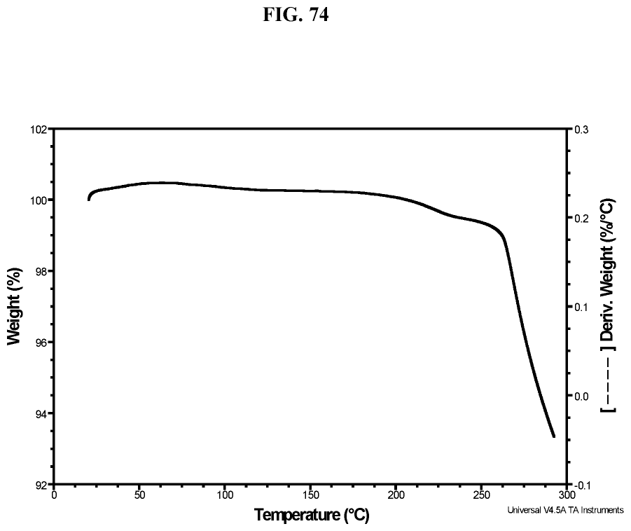

Thermal gravimetric analysis of Compound 4 free form Form D was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 74) showed negligible weight loss from ambient temperature up to 200° C.

### 4. Differential Scanning Calorimetry Analysis

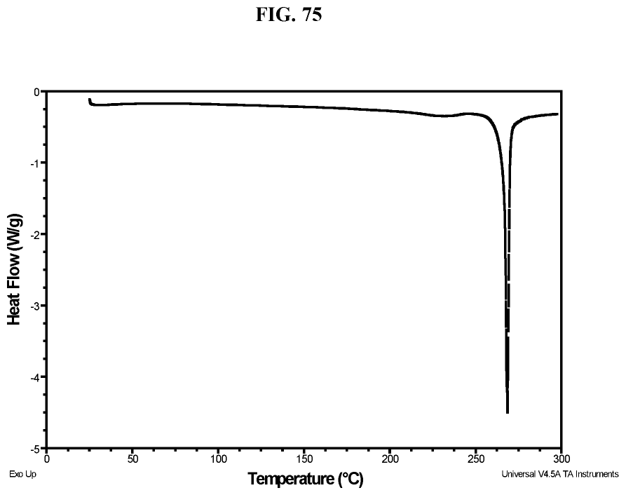

DSC of Compound 4 free form Form D was measured using a TA D2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 75) showed an endothermic peak around 268° C.

### 5. Solid State NMR

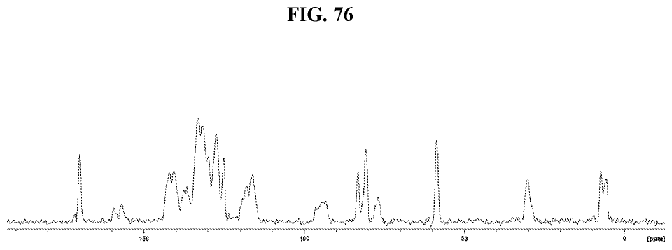

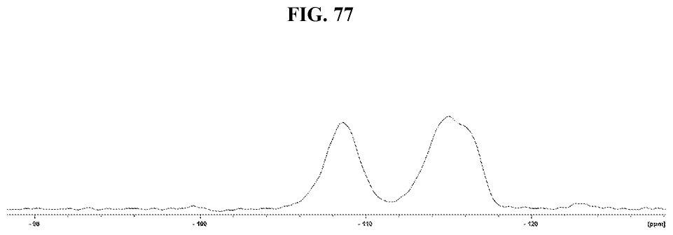

The ¹³C CPMAS and ¹⁹F MAS on Compound 4 free form Form D results are shown in FIG. 76 and FIG. 77, respectively, and in the tables below:

¹³C CPMAS on Compound 4 free form Form D

| Peak # | Chem Shift [ppm] | Intensity [rel] |
|---|---|---|
| 1 | 170.0 | 6.5 |
| 2 | 159.3 | 1.4 |
| 3 | 156.8 | 1.8 |
| 4 | 142.9 | 3.7 |
| 5 | 141.9 | 4.8 |
| 6 | 140.6 | 4.9 |
| 7 | 137.4 | 3.1 |
| 8 | 136.5 | 3.5 |
| 9 | 132.9 | 10.0 |
| 10 | 131.8 | 9.3 |
| 11 | 129.8 | 6.3 |
| 12 | 127.4 | 8.4 |
| 13 | 125.0 | 6.2 |
| 14 | 119.1 | 2.3 |
| 15 | 117.9 | 3.5 |
| 16 | 116.0 | 4.5 |
| 17 | 96.3 | 1.4 |
| 18 | 94.9 | 1.7 |
| 19 | 94.0 | 2.0 |
| 20 | 93.2 | 2.0 |
| 21 | 83.1 | 4.8 |
| 22 | 80.6 | 7.0 |
| 23 | 77.8 | 1.4 |
| 24 | 76.9 | 2.5 |
| 25 | 58.5 | 7.9 |
| 26 | 30.2 | 4.2 |
| 27 | 29.0 | 1.6 |
| 28 | 7.3 | 4.9 |
| 29 | 5.7 | 4.1 |

¹⁹F MAS on Compound 4 free form Form D

| Peak # | Chem Shift [ppm] | Intensity [rel] |
|---|---|---|
| 1 | −108.6 | 9.3 |
| 2 | −115.0 | 10.0 |
| 3 | −115.9 | 9.0 |

### E. Compound 4 Free Form Hydrate Form A

### 1. Synthetic Procedure

First, 40 mg of amorphous Compound 4 was dispensed into 1.5 mL glass vial, followed by dispensing 0.4 ml water. The vial was crimped sealed and allowed to shake on a shaker block at 800 rpms for 2 weeks. The solid was isolated as the seeds.

Second, about 60 mg Compound 4 free form Form B and 0.3 ml deionized water were mixed in 1.5 mL glass vial, bath sonicated for 3 min, followed by adding the seed and shaking on 30° C. shaker overnight. 0.2 ml more water was added to the vial and kept shaken on 30° C. shaker for 4 more days until the solids were isolated for analysis.

### 2. X-Ray Powder Diffraction

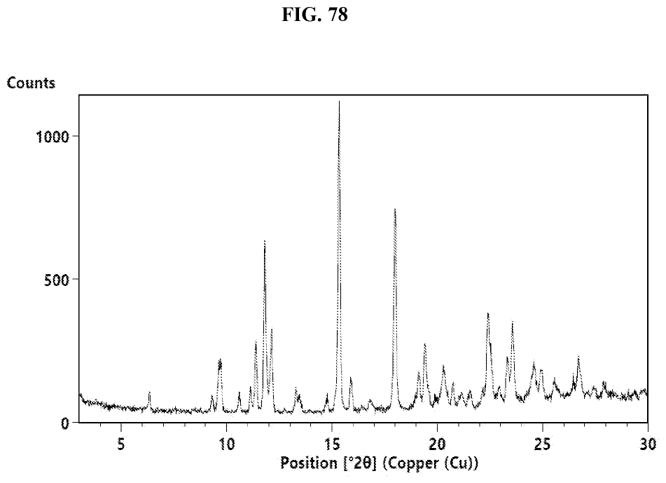

XRPD spectra results for Compound 4 free form Hydrate Form A are shown in FIG. 78 and the table below.

XRPD Signals for Compound 4 free form Hydrate Form A

| No. | Pos. [±0.2, °2θ] | Rel. Int. [%] |
|---|---|---|
| 1 | 15.3 | 100.0 |
| 2 | 18.0 | 66.0 |
| 3 | 11.8 | 49.2 |
| 4 | 23.6 | 27.1 |
| 5 | 22.4 | 26.9 |
| 6 | 12.1 | 23.4 |
| 7 | 11.4 | 22.1 |
| 8 | 19.4 | 21.5 |
| 9 | 9.7 | 16.5 |
| 10 | 23.3 | 13.8 |
| 11 | 20.3 | 12.0 |
| 12 | 19.1 | 11.8 |
| 13 | 24.5 | 11.3 |
| 14 | 26.7 | 10.6 |
| 15 | 15.9 | 10.1 |

### 3. Thermogravimetric Analysis

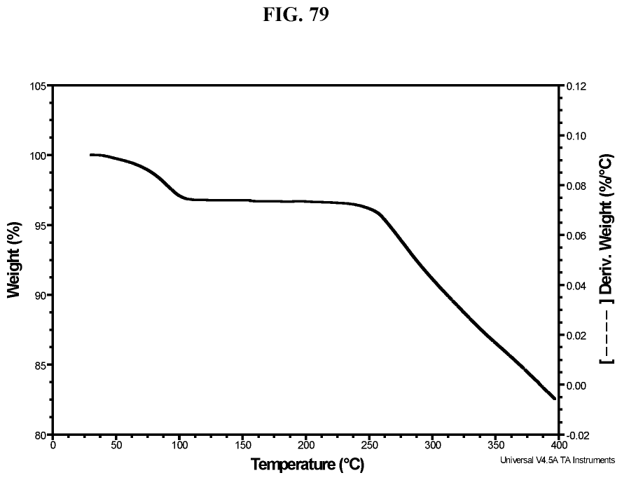

Thermal gravimetric analysis of Compound 4 free form Hydrate Form A was measured using TA Discovery 550 TGA from TA Instrument. A sample with weight of approximately 1-10 mg was scanned from 25° C. to 400° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 79) showed 3.3% weight loss from ambient temperature up to 200° C.

### 4. Differential Scanning Calorimetry Analysis

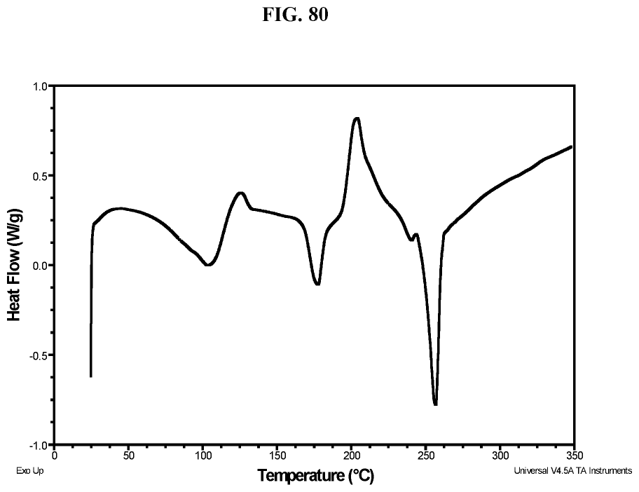

DSC of Compound 4 free form Hydrate Form A measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 350° C. When the run was completed, the data were analyzed by Trios and/or Universal Analysis software (TA Instruments, New Castle, DE). The thermogram (FIG. 80) showed endothermic peaks around 103, 178, and 257° C. and exothermic peaks around 126 and 204° C.

### 5. Solid State NMR

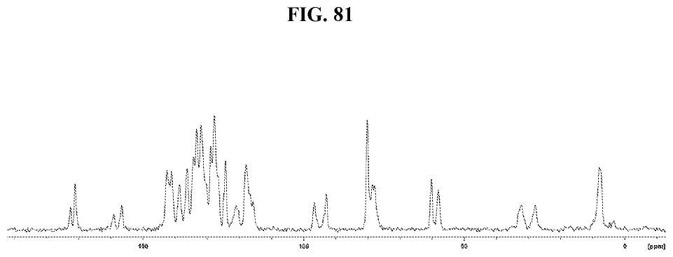

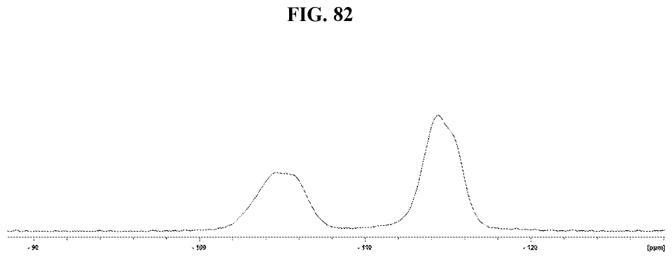

The ¹³C CPMAS and ¹⁹F MAS on Compound 4 free form Hydrate Form A results are shown in FIG. 81 and FIG. 82, respectively, and in the tables below:

¹³C CPMAS on Compound 4 free form Hydrate Form A

| Peak # | Chem Shift [ppm] | Intensity [rel] |
|---|---|---|
| 1 | 172.5 | 2.0 |
| 2 | 171.0 | 4.0 |
| 3 | 159.0 | 1.4 |
| 4 | 156.5 | 2.2 |
| 5 | 142.4 | 5.2 |
| 6 | 141.0 | 5.1 |
| 7 | 138.6 | 4.0 |
| 8 | 136.2 | 5.3 |
| 9 | 134.1 | 6.4 |
| 10 | 133.2 | 8.8 |
| 11 | 131.8 | 9.1 |
| 12 | 130.6 | 4.2 |
| 13 | 128.8 | 7.3 |
| 14 | 127.7 | 10.0 |
| 15 | 126.7 | 4.7 |
| 16 | 124.2 | 6.0 |
| 17 | 120.9 | 2.1 |
| 18 | 120.6 | 1.8 |
| 19 | 117.9 | 5.7 |
| 20 | 116.9 | 3.2 |
| 21 | 115.6 | 2.5 |
| 22 | 96.6 | 2.4 |
| 23 | 92.8 | 3.2 |
| 24 | 80.1 | 9.6 |
| 25 | 78.5 | 3.9 |
| 26 | 77.8 | 3.9 |
| 27 | 60.1 | 4.5 |
| 28 | 58.0 | 3.5 |
| 29 | 33.0 | 1.5 |
| 30 | 32.2 | 2.1 |
| 31 | 27.8 | 2.2 |
| 32 | 7.9 | 5.5 |
| 33 | 7.4 | 5.2 |

¹⁹F MAS on Compound 4 free form Hydrate Form A

| Peak # | Chem Shift [ppm] | Intensity [rel] |
|---|---|---|
| 1 | −104.7 | 5.0 |
| 2 | −105.5 | 4.9 |
| 3 | −114.4 | 10.0 |
| 4 | −115.2 | 8.5 |

### F. Compound 4 Free Form Hydrate Form B

### 1. Synthetic Procedure

About 20 mg of Compound 4 free form Form B was suspended in 0.2 mL of acetone/water mixture (0.604/0.396 V/V) in a 2-mL glass vial. The slurry sample was kept at room temperature under constant stirring. After 3 days of stirring the solids were isolated for analysis.

### 2. X-Ray Powder Diffraction

XRPD was performed with a Panalytical X′Pert³ Powder XRPD on a Si zero-background holder. The 2θ position was calibrated against a Panalytical Si reference standard disc. The parameters used are listed in the following table.

Table of Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

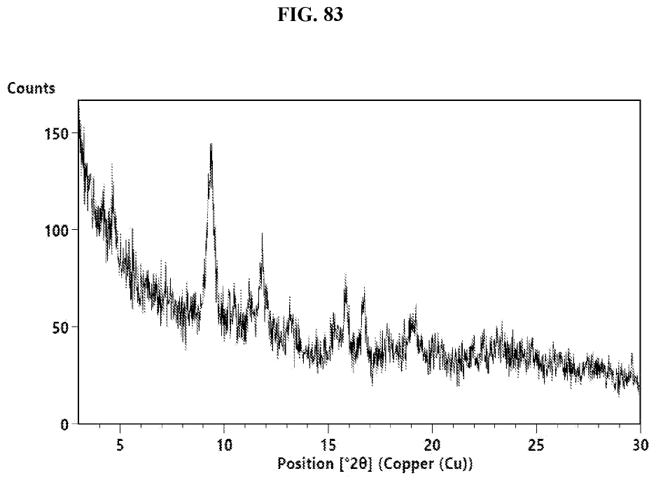

XRPD spectra results for Compound 4 free form Hydrate Form B are shown in FIG. 83 and the table below.

XRPD Signals for Compound 4 free form Hydrate Form B

| No. | Pos. [±0.2, °2θ] | Rel. Int. [%] |
|---|---|---|
| 1 | 9.4 | 100.0 |
| 2 | 11.8 | 46.8 |
| 3 | 15.8 | 40.0 |
| 4 | 16.7 | 34.6 |
| 5 | 19.1 | 21.2 |
| 6 | 15.3 | 14.3 |
| 7 | 13.2 | 14.1 |

### 3. Thermogravimetric Analysis

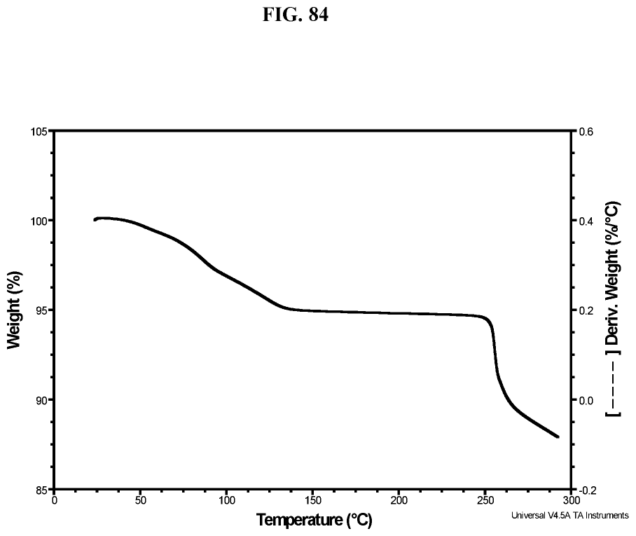

Thermal gravimetric analysis of Compound 4 free form Hydrate Form B was measured using TA Discovery 550 TGA from TA Instrument. A sample with weight of approximately 1-10 mg was scanned from 25° C. to 300° C. at a heating rate of 10° C./min with nitrogen purge. Data were collected by Thermal Advantage Q Series™ software and analyzed by Trios and/or Universal Analysis software (TA Instruments, New Castle, DE). The thermogram (FIG. 84) showed 5.2% weight loss from ambient temperature up to 200° C.

### 4. Differential Scanning Calorimetry Analysis

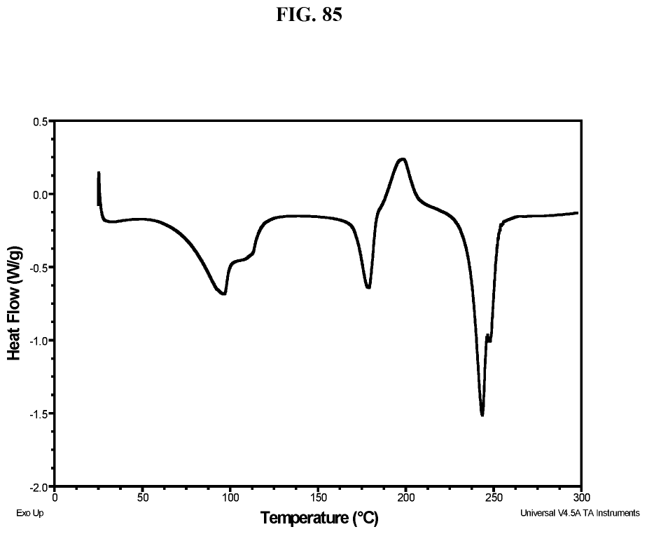

DSC of Compound 4 free form Hydrate Form B was measured using the TA Discovery 550 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 85) showed endothermic peaks around 96, 179, and 244° C. and exothermic peaks around 198° C.

### G. Compound 4 Free Form Hydrate Form C

### 1. Synthetic Procedure

About 30 mg of Compound 4 free form Form B was suspended in 0.2-0.3 mL of chlorobenzene or methyl cyclohexane in a 2-mL glass vial. After stirring the suspension at RT for 8 days, solids were isolated for analysis.

### 2. X-Ray Powder Diffraction

XRPD was performed with a Panalytical X′Pert³ Powder XRPD on a Si zero-background holder. The 20 position was calibrated against a Panalytical Si reference standard disc. The parameters used are listed in the following table.

Table of Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2TH) | 3-40 |
| Scan step time [s] | 18.87 |
| Step size (°2TH) | 0.0131 |
| Test Time | 4 min 15 s |

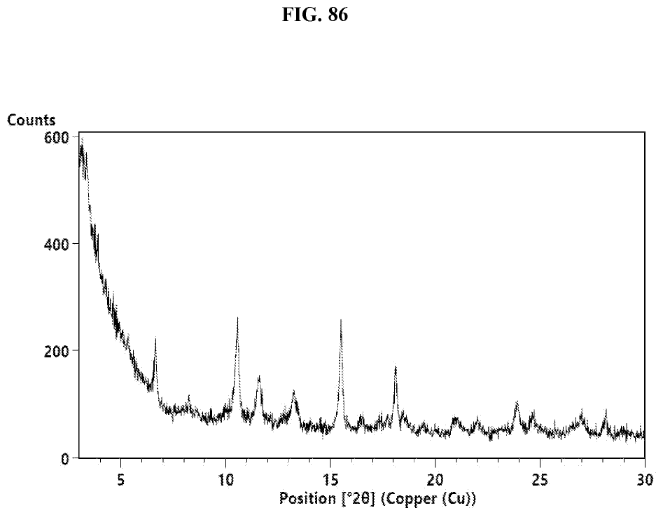

XRPD spectra results for Compound 4 free form Hydrate Form C are shown in FIG. 86 and the table below.

XRPD Signals for Compound 4 free form Hydrate Form C

| No. | Pos. [±0.2, °2θ] | Rel. Int. [%] |
|---|---|---|
| 1 | 15.5 | 100.0 |
| 2 | 10.5 | 99.3 |
| 3 | 6.6 | 69.0 |
| 4 | 3.0 | 54.5 |
| 5 | 11.6 | 52.4 |
| 6 | 18.1 | 47.6 |
| 7 | 13.2 | 36.4 |
| 8 | 23.9 | 30.9 |
| 9 | 28.1 | 22.8 |
| 10 | 24.6 | 17.7 |
| 11 | 21.0 | 14.9 |
| 12 | 26.8 | 13.4 |
| 13 | 22.0 | 11.5 |

### 3. Differential Scanning Calorimetry Analysis

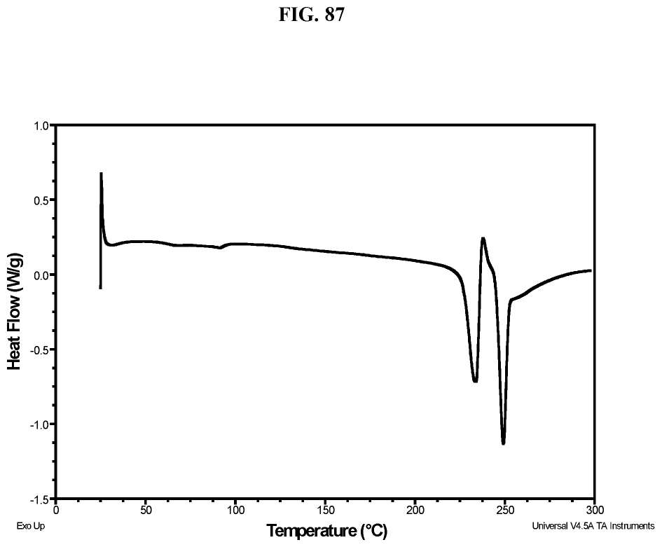

DSC of Compound 4 free form Hydrate Form C was measured using a TA D2500 DSC. A sample with a weight between 1-10 mg was weighed into an aluminum crimp sealed pan with a pinhole. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram (FIG. 87) showed endothermic peaks around 234 and 249° C.

### OTHER EMBODIMENTS

This description provides merely exemplary embodiments of the disclosed subject matter. One skilled in the art will readily recognize from the disclosure and accompanying claims, that various changes, modifications and variations can be made therein without departing from the spirit and scope of the disclosure as defined in the following claims.

What is claimed is:

1. A compound of Formula I: 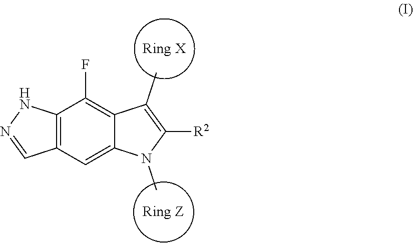
or a tautomer thereof, a deuterated derivative of the compound or tautomer, or a pharmaceutically acceptable salts of the compound, tautomer, or deuterated derivative, wherein:
Ring X is selected from: 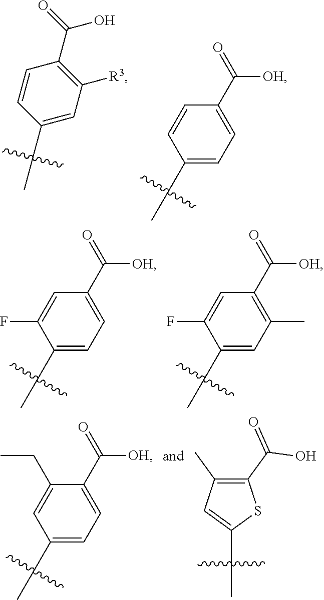
Ring Z is selected from: 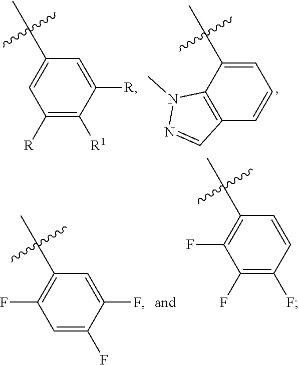
Each R is independently selected from: F, H, Cl, —CH₃, —OCH₃, and —OCD₃;
R¹ is H or F
R² is selected from: 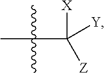
wherein
one of X, Y, and Z is —OH;
one of X, Y, and Z is chosen from —CH₂CH₃ and cyclopropyl;
and one of X, Y, and Z is chosen from —CH₂OCH₃, —CH₂CH₂OCH₃, —CH₂OCH₂CH₃, —CH₂O-cyclopropyl, and —CH₂O-isopropyl; 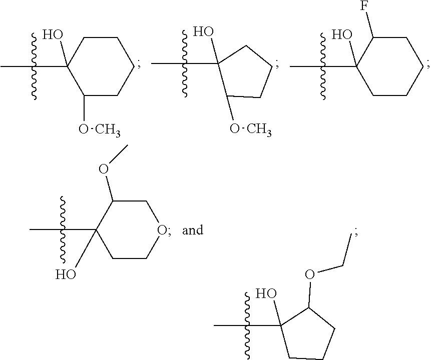
and
R³ is chosen from H and —CH₃.

2. The compound, tautomer, deuterated derivative, or pharmaceutically acceptable salt according to claim 1, wherein the compound of Formula I is selected from compounds of Formula Ia: 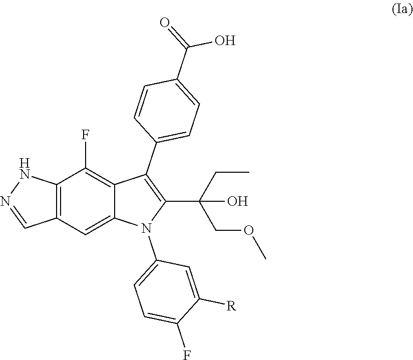
and tautomers thereof, deuterated derivatives of those compounds or tautomers, and pharmaceutically acceptable salts of the foregoing compounds, tautomers, and deuterated derivatives, wherein R is selected from F, H, Cl, CH₃, and OCH₃.

3. The compound, tautomer, deuterated derivative, or pharmaceutically acceptable salt according to claim 2 wherein the compound of Formula Ia is selected from:
Compound 1 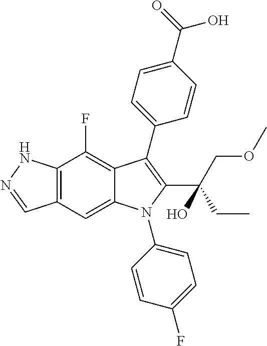 [SMILES: CC[C@@](O)(COC)c1c(-c2ccc(C(=O)O)cc2)c2c(F)c3[nH]ncc3cc2n1-c1ccc(F)cc1]
Compound 2 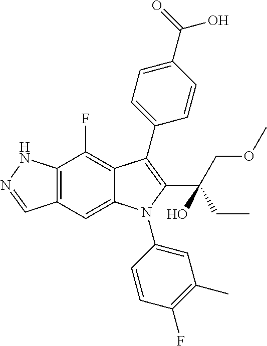 [SMILES: CC[C@@](O)(COC)c1c(-c2ccc(C(=O)O)cc2)c2c(F)c3[nH]ncc3cc2n1-c1ccc(F)c(C)c1]
Compound 3 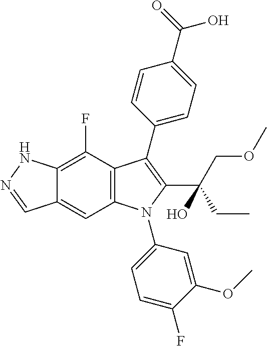 [SMILES: CC[C@@](O)(COC)c1c(-c2ccc(C(=O)O)cc2)c2c(F)c3[nH]ncc3cc2n1-c1ccc(F)c(OC)c1]
Compound 4 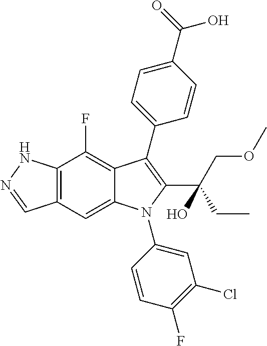 [SMILES: CC[C@@](O)(COC)c1c(-c2ccc(C(=O)O)cc2)c2c(F)c3[nH]ncc3cc2n1-c1ccc(F)c(Cl)c1]
Compound 5 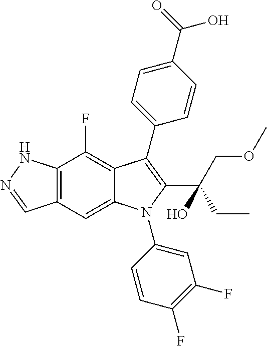 [SMILES: CC[C@@](O)(COC)c1c(-c2ccc(C(=O)O)cc2)c2c(F)c3[nH]ncc3cc2n1-c1ccc(F)c(F)c1]
and tautomers of those compounds, deuterated derivatives of those compounds and tautomers, and pharmaceutically acceptable salts of those compounds, tautomers, and deuterated derivatives.

4. The compound, tautomer, deuterated derivative, or pharmaceutically acceptable salt according to claim 1, wherein the compound of Formula I is selected from compounds of Formula Ib: 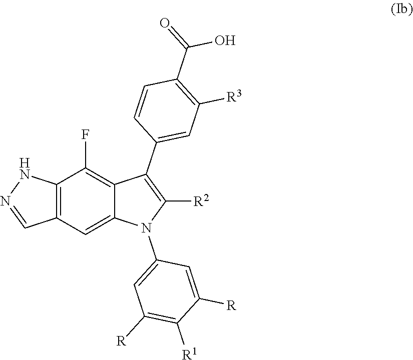
and tautomers thereof, deuterated derivatives of those compounds or tautomers, and pharmaceutically acceptable salts of the foregoing compounds, tautomers, and deuterated derivatives, wherein:
each R is independently selected from F, H, Cl, CH₃, OCH₃, and OCD₃;
R¹ is H or F;
R² is selected from 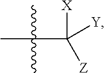
wherein
one of X, Y, and Z is OH;
one of X, Y, and Z is chosen from CH₂CH₃ and cyclopropyl;
and one of X, Y, and Z is chosen from CH₂OCH₃, CH₂CH₂OCH₃, CH₂OCH₂CH₃, CH₂O-cyclopropyl, and CH₂O-isopropyl; and 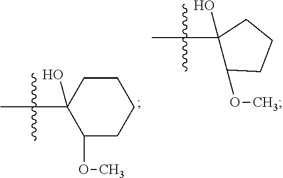
and
R³ is chosen from H and CH₃.

5. The compound, tautomer, deuterated derivative, or pharmaceutically acceptable salt according to claim 4 wherein the compound of Formula Ib is selected from:
Compound 6 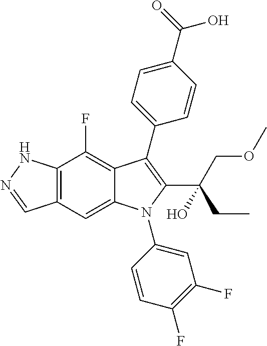 [SMILES: CC[C@](O)(COC)c1c(-c2ccc(C(=O)O)cc2)c2c(F)c3[nH]ncc3cc2n1-c1ccc(F)c(F)c1]
Compound 7 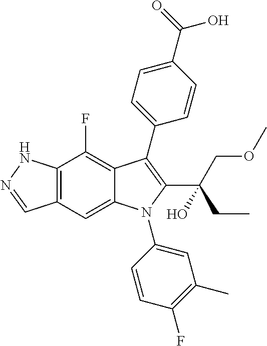 [SMILES: CC[C@](O)(COC)c1c(-c2ccc(C(=O)O)cc2)c2c(F)c3[nH]ncc3cc2n1-c1ccc(F)c(C)c1]
Compound 8 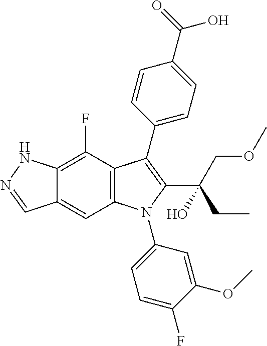 [SMILES: CC[C@](O)(COC)c1c(-c2ccc(C(=O)O)cc2)c2c(F)c3[nH]ncc3cc2n1-c1ccc(F)c(OC)c1]
Compound 9 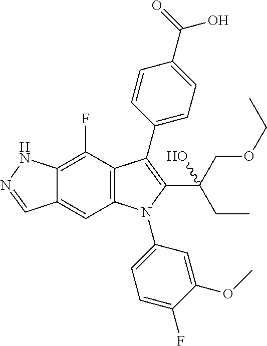 [SMILES: CCOCC(O)(CC)c1c(-c2ccc(C(=O)O)cc2)c2c(F)c3[nH]ncc3cc2n1-c1ccc(F)c(OC)c1]
Compound 10 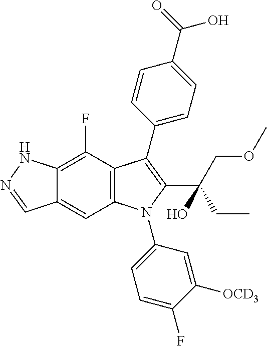
Compound 11 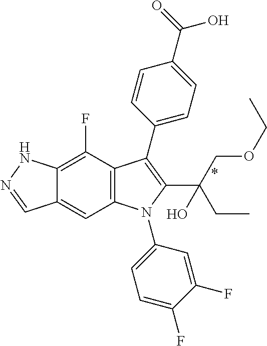 [SMILES: CCOCC(O)(CC)c1c(-c2ccc(C(=O)O)cc2)c2c(F)c3[nH]ncc3cc2n1-c1ccc(F)c(F)c1]
Compound 12 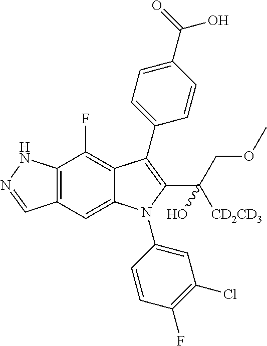
Compound 13 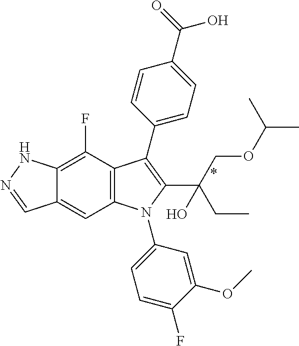 [SMILES: CCC(O)(COC(C)C)c1c(-c2ccc(C(=O)O)cc2)c2c(F)c3[nH]ncc3cc2n1-c1ccc(F)c(OC)c1]
Compound 14 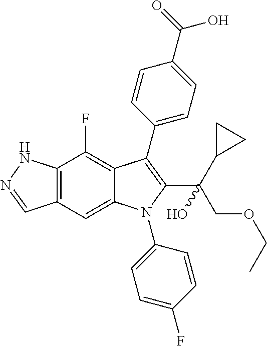 [SMILES: CCOCC(O)(c1c(-c2ccc(C(=O)O)cc2)c2c(F)c3[nH]ncc3cc2n1-c1ccc(F)cc1)C1CC1]
Compound 15 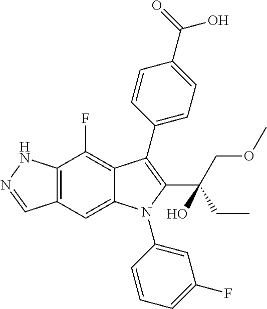 [SMILES: CC[C@@](O)(COC)c1c(-c2ccc(C(=O)O)cc2)c2c(F)c3[nH]ncc3cc2n1-c1cccc(F)c1]
Compound 16 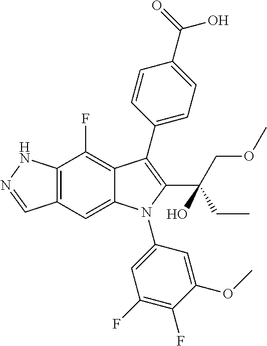 [SMILES: CC[C@@](O)(COC)c1c(-c2ccc(C(=O)O)cc2)c2c(F)c3[nH]ncc3cc2n1-c1cc(F)c(F)c(OC)c1]
Compound 17 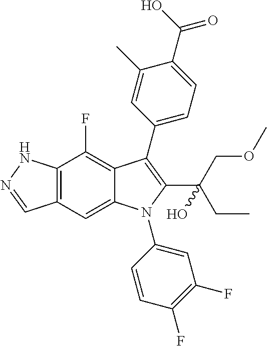 [SMILES: CCC(O)(COC)c1c(-c2ccc(C(=O)O)c(C)c2)c2c(F)c3[nH]ncc3cc2n1-c1ccc(F)c(F)c1]
Compound 18 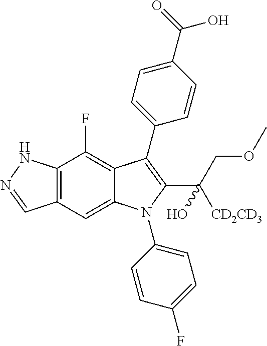
Compound 19 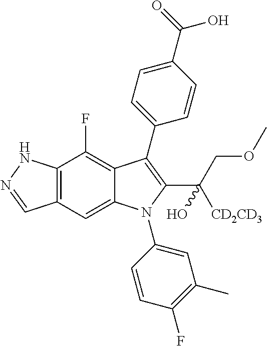
Compound 20 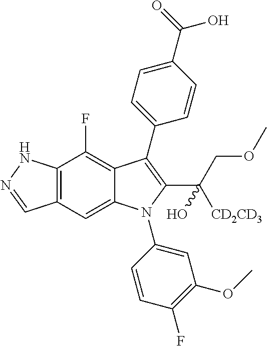
Compound 21 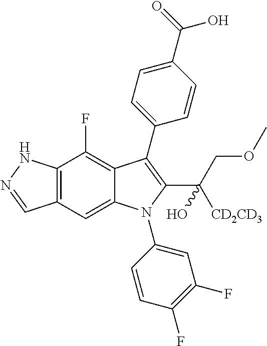
Compound 22 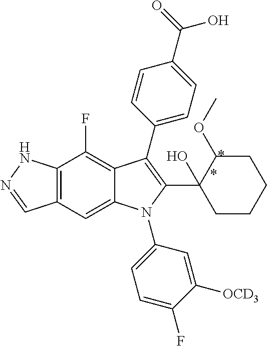
Compound 23 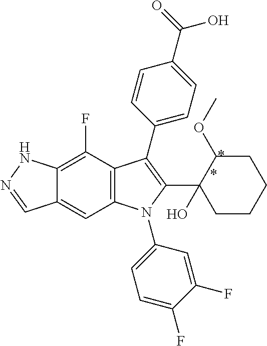 [SMILES: COC1CCCCC1(O)c1c(-c2ccc(C(=O)O)cc2)c2c(F)c3[nH]ncc3cc2n1-c1ccc(F)c(F)c1]
Compound 24 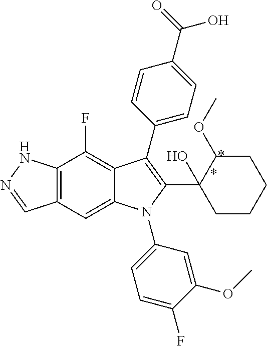 [SMILES: COc1cc(-n2c(C3(O)CCCCC3OC)c(-c3ccc(C(=O)O)cc3)c3c(F)c4[nH]ncc4cc32)ccc1F]
Compound 25 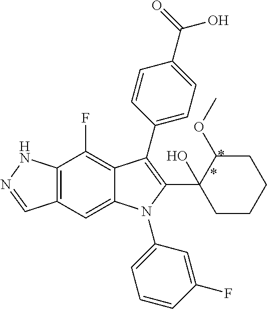 [SMILES: COC1CCCCC1(O)c1c(-c2ccc(C(=O)O)cc2)c2c(F)c3[nH]ncc3cc2n1-c1cccc(F)c1]
Compound 26 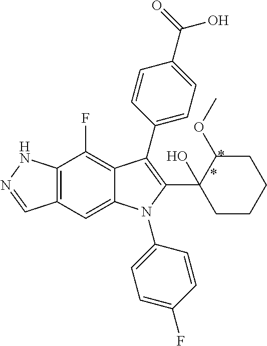 [SMILES: COC1CCCCC1(O)c1c(-c2ccc(C(=O)O)cc2)c2c(F)c3[nH]ncc3cc2n1-c1ccc(F)cc1]
Compound 27 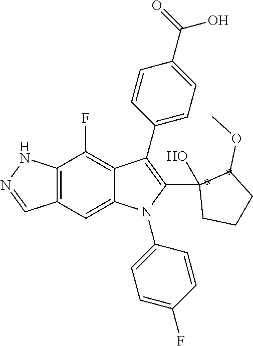 [SMILES: COC1CCCC1(O)c1c(-c2ccc(C(=O)O)cc2)c2c(F)c3[nH]ncc3cc2n1-c1ccc(F)cc1]
Compound 28 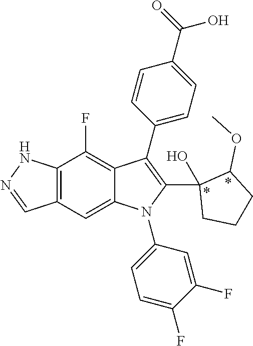 [SMILES: COC1CCCC1(O)c1c(-c2ccc(C(=O)O)cc2)c2c(F)c3[nH]ncc3cc2n1-c1ccc(F)c(F)c1]
Compound 29 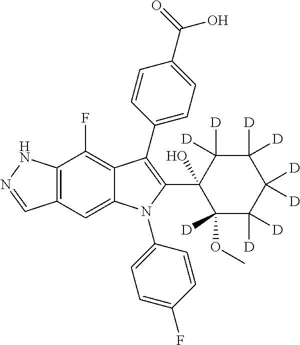 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)cc2)[C@@]([2H])(OC)C1([2H])[2H]]
Compound 30 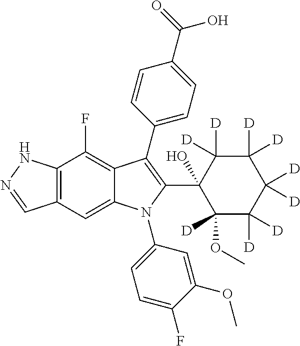 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)c(OC)c2)[C@@]([2H])(OC)C1([2H])[2H]]
Compound 31 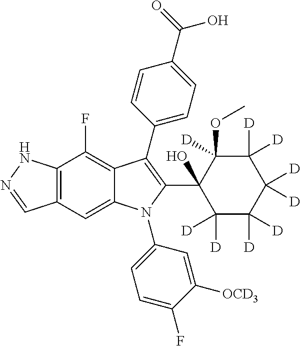
Compound 32 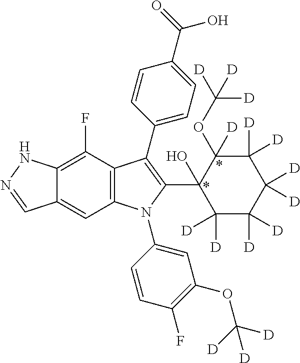 [SMILES: [2H]C([2H])([2H])Oc1cc(-n2c(C3(O)C([2H])([2H])C([2H])([2H])C([2H])([2H])C([2H])([2H])C3([2H])OC([2H])([2H])[2H])c(-c3ccc(C(=O)O)cc3)c3c(F)c4[nH]ncc4cc32)ccc1F]
Compound 33 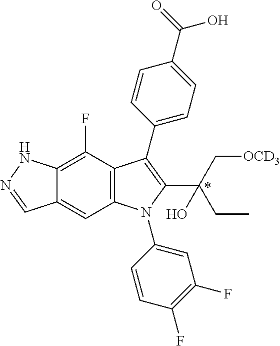
Compound 34 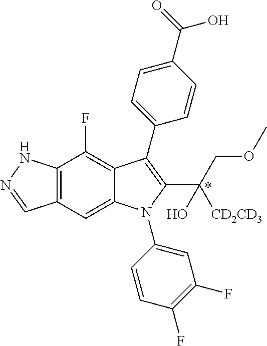
Compound 35 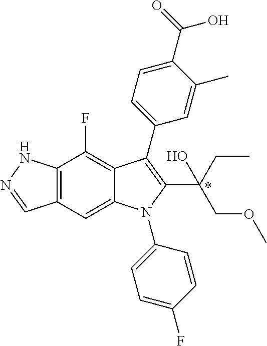 [SMILES: CCC(O)(COC)c1c(-c2ccc(C(=O)O)c(C)c2)c2c(F)c3[nH]ncc3cc2n1-c1ccc(F)cc1]
Compound 36 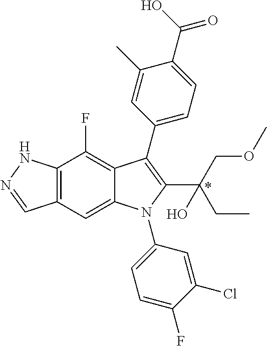 [SMILES: CCC(O)(COC)c1c(-c2ccc(C(=O)O)c(C)c2)c2c(F)c3[nH]ncc3cc2n1-c1ccc(F)c(Cl)c1]
Compound 37 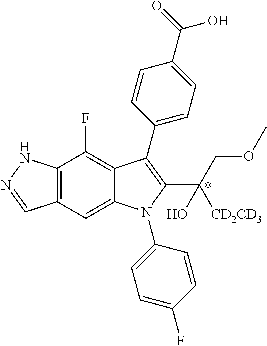
Compound 38 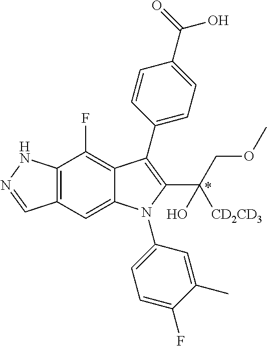
Compound 39 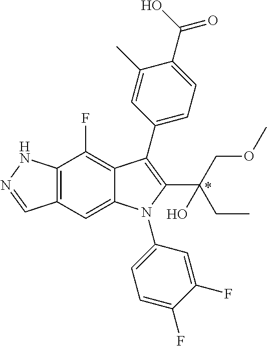 [SMILES: CCC(O)(COC)c1c(-c2ccc(C(=O)O)c(C)c2)c2c(F)c3[nH]ncc3cc2n1-c1ccc(F)c(F)c1]
Compound 40 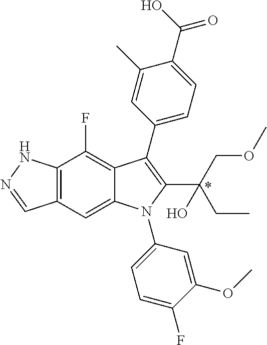 [SMILES: CCC(O)(COC)c1c(-c2ccc(C(=O)O)c(C)c2)c2c(F)c3[nH]ncc3cc2n1-c1ccc(F)c(OC)c1]
Compound 41 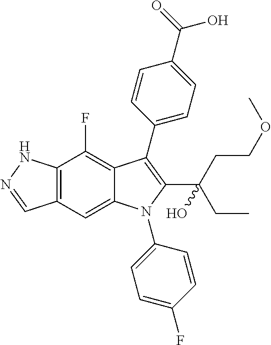 [SMILES: CCC(O)(CCOC)c1c(-c2ccc(C(=O)O)cc2)c2c(F)c3[nH]ncc3cc2n1-c1ccc(F)cc1]
Compound 42 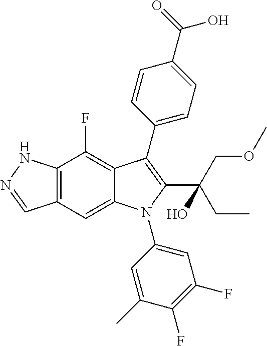 [SMILES: CC[C@@](O)(COC)c1c(-c2ccc(C(=O)O)cc2)c2c(F)c3[nH]ncc3cc2n1-c1cc(C)c(F)c(F)c1]
Compound 43 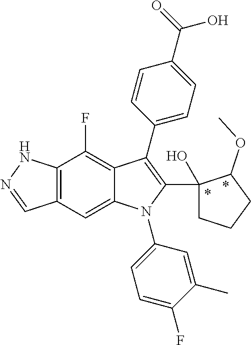 [SMILES: COC1CCCC1(O)c1c(-c2ccc(C(=O)O)cc2)c2c(F)c3[nH]ncc3cc2n1-c1ccc(F)c(C)c1]
Compound 44 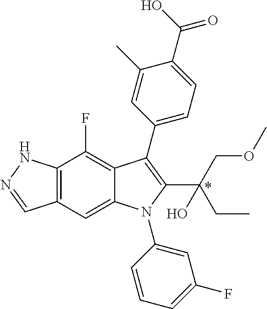 [SMILES: CCC(O)(COC)c1c(-c2ccc(C(=O)O)c(C)c2)c2c(F)c3[nH]ncc3cc2n1-c1cccc(F)c1]
Compound 45 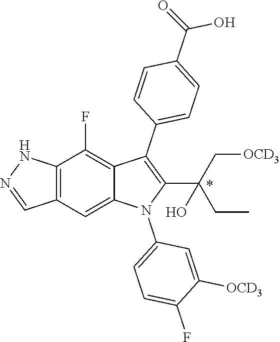
Compound 46 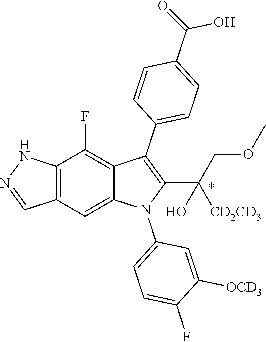
Compound 47 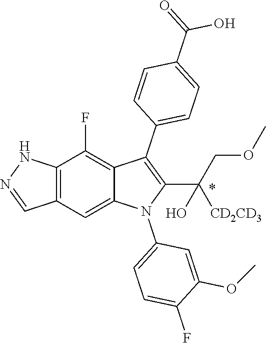
Compound 48 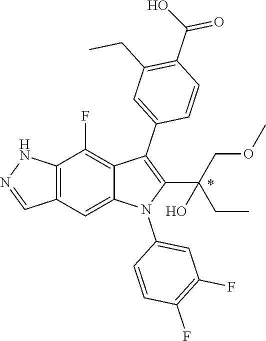 [SMILES: CCc1cc(-c2c(C(O)(CC)COC)n(-c3ccc(F)c(F)c3)c3cc4cn[nH]c4c(F)c23)ccc1C(=O)O]
Compound 49 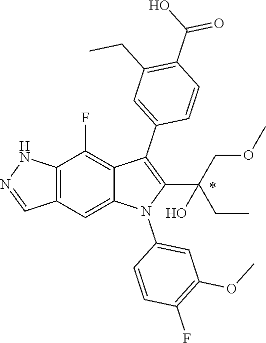 [SMILES: CCc1cc(-c2c(C(O)(CC)COC)n(-c3ccc(F)c(OC)c3)c3cc4cn[nH]c4c(F)c23)ccc1C(=O)O]
Compound 50 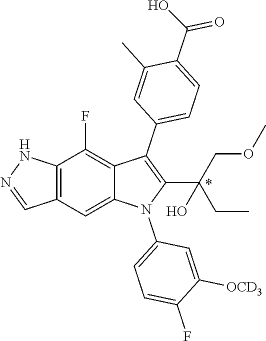
Compound 51 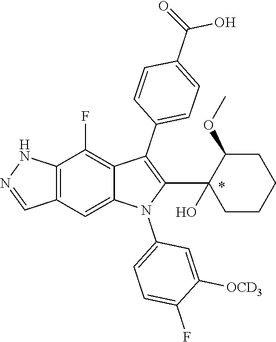
Compound 52 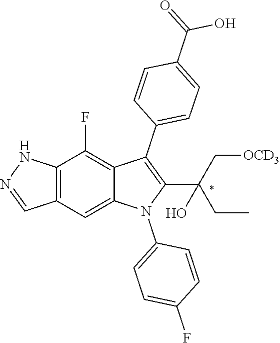
Compound 53 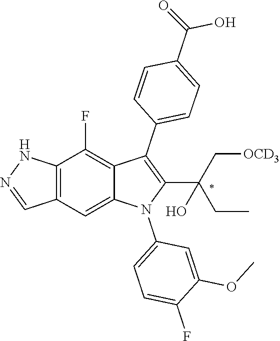
Compound 54 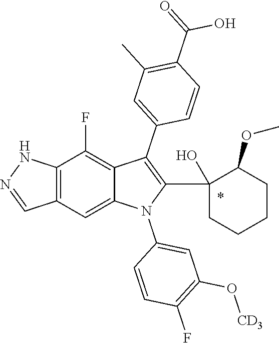 [SMILES: [2H]C([2H])([2H])Oc1cc(-n2c(C3(O)CCCC[C@@H]3OC)c(-c3ccc(C(=O)O)c(C)c3)c3c(F)c4[nH]ncc4cc32)ccc1F]
Compound 55 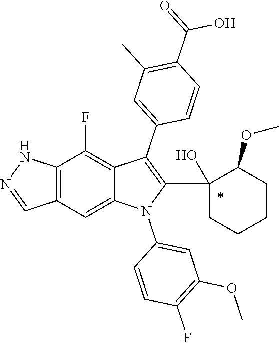 [SMILES: COc1cc(-n2c(C3(O)CCCC[C@@H]3OC)c(-c3ccc(C(=O)O)c(C)c3)c3c(F)c4[nH]ncc4cc32)ccc1F]
Compound 56 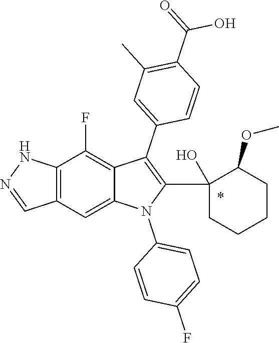 [SMILES: CO[C@H]1CCCCC1(O)c1c(-c2ccc(C(=O)O)c(C)c2)c2c(F)c3[nH]ncc3cc2n1-c1ccc(F)cc1]
Compound 57 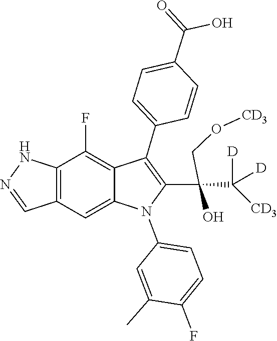 [SMILES: [2H]C([2H])([2H])OC[C@@](O)(c1c(-c2ccc(C(=O)O)cc2)c2c(F)c3[nH]ncc3cc2n1-c1ccc(F)c(C)c1)C([2H])([2H])C([2H])([2H])[2H]]
and tautomers of those compounds, deuterated derivatives of those compounds and tautomers, and pharmaceutically acceptable salts of those compounds, tautomers, and deuterated derivatives.

6. The compound, tautomer, deuterated derivative, or pharmaceutically acceptable salt according to claim 4, wherein the compound of Formula b is selected from compounds of Formula Ib-i: 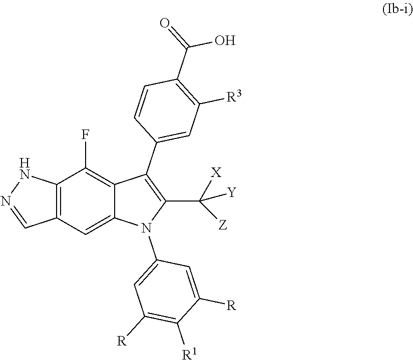
and tautomers thereof, deuterated derivatives of those compounds and tautomers, and pharmaceutically acceptable salts of those compounds, tautomers, and deuterated derivatives, wherein:
each R is independently selected from F, H, Cl, CH₃, OCH₃, and OCD₃;
R¹ is H or F;
X, Y, and Z are defined as follows:
one of X, Y, and Z is OH;
one of X, Y, and Z is chosen from CH₂CH₃ and cyclopropyl;
and one of X, Y, and Z is chosen from CH₂OCH₃, CH₂CH₂OCH₃, CH₂OCH₂CH₃, CH₂O-cyclopropyl, and CH₂O-isopropyl; and
R³ is chosen from H and CH₃.

7. The compound, tautomer, deuterated derivative, or pharmaceutically acceptable salt according to claim 6, wherein the compound of Formula Ib-i is selected from:
Compound 6 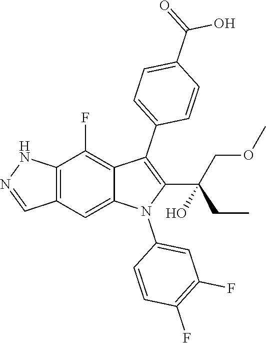 [SMILES: CC[C@](O)(COC)c1c(-c2ccc(C(=O)O)cc2)c2c(F)c3[nH]ncc3cc2n1-c1ccc(F)c(F)c1]
Compound 7 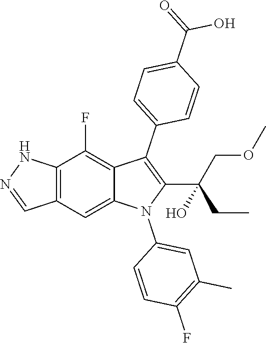 [SMILES: CC[C@](O)(COC)c1c(-c2ccc(C(=O)O)cc2)c2c(F)c3[nH]ncc3cc2n1-c1ccc(F)c(C)c1]
Compound 8 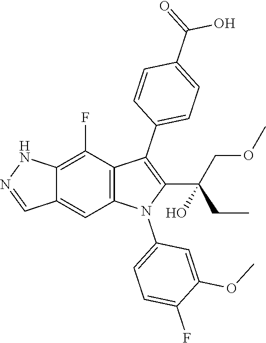 [SMILES: CC[C@](O)(COC)c1c(-c2ccc(C(=O)O)cc2)c2c(F)c3[nH]ncc3cc2n1-c1ccc(F)c(OC)c1]
Compound 9 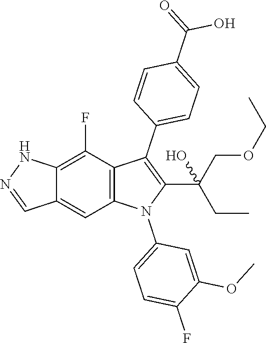 [SMILES: CCOCC(O)(CC)c1c(-c2ccc(C(=O)O)cc2)c2c(F)c3[nH]ncc3cc2n1-c1ccc(F)c(OC)c1]
Compound 10 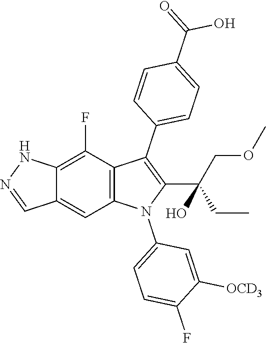
Compound 11 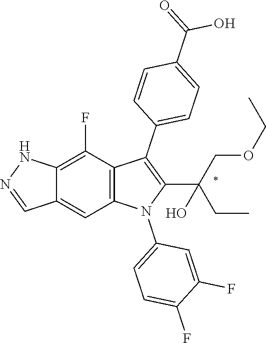 [SMILES: CCOCC(O)(CC)c1c(-c2ccc(C(=O)O)cc2)c2c(F)c3[nH]ncc3cc2n1-c1ccc(F)c(F)c1]
Compound 12 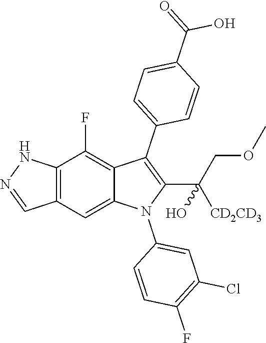
Compound 13 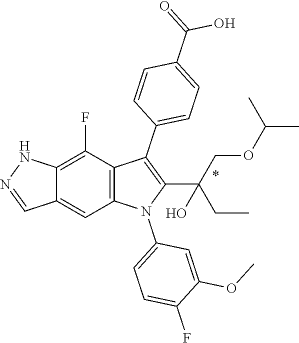 [SMILES: CCC(O)(COC(C)C)c1c(-c2ccc(C(=O)O)cc2)c2c(F)c3[nH]ncc3cc2n1-c1ccc(F)c(OC)c1]
Compound 14 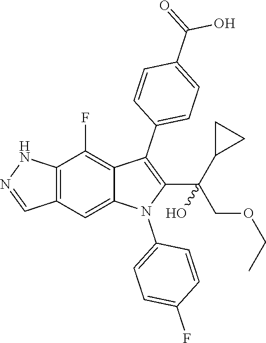 [SMILES: CCOCC(O)(c1c(-c2ccc(C(=O)O)cc2)c2c(F)c3[nH]ncc3cc2n1-c1ccc(F)cc1)C1CC1]
Compound 15 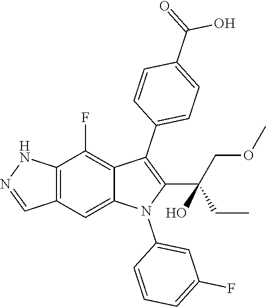 [SMILES: CC[C@@](O)(COC)c1c(-c2ccc(C(=O)O)cc2)c2c(F)c3[nH]ncc3cc2n1-c1cccc(F)c1]
Compound 16 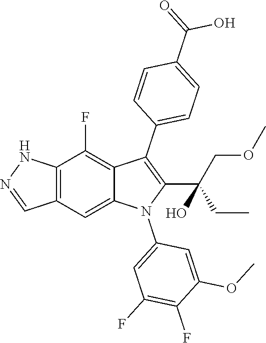 [SMILES: CC[C@@](O)(COC)c1c(-c2ccc(C(=O)O)cc2)c2c(F)c3[nH]ncc3cc2n1-c1cc(F)c(F)c(OC)c1]
Compound 17 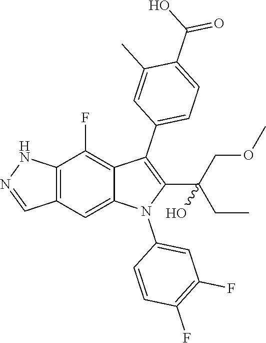 [SMILES: CCC(O)(COC)c1c(-c2ccc(C(=O)O)c(C)c2)c2c(F)c3[nH]ncc3cc2n1-c1ccc(F)c(F)c1]
Compound 18 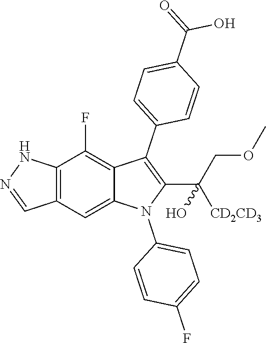
Compound 19 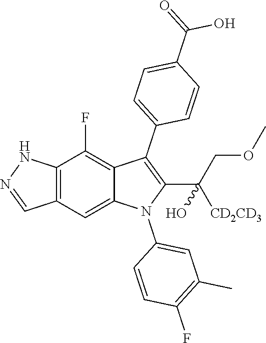
Compound 20 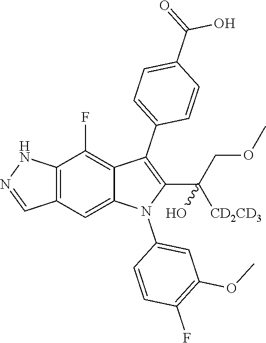
Compound 21 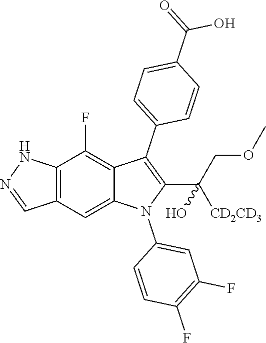
Compound 33 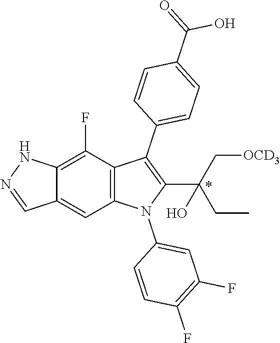
Compound 34 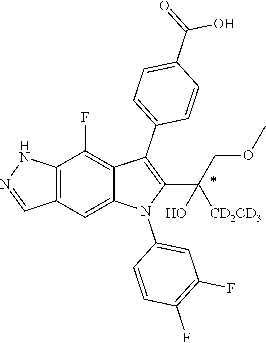
Compound 35 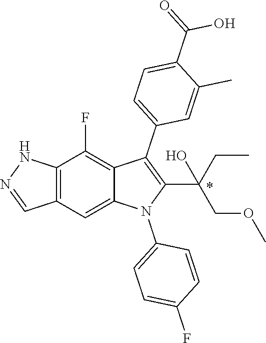 [SMILES: CCC(O)(COC)c1c(-c2ccc(C(=O)O)c(C)c2)c2c(F)c3[nH]ncc3cc2n1-c1ccc(F)cc1]
Compound 36 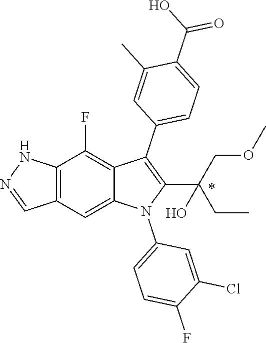 [SMILES: CCC(O)(COC)c1c(-c2ccc(C(=O)O)c(C)c2)c2c(F)c3[nH]ncc3cc2n1-c1ccc(F)c(Cl)c1]
Compound 37 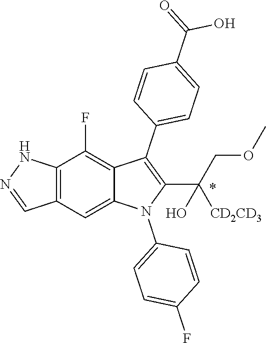
Compound 38 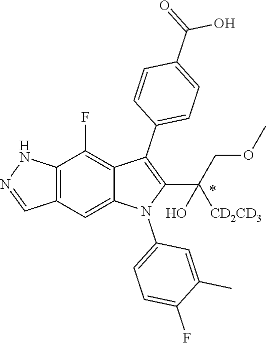
Compound 39 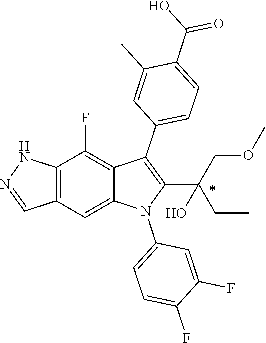 [SMILES: CCC(O)(COC)c1c(-c2ccc(C(=O)O)c(C)c2)c2c(F)c3[nH]ncc3cc2n1-c1ccc(F)c(F)c1]
Compound 40 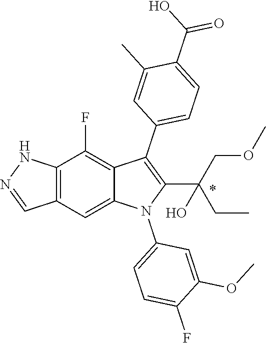 [SMILES: CCC(O)(COC)c1c(-c2ccc(C(=O)O)c(C)c2)c2c(F)c3[nH]ncc3cc2n1-c1ccc(F)c(OC)c1]
Compound 41 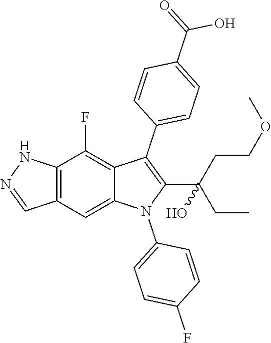 [SMILES: CCC(O)(CCOC)c1c(-c2ccc(C(=O)O)cc2)c2c(F)c3[nH]ncc3cc2n1-c1ccc(F)cc1]
Compound 42 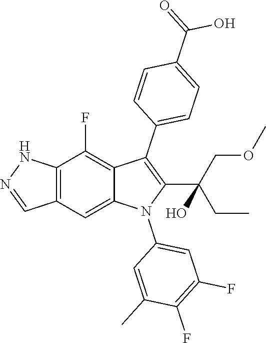 [SMILES: CC[C@@](O)(COC)c1c(-c2ccc(C(=O)O)cc2)c2c(F)c3[nH]ncc3cc2n1-c1cc(C)c(F)c(F)c1]
Compound 44 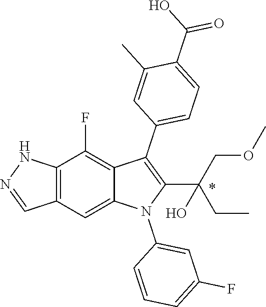 [SMILES: CCC(O)(COC)c1c(-c2ccc(C(=O)O)c(C)c2)c2c(F)c3[nH]ncc3cc2n1-c1cccc(F)c1]
Compound 45 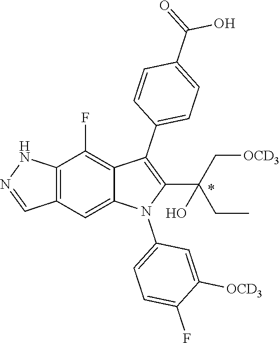
Compound 46 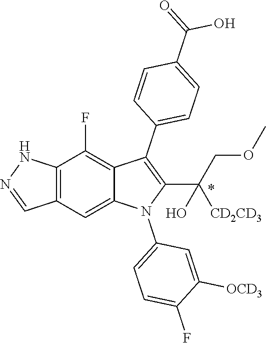
Compound 47 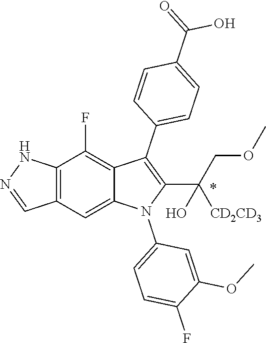
Compound 48 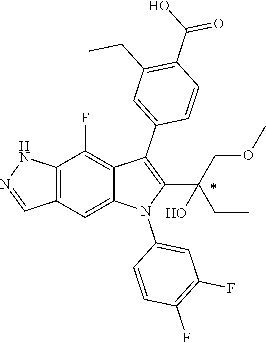 [SMILES: CCc1cc(-c2c(C(O)(CC)COC)n(-c3ccc(F)c(F)c3)c3cc4cn[nH]c4c(F)c23)ccc1C(=O)O]
Compound 49 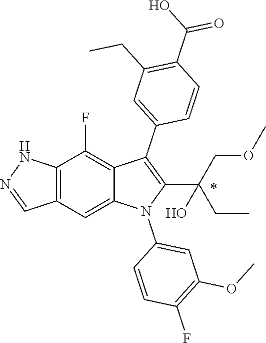 [SMILES: CCc1cc(-c2c(C(O)(CC)COC)n(-c3ccc(F)c(OC)c3)c3cc4cn[nH]c4c(F)c23)ccc1C(=O)O]
Compound 50 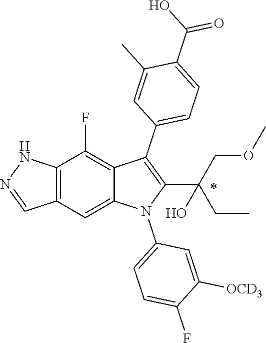
Compound 52 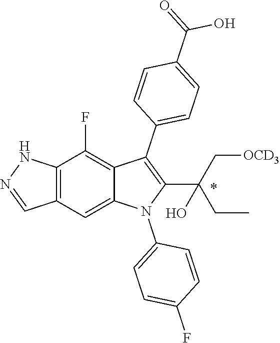
Compound 53 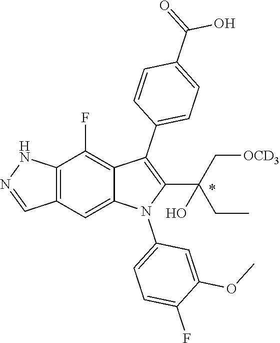
Compound 57 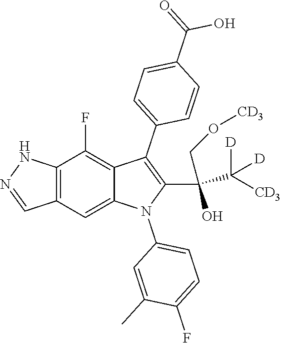 [SMILES: [2H]C([2H])([2H])OC[C@@](O)(c1c(-c2ccc(C(=O)O)cc2)c2c(F)c3[nH]ncc3cc2n1-c1ccc(F)c(C)c1)C([2H])([2H])C([2H])([2H])[2H]]
and tautomers thereof, deuterated derivatives of those compounds and tautomers, and pharmaceutically acceptable salts of those compounds, tautomers, and deuterated derivatives.

8. The compound, tautomer, deuterated derivative, or pharmaceutically acceptable salt according to claim 4, wherein the compound of Formula Ib is selected from compounds of Formula Ib-ii: 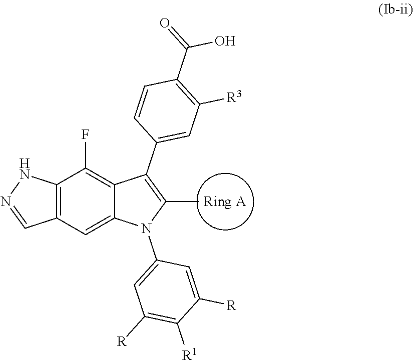
and tautomers thereof, deuterated derivatives of those compounds and tautomers, and pharmaceutically acceptable salts of those compounds, tautomers, and deuterated derivatives, wherein:
each R is independently selected from F, H, Cl, CH₃, OCH₃, and OCD₃;
R¹ is H or F;
R³ is chosen from H and CH₃; and
Ring A is selected from

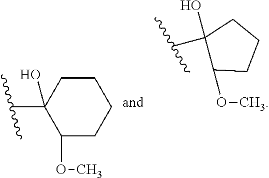

9. The compound, tautomer, deuterated derivative, or pharmaceutically acceptable salt according to claim 8, wherein the compound of Formula Ib-ii:
Compound 22 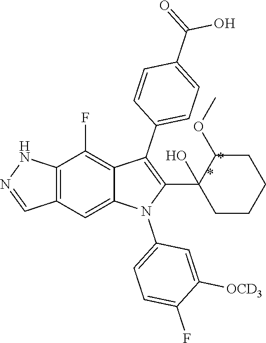
Compound 23 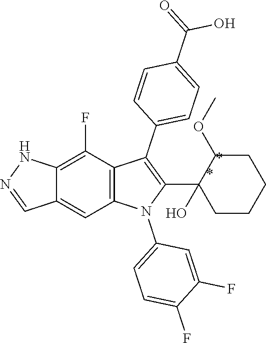 [SMILES: COC1CCCCC1(O)c1c(-c2ccc(C(=O)O)cc2)c2c(F)c3[nH]ncc3cc2n1-c1ccc(F)c(F)c1]
Compound 24 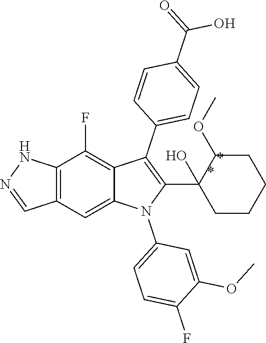 [SMILES: COc1cc(-n2c(C3(O)CCCCC3OC)c(-c3ccc(C(=O)O)cc3)c3c(F)c4[nH]ncc4cc32)ccc1F]
Compound 25 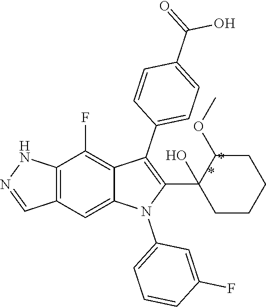 [SMILES: COC1CCCCC1(O)c1c(-c2ccc(C(=O)O)cc2)c2c(F)c3[nH]ncc3cc2n1-c1cccc(F)c1]
Compound 26 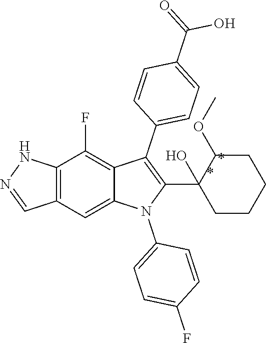 [SMILES: COC1CCCCC1(O)c1c(-c2ccc(C(=O)O)cc2)c2c(F)c3[nH]ncc3cc2n1-c1ccc(F)cc1]
Compound 27 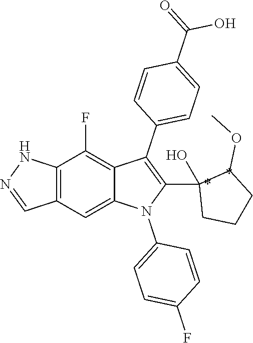 [SMILES: COC1CCCC1(O)c1c(-c2ccc(C(=O)O)cc2)c2c(F)c3[nH]ncc3cc2n1-c1ccc(F)cc1]
Compound 28 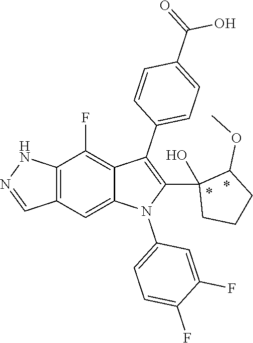 [SMILES: COC1CCCC1(O)c1c(-c2ccc(C(=O)O)cc2)c2c(F)c3[nH]ncc3cc2n1-c1ccc(F)c(F)c1]
Compound 29 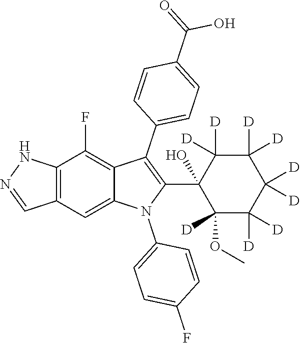 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)cc2)[C@@]([2H])(OC)C1([2H])[2H]]
Compound 30 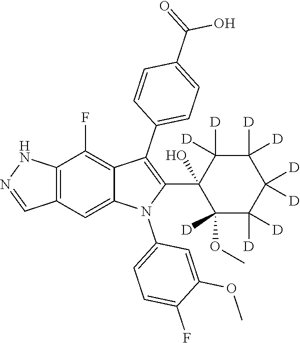 [SMILES: [2H]C1([2H])C([2H])([2H])C([2H])([2H])[C@](O)(c2c(-c3ccc(C(=O)O)cc3)c3c(F)c4[nH]ncc4cc3n2-c2ccc(F)c(OC)c2)[C@@]([2H])(OC)C1([2H])[2H]]
Compound 31 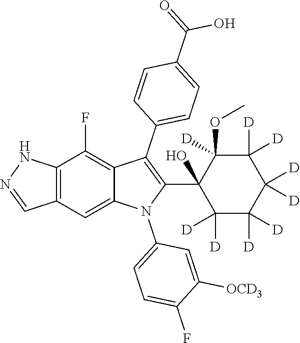
Compound 32 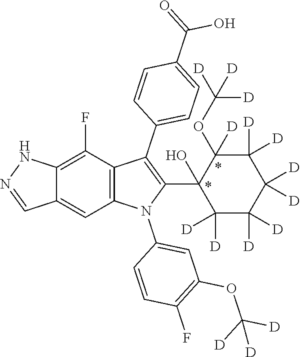 [SMILES: [2H]C([2H])([2H])Oc1cc(-n2c(C3(O)C([2H])([2H])C([2H])([2H])C([2H])([2H])C([2H])([2H])C3([2H])OC([2H])([2H])[2H])c(-c3ccc(C(=O)O)cc3)c3c(F)c4[nH]ncc4cc32)ccc1F]
Compound 43 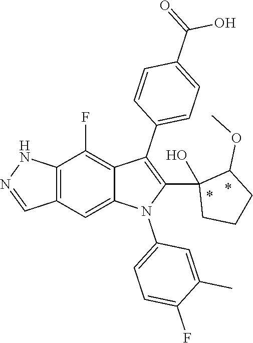 [SMILES: COC1CCCC1(O)c1c(-c2ccc(C(=O)O)cc2)c2c(F)c3[nH]ncc3cc2n1-c1ccc(F)c(C)c1]
Compound 51 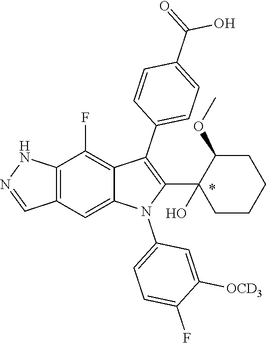
Compound 54 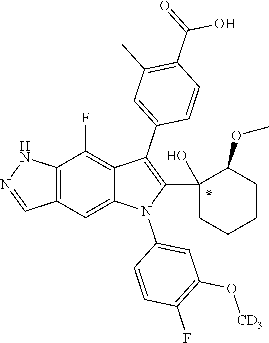 [SMILES: [2H]C([2H])([2H])Oc1cc(-n2c(C3(O)CCCC[C@@H]3OC)c(-c3ccc(C(=O)O)c(C)c3)c3c(F)c4[nH]ncc4cc32)ccc1F]
Compound 55 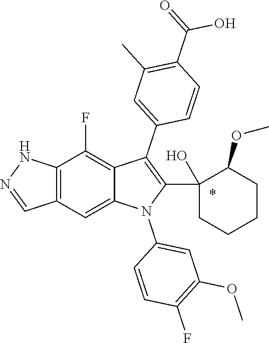 [SMILES: COc1cc(-n2c(C3(O)CCCC[C@@H]3OC)c(-c3ccc(C(=O)O)c(C)c3)c3c(F)c4[nH]ncc4cc32)ccc1F]
Compound 56 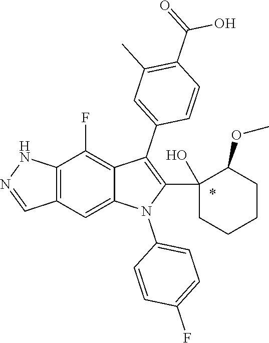 [SMILES: CO[C@H]1CCCCC1(O)c1c(-c2ccc(C(=O)O)c(C)c2)c2c(F)c3[nH]ncc3cc2n1-c1ccc(F)cc1]
and tautomers thereof, deuterated derivatives of those compounds and tautomers, and pharmaceutically acceptable salts of those compounds, tautomers, and deuterated derivatives.

10. The compound, tautomer, deuterated derivative, or pharmaceutically acceptable salt according to claim 1 wherein the compound of Formula I is selected from compounds of Formula Ic: 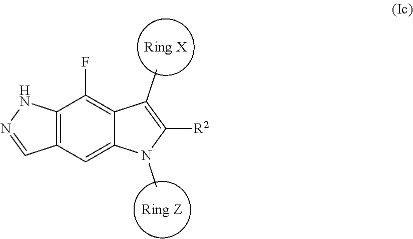
and tautomers thereof, deuterated derivatives of those compounds or tautomers, and pharmaceutically acceptable salts of the foregoing compounds, tautomers, and deuterated derivatives, wherein:
Ring X is selected from: 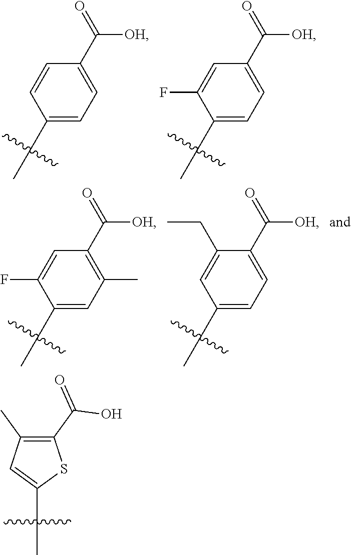
R² is selected from; 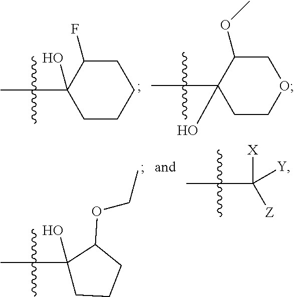
wherein
one of X, Y, and Z is —OH;
one of X, Y, and Z is —CH₂CH₃;
and one of X, Y, and Z is chosen from —CH₂OCH₃, and —CH₂OCH₂CH₃; and
Ring Z is selected from:

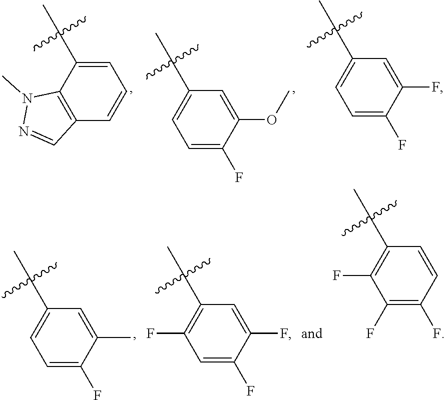

11. The compound, tautomer, deuterated derivative, or pharmaceutically acceptable salt according to claim 10, wherein the compounds of Formula Ic are selected from:
Compound 58 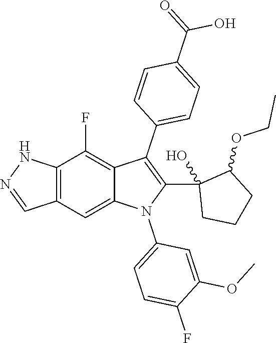 [SMILES: CCOC1CCCC1(O)c1c(-c2ccc(C(=O)O)cc2)c2c(F)c3[nH]ncc3cc2n1-c1ccc(F)c(OC)c1]
Compound 59 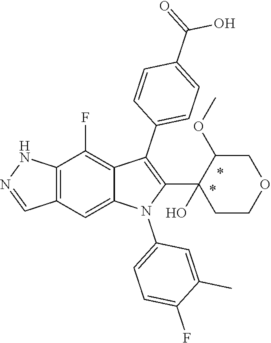 [SMILES: COC1COCCC1(O)c1c(-c2ccc(C(=O)O)cc2)c2c(F)c3[nH]ncc3cc2n1-c1ccc(F)c(C)c1]
Compound 60 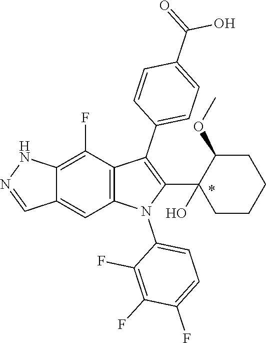 [SMILES: CO[C@H]1CCCCC1(O)c1c(-c2ccc(C(=O)O)cc2)c2c(F)c3[nH]ncc3cc2n1-c1ccc(F)c(F)c1F]
Compound 61 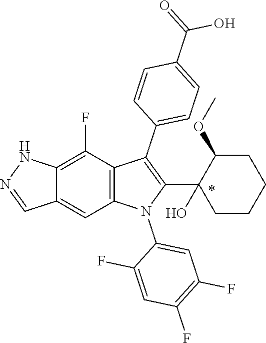 [SMILES: CO[C@H]1CCCCC1(O)c1c(-c2ccc(C(=O)O)cc2)c2c(F)c3[nH]ncc3cc2n1-c1cc(F)c(F)cc1F]
Compound 62 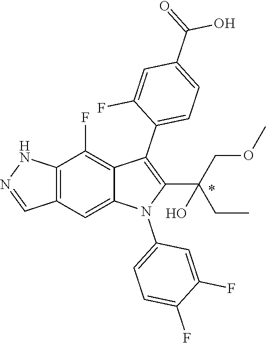 [SMILES: CCC(O)(COC)c1c(-c2ccc(C(=O)O)cc2F)c2c(F)c3[nH]ncc3cc2n1-c1ccc(F)c(F)c1]
Compound 63 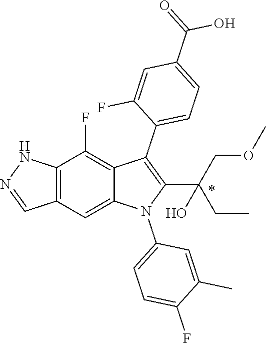 [SMILES: CCC(O)(COC)c1c(-c2ccc(C(=O)O)cc2F)c2c(F)c3[nH]ncc3cc2n1-c1ccc(F)c(C)c1]
Compound 64 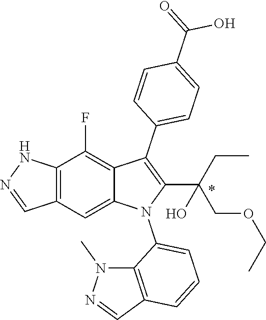 [SMILES: CCOCC(O)(CC)c1c(-c2ccc(C(=O)O)cc2)c2c(F)c3[nH]ncc3cc2n1-c1cccc2cnn(C)c12]
Compound 65 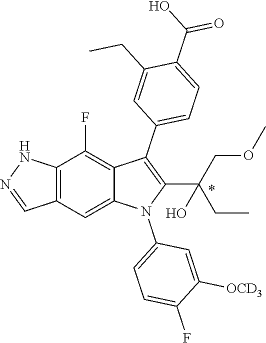
Compound 66 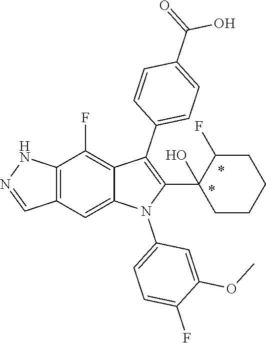 [SMILES: COc1cc(-n2c(C3(O)CCCCC3F)c(-c3ccc(C(=O)O)cc3)c3c(F)c4[nH]ncc4cc32)ccc1F]
Compound 67 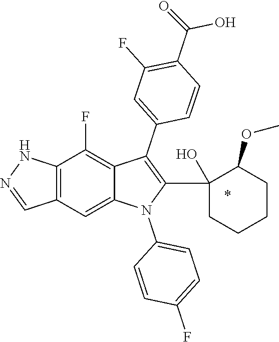 [SMILES: CO[C@H]1CCCCC1(O)c1c(-c2ccc(C(=O)O)c(F)c2)c2c(F)c3[nH]ncc3cc2n1-c1ccc(F)cc1]
and tautomers of those compounds, deuterated derivatives of those compounds and tautomers, and pharmaceutically acceptable salts of those compounds, tautomers, and deuterated derivatives.

12. A pharmaceutical composition comprising the compound, tautomer, deuterated derivative or pharmaceutically acceptable salt according to any one of claims 1 to 11 and a pharmaceutically acceptable carrier.

13. A compound, tautomer, deuterated derivative or pharmaceutically acceptable salt according to any one of claims 1 to 11, or a pharmaceutical composition according to claim 12, for use in the treatment of alpha anti-trypsin deficiency (AATD).

14. A compound tautomer, deuterated derivative or pharmaceutically acceptable salt according to any one of claims 1 to 11, or a pharmaceutical composition according to claim 12, for use in the manufacture of a medicament for the treatment of AATD.

15. A method of treating a patient suffering from alpha anti-trypsin deficiency (AATD), comprising administering a compound, tautomer, deuterated derivative or pharmaceutically acceptable salt according to any one of claims 1 to 4, or a pharmaceutical composition according to claim 5.

16. A substantially crystalline form of Compound 5 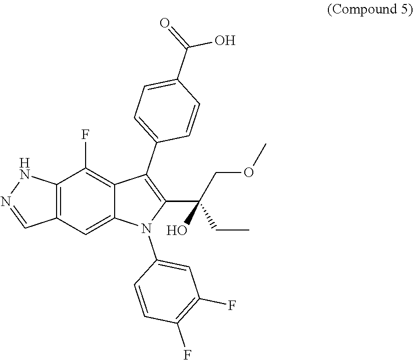 [SMILES: CC[C@@](O)(COC)c1c(-c2ccc(C(=O)O)cc2)c2c(F)c3[nH]ncc3cc2n1-c1ccc(F)c(F)c1]
selected from Compound 5 free form Monohydrate Form A, Compound 5 free form Form A, Compound 5 free form Form B, Compound 5 free form NPA Solvate Form A, Compound 5 free form EtOH Solvate Form A, Compound 5 free form MeOH Solvate Hydrate Form A, Compound 5 free form DCM Solvate Form A, and Compound 5 free form EtOAc Heptane Solvate Form A.

17. A substantially crystalline form of Compound 3 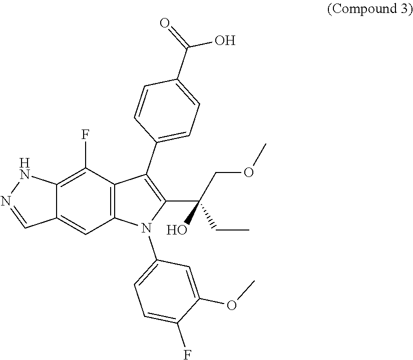 [SMILES: CC[C@@](O)(COC)c1c(-c2ccc(C(=O)O)cc2)c2c(F)c3[nH]ncc3cc2n1-c1ccc(F)c(OC)c1]
selected from Compound 3 free form Form A, Compound 3 free form Form B, Compound 3 free form Hydrate Form A, Compound 3 free form Hydrate Form B, Compound 3 free form Hydrate Form C, and Compound 3 free form MTBE Solvate Form A.

18. A substantially crystalline form of Compound 4 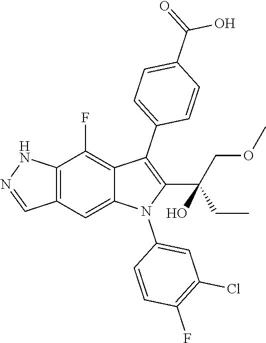 [SMILES: CC[C@@](O)(COC)c1c(-c2ccc(C(=O)O)cc2)c2c(F)c3[nH]ncc3cc2n1-c1ccc(F)c(Cl)c1]
Selected from Compound 4 free form Form A, Compound 4 free form Form B, Compound 4 free form Form C, Compound 4 free form Form D, Compound 4 free form Hydrate Form A, Compound 4 free form Hydrate Form B, and Compound 4 free form Hydrate Form C.

19. A pharmaceutical composition comprising the substantially crystalline form according to any one of claims 16-18, and a pharmaceutically acceptable carrier.

20. The substantially crystalline form of Compound 5 according to claim 16, the substantially crystalline form of Compound 3 according to claim 17, the substantially crystalline form of Compound 4 according to claim 18, or the pharmaceutical composition according to claim 19, for use in the treatment of alpha anti-trypsin deficiency (AATD).

21. Use of the substantially crystalline form of Compound 5 according to claim 16, the substantially crystalline form of Compound 3 according to claim 17, the substantially crystalline form of Compound 4 according to claim 18, or the pharmaceutical composition according to claim 19, in the manufacture of a medicament for the treatment of AATD.

22. A method of treating a patient suffering from alpha anti-trypsin deficiency (AATD), comprising administering the substantially crystalline form of Compound 5 according to claim 16, the substantially crystalline form of Compound 3 according to claim 17, the substantially crystalline form of Compound 4 according to claim 18, or the pharmaceutical composition according to claim 11.

23. A process for preparing (S)-4-(5-(3,4-Difluorophenyl)-8-fluoro-6-(2-hydroxy-1-meth-oxybutan-2-yl)-1,5-dihydropyrrolo[2,3-f]indazol-7-yl)benzoic acid (Compound 5) 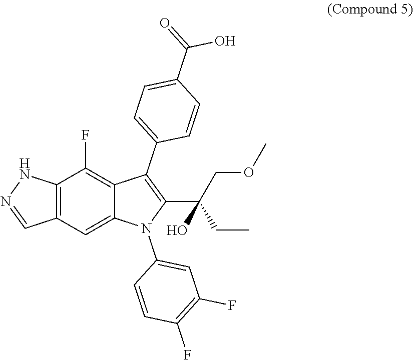 [SMILES: CC[C@@](O)(COC)c1c(-c2ccc(C(=O)O)cc2)c2c(F)c3[nH]ncc3cc2n1-c1ccc(F)c(F)c1]
wherein the process comprises the steps of
(a) preparing intermediate S14 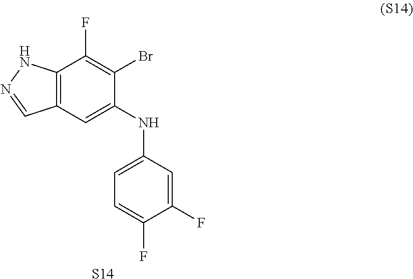 [SMILES: Fc1ccc(Nc2cc3cn[nH]c3c(F)c2Br)cc1F ; "S14"]
by reductive coupling of 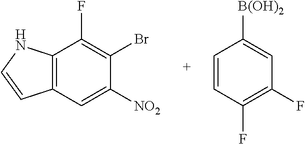 [SMILES: O=[N+]([O-])c1cc2cc[nH]c2c(F)c1Br ; OB(O)c1ccc(F)c(F)c1]
(b) reacting intermediate S14 with reagent N6 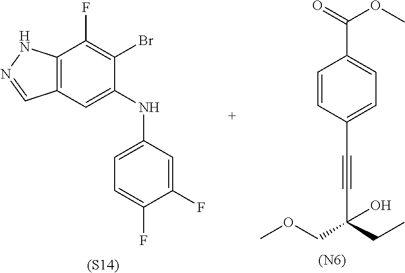
via Larock annulation to provide Compound 5.

24. A process for preparing Compound 3 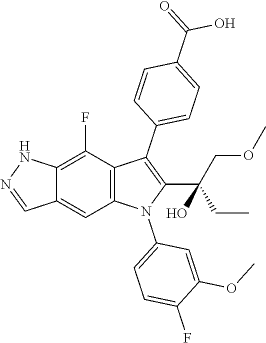 [SMILES: CC[C@@](O)(COC)c1c(-c2ccc(C(=O)O)cc2)c2c(F)c3[nH]ncc3cc2n1-c1ccc(F)c(OC)c1]
comprising the steps of
(a) reacting 6-bromo-7-fluoro-5-iodo-1H-indazole and 4-fluoro-3-methoxyaniline via Buchwald coupling to produce intermediate S1 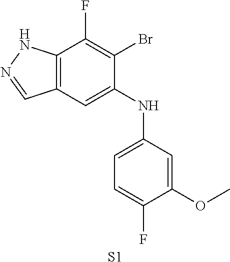
(b) reacting intermediate S1 with reagent via Larock annulation produce (methyl (S)-4-(8-fluoro-5-(4-fluoro-3-methoxyphenyl)-6-(2-hydroxy-1-methoxybutan-2-yl)-1,5-dihydropyrrolo[2,3-f]indazol-7-yl)benzoate: 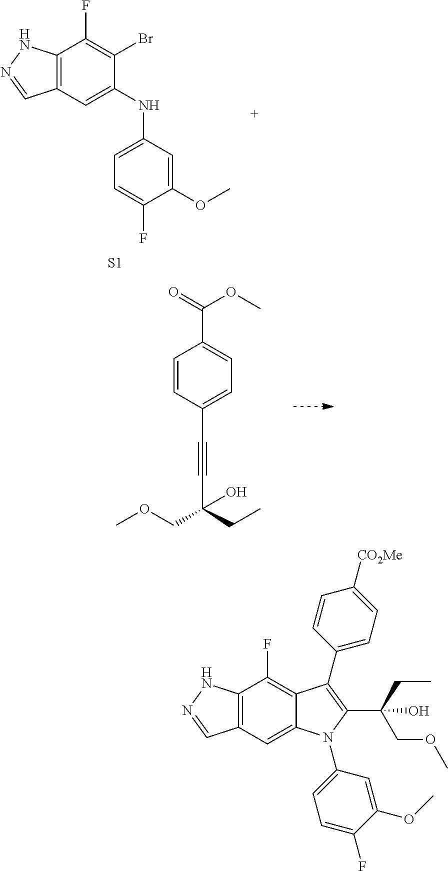
(c) subjecting (methyl (S)-4-(8-fluoro-5-(4-fluoro-3-methoxyphenyl)-6-(2-hydroxy-1-methoxybutan-2-yl)-1,5-dihydropyrrolo[2,3-f]indazol-7-yl)benzoate to ester hydrolysis to produce Compound 3.
